(19) United States
(12) Patent Application Publication
(10) Pub. No.: US 2024/0238304 A1
(43) Pub. Date: Jul. 18, 2024

(54) Small Molecule Degraders of DOT1L and Uses Thereof

(71) Applicant: Northwestern University, Evanston, IL (US)

(72) Inventors: Gary E. Schiltz, Evanston, IL (US); Sarki Abba Abdulkadir, Evanston, IL (US)

(21) Appl. No.: 18/512,950

(22) Filed: Nov. 17, 2023

Related U.S. Application Data
(60) Provisional application No. 63/384,204, filed on Nov. 17, 2022.

(51) Int. Cl. A61K 31/53

(52) U.S. Cl. CPC A61K 31/53

(57) ABSTRACT

Disclosed herein is a compound, or a pharmaceutically acceptable salt thereof, that is a proteolysis-targeting chimeric molecule (PROTAC) that induces degradation of DOT1L protein. Disclosed herein are also the uses of the compound, or a pharmaceutically acceptable salt thereof, and a pharmaceutical composition comprising the same, in a method of treating a disease or disorder associated with DOT1L activity or in a method of inhibiting cell proliferation.

### CROSS-REFERENCE TO RELATED APPLICATIONS

This application claims benefit of priority to U.S. Application Ser. No. 63/384,204, filed Nov. 17, 2022, the contents of which are incorporated by reference in its entirety.

### BACKGROUND

Disruptor of telomeric silencing I-like (DOT1L) is a versatile histone H3K79 methyltransferase which plays a prominent role in MLL-fusion leukemia. Genetic inactivation of DOT1L led to downregulation of target genes and loss of the leukemic phenotype with minimal effects on global gene expression. Accordingly, DOT1L inhibition could profoundly affect cancer growth or survival in MLL-rearranged leukemia models.

Proteolysis-targeting chimeric molecules (PROTACs) are an emerging technology that may be utilized to target previously “undruggable” targets, transcription factors and non-enzymatic proteins. (See, e.g., An et al., “Small-molecule PROTACs: An emerging and promising approach for the development of targeted therapy drugs,” EBioMedicine. 2018 October; 36: 553-562; and Gu et al., “PROTACs: An Emerging Targeting Technique for Protein Degradation in Drug Discovery,” Bioessays. 2018 April; 40(4): el 700247, the contents of which are incorporated herein by reference in their entireties). PROTACs are chimeric molecules that may be characterized as “hetero-bifunctional” in that PROTACs include a ligand for recruiting an E3 ubiquitin ligase, a linker, and another ligand to bind with the protein targeted for degradation. Designed as such, PROTACs “hijack” the E3 ubiquitin ligase to the protein which is targeted for protein degradation via ubiquitination, even if the targeted protein is not a physiological substrate for degradation via the ubiquitin-proteasome system.

As such, novel PROTACs that induce degradation of DOT1L protein, as well as their use for treating leukemia by selectively targeting DOT1L-driven cell proliferation, need to be developed.

### SUMMARY OF THE INVENTION

Disclosed herein is a compound having a formula M_{DOT1L}-L-M_{E3} and methods of using the same.

One aspect of the invention provides for a compound that has a formula I(a) or I(b), or a pharmaceutically acceptable salt thereof:

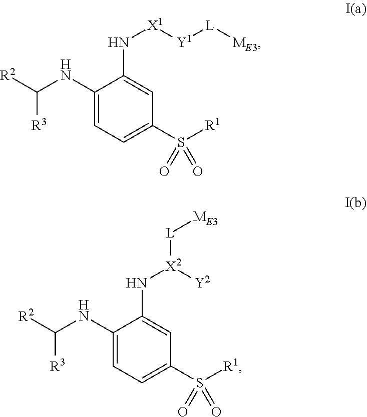

wherein
   R¹ is selected from the group consisting of hydrogen, alkyl, cycloalkyl, heterocyclyl, aryl, and heteroaryl;
   R² and R³ are each independently an optionally substituted aryl or an optionally substituted heteroaryl;
   X¹ is an optionally substituted heteroarylene;
   Y¹ is an optionally substituted heterocyclylene;
   X¹ is an optionally substituted trivalent heteroaryloxyl;
   Y² is an optionally substituted heterocyclyl;
   L is a bond or a linker; and
   M_{E3} is a moiety that binds to an E3 ubiquitin ligase.

Another aspect of the invention provides for a pharmaceutical composition comprising a therapeutically effective amount of the compound as disclosed herein and a pharmaceutically acceptable carrier, excipient, or diluent.

Another aspect of the invention provides for a method of treating a disease or disorder associated with DOT1L activity in a subject in need thereof. The method comprises administering to the subject the compound as disclosed herein or the pharmaceutical composition as disclosed herein.

Another aspect of the invention provides for a method of inhibiting cell proliferation. The method comprises contacting cells with the compound as disclosed herein, or a pharmaceutically acceptable salt thereof.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows that exemplary PROTAC degraders of DOT1L are classified into four groups. Group A (15 compounds), cereblon ligand is bound to parent compound 553 through the linker at the site indicated by the bigger arrow. Group B (3 compounds), VHL ligand 1 is bound to parent compound 553 through the linker at the site indicated by the bigger arrow. Group C (3 compounds), VHL ligand 2 is bound to parent compound 553 through the linker at the site indicated by the bigger arrow. Group D (4 compounds), VHL ligand 2 is bound to parent compound 553 through the linker at the site indicated by the bigger arrow. The difference within each group is the length/structure of the linker.

FIG. 2 illustrates that knockdown of DOT1L by RNAi identifies DOT1L protein band on western blot. After knockdown of DOT1L, protein amount of 185 kDa size, which is expected as DOT1L protein, significantly decreased. Lamin B was used as a loading control.

FIG. 3 shows western blotting using nuclear extraction after 1 μM DOT1L inhibitor treatment for 2 days in LNCaP cell line. Group D of PROTAC degraders of DOT1L accelerates DOT1L degradation than other groups. While group B of PROTAC degraders of DOT1L, parent compound 534, EPZ5676, and DMSO didn't degrade DOT1L protein by 48 h treatment, groups A, C, and D accelerated DOT1L degradation. Among groups A, C, and D, 588, 593, and 705, which accelerated the most DOT1L degradation, respectively, were selected for the following experiments. Lamin B was used as a loading control. A stands for Group A; B stands for Group B; C stands for Group C; D stands for Group D; V stands for vehicle; E stands for EPZ5676; and P stands for Parent.

FIG. 4 shows western blotting using nuclear extraction after 1 μM DOT1L inhibitor treatment in LNCaP cell line at multiple time points. Compound 705 causes 70% reduction of DOT1L protein at 1h treatment. Compounds 705 in group D, 593 in group C, and 588 in group A degraded DOT1L protein in this order while parent compound 534, EPZ5676, and DMSO didn't degrade DOT1L until 72 h. Lamin B was used as a loading control.

FIG. 5 shows dose-response of DOT1L degradation of nuclear fraction after 705 treatment with or without 1 μM MG132. Compound 705 degrades DOT1L in a dose-dependent manner at 1 h. Compound 705 degraded DOT1L in a dose-dependent manner starting around 500 nM and most degraded at 10 μM while effect of proteasome inhibitor MG132 was not observed in nuclear fraction. Lamin B was used as a loading control.

FIG. 6 shows Viability assay by MTS. Compounds belonging to Group D show higher selectivity for MV4-11 cells than the other groups. Compounds belonging to all groups showed no significant difference in selectivity against MV4-11 cells at 1 μM, but at 5 μM, only compounds belonging to group D showed high selectivity. Jurkat cells were used as a negative control. A stands for Group A; B stands for Group B; C stands for Group C; D stands for Group D; V stands for vehicle: E stands for EPZ5676; and P stands for Parent.

FIG. 7 shows dose-response by CellTiter-Glo luminescent cell viability assay (4 days treatment).

FIG. 8 illustrates dose-response by CellTiter-Glo luminescent cell viability assay (7 days treatment).

FIG. 9 shows western blotting of 22Rv1 cells after 2 hours of treatment with the indicated compounds and concentrations.

FIG. 10 shows dose-response cell viability of MV4-11 cells using CellTiter-Glo luminescent protocol. 1×10⁴ cell in round bottom 96 well plate. Add 100 ul/well media with indicated concentration for 4 days. Add 50 uL of mixed media (25 uL IMIDM+25 uL CellTiter-Glo), transfer into white plate. Cells were treated with the indicated compounds and concentrations for 4 days. Results show the degraders produce significant reductions in cell viability of MV4-11 cells.

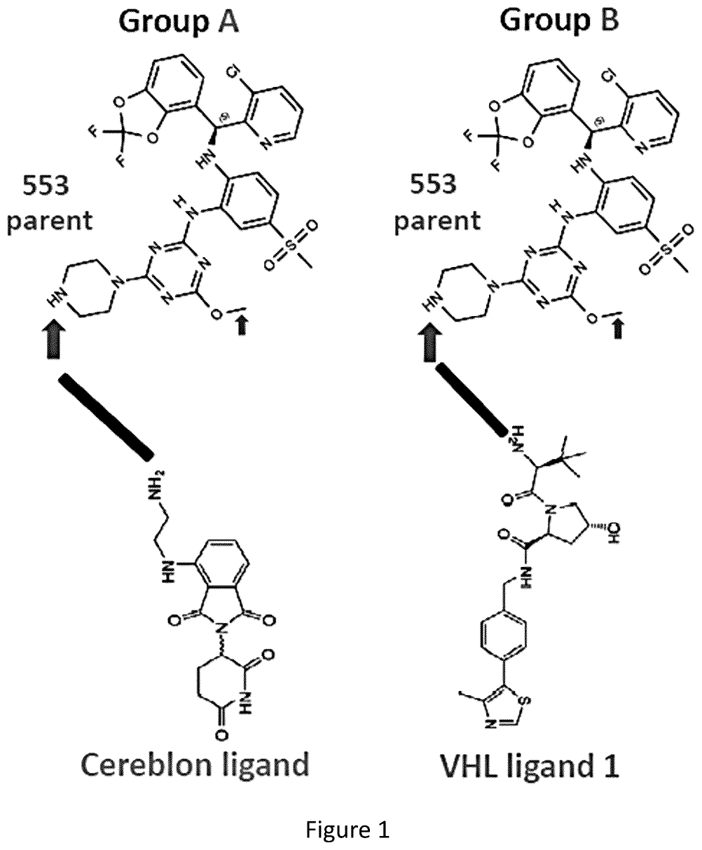

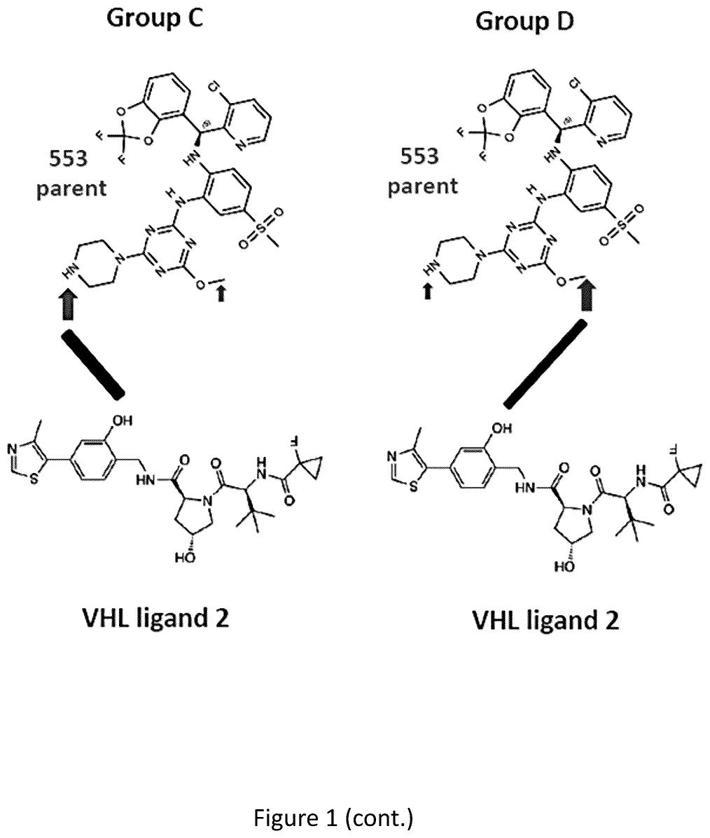

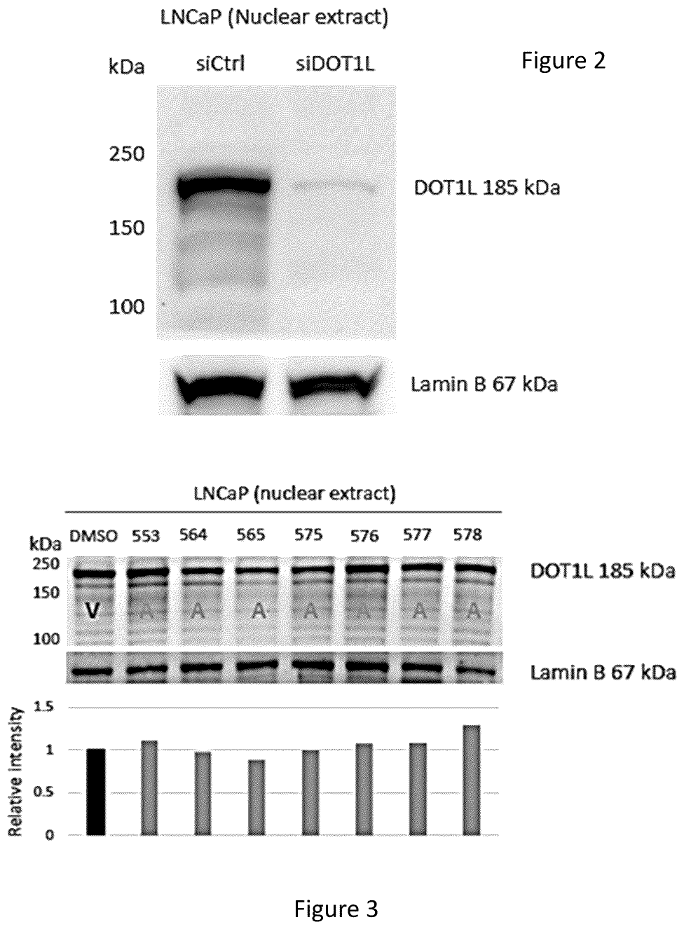

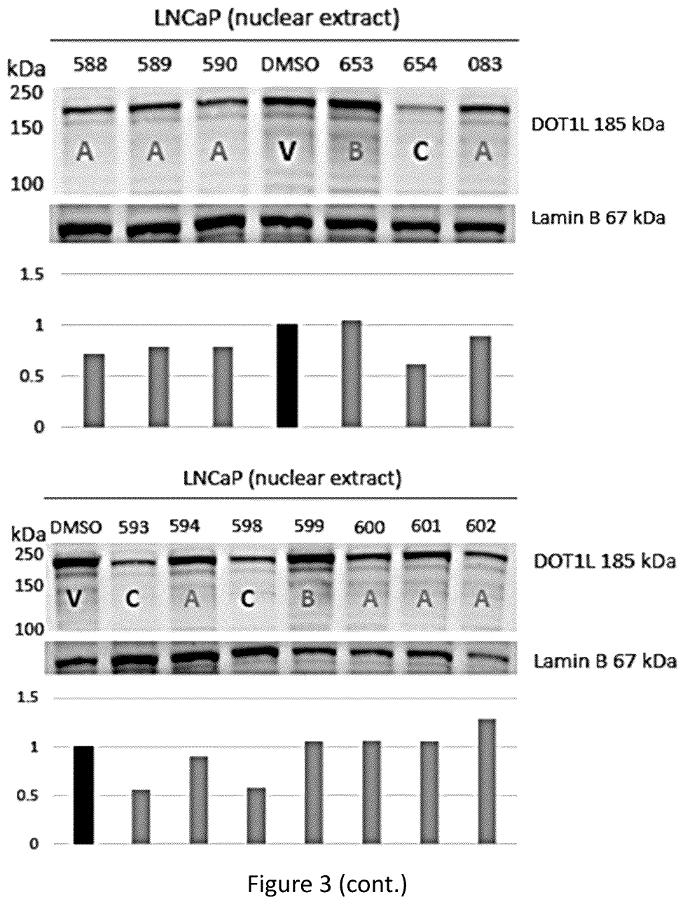

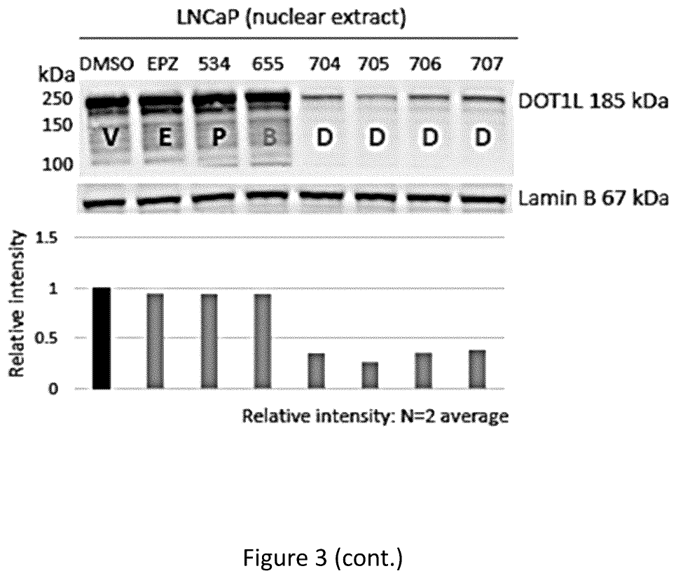

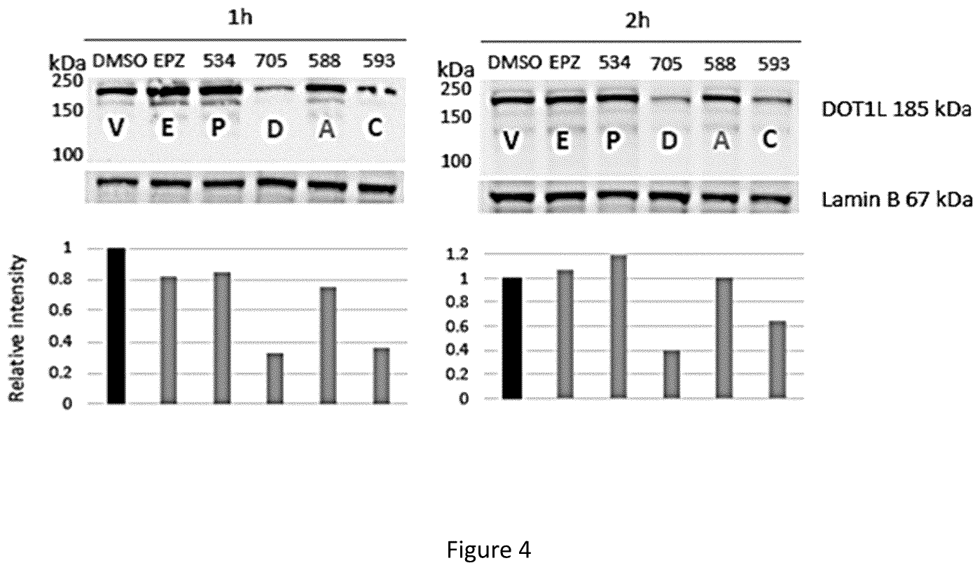

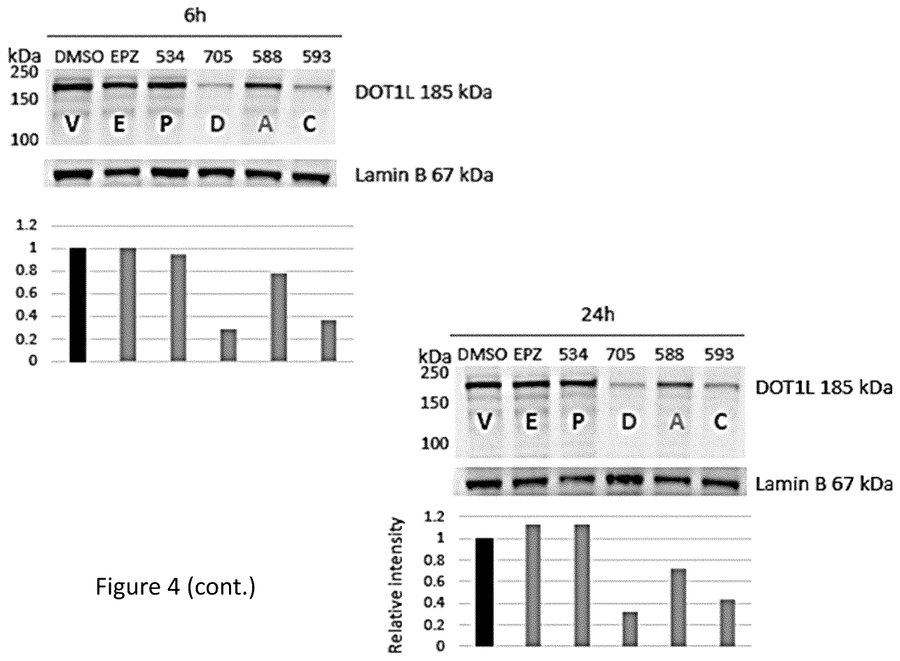

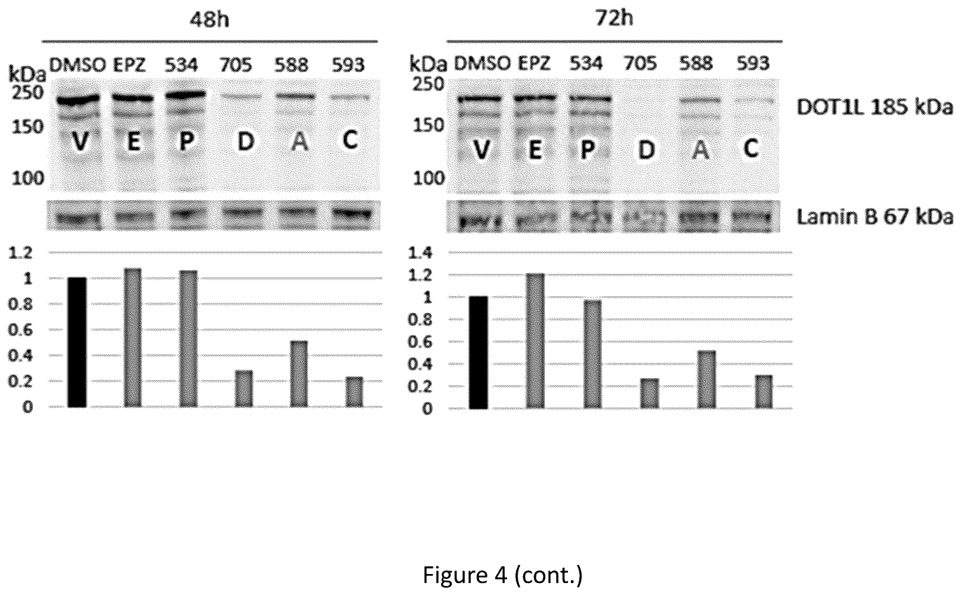

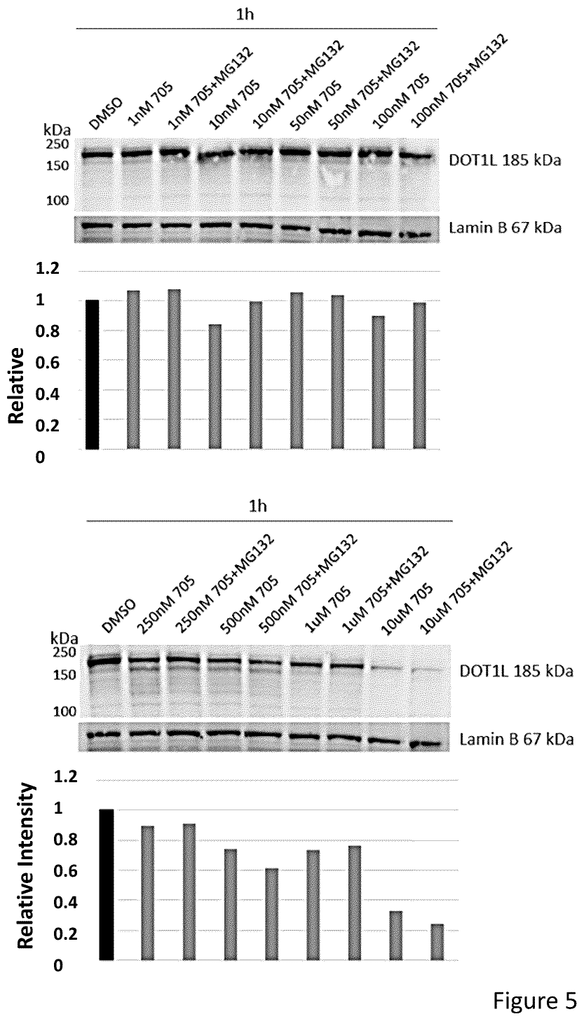

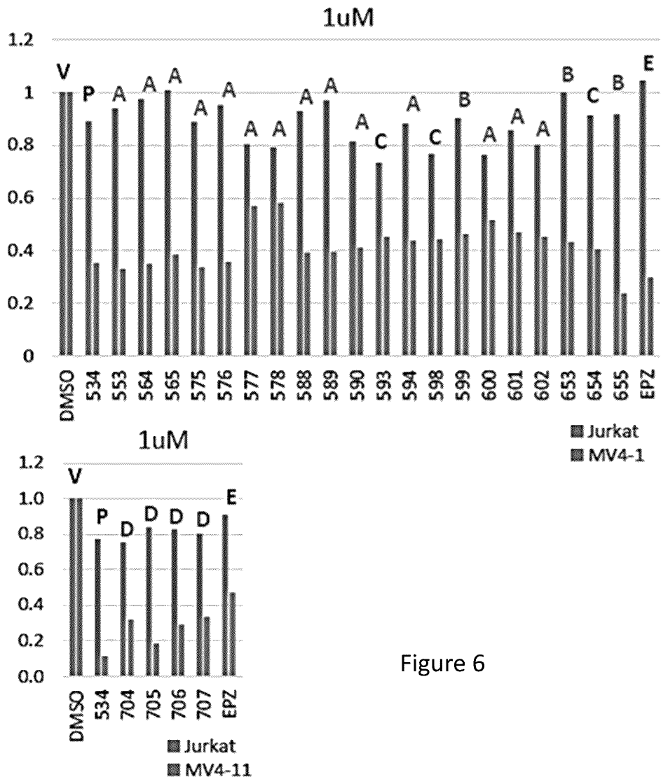

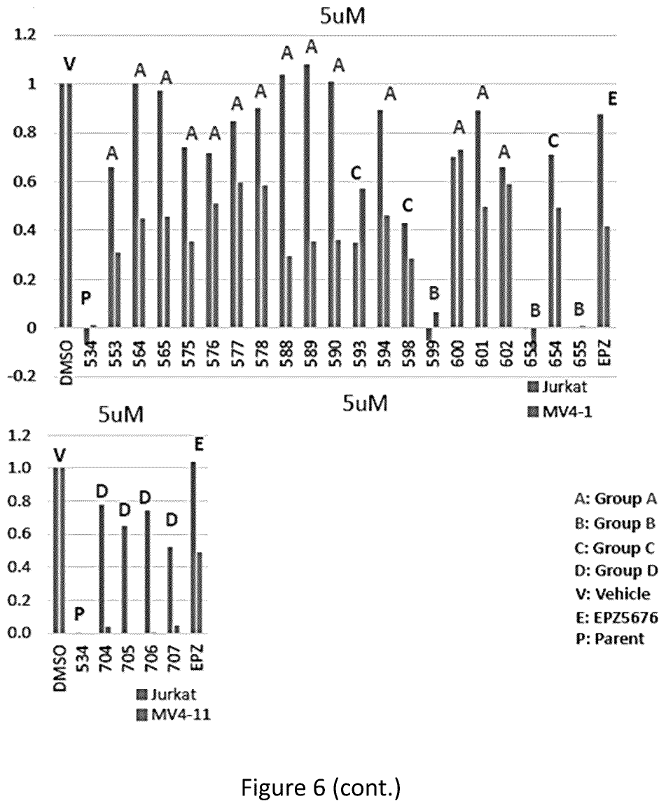

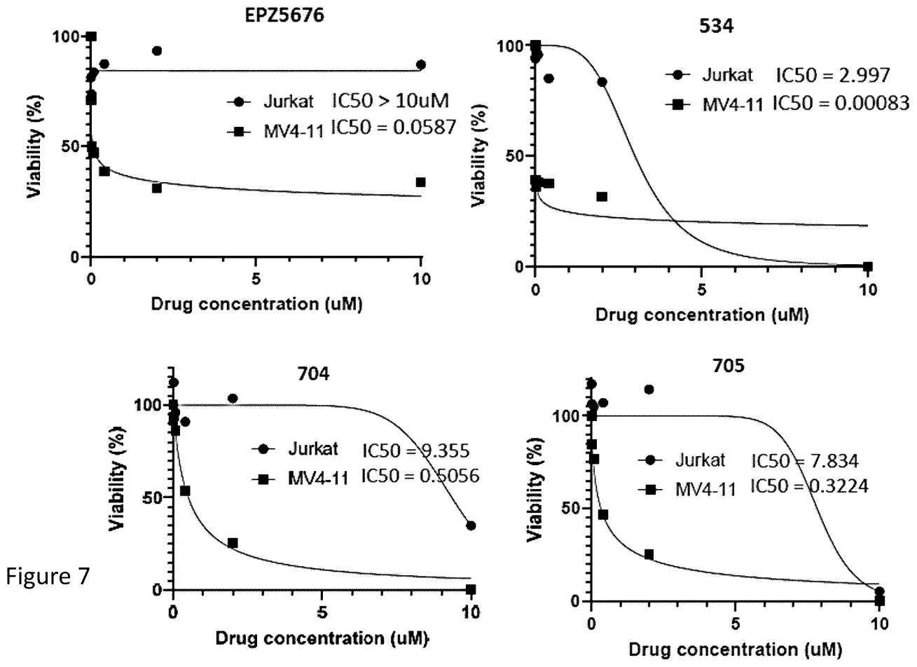

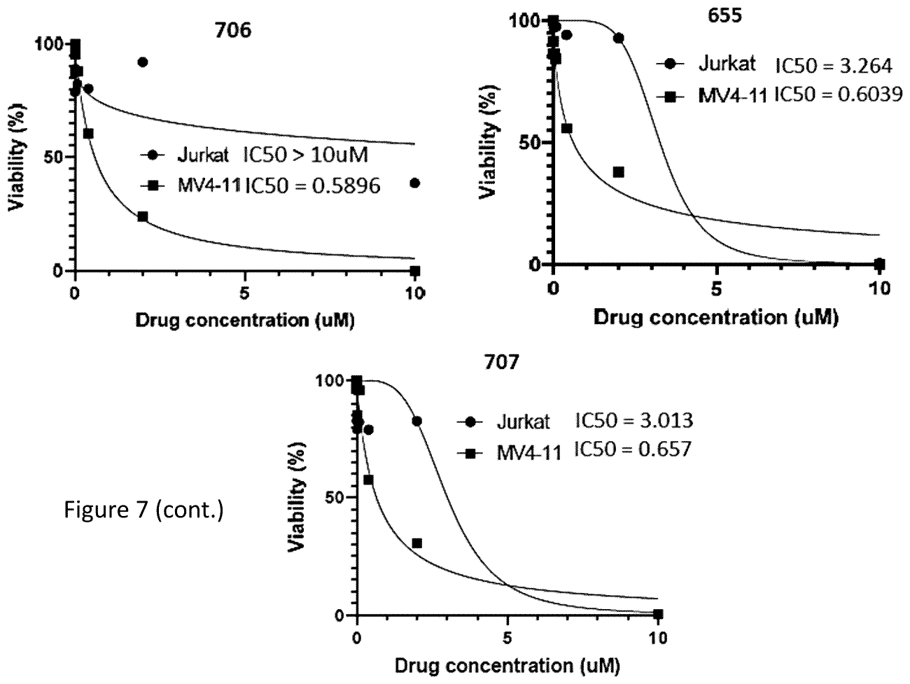

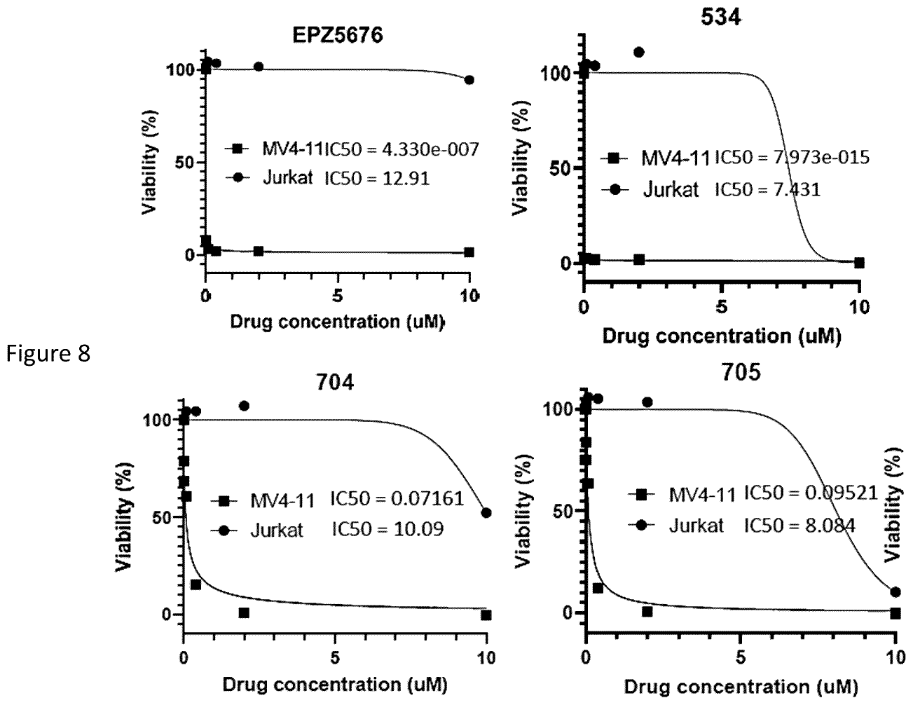

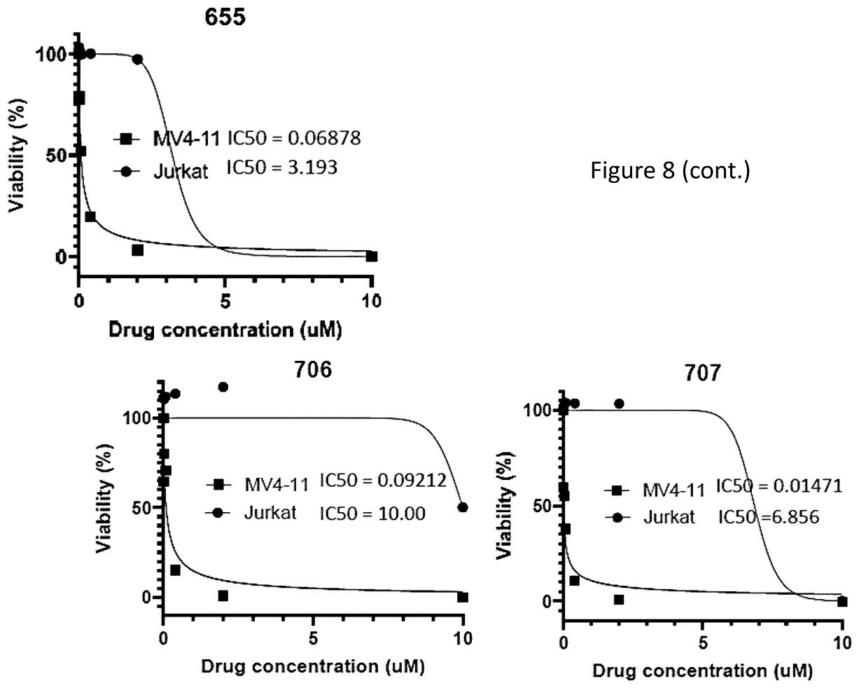

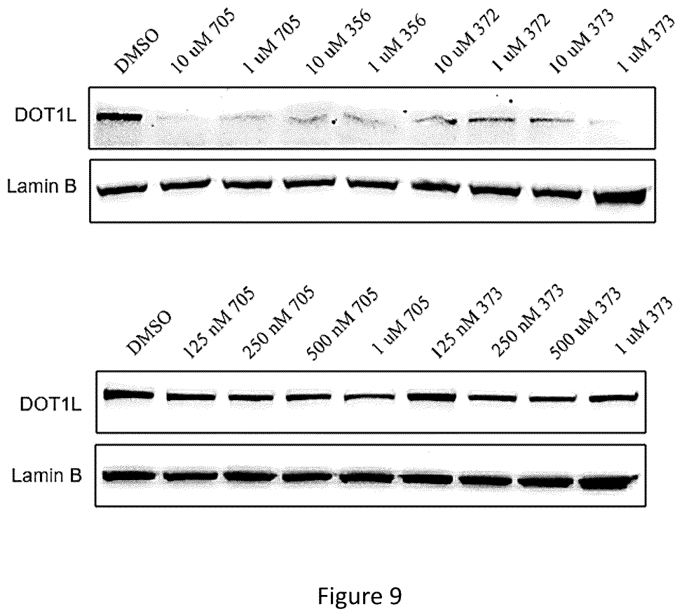

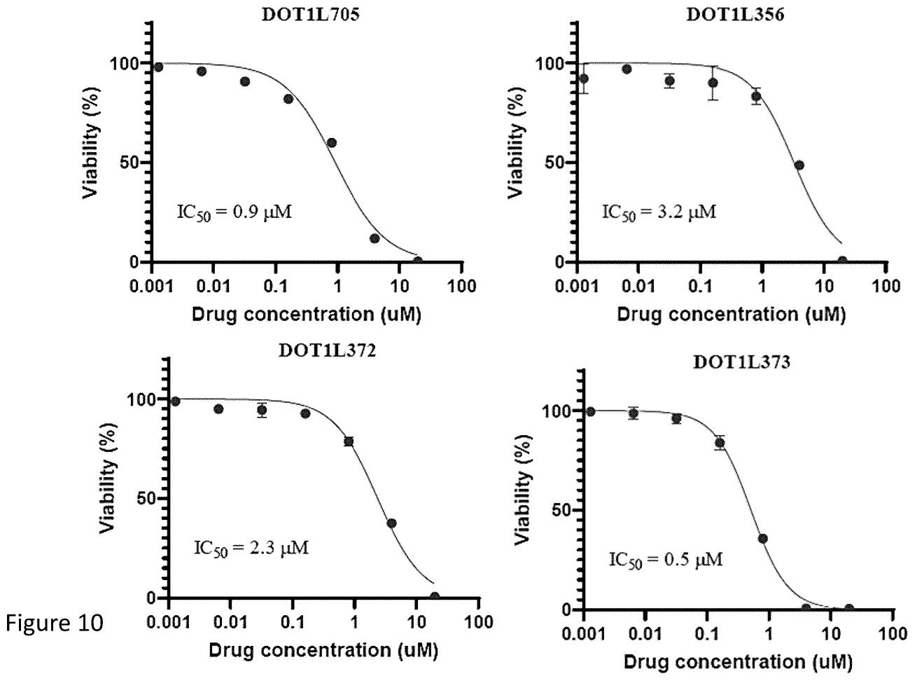

### DETAILED DESCRIPTION OF THE INVENTION

The present invention is described herein using several definitions, as set forth below and throughout the application.

As used in this specification and the claims, the singular forms “a,” “an,” and “the” include plural forms unless the context clearly dictates otherwise. For example, the term “a compound” should be interpreted to mean “one or more compounds” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

### Compounds

Disclosed herein are compounds, or pharmaceutically acceptable salts thereof, wherein the compounds have a formula I(a) or I(b):

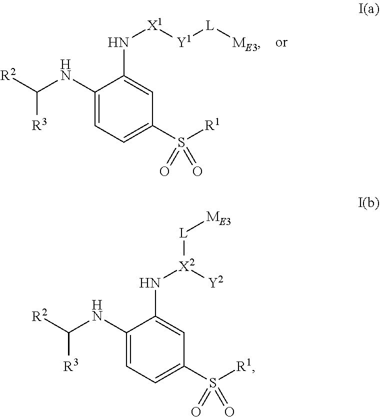

wherein
R¹ is selected from the group consisting of hydrogen, alkyl, cycloalkyl, heterocyclyl, aryl, and heteroaryl;
R² and R³ are each independently an optionally substituted aryl or an optionally substituted heteroaryl;
X¹ is an optionally substituted heteroarylene;
Y¹ is an optionally substituted heterocycloalkylene;
X² is an optionally substituted trivalent heteroaryloxyl;
Y² is an optionally substituted heterocyclyl;
L is a bond or a linker; and
M_{E3} is a moiety that binds to an E3 ubiquitin ligase.

As used herein, the term “alkyl” includes a straight-chain or branched alkyl radical in all of its isomeric forms, such as a straight or branched group of 1-12, 1-10, or 1-6 carbon atoms, referred to herein as C₁-C₁₂ alkyl, C₁-C₁₀ alkyl, and C₁-C₆ alkyl, respectively.

As used herein, the term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C4-8-cycloalkyl,” derived from a cycloalkane.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using “x-membered to y-membered” nomenclature where x and y are integers specifying the number of ring atoms. For example, a 3-membered to 7-membered heterocyclyl group refers to a saturated or partially unsaturated 3- to 7-membered ring structure containing one to four heteroatoms, such as nitrogen, oxygen, and sulfur. The designation “3-membered to 7-membered” indicates that the heterocyclic ring contains a total of from 3 to 7 ring atoms, inclusive of any heteroatoms that occupy a ring atom position.

As used herein, the term “aryl” is art-recognized and refers to a carbocyclic aromatic group. Representative aryl groups include phenyl, naphthyl, anthracenyl, and the like. The term “aryl” includes polycyclic ring systems having two or more rings in which two or more carbons are common to two adjoining rings (the rings are “fused rings”) wherein at least one of the rings is aromatic and, e.g., the other ring(s) may be cycloalkyls, cycloalkenyls, cycloalkynyls, heterocyclyl, and/or aryls. In certain embodiments, the aryl group is a 6-10 membered ring structure.

The term “heteroaryl” as used herein refers to a monocyclic heteroaryl and a bicyclic heteroaryl. The monocyclic heteroaryl is a five- or six-membered ring. The five-membered ring contains two double bonds. The five membered ring may contain one heteroatom selected from O or S; or one, two, three, or four nitrogen atoms and optionally one oxygen or one sulfur atom. The six-membered ring contains three double bonds and one, two, three or four nitrogen atoms.

Representative examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl. The bicyclic heteroaryl consists of a monocyclic heteroaryl fused to a phenyl, or a monocyclic heteroaryl fused to a monocyclic cycloalkyl, or a monocyclic heteroaryl fused to a monocyclic cycloalkenyl, or a monocyclic heteroaryl fused to a monocyclic heteroaryl, or a monocyclic heteroaryl fused to a monocyclic heterocycle. Representative examples of bicyclic heteroaryls include, but are not limited to, benzofuranyl, benzothienyl, benzoxazolyl, benzimidazolyl, benzoxadiazolyl, phthalazinyl, 2,6-dihydropyrrolo[3,4-c]pyrazol-5(4H)-yl, 6,7-dihydro-pyrazolo[1,5-a]pyrazin-5(4H)-yl, 6,7-dihydro-1,3-benzothiazolyl, imidazo[1,2-a]pyridinyl, indazolyl, indolyl, isoindolyl, isoquinolinyl, naphthyridinyl, pyridoimidazolyl, quinolinyl, 2,4,6,7-tetrahydro-5H-pyrazolo[4,3-c]pyridin-5-yl, thiazolo[5,4-b]pyridin-2-yl, thiazolo[5,4-d]pyrimidin-2-yl, and 5,6, 7,8-tetrahydroquinolin-5-yl. The monocyclic and bicyclic heteroaryls, including exemplary rings, are optionally substituted unless otherwise indicated. The monocyclic and bicyclic heteroaryls are connected to the parent molecular moiety through any substitutable carbon atom or any substitutable nitrogen atom contained within the ring systems. The nitrogen atom in the heteroaryl rings may optionally be oxidized and may optionally be quartemized.

The term “optionally substituted” refers to a group (e.g. alkyl, aryl, and heteroaryl) that is unsubstituted or substituted with one or more substituents independently selected from the group consisting of halo, alkyl, alkenyl, alkynyl, cycloalkyl, heterocyclyl, hydroxyl, carboxyl, alkoxy, amino, aryl, and haloalkyl.

The term “halo” refers to a halogen atom or halogen radical (e.g., —F, —Cl, —Br, or —I).

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₆-alkynyl, and C₂-C₆-alkynyl, respectively.

The terms “alkoxy” or “alkoxyl” are art-recognized and refer to an alkyl group, as defined above, having an oxygen radical attached thereto. Representative alkoxy groups include methoxy, ethoxy, tert-butoxy and the like.

The terms “amine” and “amino” are art-recognized and refer to both unsubstituted and substituted amines (e.g., mono-substituted amines or di-substituted amines), wherein substituents may include, for example, alkyl, cycloalkyl, heterocyclyl, alkenyl, and aryl.

The term “carboxyl” as used herein refers to the radical —COOH.

The term “hydroxyl” as used herein refers to the radical —OH.

The term “haloalkyl” refers to an alkyl group that is substituted with at least one halogen. For example, —CH₂F, —CHF₂, —CF₃, —CH₂CF₃, —CF₂CF₃, and the like.

As used herein, the term “heteroarylene” refers to a diradical of a heteroaryl group as defined herein.

As used herein, the term “heterocyclylene” refers to a diradical of a heterocyclyl group as defined herein.

As used herein, the term “heteroaryloxyl” refers to a heteroaryl group, as defined herein, having an oxygen radical attached thereto.

In some embodiments, R¹ is alkyl in Formula I(a) or Formula I(b).

In some embodiments, R² is pyridinyl substituted with one or more halo in Formula I(a) or Formula I(b).

As used herein, the term “pyridinyl” refers to the radical

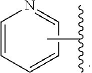

As used herein, a wavy line “ [symbol] ” may be used to designate the point of attachment for any radical group or substituent group.

In some embodiments, R³ is 1,3-benzodioxlyl substituted with one or more halo in Formula I(a) or Formula I(b).

As used herein, the term “1,3-benzodioxlyl” refers to the radical

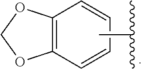

In some embodiments, X¹ is 1,3,5-triazinylene substituted with alkoxy, or X² is a trivalent 1,3,5-triazin-2-oxyl in Formula I(a) or Formula I(b).

As used herein, the term “1,3,5-triazinylene” refers to the diradical

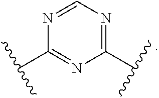 [SMILES: *c1ncnc(*)n1]

As used herein, the term “trivalent 1,3,5-triazin-2-oxyl” refers to the triradical

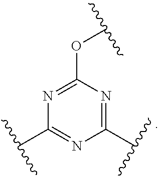 [SMILES: *Oc1nc(*)nc(*)n1]

In some embodiments, Y¹ is piperazinylene or Y² is piperazinyl in Formula I(a) or Formula I(b).

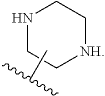

As used herein, the term “piperazinyl” refers to the radical. The term “piperazinylene” refers to the diradical of piperazinyl.

In some embodiments, the compound has a Formula I(aa) or I(bb):

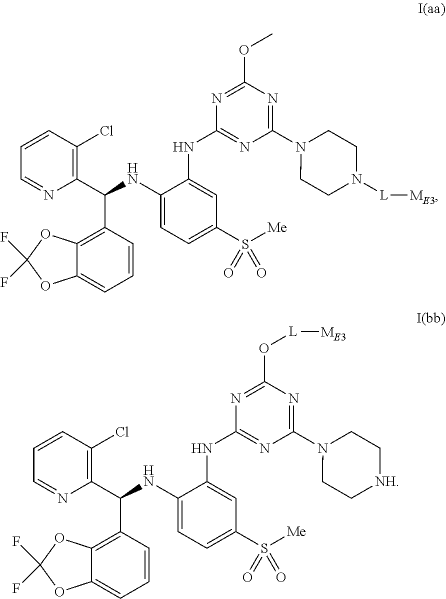

In some embodiments, L in the compounds disclosed herein is a bond or a linker selected from —(CH₂—C(O)NH)ₘ—(CH₂)₂—(OCH₂)ₙ—,

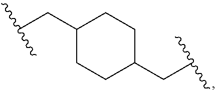 [SMILES: *CC1CCC(C*)CC1]

and —(CH₂)ₚ—, wherein m is 0 or 1, n is an integer selected from 1 to 20, and p is an integer from 1 to 10. In some such embodiments, m is 0 and n is an integer selected from 1 to 20, from 1 to 15, from 1 to 10, or from 1 to 5. In some such embodiments, m is 1 and n is an integer selected from 1 to 20, from 1 to 15, from 1 to 10, or from 1 to 5. In some such embodiments, p is an integer selected from 1 to 5 or from 3 to 7.

In some embodiments, M_{E3} is a moiety that binds to an E3 ubiquitin ligase selected from Von Hippel-Lindau (VHL) E3 ubiquitin ligase and cereblon (CRBN) E3 ubiquitin ligase.

In some embodiments, M_{E3} is selected from the group consisting of

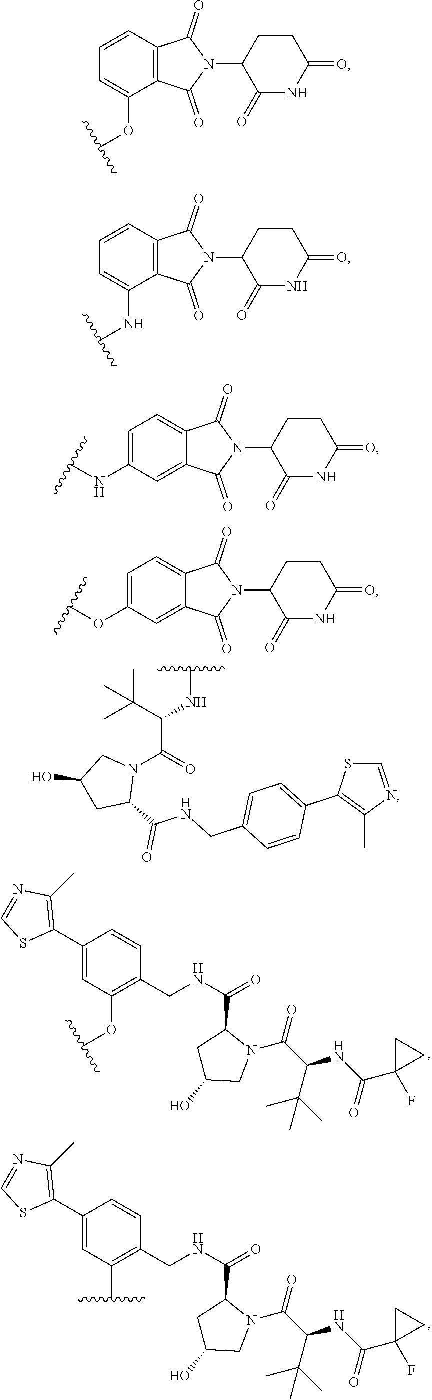

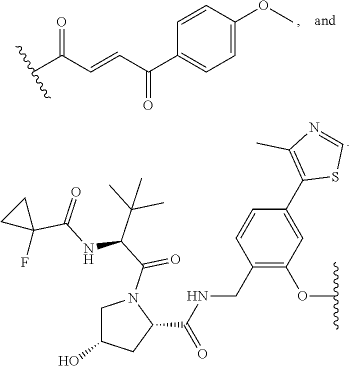 [SMILES: *C(=O)/C=C/C(=O)c1ccc(OC)cc1 ; *Oc1cc(-c2scnc2C)ccc1CNC(=O)[C@@H]1C[C@H](O)CN1C(=O)[C@@H](NC(=O)C1(F)CC1)C(C)(C)C]

In some embodiments, the compound as disclosed herein is selected from the group consisting of

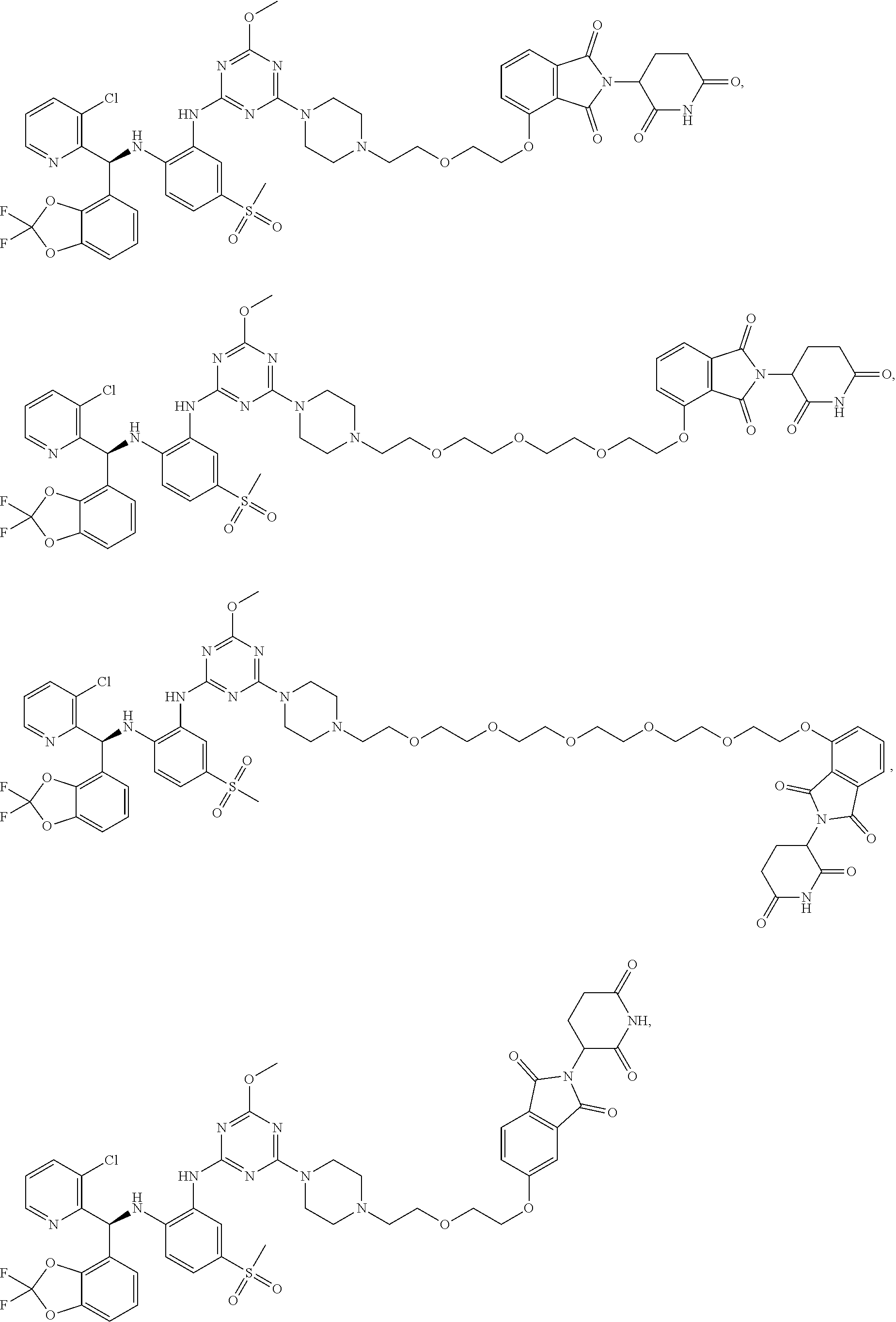

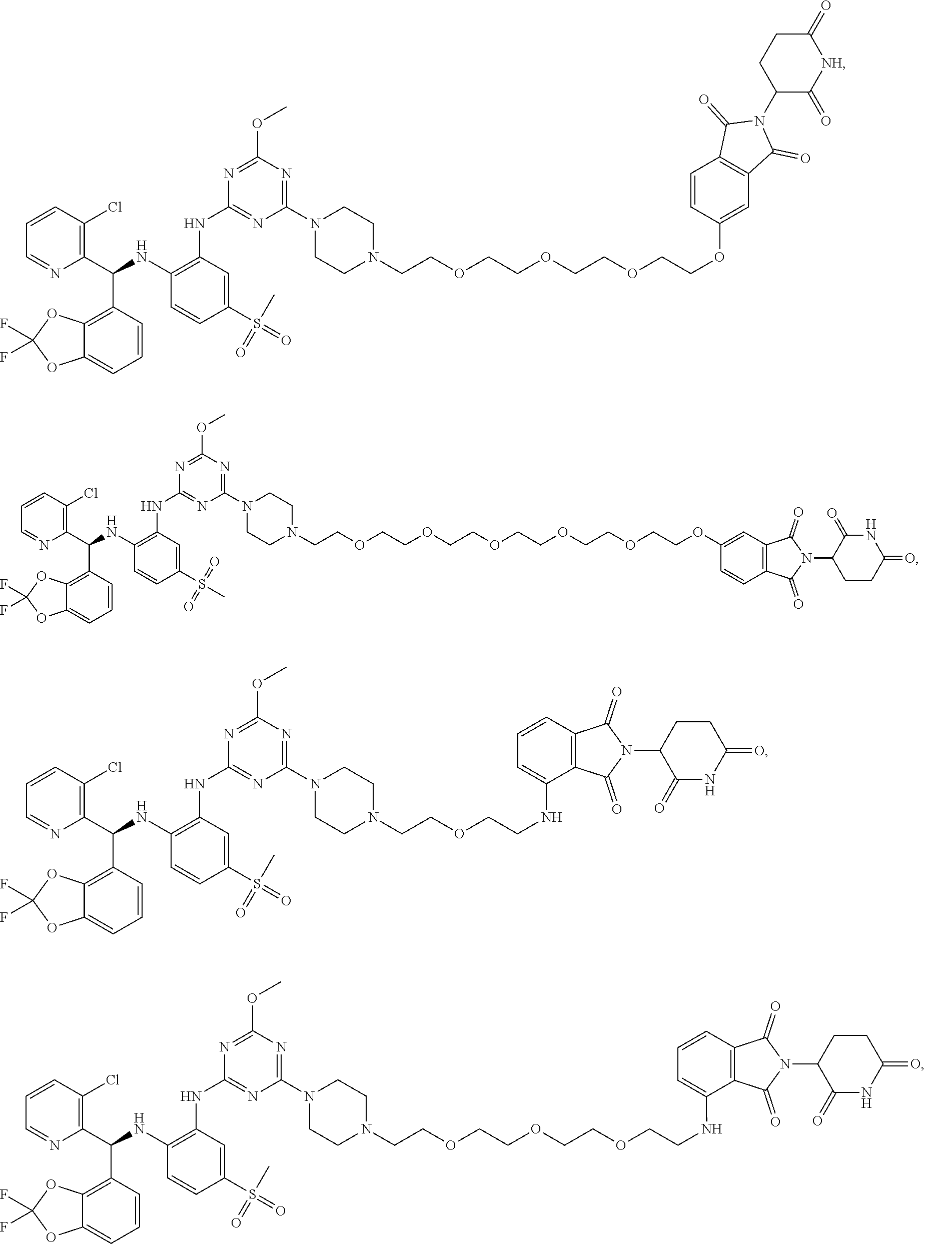

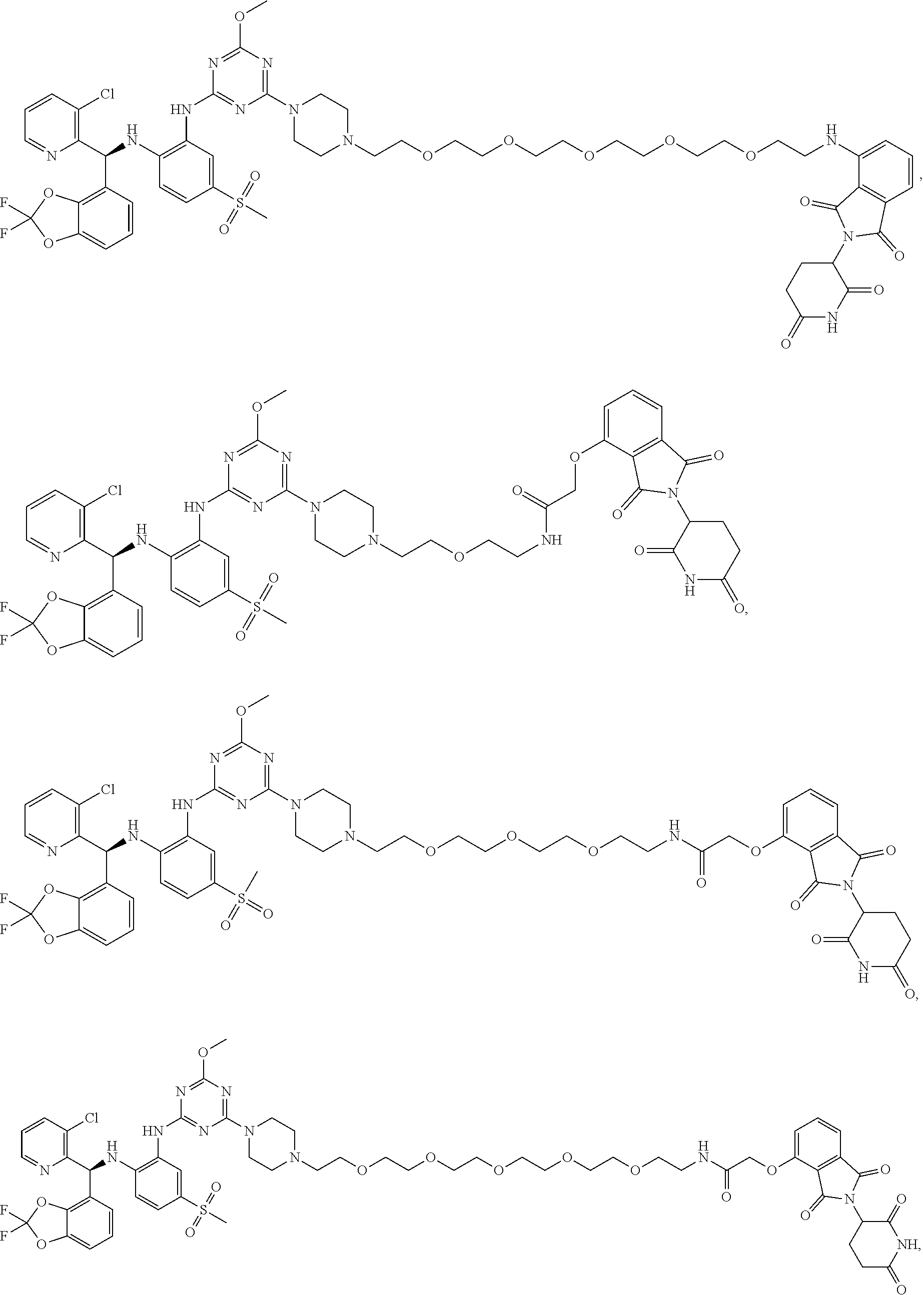

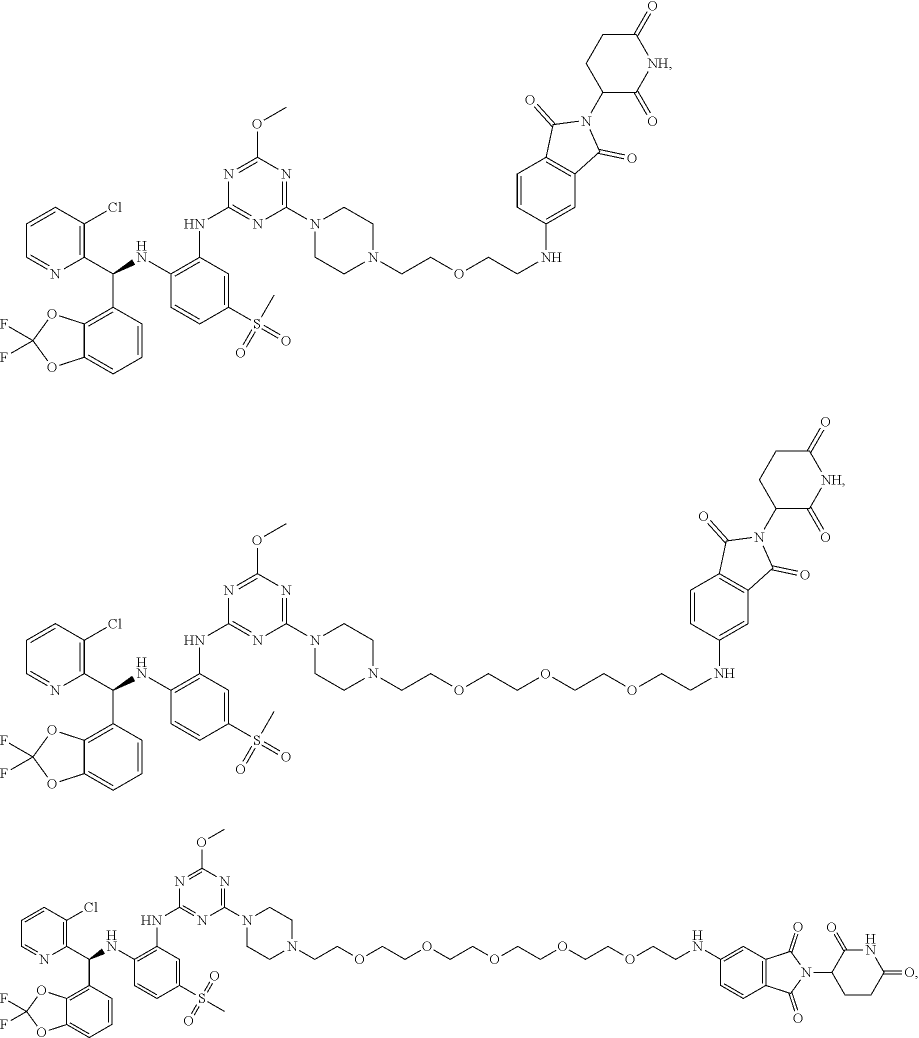 [SMILES: COc1nc(Nc2cc(S(C)(=O)=O)ccc2N[C@@H](c2cccc3c2OC(F)(F)O3)c2ncccc2Cl)nc(N2CCN(CCOCCNc3ccc4c(c3)C(=O)N(C3CCC(=O)NC3=O)C4=O)CC2)n1 ; COc1nc(Nc2cc(S(C)(=O)=O)ccc2N[C@@H](c2cccc3c2OC(F)(F)O3)c2ncccc2Cl)nc(N2CCN(CCOCCOCCOCCNc3ccc4c(c3)C(=O)N(C3CCC(=O)NC3=O)C4=O)CC2)n1 ; COc1nc(Nc2cc(S(C)(=O)=O)ccc2N[C@@H](c2cccc3c2OC(F)(F)O3)c2ncccc2Cl)nc(N2CCN(CCOCCOCCOCCOCCOCCNc3ccc4c(c3)C(=O)N(C3CCC(=O)NC3=O)C4=O)CC2)n1]

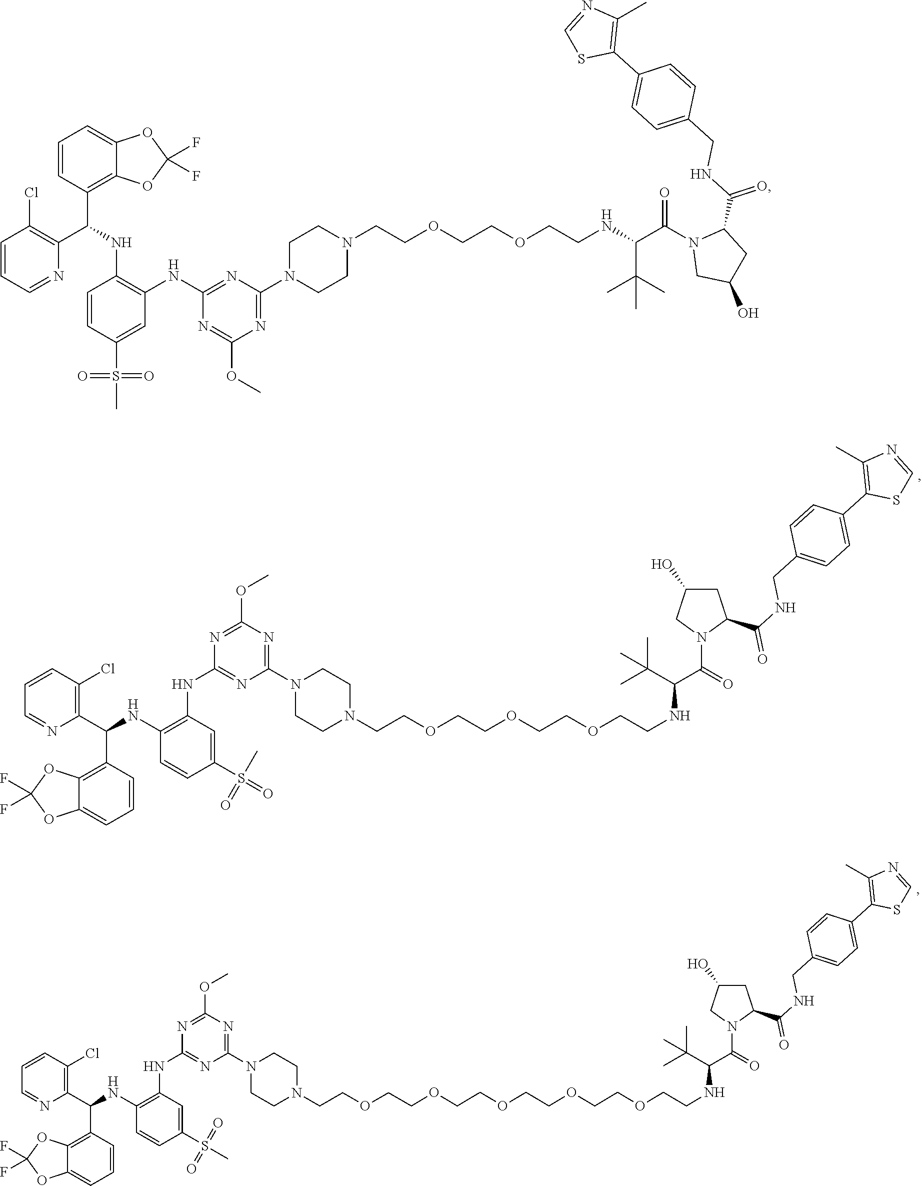 [SMILES: COc1nc(Nc2cc(S(C)(=O)=O)ccc2N[C@@H](c2cccc3c2OC(F)(F)O3)c2ncccc2Cl)nc(N2CCN(CCOCCOCCN[C@H](C(=O)N3C[C@H](O)C[C@H]3C(=O)NCc3ccc(-c4scnc4C)cc3)C(C)(C)C)CC2)n1 ; COc1nc(Nc2cc(S(C)(=O)=O)ccc2N[C@@H](c2cccc3c2OC(F)(F)O3)c2ncccc2Cl)nc(N2CCN(CCOCCOCCOCCN[C@H](C(=O)N3C[C@H](O)C[C@H]3C(=O)NCc3ccc(-c4scnc4C)cc3)C(C)(C)C)CC2)n1 ; COc1nc(Nc2cc(S(C)(=O)=O)ccc2N[C@@H](c2cccc3c2OC(F)(F)O3)c2ncccc2Cl)nc(N2CCN(CCOCCOCCOCCOCCOCCN[C@H](C(=O)N3C[C@H](O)C[C@H]3C(=O)NCc3ccc(-c4scnc4C)cc3)C(C)(C)C)CC2)n1]

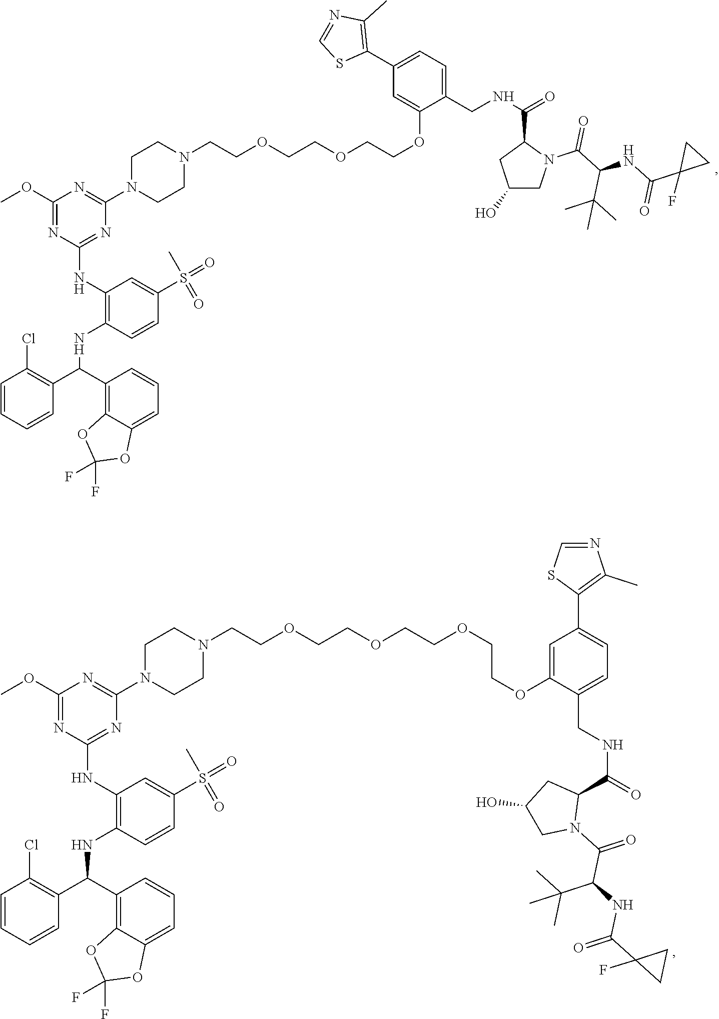 [SMILES: COc1nc(Nc2cc(S(C)(=O)=O)ccc2NC(c2ccccc2Cl)c2cccc3c2OC(F)(F)O3)nc(N2CCN(CCOCCOCCOc3cc(-c4scnc4C)ccc3CNC(=O)[C@@H]3C[C@@H](O)CN3C(=O)[C@@H](NC(=O)C3(F)CC3)C(C)(C)C)CC2)n1 ; COc1nc(Nc2cc(S(C)(=O)=O)ccc2N[C@H](c2ccccc2Cl)c2cccc3c2OC(F)(F)O3)nc(N2CCN(CCOCCOCCOCCOc3cc(-c4scnc4C)ccc3CNC(=O)[C@@H]3C[C@@H](O)CN3C(=O)[C@@H](NC(=O)C3(F)CC3)C(C)(C)C)CC2)n1]

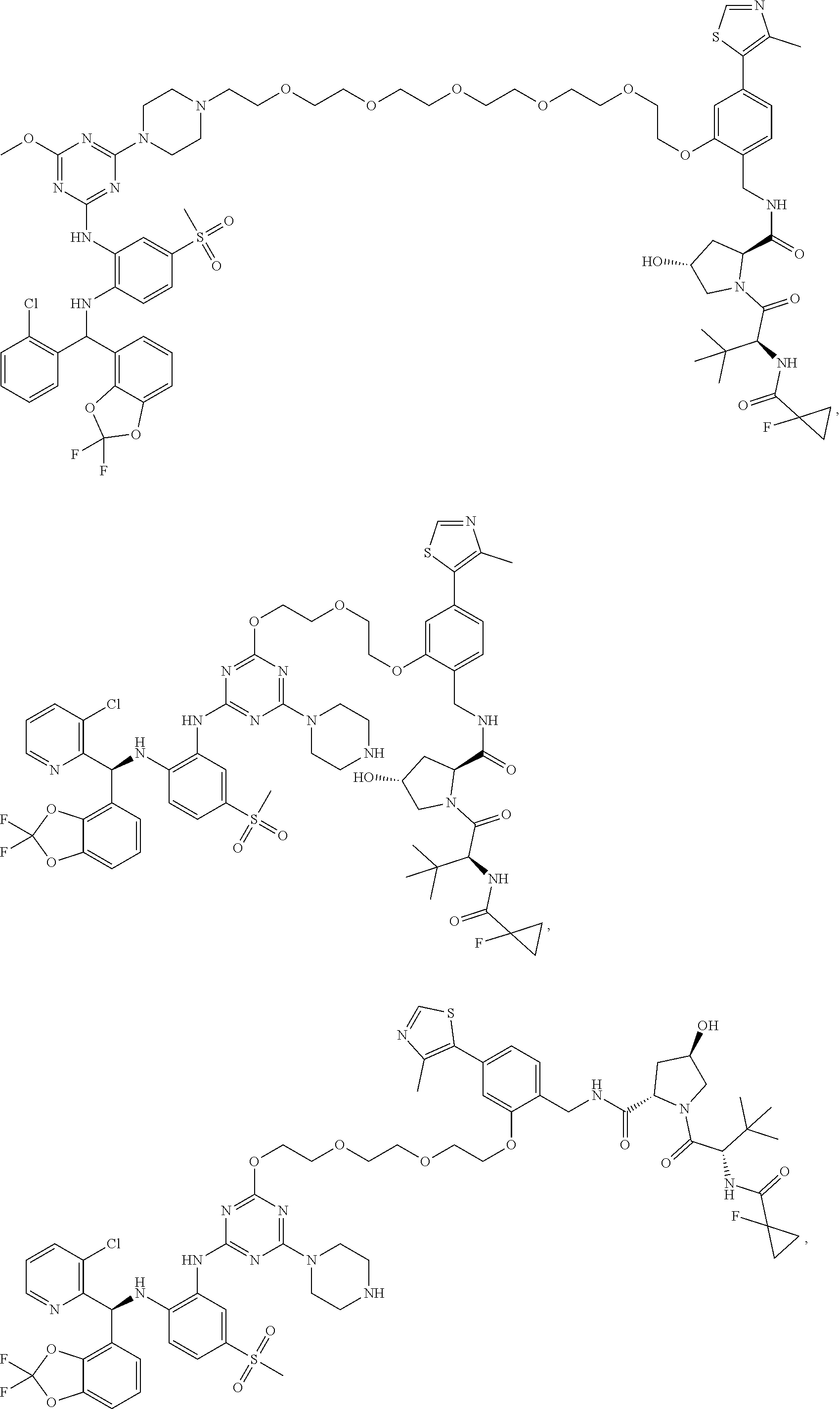

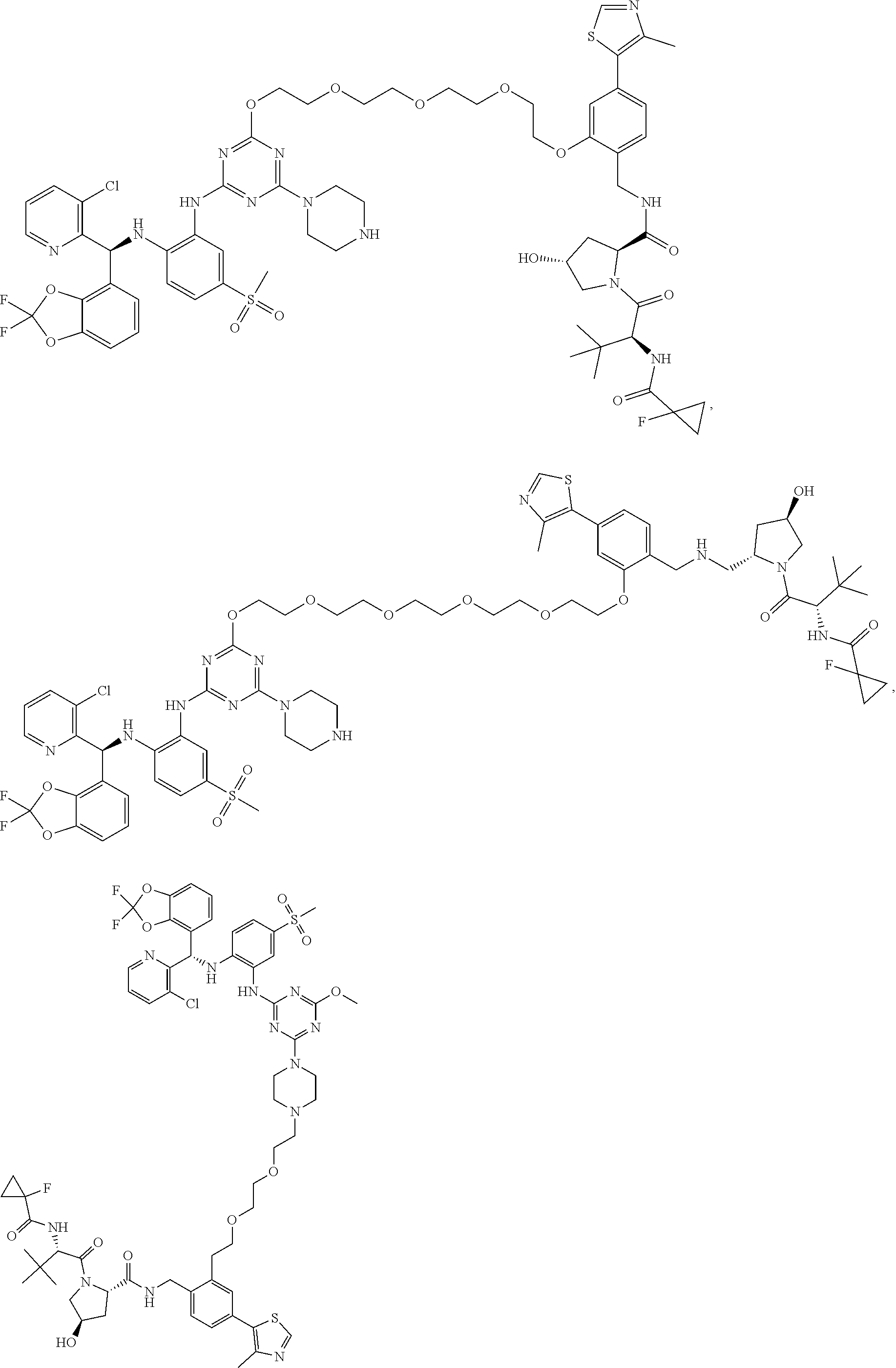

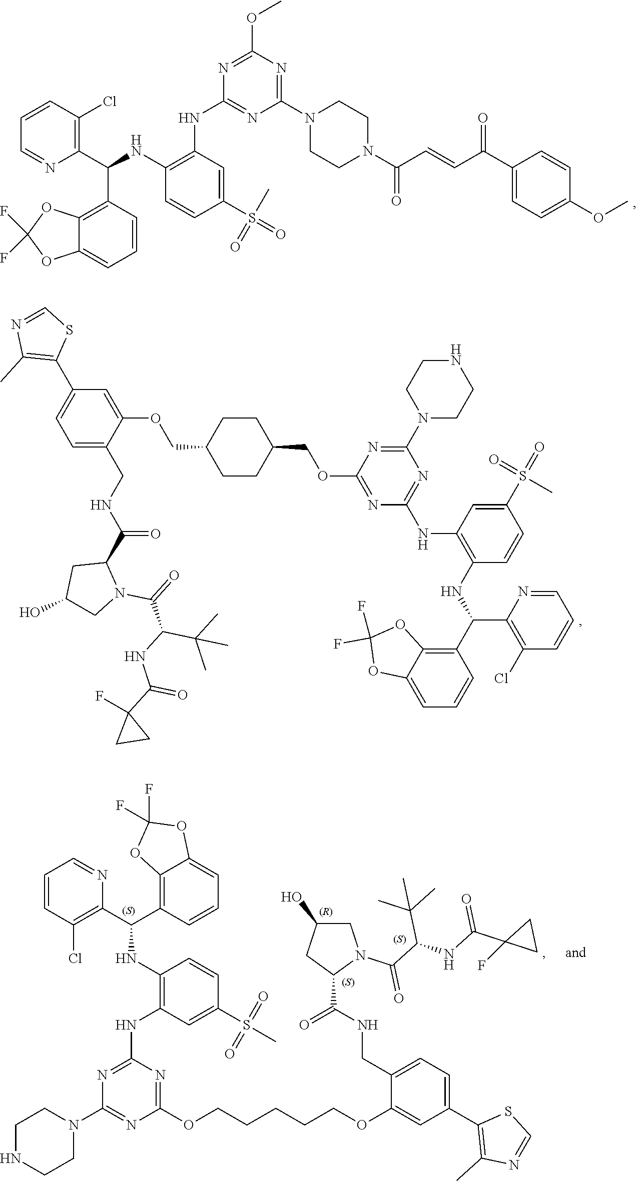

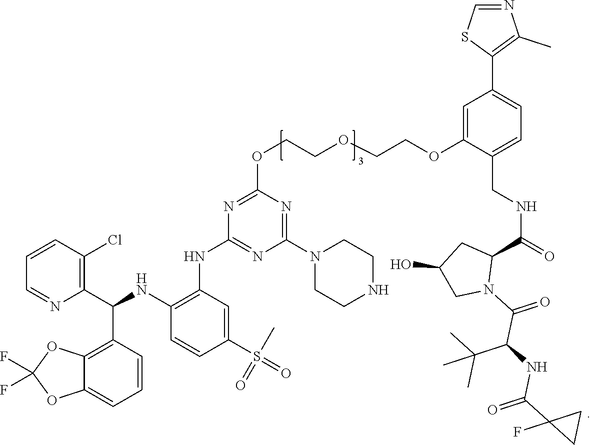

In some embodiments, the compound as disclosed herein is selected from the group consisting of

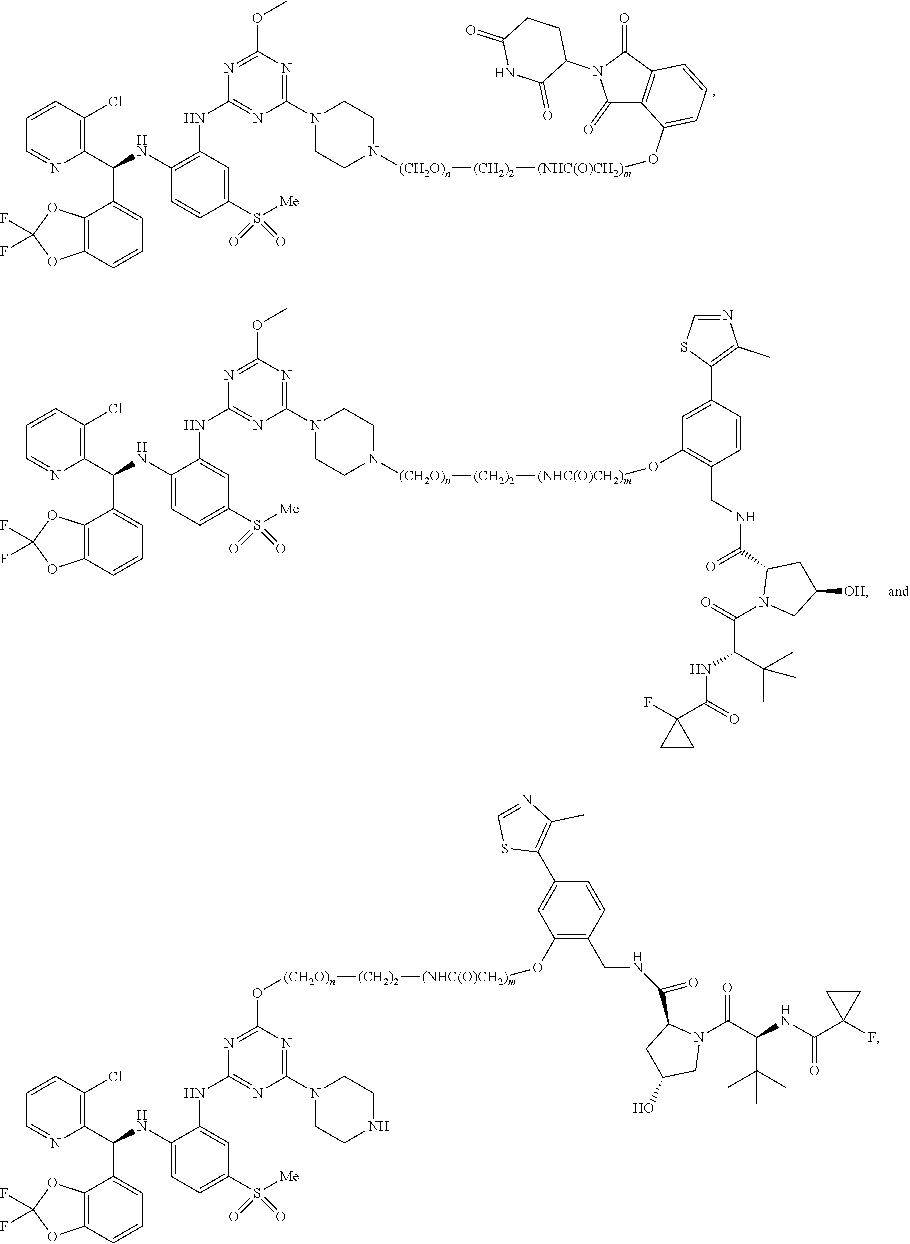

and wherein m is 0 or 1 and n is an integer selected from 1 to 20.

In some embodiments, the compound as disclosed herein is selected from the group consisting of

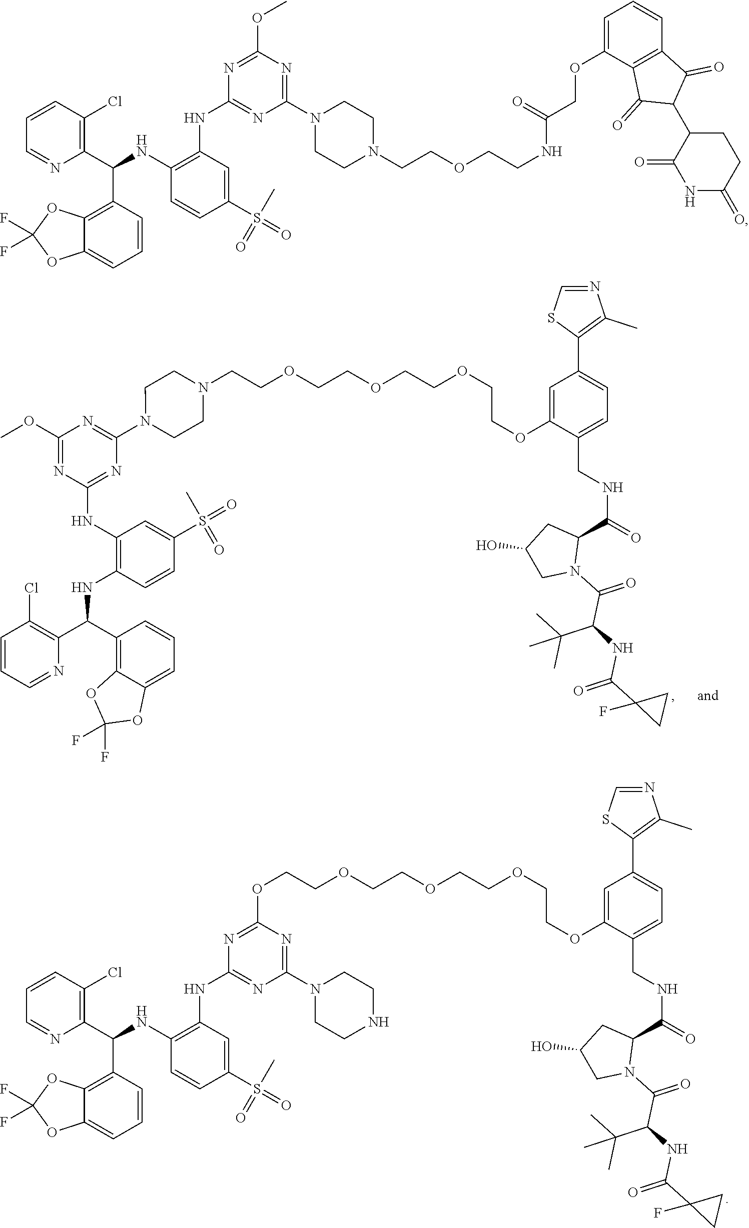 [SMILES: COc1nc(Nc2cc(S(C)(=O)=O)ccc2N[C@@H](c2cccc3c2OC(F)(F)O3)c2ncccc2Cl)nc(N2CCN(CCOCCNC(=O)COc3cccc4c3C(=O)C(C3CCC(=O)NC3=O)C4=O)CC2)n1 ; COc1nc(Nc2cc(S(C)(=O)=O)ccc2N[C@@H](c2cccc3c2OC(F)(F)O3)c2ncccc2Cl)nc(N2CCN(CCOCCOCCOCCOc3cc(-c4scnc4C)ccc3CNC(=O)[C@@H]3C[C@@H](O)CN3C(=O)[C@@H](NC(=O)C3(F)CC3)C(C)(C)C)CC2)n1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOc2nc(Nc3cc(S(C)(=O)=O)ccc3N[C@@H](c3cccc4c3OC(F)(F)O4)c3ncccc3Cl)nc(N3CCNCC3)n2)c1]

### Pharmaceutical Compositions

Also disclosed herein are pharmaceutical composition comprising a therapeutically effective amount of the compounds as disclosed herein and a pharmaceutically acceptable carrier, excipient, or diluent.

As used herein, the phrase “effective amount” shall mean that drug dosage that provides the specific pharmacological response for which the drug is administered in a significant number of subject in need of such treatment. An effective amount of a drug that is administered to a particular subject in a particular instance will not always be effective in treating the conditions/diseases described herein, even though such dosage is deemed to be a therapeutically effective amount by those of skill in the art.

The pharmaceutical composition may include the compound in a range of about 0.1 to 2000 mg. In some embodiments, the pharmaceutical composition may include the compound in a range of from about 0.5 to 500 mg. In some embodiments, the pharmaceutical composition may include the compound in a range of from about 1 to 100 mg. The pharmaceutical composition may be administered to provide the compound at a daily dose of about 0.1 to about 1000 mg/kg body weight. In some embodiments, the pharmaceutical composition may be administered to provide the compound at a daily dose of about 0.5 to about 500 mg/kg body weight. In some embodiments, the pharmaceutical composition may be administered to provide the compound at a daily dose of about 50 to about 100 mg/kg body weight. In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 μM, 0.005 μM, 0.01 μM, 0.5 μM, 0.1 μM, 1.0 μM, 10 μM, and 100 μM (e.g., 0.1 μM-1.0 μM).

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and crosslinked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, may include colloidal silicon dioxide, such as Aerosil® 200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame.

Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are in some embodiments applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

Optionally, the disclosed compounds or pharmaceutical compositions comprising the disclosed compounds may be administered with additional therapeutic agents, optionally in combination, in order to treat cell proliferative diseases and disorders. In some embodiments of the disclosed methods, one or more additional therapeutic agents are administered with the disclosed compounds or with pharmaceutical compositions comprising the disclosed compounds, where the additional therapeutic agent is administered prior to, concurrently with, or after administering the disclosed compounds or the pharmaceutical compositions comprising the disclosed compounds. In some embodiments, the disclosed pharmaceutical composition are formulated to comprise the disclosed compounds and further to comprise one or more additional therapeutic agents, for example, one or more additional therapeutic agents for treating cell proliferative diseases and disorders.

### Methods

Disclosed herein are also methods of treating a disease or disorder associated with DOT1L activity in a subject in need thereof. The methods comprise administering to the subject the compound or the pharmaceutical composition as disclosed herein.

As used herein, a “subject” may be interchangeable with “patient” or “individual” and means an animal, which may be a human or non-human animal, in need of treatment.

A “subject in need of treatment” may include a subject having a disease, disorder, or condition that is responsive to therapy with the compounds as disclosed herein. For example, a “subject in need of treatment” may include a subject having a cell proliferative disease, disorder, or condition such as cancer. In some embodiments, the cancer is a leukemia. In some embodiment, the leukemia is MLL-rearranged leukemia, such as acute myeloid leukemia (AML) or acute lymphoblastic leukemia.

In some embodiments of the disclosed treatment methods, the subject may be administered a dose of a compound as low as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg once daily, twice daily, three times daily, four times daily, once weekly, twice weekly, or three times per week in order to treat the disease or disorder in the subject. In some embodiments, the subject may be administered a dose of a compound as high as 1.25 mg, 2.5 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, 60 mg, 62.5 mg, 65 mg, 67.5 mg, 70 mg, 72.5 mg, 75 mg, 77.5 mg, 80 mg, 82.5 mg, 85 mg, 87.5 mg, 90 mg, 100 mg, 200 mg, 500 mg, 1000 mg, or 2000 mg, once daily, twice daily, three times daily, four times daily, once weekly, twice weekly, or three times per week in order to treat the disease or disorder in the subject. Minimal and/or maximal doses of the compounds may include doses falling within dose ranges having as endpoints any of these disclosed doses (e.g., 2.5 mg-200 mg).

In some embodiments, a minimal dose level of a compound for achieving therapy in the disclosed methods of treatment may be at least about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. In some embodiments, a maximal dose level of a compound for achieving therapy in the disclosed methods of treatment may not exceed about 10, 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, 1900, 2000, 3000, 4000, 5000, 6000, 7000, 8000, 9000, 10000, 15000, or 20000 ng/kg body weight of the subject. Minimal and/or maximal dose levels of the compounds for achieving therapy in the disclosed methods of treatment may include dose levels falling within ranges having as endpoints any of these disclosed dose levels (e.g., 500-2000 ng/kg body weight of the subject)

In some embodiments, the disease or disorder is a cell proliferative disease or disorder. In some such embodiments, the cell proliferative disease or disorder is leukemia.

In some embodiments, the leukemia is MLL-rearranged leukemia.

In some embodiments, the leukemia is acute myeloid leukemia or acute lymphoblastic leukemia.

Discloses herein are also methods of inhibiting cell proliferation. The methods comprise contacting cells with the compound as described herein, or a pharmaceutically acceptable salt thereof. In some such embodiments, the cells have mixed lineage leukemia (MLL) rearrangements.

In some embodiments, the disclosed compound may be effective in inhibiting cell proliferation of one or more types of cancer cells including leukemia cells, such as MV-4-11. In some embodiments, the cells are acute myeloid leukemia cells or acute lymphoblastic leukemia cells.

In some embodiments, the contact is in vivo.

Cell proliferation and inhibition thereof by the presently disclosed compounds may be assessed by cell viability methods disclosed in the art including colorimetric assays that utilize dyes such as MTT, XTT, and MTS to assess cell viability. In some embodiments, the disclosed compounds have an IC₅₀ of less than about 10 μM, 5 μM, 1 μM, or 0.5 μM in the selected assay.

### EXAMPLES

The following Examples are illustrative and are not intended to limit the scope of the claimed subject matter.

### Example 1

### Synthesis of Compound 10

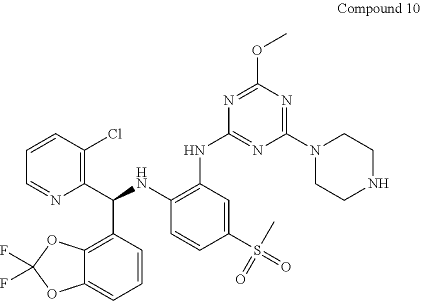 [SMILES: COc1nc(Nc2cc(S(C)(=O)=O)ccc2N[C@@H](c2cccc3c2OC(F)(F)O3)c2ncccc2Cl)nc(N2CCNCC2)n1]

### Original Scheme

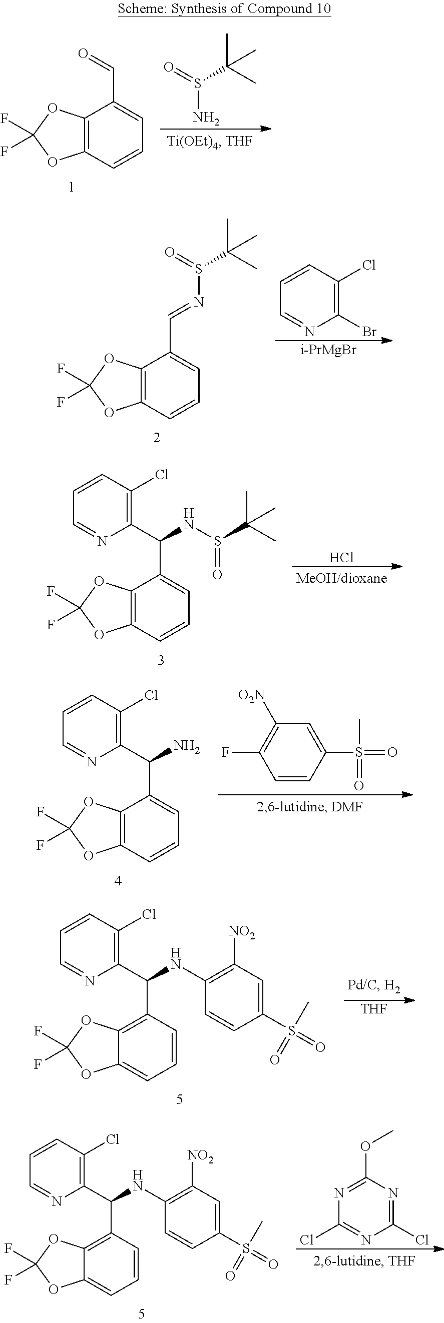

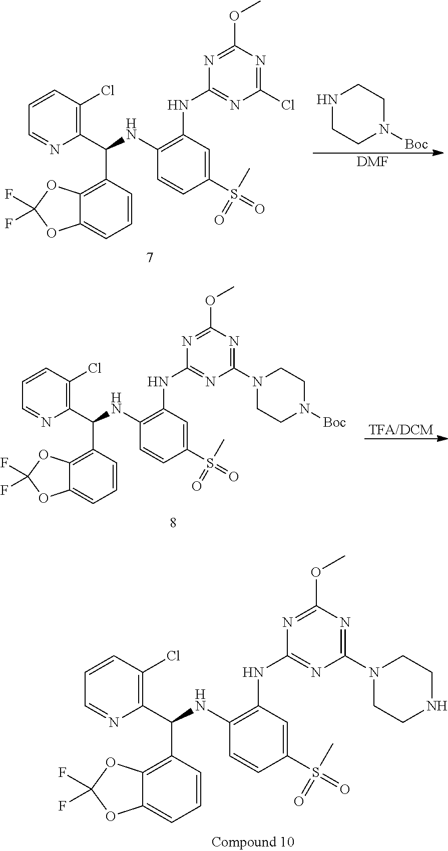

### Chemical Synthesis

All chemical reagents were obtained from commercial suppliers and used without further purification, unless otherwise stated. Reactions were run without taking precautions to exclude air or moisture, unless otherwise noted. Normal phase column chromatography was performed using silica gel columns and ACS grade solvents. Analytical TLC was performed on EM Reagent 0.25 mm silica gel 60 F254 plates and visualized by UV light. Compound identities were confirmed by ¹H and F (NMR) spectroscopy which were recorded on a Bruker 400 MHz spectrometer using the corresponding residual solvent peak (CDCl₃,1H δ=7.27: CD₃OD, 1H δ=3.31; DMSO-d₆, 1H δ=2.50) as an internal standard. The chemical shifts for ¹H-NMR is reported to the second decimal place. Proton coupling constants are expressed in hertz (Hz). Standard abbreviations were used to denote spin multiplicity for ¹H NMR data.

### LCMS Methods:

Method: 5_95AB 6min-220-254-ELSD

LC/MS (The gradient was 5% B in 0.40 min and 5-95% B in 2.60 min, hold on 95% B in 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Kinetex C18 2.1*50 mm, 5 um. Detection methods are diode array (DAD), and evaporative light scattering detection (ELSD). MS mode was positive electrospray ionization. MS range was 100-1000.

SFC methods:

### Method 1: OD IPA IPAm 10_50_25_35_5 min

Column: Chiralpak OD-3, 150×4.6 mm I.D., 3 um
Mobile phase: A: CO₂ B: IPA (0.1% IPAm, v/v)
Gradient:
Time A % B %
0.0 90 10
0.5 90 10
3.5 50 50
4.5 50 50
5.0 90 10
Flow rate: 2.5 mL/min
Column temp.: 35° C.
ABPR: 2000 psi

### Method 2: OJ_MeOH_IPAm_5_50_34_35_3min

Column: Chiralcel OJ-3, 50×4.6 mm I.D., 3 um
Mobile phase: A: CO₂ B: MeOH (0.1% IPAm, v/v)
Gradient:
Time A % B %
0.0 95 5
0.2 95 5
1.2 50 50
2.2 50 50
2.6 95 5
3.0 95 5
Flow rate: 3.4 mL/min
Column temp.: 35° C.
ABPR: 1800 psi

### Method 3: OD_MeOH_IPAm_5_50_34_35_3min

Column: Chiralcel OD-3, 50×4.6 mm I.D., 3 um
Mobile phase: A: CO₂ B: MeOH (0.1% IPAm, v/v)
Gradient:
Time A % B %
0.0 95 5
0.2 95 5
1.2 50 50
2.2 50 50
2.6 95 5
3.0 95 5
Flow rate: 3.4 mL/min
Column temp.: 35° C.
ABPR: 1800 psi

### Experimentals for Largest Scale Run:

### General Procedure for Preparation of Compound 2—Notebook Page: ET42365-48.

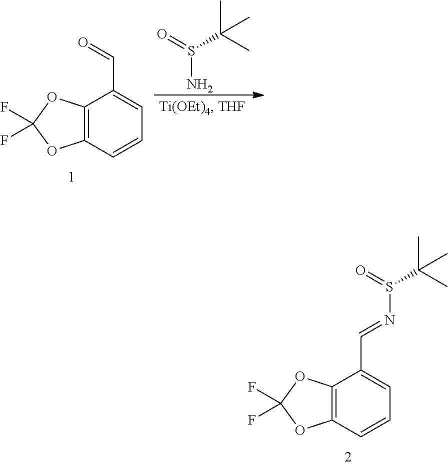

To a solution of Compound 1 (45 g, 241.79 mmol, 1 eq) and (R)-2-methylpropane-2-sulfinamide (35.17 g, 290.15 mmol, 1.2 eq) in THF (450 mL) was added Ti(OEt)₄ (82.73 g, 362.68 mmol, 1.5 eq) and the reaction was stirred at 45° C. for 2 hrs. TLC (petroleum ether/ethyl acetate=5/1) showed the starting material was consumed and a new peak was detected. The reaction was cooled and quenched with EtOAc (1 L) and brine (1 L). The orange suspension was filtered through the celite and the solid was washed with EtOAc (1 L). The aqueous layer was extracted with EtOAc (1 L). The combined organic layers were washed with brine (500 mL), dried over Na₂SO₄, filtered, evaporated and dried to give crude Compound 2 (68 g, yield 97.21%) as a yellow oil, which was used to the next step directly without further purification.

¹H NMR (ET42365-48-p1, 400 MHz, DMSO-d6) δ 1.12-1.29 (s, 9H) 7.37 (t, J=8.11 Hz, 1H) 7.64 (dd, J=8.00, 0.99 Hz, 1H) 7.72-7.80 (m, 1H) 8.58 (s, 1H)

### General Procedure for Preparation of Compound 3—Notebook Page: ET42365-52.

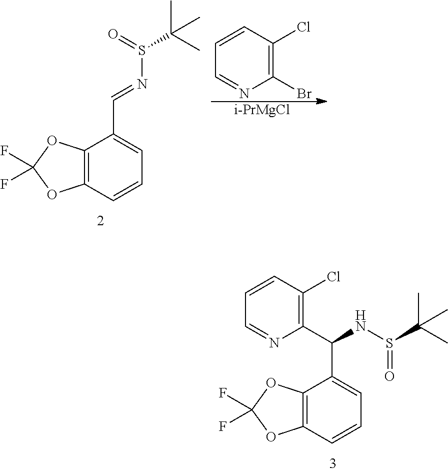

To a solution of 2-bromo-3-chloro-pyridine (54.28 g, 282.06 mmol, 1.2 eq) in THF (200 mL) was added isopropylmagnesium chloride (2 M, 141.03 mL, 1.2 eq) at −55° C. and the reaction was stirred at 25° C. for 2 hrs. Then the above reaction mixture was cooled to −55° C. and Compound 2 (68 g, 235.05 mmol, 1 eq) in toluene (700 mL) was added. The reaction was stirred at 25° C. for another one hour. TLC (petroleum ether/ethyl acetate=1/1) showed the starting material was consumed and a new spot was generated. The reaction was quenched with NH₄Cl (500 mL) and the mixture was extracted with EtOAc (500 mL). The organic layer was washed with brine (500 mL), dried over Na₂SO₄, filtered and concentrated to give the residue, which was purified by column chromatography on silica gel (eluted with petroleum ether/ethyl acetate=1/1) to give Compound 3 (60 g, yield 63.37%) as a yellow solid.

¹H NMR (ET42365-52-p1, 400 MHz, DMSO-d₆) δ 1.10 (s, 9H) 6.15 (d, J=8.75 Hz, 1H) 6.45 (d, J=8.75 Hz, 1H) 7.13-7.24 (m, 1H) 7.28-7.38 (m, 2H) 7.41 (dd, J=8.13, 4.63 Hz, 1H) 7.97 (dd, J8.07, 1.31 Hz, 1H) 8.56 (dd, J=4.63, 1.25 Hz, 1H)

### General Procedure for Preparation of Compound 4—Notebook Page: ET42365-62.

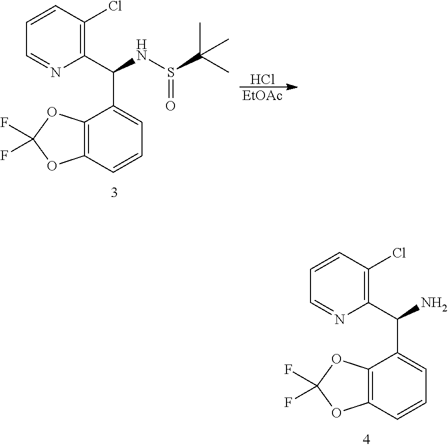 [SMILES: "EtOAc" ; CC(C)(C)[S@@](=O)N[C@@H](c1cccc2c1OC(F)(F)O2)c1ncccc1Cl ; Cl ; N[C@@H](c1cccc2c1OC(F)(F)O2)c1ncccc1Cl]

To a solution of Compound 3 (60 g, 148.94 mmol, 1 eq) in EtOAc (500 mL) was added HCl/EtOAc (4 M, 500.00 mL) and the reaction was stirred at 25° C. for one hour. TLC (petroleum ether/ethyl acetate=1/1) showed the starting material was consumed and a new spot was generate. The reaction was concentrated under reduced pressure to give a residue, which was triturated with ethyl acetate (200 mL) to give Compound 4 (40 g, yield 80.14%) as white solid.

¹H NMR (ET42365-62-p1, 400 MHz, DMSO-d₆) δ 6.03 (s, 1H) 7.14 (d, J=8.11 Hz, 1H) 7.25 (t, J=8.11 Hz, 1H) 7.47 (dd, J=8.11, 0.88 Hz, 1H) 7.59 (dd, J=8.11, 4.60 Hz, 1H) 8.07 (dd, J=8.11, 1.10 Hz, 1H) 8.73 (dd, J=4.71, 1.21 Hz, 1H) 9.23 (br s, 3H)

### General Procedure for Preparation of Compound 5—Notebook Page: ET43587-26.

The reactions were conducted in parallel but combined for purification.

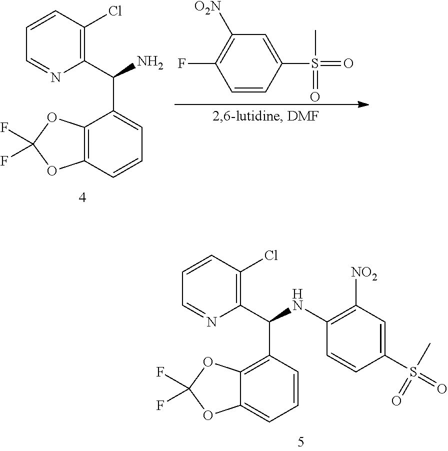

To a solution of 1-fluoro-4-methylsulfonyl-2-nitro-benzene (7.19 g, 32.82 mmol, 1.1 eq) and Compound 4 (10 g, 29.84 mmol, 1 eq, HCl) in DMF (100 mL) was added 2, 6-lutidine (7.99 g, 74.60 mmol, 8.69 mL, 2.5 eq). The mixture was stirred at 50° C. for 12 hrs. LCMS showed all the starting material was consumed and a new peak with desired product MS was detected. Two additional reactions were set up as detailed above and all three reaction mixtures were combined. The combined reaction mixture was diluted with water (300 mL) and stirred for one hour. The solid was filtered and dried under high vacuum to obtain Compound 5 (40 g, yield 85.27%) as a yellow solid, which was used to the next step directly without further purification.

¹H NMR (ET43587-26-p1c1, 400 MHz, CHLOROFORM-d) δ 3.04 (s, 3H), 6.42 (d, J=6.65 Hz, 1H), 6.96-7.04 (m, 2H), 7.04-7.09 (m, 1H), 7.13 (d, J=9.03 Hz, 1H), 7.34 (dd, J=8.03, 4.64 Hz, 1H), 7.76 (dd, J=8.09, 1.19 Hz, 1H), 8.71 (dd, J=4.64, 1.13 Hz, 1H), 8.80 (d, J=2.13 Hz, 1H), 10.59 (br d, J=6.53 Hz, 1H)

### General Procedure for Preparation of Compound 6—Notebook Page: ET43587-62.

The reactions were conducted in parallel but combined for purification.

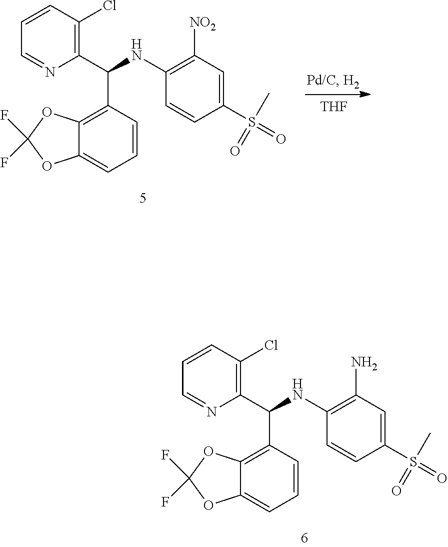 [SMILES: CS(=O)(=O)c1ccc(N[C@@H](c2cccc3c2OC(F)(F)O3)c2ncccc2Cl)c([N+](=O)[O-])c1 ; "THF" ; CS(=O)(=O)c1ccc(N[C@@H](c2cccc3c2OC(F)(F)O3)c2ncccc2Cl)c(N)c1]

To a solution of Pd/C (10 g, 9.43 mmol, 10% purity) in THF (600 mL) was added Compound 5 (20 g, 40.17 mmol, 1 eq). The mixture was stirred at 20° C. under H₂ (15 psi) for 12 hrs. TLC (petroleum ether/ethyl acetate=1/1) showed the starting material was consumed and a new spot was generated. One additional reaction was set up as detailed above and all two reaction mixtures were combined. The combined reaction mixture was filtered and concentrated under reduced pressure to give a residue, which was purified by chromatography on silica gel (eluted with petroleum ether/ethyl acetate=20/1 to 1/4) to give Compound 6 (34 g, yield 81.40%) as a yellow solid.

¹H NMR (ET43587-62-P1A, 400 MHz, CHLOROFORM-d) δ 2.97 (s, 3H), 6.29 (s, 1H), 6.76 (d, J=8.33 Hz, 1H), 6.90-7.08 (m, 3H), 7.24-7.30 (m, 2H), 7.34 (br d, J=8.33 Hz, 1H), 7.73 (d, J=7.89 Hz, 1H), 8.60 (d, J=4.39 Hz, 1H)

### General Procedure for Preparation of Compound 7—Notebook Page: ET43587-63.

The reactions were conducted in parallel but combined for purification.

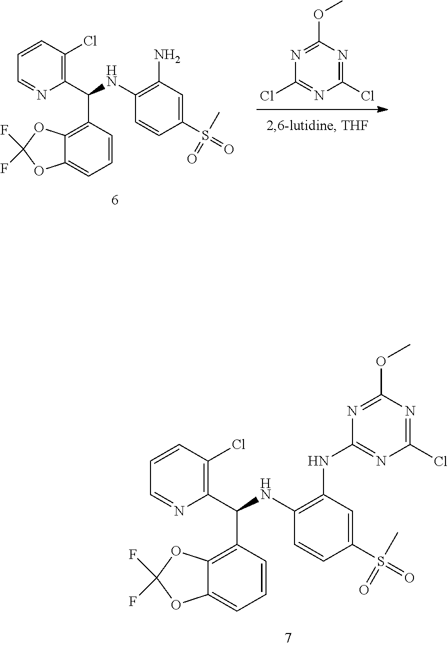

To a solution of 2,4-dichloro-6-methoxy-1,3,5-triazine (7.19 g, 39.97 mmol, 1.1 eq) and 2,6-lutidine (3.89 g, 36.33 mmol, 1 eq) in THF (170 mL) was added Compound 6 (17 g, 36.33 mmol, 1 eq) dropwise at 0° C. The mixture was stirred at 25° C. for 12 hrs. LCMS showed all the starting material was consumed and a new peak with desired product MS was detected. One additional reaction was set up as detailed above and all two reaction mixtures were combined. The combined reaction mixture was diluted with water (250 mL) and extracted with ethyl acetate (3×200 mL). The organic layer was separated and the combined organic layer was washed with brine (200 mL), dried over Na₂SO₄, filtered and concentrated under reduced pressure to give a residue, which was purified by chromatography on silica gel (eluted with petroleum ether/ethyl acetate=20/1 to 1/4) to give Compound 7 (35 g, yield 74.84%) as a yellow solid.

¹H NMR (ET43587-63-P1A, 400 MHz, CHLOROFORM-d) δ 3.03 (s, 3H), 3.80-4.05 (m, 3H), 6.22 (br d, J=5.70 Hz, 1H), 6.86-7.03 (m, 5H), 7.21 (dd, J=8.00, 4.71 Hz, 1H), 7.67-7.75 (m, 2H), 8.00 (br s, 1H), 8.21-8.28 (m, 1H), 8.38 (br d, J=4.17 Hz, 1H)

### General Procedure for Preparation of Compound 8—Notebook Page: ET43587-65.

The reactions were conducted in parallel but combined for purification.

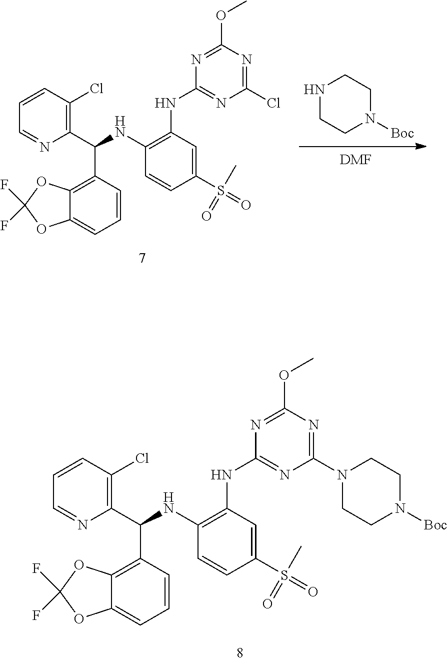

To a solution of Compound 7 (17.5 g, 28.62 mmol, 1 eq) and tert-butyl piperazine-1-carboxylate (6.40 g, 34.35 mmol, 1.2 eq) in DMF (175 mL) was added Et₃N (5.79 g, 57.25 mmol, 2 eq). The mixture was stirred at 90° C. for 2 hrs. LCMS showed the starting material was consumed and a new peak with desired product MS was detected. One additional reaction was set up as detailed above and both two reaction mixtures were combined. The combined reaction mixture was concentrated under reduced pressure to give a residue, which was purified by chromatography on silica gel (eluted with petroleum ether/ethyl acetate=50/1 to 0/1) to give Compound 8 (40 g, yield 82.62%) as a yellow solid.

¹H NMR (ET43587-65-p1a, 400 MHz, CHLOROFORM-d) δ 1.46 (s, 9H), 2.99 (s, 3H), 3.34-3.45 (m, 4H), 3.65 (br s, 2H), 3.76 (br s, 2H), 3.84 (s, 3H), 6.17 (br d, J=6.78 Hz, 1H), 6.78 (br d, J=7.03 Hz, 1H), 6.86-6.99 (m, 4H), 7.17 (dd, J=7.97, 4.71 Hz, 1H), 7.60-7.69 (m, 2H), 8.02 (s, 1H), 8.10 (br s, 1H), 8.37 (br d, J=4.27 Hz, 1H)

### General Procedure for Preparation of Compound 10—Notebook Page: ET43587-6.

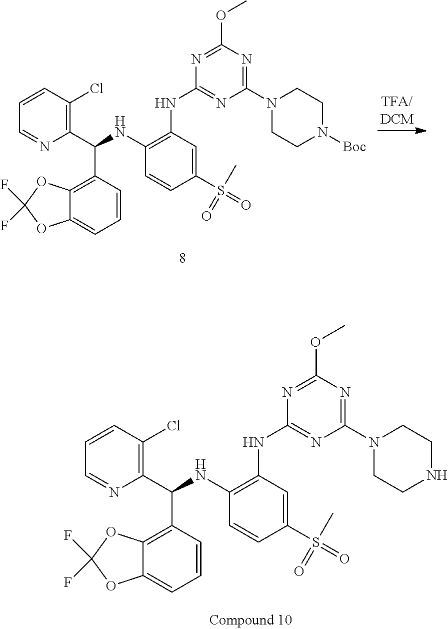 [SMILES: COc1nc(Nc2cc(S(C)(=O)=O)ccc2N[C@@H](c2cccc3c2OC(F)(F)O3)c2ncccc2Cl)nc(N2CCN(C(=O)OC(C)(C)C)CC2)n1 ; COc1nc(Nc2cc(S(C)(=O)=O)ccc2N[C@@H](c2cccc3c2OC(F)(F)O3)c2ncccc2Cl)nc(N2CCNCC2)n1]

To a solution of Compound 8 (400 mg, 525.49 μmol, 1 eq) in DCM (3.5 mL) was added TFA (2.31 g, 20.26 mmol, 38.55 eq). The mixture was stirred at 20° C. for 12 hrs. LCMS showed all the starting material was consumed and a new peak with desired product MS was detected. The reaction mixture was concentrated under reduced pressure to give a residue, which was purified by Prep-HPLC (column: Phenomenex Luna C18 100*30 mm*5 um; mobile phase: [water (0.1% TFA)-ACN]; B %: 25%-55%, 10 mins) to give Compound 10 (300 mg, yield 77.72%) as a white solid.

¹H NMR (ET43587-6-P1J2, 400 MHz, DMSO-d₆) δ 3.02-3.17 (m, 7H), 3.83 (br s, 7H), 6.36 (br d, J=7.25 Hz, 1H), 6.81 (br d, J=8.76 Hz, 1H), 6.95 (br d, J=5.88 Hz, 1H), 7.13-7.19 (m, 1H), 7.20-7.24 (m, 1H), 7.32 (d, J=7.88 Hz, 1H), 7.48 (dd, J=8.07, 4.69 Hz, 2H), 7.92-8.08 (m, 2H), 8.55 (d, J=4.00 Hz, 1H), 8.85 (br s, 2H), 9.45 (br s, 1H)

LCMS (ESI+): RT=2.258 min, m/z 661.1 (M+H)⁺.

5_95AB_6min-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1000.

### Synthesis of A1, A3 and A5

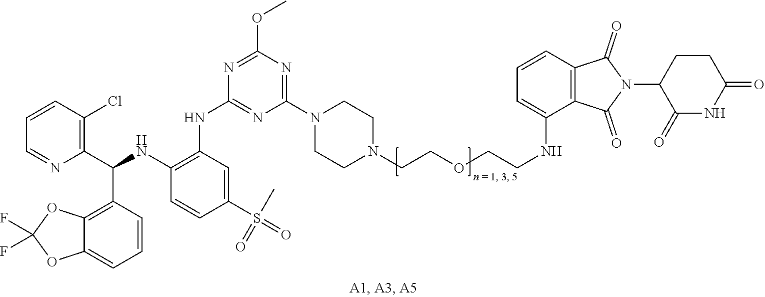

| Compound ID | Synonym | Synonym |
|---|---|---|
| NUCC-0226578 | A1 | ET43318-88 |
| NUCC-0226577 | A3 | ET43587-99 |
| NUCC-0227083 | A5 | ET43587-100 |

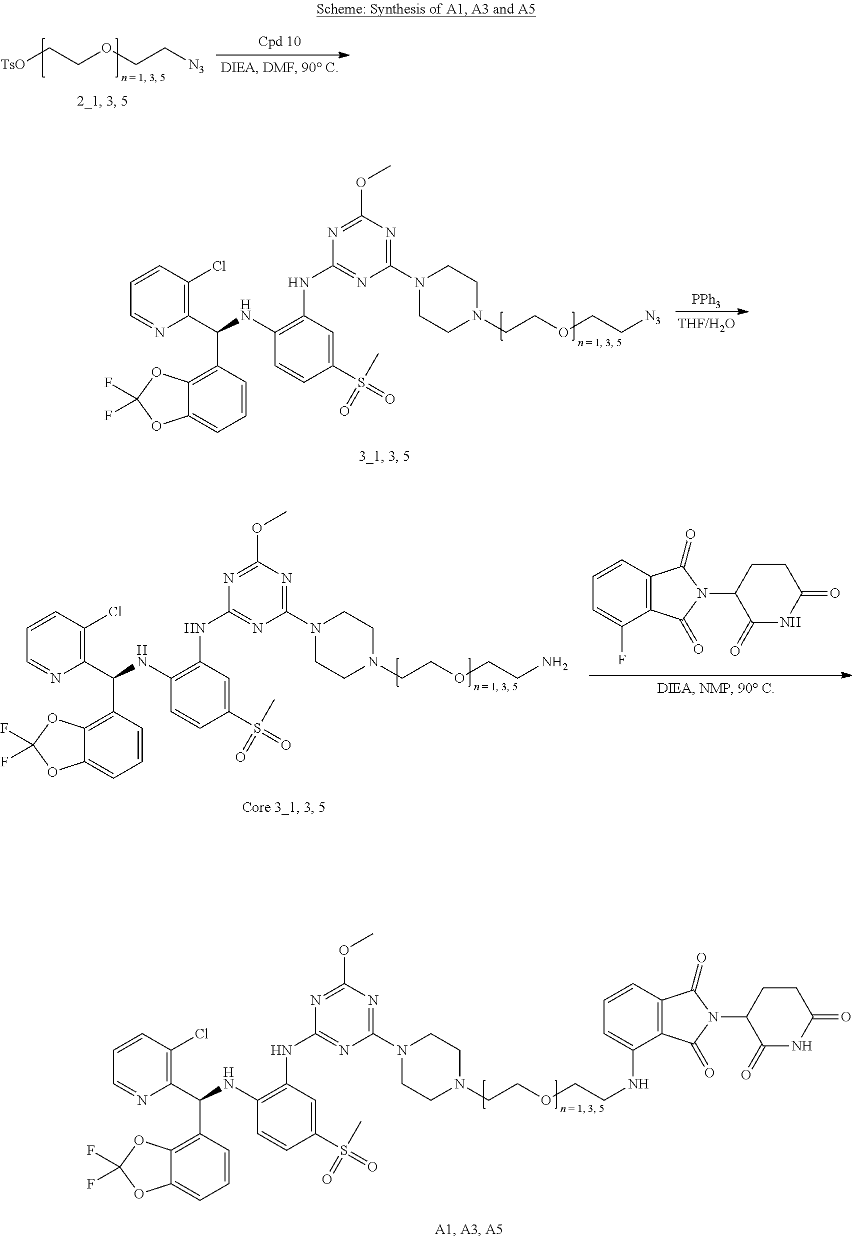

### Chemical Synthesis

All chemical reagents were obtained from commercial suppliers and used without further purification, unless otherwise stated. Reactions were run without taking precautions to exclude air or moisture, unless otherwise noted. Normal phase column chromatography was performed using silica gel columns and ACS grade solvents. Analytical TLC was performed on EM Reagent 0.25 mm silica gel 60 F254 plates and visualized by UV light. Compound identities were confirmed by ¹H and F (NMR) spectroscopy which were recorded on a Bruker 400 MHz spectrometer using the corresponding residual solvent peak (CDCl₃,1H5S=7.27, CD₃OD, 1H δ=3.31; DMSO-d₆, 1H δ=2.50) as an internal standard. The chemical shifts for ¹H-NMR is reported to the second decimal place. Proton coupling constants are expressed in hertz (Hz). Standard abbreviations were used to denote spin multiplicity for ¹H NMR data.

### LCMS Methods:

### Method: 5_95AB_6min-220-MS1500

LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-1500.

### Experimentals for Largest Scale Run:

### General Procedure for Preparation of Compound 3_1—Notebook Page: ET43318-70.

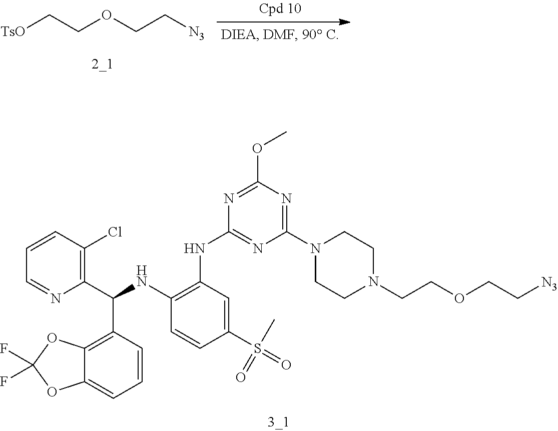 [SMILES: Cc1ccc(S(=O)(=O)OCCOCCN=[N+]=[N-])cc1 ; COc1nc(Nc2cc(S(C)(=O)=O)ccc2N[C@@H](c2cccc3c2OC(F)(F)O3)c2ncccc2Cl)nc(N2CCN(CCOCCN=[N+]=[N-])CC2)n1]

To a solution of Compound 10 (1 g, 1.29 mmol, 1 eq) and Compound 2_1 (736.21 mg, 2.58 mmol, 2 eq) in DMF (10 mL) was added DIEA (500.23 mg, 3.87 mmol, 3 eq) at 20° C. The mixture was heated to 90° C. and stirred for 12 hrs. LCMS showed the starting material was consumed and a new peak (Rt=0.708 min, 66.1%) with desired MS was detected. TLC (ethyl acetate/methanol=5/1, Rf=0.6) showed a new spot generated. The reaction mixture was poured into water (100 mL) and extracted with ethyl acetate (3×30 mL). The organic layers were combined, washed with brine (2×20 mL), dried over Na₂SO₄, filtered and concentrated under reduced pressure to give a residue, which was purified by column chromatography on silica gel (eluting with ethyl acetate/methanol=I/O to 10/1) to give Compound 3_1 (0.6 g, yield 54.06%) as a yellow solid.

¹H NMR (ET43318-70-P1B, 400 MHz, DMSO-d6) δ 2.39 (br s, 4H), 3.06 (s, 3H), 3.36-3.40 (m, 2H), 3.41-3.75 (m, 10H), 3.79 (br s, 3H), 6.34 (br d, J=7.00 Hz, 1H), 6.81 (br d, J=8.50 Hz, 1H), 6.97 (br d, J=6.75 Hz, 1H), 7.10-7.16 (m, 1H), 7.22 (br d, J=7.75 Hz, 1H), 7.31 (br d, J=7.75 Hz, 1H), 7.44-7.53 (m, 2H), 7.92 (br s, 1H), 8.01 (br d, J=8.00 Hz, 1H), 8.53 (br d, J=4.25 Hz, 1H), 9.27 (br s, 1H)

### General Procedure for Preparation of Compound Core 3_1—Notebook Page: ET43318-79.

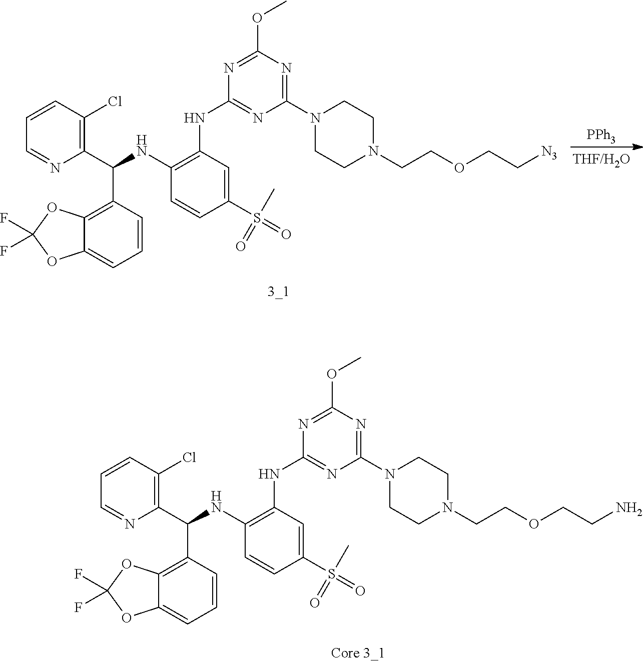 [SMILES: COc1nc(Nc2cc(S(C)(=O)=O)ccc2N[C@@H](c2cccc3c2OC(F)(F)O3)c2ncccc2Cl)nc(N2CCN(CCOCCN=[N+]=[N-])CC2)n1 ; COc1nc(Nc2cc(S(C)(=O)=O)ccc2N[C@@H](c2cccc3c2OC(F)(F)O3)c2ncccc2Cl)nc(N2CCN(CCOCCN)CC2)n1 ; "PPh3"]

To a solution of Compound 3_1 (240 mg, 310.00 μmol, 1 eq) in THF (3 mL) were added PPh₃ (121.96 mg, 465.00 μmol, 2 eq) and H₂O (55.85 mg, 3.10 mmol, 10 eq) at 20° C. The mixture was heated to 60° C. and stirred for 12 hrs. LCMS showed the starting material was consumed and a new peak (Rt=0.622 min, 46.1%) with desired MS was detected. TLC (ethyl acetate/methanol=0/1, Rf=0.1) showed a new spot generated. The reaction mixture was concentrated under reduced pressure to give a residue, which was diluted with DCM (15 mL), poured into HCl solution (0.5 N, 30 mL) and extracted with DCM (2×10 mL). Then to the aqueous was added Na₂CO₃ and basified pH=10. The aqueous was extracted with ethyl acetate (3×15 mL). The organic layers were combined, washed with brine (2×10 mL), dried over Na₂SO₄, filtered and concentrated under reduced pressure to give Compound Core 3_1 (200 mg, yield 77.61%) as a white solid, which was used to the next step directly without further purification.

¹H NMR (ET43318-79-P1A, 400 MHz, DMSO-d6) δ2.31-2.48 (m, 6H), 2.65 (br t, J=5.57 Hz, 2H), 3.06 (s, 3H), 3.35 (br t, J=5.69 Hz, 3H), 3.49 (br t, 0.1=5.50 Hz, 3H), 3.67 (br s, 2H), 3.79 (br s, 3H), 6.34 (d, J=7.25 Hz, 1H), 6.80 (d, J=8.88 Hz, 1H), 6.97 (br d, J=7.13 Hz, 1H), 7.11-7.18 (m, 1H), 7.20-7.25 (m, 1H), 7.28-7.34 (m, 1H), 7.47 (dd, J=8.00, 4.75 Hz, 2H), 7.93 (br s, 1H), 8.02 (dd, J=8.13, 1.25 Hz, 1H), 8.53 (dd, J=4.63, 1.13 Hz, 1H)

### General Procedure for Preparation of A1—Notebook Page: ET43318-88.

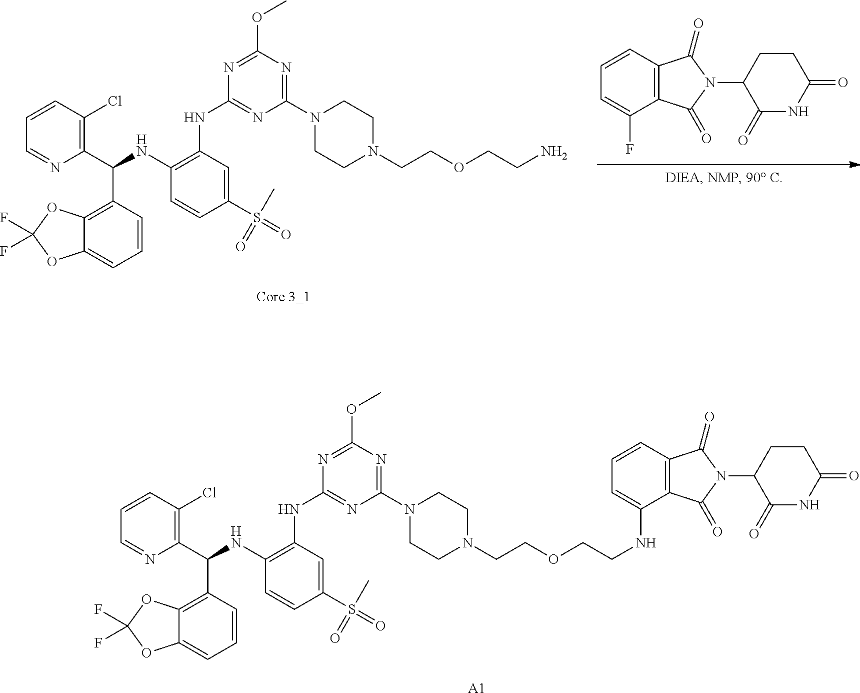 [SMILES: COc1nc(Nc2cc(S(C)(=O)=O)ccc2N[C@@H](c2cccc3c2OC(F)(F)O3)c2ncccc2Cl)nc(N2CCN(CCOCCN)CC2)n1 ; O=C1CCC(N2C(=O)c3cccc(F)c3C2=O)C(=O)N1 ; COc1nc(Nc2cc(S(C)(=O)=O)ccc2N[C@@H](c2cccc3c2OC(F)(F)O3)c2ncccc2Cl)nc(N2CCN(CCOCCNc3cccc4c3C(=O)N(C3CCC(=O)NC3=O)C4=O)CC2)n1 ; "A1"]

To a solution of 2-(2,6-dioxo-3-piperidyl)-4-fluoro-isoindoline-1,3-dione (81.59 mg, 295.38 μmol, 1.3 eq) and Compound Core 31 (170 mg, 227.21 μmol, 1 eq) in NMP (1.5 mL) was added DIEA (88.10 mg, 681.64 μmol, 2.31 eq) at 20° C. The mixture was heated to 90° C. and stirred for 12 hrs. LCMS showed the starting material was consumed and a new peak (Rt=0.719 min, 54.1%) with desired MS was detected. The mixture purified by Prep-HPLC (column: Waters Xbridge BEH C18 100*30 mm*10 um; mobile phase: [water (NH₄HCO₃)-ACN]; B %: 35%-65%, 8 mins) to give A1 (26.5 mg, yield 11.07%) as a yellow solid.

¹H NMR (ET43318-88-P1A, 400 MHz, DMSO-d₆) δ 1.94-2.12 (m, 1H), 2.52-2.75 (m, 5H), 2.82-2.92 (m, 1H), 2.98-3.16 (m, 5H), 3.34-3.47 (m, 2H), 3.54 (br s, 3H), 3.64 (br d, J=4.63 Hz, 2H), 3.71-4.00 (m, 5H), 4.33-4.71 (m, 2H), 4.98-5.11 (m, 1H), 6.36 (s, 1H), 6.50-6.70 (m, 2H), 6.81 (br d, J=8.76 Hz, 1H), 7.04 (br d, J=7.00 Hz, 1H), 7.11-7.25 (m, 3H), 7.32 (br d, J=8.50 Hz, 1H), 7.47 (br dd, J=7.88, 4.88 Hz, 2H), 7.53-7.62 (m, 1H), 8.00-8.06 (m, 1H), 8.47-8.64 (m, 1H), 9.27-9.66 (m, 1H), 11.09 (s, 1H), 11.15-11.50 (m, 1H)

LCMS (ESI+): RT=2.421 min, m/z 502.7, 1004.2 (M/2+H, M+H)⁺.

5_95AB_6min-220-MS1500: LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-1500.

### General Procedure for Preparation of Compound 3_3—Notebook Page: ET43318-74.

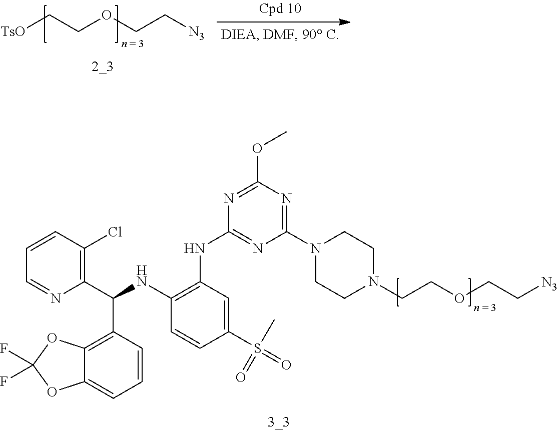

To a solution of Compound 10 (1.8 g, 2.32 mmol, 1 eq) and Compound 2_3 (1.30 g, 3.48 mmol, 1.5 eq) in DMF (15 mL) was added DIEA (900.41 mg, 6.97 mmol, 1.21 mL, 3 eq). The mixture was heated to 90° C. and stirred for 12 hrs. LCMS showed the starting material was consumed and 63% peak (Rt=0.726 min) with desired MS was detected. The reaction mixture was poured into water (150 mL) and extracted with ethyl acetate (3×40 mL). The organic layers were combined, washed with brine (2×20 mL), dried over Na₂SO₄, filtered and concentrated under reduced pressure to give a residue, which was purified by column chromatography on silica gel (eluting with ethyl acetate/methanol=I/O to 10/1) to give Compound 33 (1.05 g, yield 47.19%) as a yellow solid.

¹H NMR (ET43318-74-P1A, 400 MHz, DMSO-d₆) δ 2.38 (br s, 4H), 3.06 (s, 3H), 3.38 (br t, J=4.75 Hz, 2H), 3.45-3.73 (m, 18H), 3.79 (br s, 3H), 6.34 (br d, J=7.25 Hz, 1H), 6.81 (br d, J=8.76 Hz, 1H), 6.96 (br d, J=7.00 Hz, 1H), 7.11-7.17 (m, 1H), 7.19-7.25 (m, 1H), 7.31 (br d, J=7.88 Hz, 1H), 7.42-7.51 (m, 2H), 7.93 (br s, 1H), 8.02 (br d, J=8.00 Hz, 1H), 8.53 (br d, J=4.50 Hz, 1H), 9.25 (br s, 1H)

### General Procedure for Preparation of Compound Core 3_3—Notebook Page: ET43318-83.

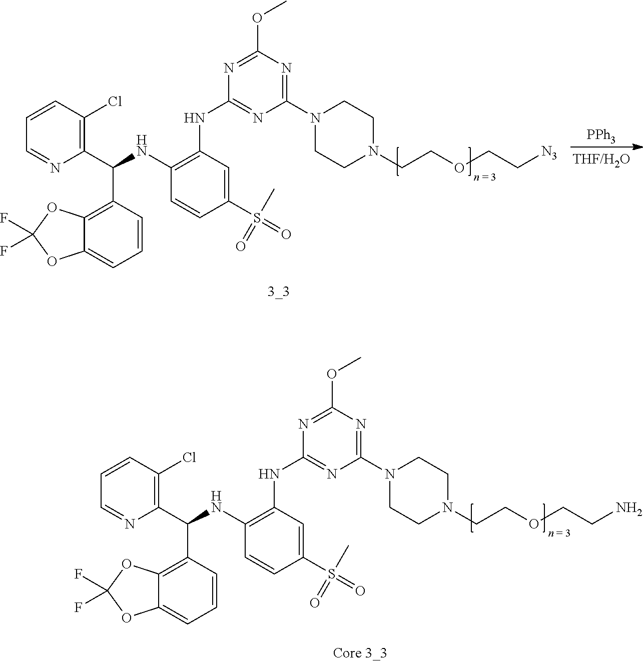

To a solution of Compound 33 (0.9 g, 1.04 mmol, 1 eq) in THF (15 mL) were added PPh₃ (355.88 mg, 1.36 mmol, 1.3 eq) and H₂O (188.08 mg, 10.44 mmol, 188.08 μL, 10 eq) at 20° C. The mixture was heated to 60° C. and stirred for 12 hrs. LCMS showed the starting material was consumed and 56.4% peak (Rt=0.620 min) with desired MS was detected. The reaction mixture was concentrated under reduced pressure to give a residue, which was diluted with DCM (50 mL), poured into HCl solution (0.5 N, 150 mL) and extracted with DCM (2×30 mL). Then to the aqueous was added Na₂CO₃ and basified pH=10. The aqueous was extracted with ethyl acetate (3×50 mL). The organic layers were combined, washed with brine (2×30 mL), dried over Na₂SO₄, filtered and concentrated under reduced pressure to give Compound Core 33 (700 mg, yield 72.18%) as a white solid, which was used to next step directly without further purification.

¹H NMR (ET43318-83-P1A, 400 MHz, DMSO-d₆) δ 2.30-2.47 (m, 6H), 2.66 (br t, J=5.57 Hz, 2H), 3.06 (s, 3H), 3.36 (t, 0.1=5.75 Hz, 2H), 3.50 (br d, J=2.63 Hz, 12H), 3.67 (br s, 2H), 3.79 (br s, 3H), 6.34 (d, J=7.25 Hz, 1H), 6.80 (d, J=8.88 Hz, 1H), 6.96 (br d, J=7.13 Hz, 1H), 7.11-7.17 (m, 1H), 7.19-7.25 (m, 1H), 7.31 (dd, J=7.88, 0.75 Hz, 1H), 7.43-7.53 (m, 2H), 7.94 (br d, J=10.63 Hz, 1H), 8.02 (dd, J=8.07, 1.19 Hz, 1H), 8.53 (dd, J=4.63, 1.13 Hz, 1H)

### General Procedure for Preparation of A3—Notebook Page: ET43587-99.

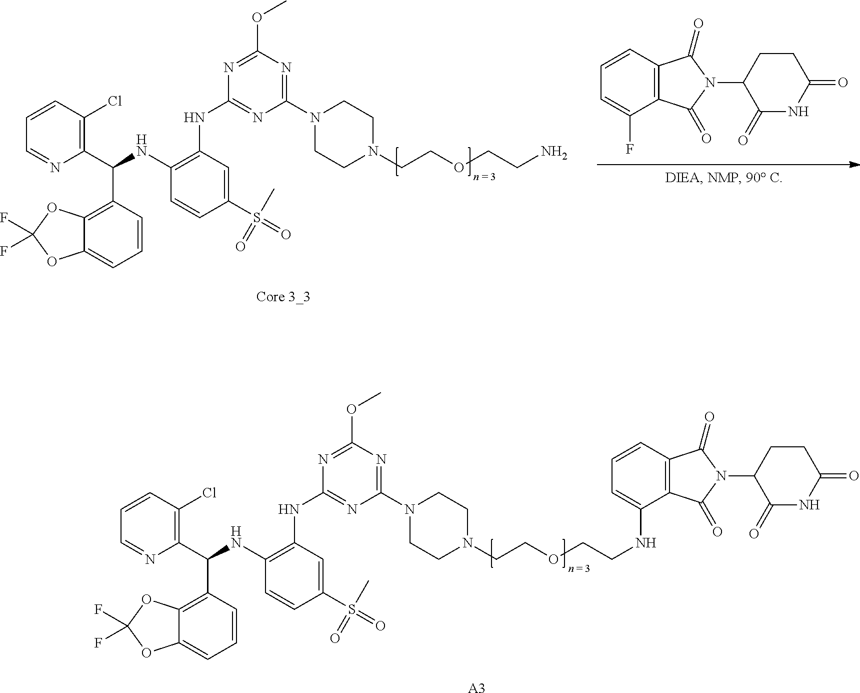

To a solution of 2-(2,6-dioxo-3-piperidyl)-4-fluoro-isoindoline-1,3-dione (59.76 mg, 216.35 μmol, 1 eq) and Compound Core 3_3 (200 mg, 216.35 μmol, 1 eq) in NMP (4 mL) was added DIEA (55.92 mg, 432.71 μmol, 2 eq). The mixture was stirred at 90° C. for 12 hrs. LCMS showed all the starting material was consumed and a new peak with desired product MS was detected. The mixture was purified by Prep-HPLC (column: Phenomenex Luna 80*30 mm*3 um; mobile phase: [water (0.1% TFA)-ACN]; B %: 30%-55%, 8 mins) to give A3 (68.5 mg, yield 26.39%) as a yellow solid.

¹H NMR (ET43587-99-P1A, 400 MHz, DMSO-d6) δ 1.98-2.10 (m, 1H), 2.57 (br s, 1H), 2.62 (br s, 1H), 2.89 (ddd, J=17.04, 13.66, 5.32 Hz, 1H), 2.97-3.16 (m, 5H), 3.31 (br s, 3H), 3.39-3.90 (m, 19H), 4.30-4.77 (m, 3H), 5.07 (dd, J=12.76, 5.38 Hz, 1H), 6.38 (br d, J=7.13 Hz, 1H), 6.58 (br s, 1H), 6.82 (br d, J=8.76 Hz, 1H), 6.93-7.07 (m, 2H), 7.08-7.21 (m, 2H), 7.21-7.28 (m, 1H), 7.34 (dd, J=7.88, 1.00 Hz, 1H), 7.46-7.61 (m, 3H), 8.05 (dd, J=8.13, 1.38 Hz, 2H), 8.57 (br d, J=3.88 Hz, 1H), 9.44-9.63 (m, 1H), 9.79-9.98 (m, 1H), 11.13 (s, 1H)

LCMS (ESI+): RT=2.443 min, m/z 546.7, 1092.2 (M/2+H, M+H)⁺.

5_95AB_6min-220-MS1500: LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-1500.

### General Procedure for Preparation of Compound 3_5—Notebook Page: ET43318-75.

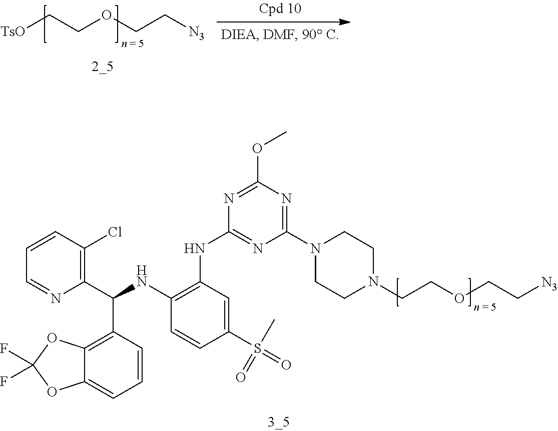

To a solution of Compound 10 (1.8 g, 2.32 mmol, 1 eq, TFA) and Compound 2_5 (1.61 g, 3.48 mmol, 1.5 eq) in DMF (15 mL) was added DIEA (900.41 mg, 6.97 mmol, 1.21 mL, 3 eq) at 20° C. The mixture was heated to 90° C. and stirred for 12 hrs. LCMS showed the starting material was consumed and 64.6% peak (Rt=0.727 min) with desired MS was detected. The reaction mixture was poured into water (150 mL) and extracted with ethyl acetate (3×40 mL). The organic layers were combined, washed with brine (2×20 mL), dried over Na₂SO₄, filtered and concentrated under reduced pressure to give a residue, which was purified by column chromatography on silica gel (eluting with ethyl acetate/methanol=I/O to 5/1) to give Compound 3_5 (1.12 g, yield 45.67%) as a yellow solid.

¹H NMR (ET43318-75-P1A, 400 MHz, DMSO-d₆) δ 2.40 (br d, J=15.88 Hz, 4H), 3.06 (s, 3H), 3.38 (br t, J=4.75 Hz, 2H), 3.44-3.71 (m, 26H), 3.79 (br s, 3H), 6.34 (br d, J=7.25 Hz, 1H), 6.80 (d, J=8.76 Hz, 1H), 6.96 (br d, J=7.00 Hz, 1H), 7.10-7.17 (m, 1H), 7.19-7.24 (m, 1H), 7.31 (d, J=7.75 Hz, 1H), 7.43-7.52 (m, 2H), 7.92 (br s, 1H), 8.02 (d, J=8.13 Hz, 1H), 8.53 (d, J=4.38 Hz, 1H), 9.25 (br s, 1H)

### General Procedure for Preparation of Compound Core 3_5—Notebook Page: ET43318-84.

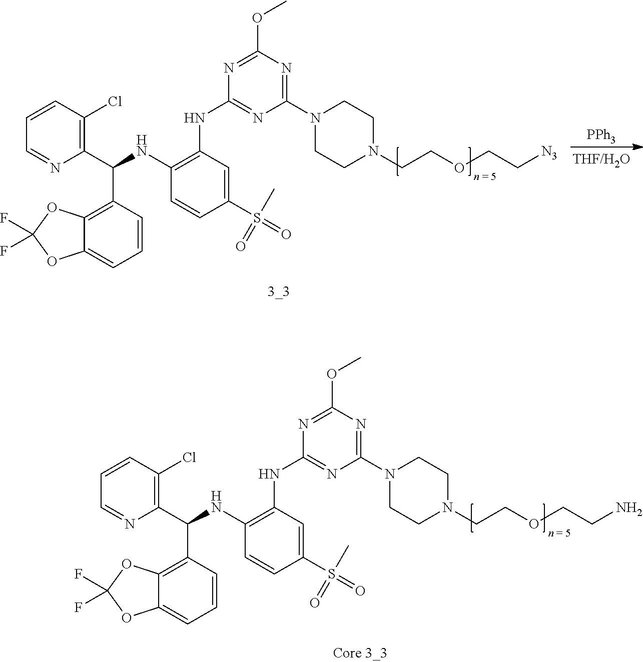

To a solution of Compound 35 (1 g, 1.05 mmol, 1 eq) in THF (15 mL) were added PPh₃ (358.77 mg, 1.37 mmol, 1.3 eq) and H₂O (189.60 mg, 10.52 mmol, 189.60 μL, 10 eq) at 20° C. The mixture was heated to 60° C. and stirred for 12 hrs. LCMS showed the starting material was consumed and 56.5% peak (Rt=0.625 min) with desired MS was detected. The reaction mixture was concentrated under reduced pressure to give a residue, which was diluted with DCM (50 mL), poured into HCl solution (0.5 N, 150 mL) and extracted with DCM (3×20 mL). Then to the aqueous was added Na₂CO₃ and basified pH=10. The aqueous was extracted with ethyl acetate (3×50 mL). The organic layers were combined, washed with brine (2×20 mL), dried over Na₂SO₄, filtered and concentrated under reduced pressure to give Compound Core 3_5 (750 mg, yield 71.71%) as an off-white solid, which was used to the next step directly without further purification.

¹H NMR (ET43318-84-P1A, 400 MHz, DMSO-d₆) δ 2.29-2.48 (m, 6H), 2.66 (br t, J=5.44 Hz, 2H), 3.06 (s, 3H), 3.36 (t, J=5.75 Hz, 2H), 3.50 (s, 20H), 3.67 (br s, 2H), 3.79 (br s, 3H), 6.34 (d, J=7.13 Hz, 1H), 6.81 (d, J=8.76 Hz, 1H), 6.97 (br d, J=7.13 Hz, 1H), 7.11-7.18 (m, 1H), 7.19-7.25 (m, 1H), 7.31 (d, J=7.88 Hz, 1H), 7.43-7.52 (m, 2H), 7.93 (br s, 1H), 8.02 (d, J=8.00 Hz, 1H), 8.53 (d, J=4.50 Hz, 1H)

### General Procedure for Preparation of A5—Notebook Page: ET43587-100.

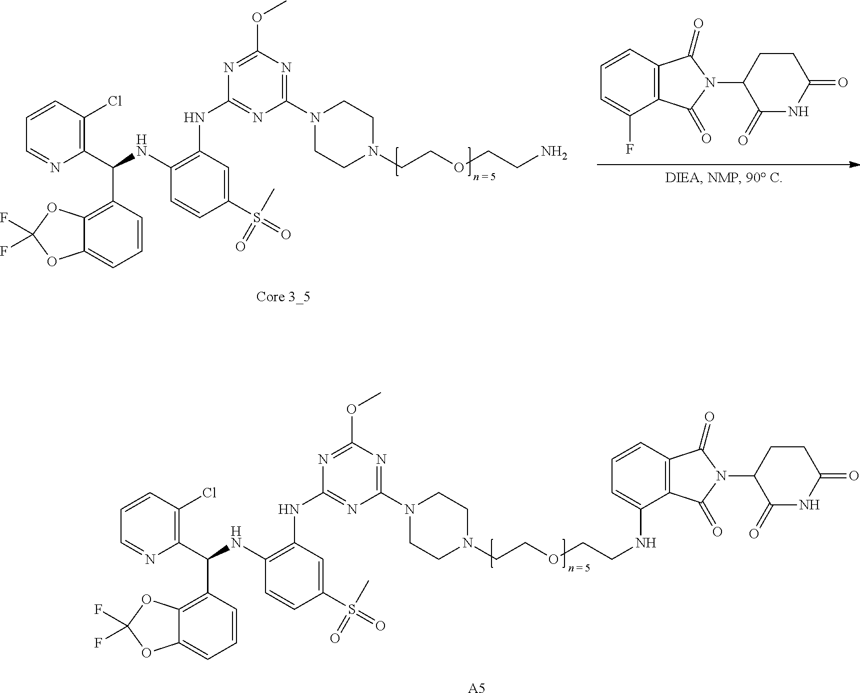

To a solution of 2-(2,6-dioxo-3-piperidyl)-4-fluoro-isoindoline-1,3-dione (59.76 mg, 216.35 μmol, 1 eq) and Compound Core 3_5 (200 mg, 216.35 μmol, 1 eq) in NMP (4 mL) was added DIEA (55.92 mg, 432.71 μmol, 2 eq). The mixture was stirred at 90° C. for 12 hrs. LCMS showed all the starting material was consumed and a new peak with desired product MS was detected. The mixture was purified by Prep-HPLC (column: Phenomenex Luna 80*30 mm*3 um; mobile phase: [water (0.1% TFA)-ACN]; B %: 30%-55%, 8 mins) to give A5 (68.5 mg, yield 26.39%) as a yellow solid.

¹H NMR (ET43587-100-P1A, 400 MHz, DMSO-d6) δ 1.97-2.07 (m, 1H), 2.56 (br s, 1H), 2.60 (br s, 1H), 2.82-2.94 (m, 1H), 3.07 (s, 5H), 3.31 (br s, 3H), 3.39-3.94 (m, 27H), 4.31-4.75 (m, 3H), 5.05 (dd, J=12.88, 5.38 Hz, 1H), 6.36 (br d, J=7.25 Hz, 1H), 6.59 (br s, 1H), 6.81 (br d, J=8.63 Hz, 1H), 6.98 (br d, J=8.88 Hz, 1H), 7.04 (d, J=7.00 Hz, 1H), 7.10-7.18 (m, 2H), 7.20-7.26 (m, 1H), 7.30-7.35 (m, 1H), 7.48 (dd, J=8.07, 4.69 Hz, 2H), 7.57 (dd, J=8.44, 7.19 Hz, 1H), 8.04 (dd, J=8.13, 1.25 Hz, 2H), 8.55 (br d, J=3.50 Hz, 1H), 9.40-9.60 (m, 1H), 9.75-9.96 (m, 1H), 11.11 (s, 1H)

LCMS (ESI+): RT=2.467 min, m/z 590.8, 1180.3 (M/2+H, M+H)⁺.

5_95AB_6min-220-MS1500: LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-1500.

### Synthesis of M1 and C1

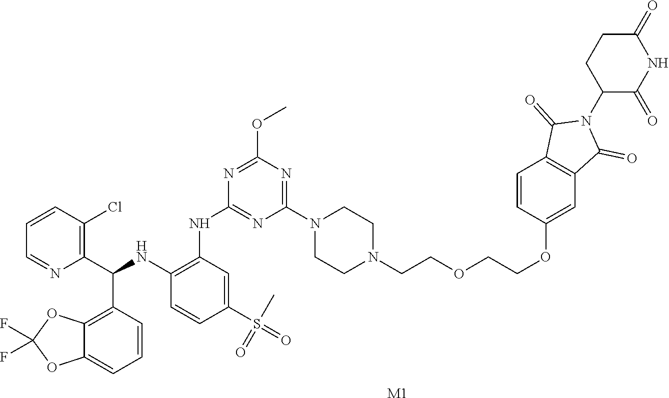 [SMILES: COc1nc(Nc2cc(S(C)(=O)=O)ccc2N[C@@H](c2cccc3c2OC(F)(F)O3)c2ncccc2Cl)nc(N2CCN(CCOCCOc3ccc4c(c3)C(=O)N(C3CCC(=O)NC3=O)C4=O)CC2)n1 ; "M1"]

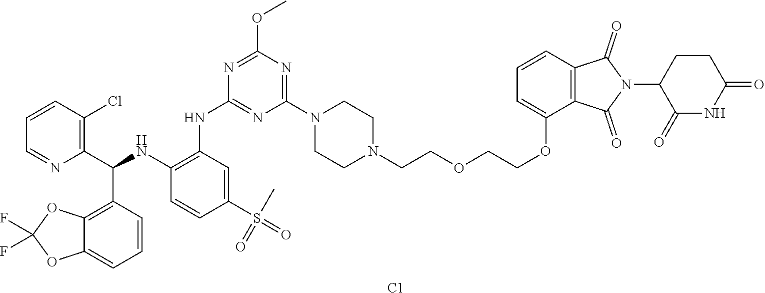 [SMILES: COc1nc(Nc2cc(S(C)(=O)=O)ccc2N[C@@H](c2cccc3c2OC(F)(F)O3)c2ncccc2Cl)nc(N2CCN(CCOCCOc3cccc4c3C(=O)N(C3CCC(=O)NC3=O)C4=O)CC2)n1 ; "C1"]

| Compound ID | Synonym | Synonym |
|---|---|---|
| NUCC-0226564 | M1 | ET43587-89 |
| NUCC-0226594 | C1 | ET43587-167 |

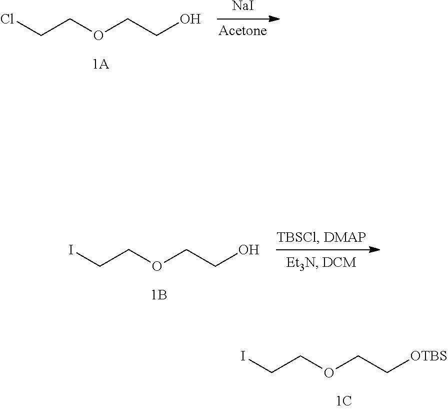

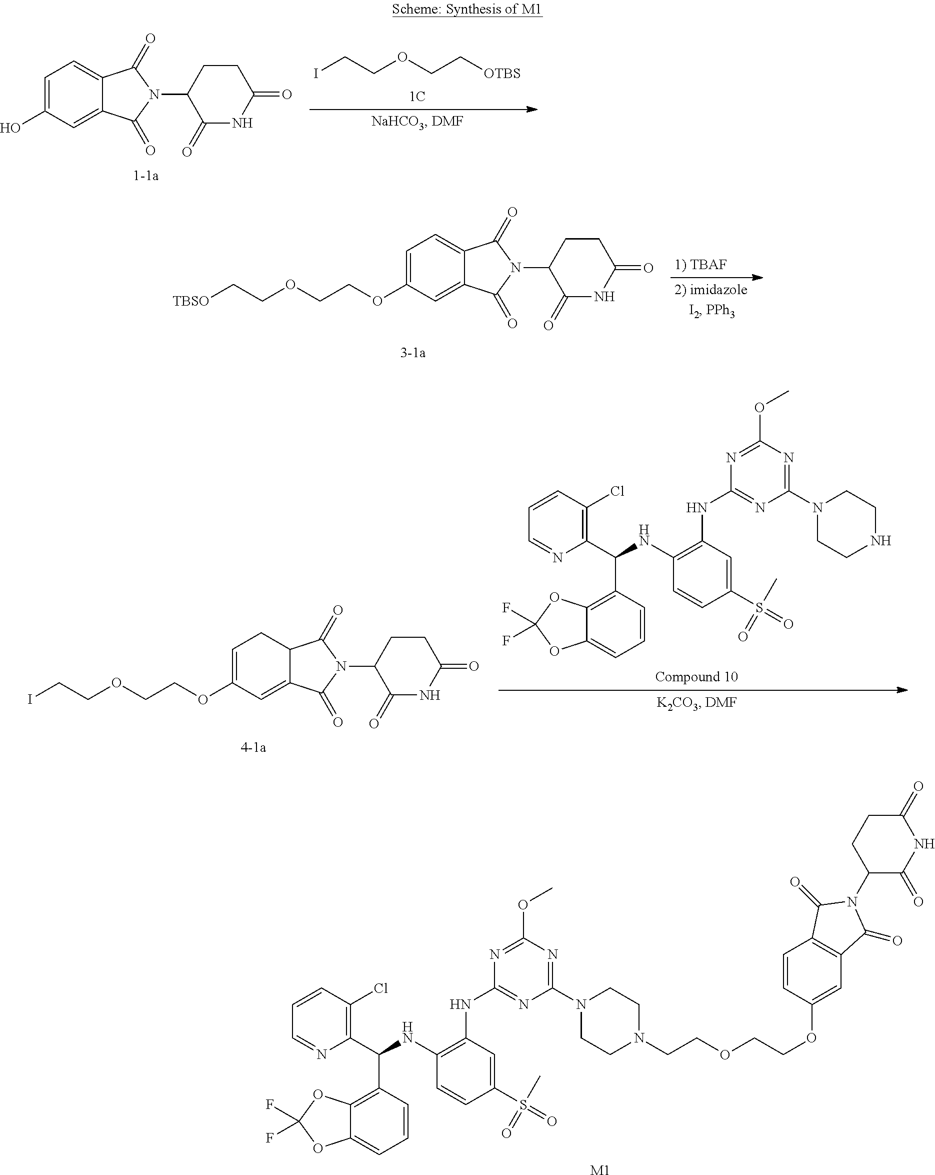

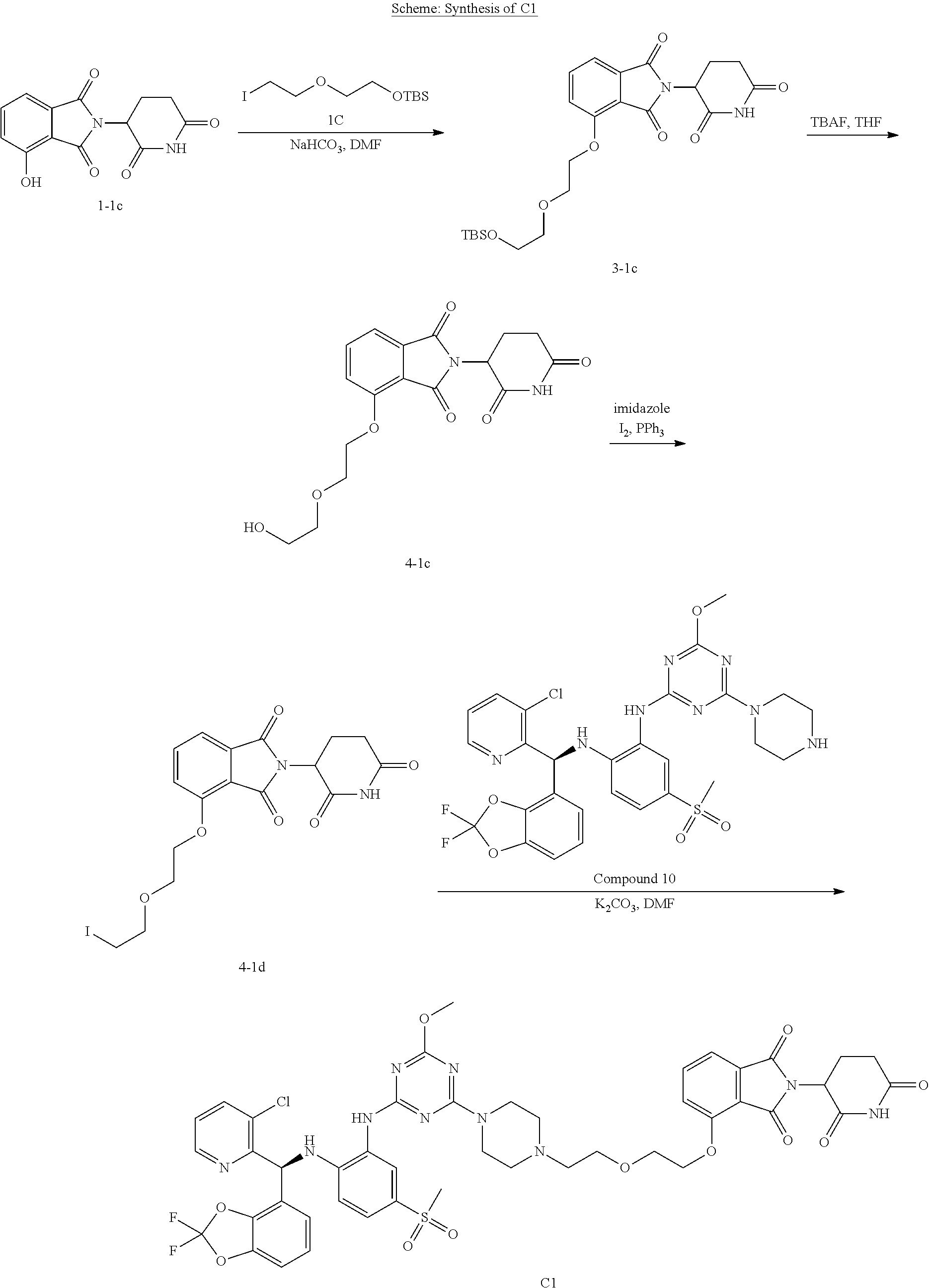

### Chemical Synthesis

All chemical reagents were obtained from commercial suppliers and used without further purification, unless otherwise stated. Reactions were run without taking precautions to exclude air or moisture, unless otherwise noted. Normal phase column chromatography was performed using silica gel columns and ACS grade solvents. Analytical TLC was performed on EM Reagent 0.25 mm silica gel 60 F254 plates and visualized by UV light. Compound identities were confirmed by ¹H and F (NMR) spectroscopy which were recorded on a Bruker 400 MHz spectrometer using the corresponding residual solvent peak (CDCl₃, 1H δ=7.27; CD₃OD, 1H δ=3.31; DMSO-d₆, 1H δ=2.50) as an internal standard. The chemical shifts for ¹H-NMR is reported to the second decimal place. Proton coupling constants are expressed in hertz (Hz). Standard abbreviations were used to denote spin multiplicity for ¹H NMR data.

### LCMS Methods:

### Method 1: 5_95AB 2 Min

LC/MS (The column used for chromatography was a Kinetex EVO C18 2.1*30 mm, 5 um.

Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### Method 2: 5_95AB_6min-ELSD-MS1500

LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1500.

### Method 3: 5_95AB_6min-220-MS1500

LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-1500.

### Experimentals for Largest Scale Run:

### General Procedure for Preparation of Compound 1B-Notebook Page: ET43587-39.

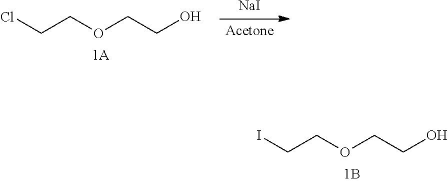 [SMILES: "NaI" ; OCCOCCCl ; "Acetone" ; OCCOCCI]

To a solution of Compound 1A (10 g, 80.28 mmol, 1 eq) in acetone (135 mL) was added NaI (60.17 g, 401.39 mmol, 5 eq). The mixture was stirred at 65° C. for 12 hrs. TLC (petroleum ether/ethyl acetate=1/1) showed the starting material was consumed and a new spot was generated. The reaction mixture was diluted with water (100 mL) and extracted with ethyl acetate (3×80 mL). The organic layer was separated and the combined organic layer was washed with brine (100 mL), dried over Na₂SO₄, filtered and concentrated under reduced pressure to give a residue, which was purified by chromatography on silica gel (eluted with petroleum ether/ethyl acetate=50/1 to 3/1) to give Compound 1B (13 g, yield 71.22%) as a red oil.

¹H NMR (ET43587-39-PIA, 400 MHz, CHLOROFORM-d) δ 2.12 (s, 1H), 3.29 (t, J=6.57 Hz, 2H), 3.60-3.65 (m, 2H), 3.73-3.79 (m, 4H)

### General Procedure for Preparation of Compound 1C-Notebook Page: ET42365-108.

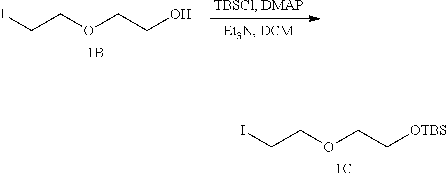

To a solution of Compound 1B (7 g, 32.40 mmol, 1 eq) and TEA (4.92 g, 48.61 mmol, 6.77 mL, 1.5 eq) in DCM (100 mL) were added DMAP (395.89 mg, 3.24 mmol, 0.1 eq) and tert-butyl-chloro-dimethyl-silane (5.86 g, 38.89 mmol, 4.76 mL, 1.2 eq) dropwise at 0° C., the reaction was stirred at 20° C. for 12 hrs. LCMS showed the starting material was consumed and a new peak with desired product MS was detected. The reaction mixture was diluted with water (200 mL) and extracted with DCM (3×100 mL). The organic layer was separated and the combined organic layer was washed with brine (100 mL), dried over Na₂SO₄, filtered and concentrated under reduced pressure to give a residue, which was purified by column chromatography on silica gel (eluted with petroleum ether/ethyl acetate=100/1 to 15/1) to give Compound 1C (9 g, yield 79.89/a) as a colorless oil.

¹H NMR (ET42365-108-P1A, 400 MHz, CHLOROFORM-d) δ 0.08 (s, 6H), 0.91 (s, 9H), 3.26 (t, J=6.94 Hz, 2H), 3.56-3.61 (m, 2H), 3.73-3.82 (m, 4H)

General Procedure for Preparation of Compound 3-1a-Notebook Page: ET43587-78.

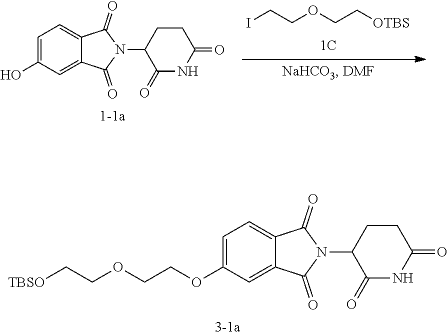

To a solution of Compound 1C (240.88 mg, 729.32 μmol, 1 eq) and Compound 1-1a (200 mg, 729.32 μmol, 1 eq) in DMF (4 mL) was added NaHCO₃ (122.54 mg, 1.46 mmol, 2 eq). The mixture was stirred at 50° C. for 4 hrs. LCMS showed all the starting material was consumed and a new peak with desired product MS was detected. The reaction mixture was diluted with water (10 mL) and extracted with ethyl acetate (3×10 mL). The organic layer was separated and the combined organic layer was washed with brine (2×10 mL), dried over Na₂SO₄, filtered and concentrated under reduced pressure to give Compound 3-1a (350 mg, crude) as a green oil, which was used to the next step directly without further purification.

LCMS (ESI+): RT=0.827 min, m/z 477.3 (M+H)⁺.

5_95AB_2 min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

General Procedure for Preparation of 4-1a-Notebook Page: ET43587-81, 87.

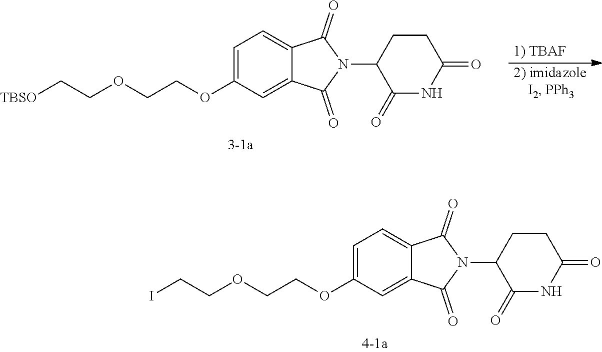

To a solution of Compound 3-1a (350 mg, 734.38 μmol, 1 eq) in THF (5 mL) was added TBAF (I M, 4.41 mL, 6 eq) dropwise at 0° C. The mixture was stirred at 20° C. for 12 hrs. LCMS showed the starting material was consumed and a new peak with desired product MS was detected. The reaction mixture was diluted with water (20 mL) and extracted with ethyl acetate (3×20 mL). The organic layer was separated and the combined organic layer was dried over Na₂SO₄, filtered and concentrated under reduced pressure to give a residue, which was purified by Prep-TLC (petroleum ether/tetrahydrofuran=1/1) to give 2-(2,6-dioxo-3-piperidyl)-5-[2-(2-hydroxyethoxy)ethoxy]isoindoline-1,3-dione (100 mg, yield 33.82%) as a green oil.

¹H NMR (ET43587-81-p1a, 400 MHz, CHLOROFORM-d) δ 1.69 (br s, 1H), 2.14-2.18 (m, 1H), 2.68-2.95 (m, 3H), 3.65-3.74 (m, 2H), 3.79 (br s, 2H), 3.88-3.95 (m, 2H), 4.22-4.33 (m, 2H), 4.92-5.01 (m, 1H), 7.24 (dd, J=8.25, 2.25 Hz, 1H), 7.39 (d, J=2.25 Hz, 1H), 7.79 (d, J=8.38 Hz, 1H), 8.31 (brs, 1H)

To a solution of 2-(2,6-dioxo-3-piperidyl)-5-[2-(2-hydroxyethoxy)ethoxy]isoindoline-1,3-dione (100 mg, 275.99 μmol, 1 eq), 12 (175.12 mg, 689.97 μmol, 2.5 eq) and imidazole (46.97 mg, 689.97 μmol, 2.5 eq) in DCM (2 mL) was added PPh₃ (180.97 mg, 689.97 μmol, 2.5 eq) at 0° C. The mixture was stirred at 20° C. for 12 hrs. LCMS showed all the starting material was consumed and a new peak with desired product MS was detected. The reaction mixture was diluted with water (10 mL) and extracted with dichloromethane (3-10 mL). The organic layer was separated and the combined organic layer was washed with brine (20 mL), dried over Na₂SO₄, filtered and concentrated under reduced pressure to give a residue, which was purified by Prep-TLC (petroleum ether/tetrahydrofuran=1/1) to give Compound 4-1a (100 mg, yield 72.89%) as a white solid.

¹H NMR (ET43587-87-P1A, 400 MHz, CHLOROFORM-d) δ 2.11-2.21 (m, 1H), 2.69-2.96 (m, 3H), 3.29 (t, 0.1=6.75 Hz, 2H), 3.84 (t, 0.1=6.75 Hz, 2H), 3.92 (br dd, J=5.25, 3.88 Hz, 2H), 4.23-4.33 (m, 2H), 4.97 (dd, J=12.32, 5.32 Hz, 1H), 7.25 (dd, J=8.32, 2.31 Hz, 1H), 7.38 (d, J=2.25 Hz, 1H), 7.80 (d, J=8.38 Hz, 1H), 8.16 (br s, 1H)

### General Procedure for Preparation of M1—Notebook Page: ET43587-89.

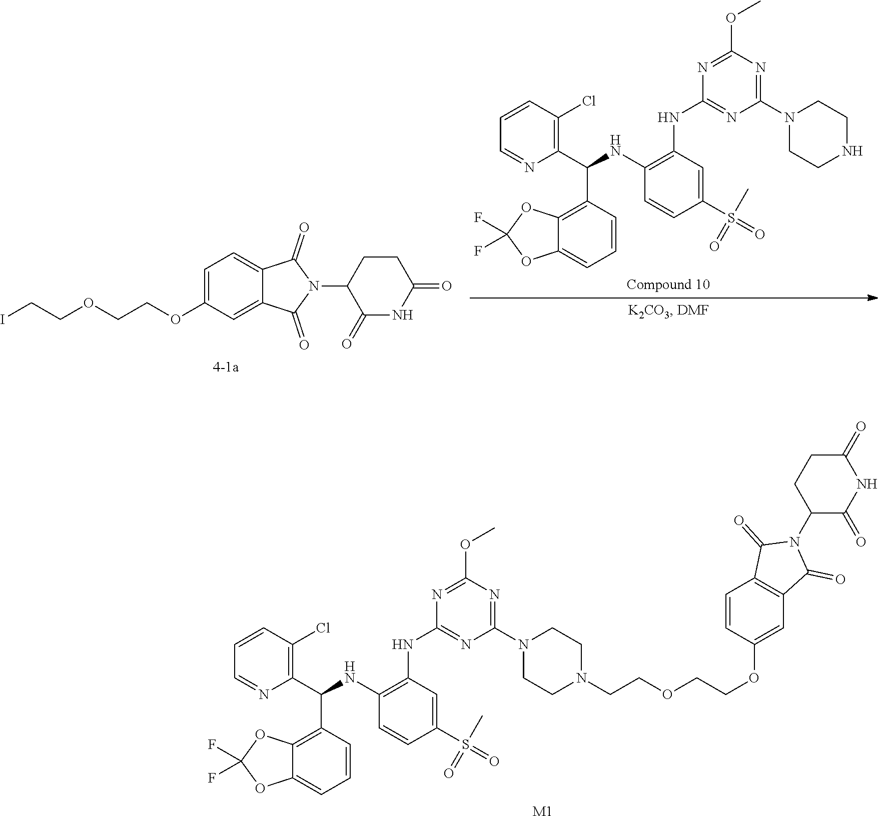

To a solution of Compound 10 (164.14 mg, 211.76 μmol, 1 eq) and Compound 4-1a (100 mg, 211.76 μmol, 1 eq) in DMF (2 mL) was added K₂CO₃ (146.34 mg, 1.06 mmol, 5 eq). The mixture was stirred at 60° C. for 12 hrs. LCMS showed all the starting material was consumed and a new peak with desired product MS was detected. The reaction mixture was purified by Prep-HPLC (column: Phenomenex Luna 80*30 mm*3 urn; mobile phase: [water (0.1% TFA)-ACN], B %: 30%-50%, 8 mins) to give M1 (38.3 mg, yield 17.45%) as a yellow solid.

¹H NMR (ET43587-89-p1a, 400 MHz, DMSO-d6) δ 2.05 (br dd, J=10.69, 5.07 Hz, 1H), 2.89 (ddd, J=17.23, 13.79, 5.25 Hz, 1H), 3.06 (s, 5H), 3.19-3.41 (m, 4H), 3.43-3.61 (m, 2H), 3.85 (br s, 7H), 4.25-4.73 (m, 6H), 5.12 (dd, J=12.76, 5.38 Hz, 1H), 6.36 (br d, J=7.25 Hz, 1H), 6.81 (br d, J=8.63 Hz, 11H), 6.97 (br d, J=10.38 Hz, 11H), 7.10-7.25 (m, 21H), 7.29-7.39 (m, 2H), 7.42-7.52 (m, 3H), 7.83 (d, J=8.25 Hz, 1H), 8.03 (dd, J=8.13, 1.25 Hz, 2H), 8.55 (br d, J=4.13 Hz, 1H), 9.49 (br s, 1H), 9.88 (br s, 1H), 11.13 (s, 1H)

LCMS (ESI+): RT=2.360 min, m/z 503.2, 1005.2 (M/2+H, M+H)⁺.

5_95AB_6min-ELSD-MS1500: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1500.

General Procedure for Preparation of Compound 3-1c-Notebook Page: ET43587-139.

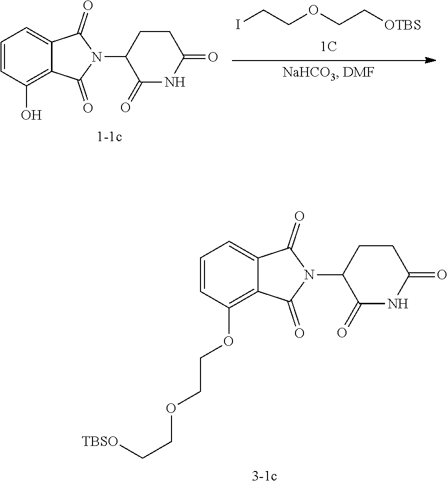

To a solution of Compound 1C (361.32 mg, 1.09 mmol, 1.5 eq) and Compound 1-1c (200 mg, 729.32 μmol, 1 eq) in DMF (4 mL) was added NaHCO₃ (183.81 mg, 2.19 mmol, 85.10 μL, 3 eq). The mixture was stirred at 60° C. for 12 hrs. LCMS showed all the starting material was consumed and a new peak with desired product MS was detected. The reaction mixture was diluted with water (10 mL) and extracted with ethyl acetate (3×10 mL). The organic layer was separated and the combined organic layer was washed with brine (10 mL), dried over Na₂SO₄, filtered and concentrated under reduced pressure to give Compound 3-1c (200 mg, yield 51.79%) as a colorless oil, which was used to the next step directly without further purification.

¹H NMR (ET43587-139-p1a, 400 MHz, CHLOROFORM-d) δ 0.07 (s, 6H), 0.89 (s, 9H), 2.09-2.16 (m, 1H), 2.68-2.95 (m, 3H), 3.65-3.70 (m, 2H), 3.76-3.80 (m, 2H), 3.93-3.97 (m, 2H), 4.36 (t, J=4.94 Hz, 2H), 4.96 (dd, J=12.26, 5.38 Hz, 1H), 7.30 (d, J=8.50 Hz, 1H), 7.48 (d, J=7.25 Hz, 1H), 7.68 (dd, J=8.38, 7.38 Hz, 1H), 8.04 (s, 1H)

General Procedure for Preparation of Compound 4-1c-Notebook Page: ET43587-153.

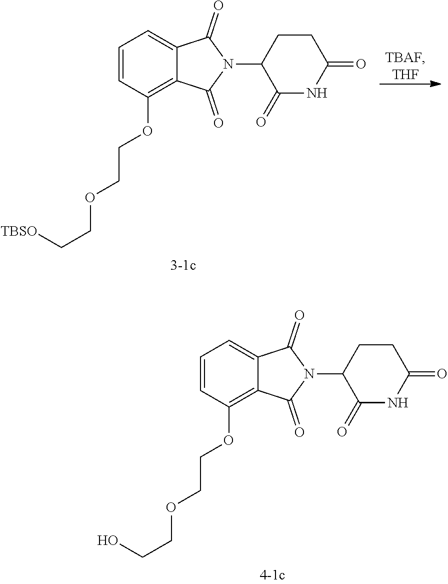

To a solution of Compound 3-1c (300 mg, 629.47 μmol, 1 eq) in THF (6 mL) was added TBAF (1 M, 3.78 mL, 6 eq) dropwise at 0° C. The mixture was stirred at 20° C. for 12 hrs. LCMS showed all the starting material was consumed and a new peak with desired product MS was detected. The reaction mixture was diluted with water (15 mL) and extracted with ethyl acetate (3×15 mL). The organic layer was separated and the combined organic layer was dried over Na₂SO₄, filtered and concentrated under reduced pressure to give a residue, which was purified by chromatography on silica gel (eluted with petroleum ether/ethyl acetate=20/1 to 1/1) to give Compound 4-1c (160 mg, yield 63.14%) as a colorless oil.

¹H NMR (ET43587-153-p1a, 400 MHz, CHLOROFORM-d) δ 1.96-2.07 (m, 1H), 2.71-2.94 (m, 3H), 3.72-3.82 (m, 4H), 3.94-4.02 (m, 3H), 4.35 (br s, 2H), 4.98 (br dd, J=11.56, 4.77 Hz, 1H), 7.24 (br s, 1H), 7.48 (br d, J=7.03 Hz, 1H), 7.64-7.74 (m, 1H), 8.25 (br s, 1H)

General Procedure for Preparation of Compound 4-1d-Notebook Page: ET43587-155.

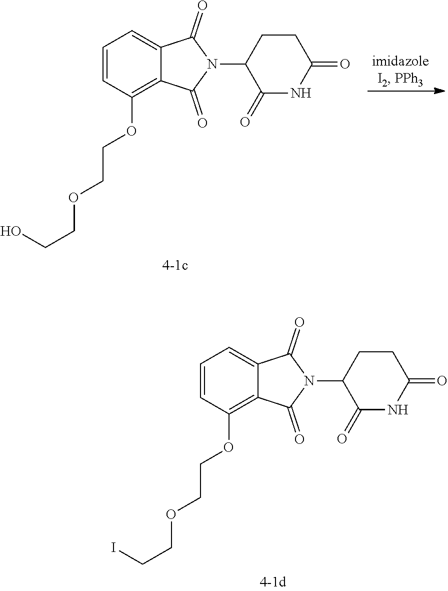 [SMILES: O=C1CCC(N2C(=O)c3cccc(OCCOCCO)c3C2=O)C(=O)N1 ; "imidazoleI2, PPh3" ; O=C1CCC(N2C(=O)c3cccc(OCCOCCI)c3C2=O)C(=O)N1]

To a solution of Compound 4-1c (160 mg, 441.58 μmol, 1 eq) and 12 (280.19 mg, 1.10 mmol, 2.5 eq) in DCM (3.5 mL) were added imidazole (75.16 mg, 1.10 mmol, 2.5 eq) and PPh₃ (289.56 mg, 1.10 mmol, 2.5 eq) at 0° C. The mixture was stirred at 20° C. for 12 hrs. LCMS showed all the starting material was consumed and a new peak with desired product MS was detected. The reaction mixture was diluted with water (10 mL) and extracted with dichloromethane (2×10 mL). The organic layer was separated and the combined organic layer was washed with brine (10 mL), dried over Na₂SO₄, filtered and concentrated under reduced pressure to give a residue, which was purified by Prep-HPLC (column: Phenomenex luna C18 100*40 mm*5 um; mobile phase: [water (0.1% TFA)-ACN]; B %: 35%-60%, 8 mins) to give Compound 4-1d (80 mg, yield 36.45%) as a white solid.

¹H NMR (ET43587-155-p1b, 400 MHz, CHLOROFORM-d) δ 2.09-2.20 (m, 1H), 2.68-2.98 (m, 3H), 3.30 (t, J=6.57 Hz, 2H), 3.90 (t, J=6.57 Hz, 2H), 3.95-3.99 (m, 2H), 4.35-4.41 (m, 2H), 4.93-5.00 (m, 1H), 7.30 (d, J=8.38 Hz, 1H), 7.49 (d, J=7.13 Hz, 1H), 7.70 (dd, J=8.38, 7.38 Hz, 1H), 8.05 (br s, 1H)

### General Procedure for Preparation of C1—Notebook Page: ET43587-167.

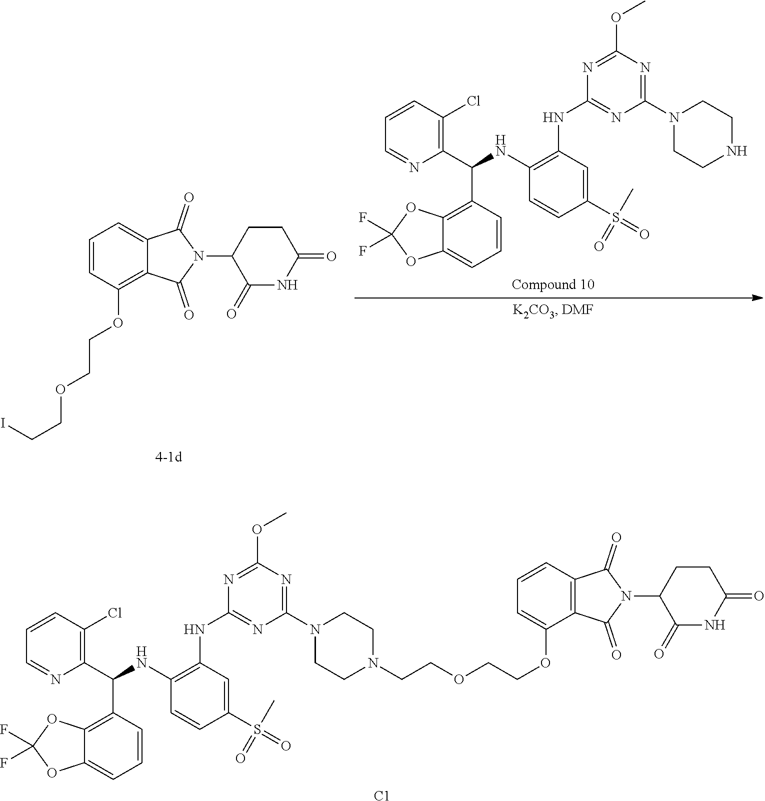

To a solution of Compound 10 (131.31 mg, 169.41 μmol, 1 eq) and Compound 4-1d (80 mg, 169.41 μmol, 1 eq) in DMF (2 mL) was added K₂CO₃ (81.95 mg, 592.93 μmol, 3.5 eq). The mixture was stirred at 60° C. for 12 hrs. LCMS showed all the starting material was consumed and a new peak with desired product MS was detected. The mixture was purified by Prep-HPLC (column: Phenomenex Luna 80*30 mm*3 um; mobile phase: [water (0.1% TFA)-ACN]; B %: 35%-60%, 8 mins) to give C1 (46.7 mg, yield 23.59%) as a pink solid.

¹H NMR (ET43587-167-P1A, 400 MHz, DMSO-d6) δ 1.97-2.09 (m, 1H), 2.60 (br s, 1H), 2.87 (br s, 1H), 3.06 (s, 5H), 3.12-3.43 (m, 6H), 3.83-3.96 (m, 8H), 4.36-4.64 (m, 4H), 5.08 (br dd, J=13.07, 5.32 Hz, 1H), 6.36 (br d, J=6.63 Hz, 1H), 6.80 (br d, J=8.76 Hz, 1H), 6.90-7.01 (m, 1H), 7.11-7.25 (m, 2H), 7.32 (dd, J=7.88, 1.13 Hz, 1H), 7.45-7.50 (m, 3H), 7.54 (d, J=8.63 Hz, 1H), 7.77-8.09 (m, 3H), 8.55 (br d, J=4.13 Hz, 1H), 9.34-9.57 (m, 1H), 9.76-9.95 (m, 1H), 11.09 (s, 1H)

LCMS (ESI+): RT=2.372 min, m/z 503.2, 1005.2 (M/2+H, M+H)⁺.

5_95AB_6min-220-MS1500: LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-1500.

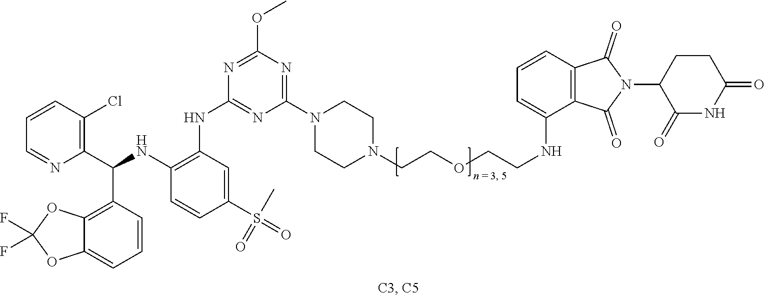

| Compound ID | Synonym | Synonym |
|---|---|---|
| NUCC-0226565 | C3 | ET43587-75 |
| NUCC-0226576 | C5 | ET43587-95 |

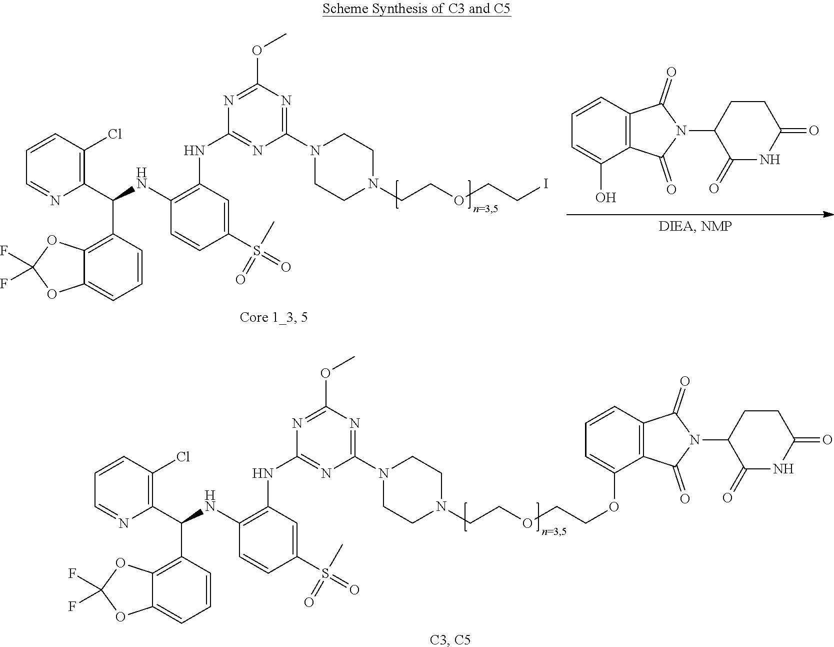

### Chemical Synthesis

All chemical reagents were obtained from commercial suppliers and used without further purification, unless otherwise stated. Reactions were run without taking precautions to exclude air or moisture, unless otherwise noted. Normal phase column chromatography was performed using silica gel columns and ACS grade solvents. Analytical TLC was performed on EM Reagent 0.25 mm silica gel 60 F254 plates and visualized by UV light. Compound identities were confirmed by ¹H and F (NMR) spectroscopy which were recorded on a Bruker 400 MHz spectrometer using the corresponding residual solvent peak (CDCl₃,1H δ=7.27; CD₃OD, 1H δ=3.31; DMSO-d₆, 1H δ=2.50) as an internal standard. The chemical shifts for ¹H-NMR is reported to the second decimal place. Proton coupling constants are expressed in hertz (Hz). Standard abbreviations were used to denote spin multiplicity for ¹H NMR data.

### LCMS Methods:

### Method: 5_95AB_6min-ELSD-MS1500

LC/MS: The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1500.

### Experimentals for Largest Scale Run:

### General Procedure for Preparation of C3—Notebook Page: ET43587-75.

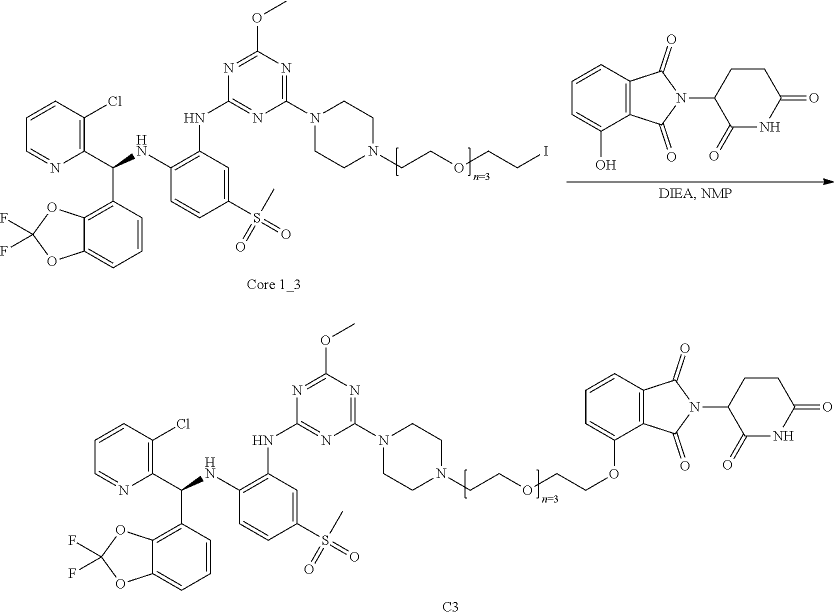

To a solution of 2-(2,6-dioxo-3-piperidyl)-4-hydroxy-isoindoline-1,3-dione (43.43 mg, 158.36 μmol, 1 eq) and Compound Core 1_3 (150 mg, 158.36 μmol, 1 eq) in DMF (3 mL) was added K₂CO₃ (65.66 mg, 475.09 μmol, 3 eq). The mixture was stirred at 50° C. for 12 hrs. LCMS showed all the starting material was consumed and a new peak with desired product MS was detected. The mixture was purified by Prep-HPLC (column: Phenomenex luna C18 80*40 mm*3 um; mobile phase: [water (0.04% HCl)-ACN]; B %: 32%-52%, 7 mins) to give C3 (35.6 mg, yield 19.5%) as a yellow solid.

¹H NMR (ET43587-75-P1B, 400 MHz, DMSO-d6) δ 1.97-2.05 (m, 1H), 2.83-2.93 (m, 1H), 2.96-3.12 (m, 5H), 3.18-3.46 (m, 5H), 3.48-3.65 (m, 11H), 3.69-3.88 (m, 7H), 4.19-4.67 (m, 4H), 5.08 (dd, J=12.69, 5.32 Hz, 1H), 6.36 (br s, 1H), 6.81 (br d, J=8.75 Hz, 1H), 6.97 (br s, 1H), 7.13-7.25 (m, 2H), 7.32 (d, J=8.50 Hz, 1H), 7.38-7.53 (m, 4H), 7.70-7.80 (m, 1H), 8.03 (br dd, J=8.07, 1.19 Hz, 2H), 8.56 (br s, 1H), 9.49 (br s, 1H), 10.77-11.00 (m, 1H), 11.12 (s, 1H)

LCMS (ESI+): RT=2.389 min, m/z 547.2, 1093.3 (M/2+H, M+H)⁺.

5_95AB_6min-ELSD-MS1500: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1500.

### General Procedure for Preparation of C5—Notebook Page: ET43587-95.

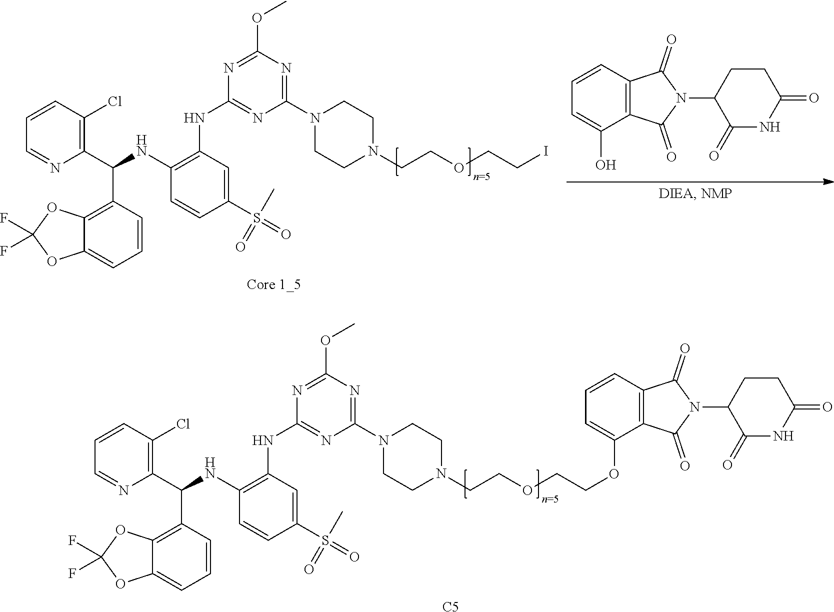

To a solution of 2-(2,6-dioxo-3-piperidyl)-4-hydroxy-isoindoline-1,3-dione (31.79 mg, 115.91 μmol, 1 eq) and Compound Core 1_5 (120 mg, 115.91 μmol, 1 eq) in DMF (2.5 mL) was added K₂CO₃ (80.10 mg, 579.55 μmol, 5 eq). The mixture was stirred at 60° C. for 12 hrs. LCMS showed all the starting material was consumed and a new peak with desired product MS was detected. The mixture was purified by Prep-HPLC (column: Phenomenex luna C18 100*40 mm*5 um; mobile phase: [water (0.1% TFA)-ACN]; B %: 40%-70%, 8 mins) to give C5 (43.6 mg, yield 28.28%) as a yellow solid.

¹H NMR (ET43587-95-PIA, 400 MHz, DMSO-d6) δ 1.97-2.07 (m, 1H), 2.80-2.94 (m, 1H), 3.07 (s, 5H), 3.31 (br s, 4H), 3.47-3.64 (m, 18H), 3.70-3.89 (m, 7H), 4.28-4.71 (m, 6H), 5.08 (dd, J=12.72, 5.50 Hz, 1H), 6.36 (br d, J=6.72 Hz, 1H), 6.81 (br d, J=8.68 Hz, 1H), 6.91-7.01 (m, 1H), 7.08-7.26 (m, 2H), 7.32 (d, J=8.07 Hz, 1H), 7.42-7.55 (m, 4H), 7.80 (t, J=7.83 Hz, 1H), 7.92-8.07 (m, 2H), 8.55 (br d, J=4.40 Hz, 1H), 9.39-9.52 (m, 1H), 9.70-9.88 (m, 1H), 11.09 (s, 1H)

LCMS (ESI+): RT=2.491 min, m/z 591.2, 1181.6 (M/2+H, M+H)⁺.

5_95AB_6min-ELSD-MS1500: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1500.

### Synthesis of E1, E3 and E5

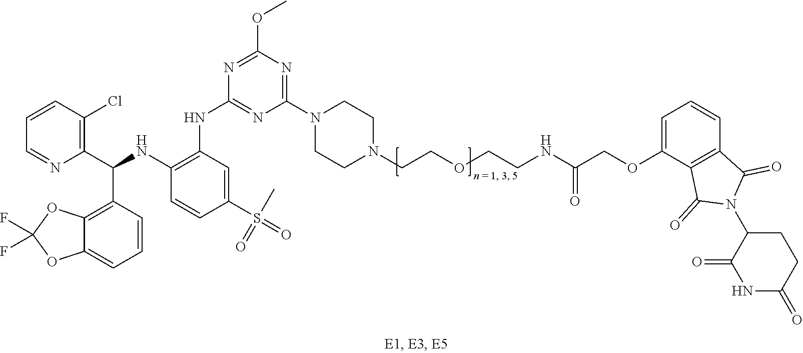

| Compound ID | Synonym | Synonym |
|---|---|---|
| NUCC-0226588 | E1 | ET43587-151 |
| NUCC-0226589 | E3 | ET43587-104 |
| NUCC-0226590 | E5 | ET43587-140 |

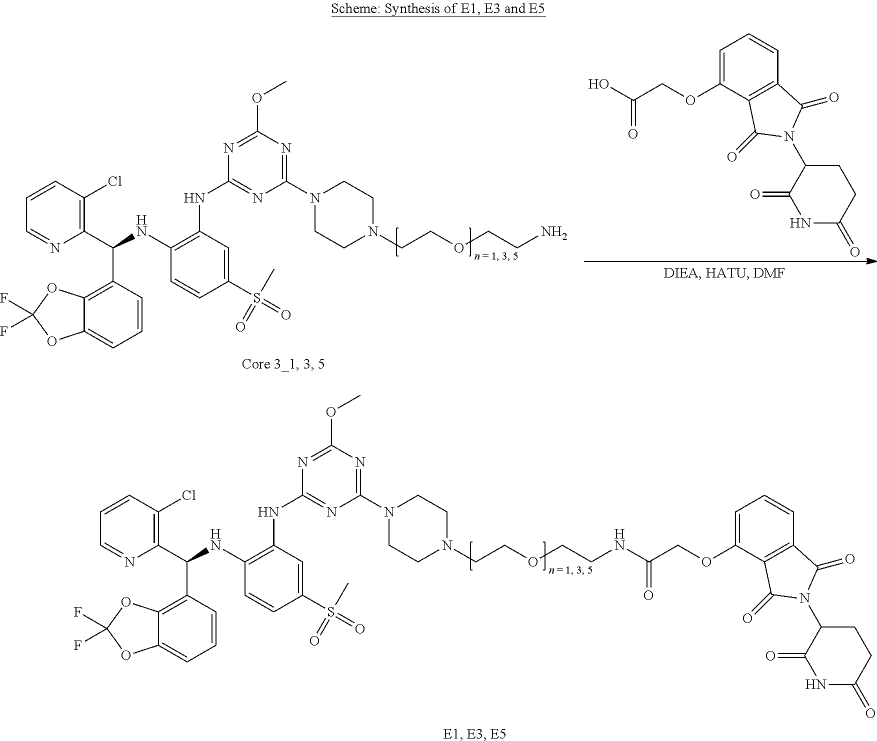

### Chemical Synthesis

All chemical reagents were obtained from commercial suppliers and used without further purification, unless otherwise stated. Reactions were run without taking precautions to exclude air or moisture, unless otherwise noted. Normal phase column chromatography was performed using silica gel columns and ACS grade solvents. Analytical TLC was performed on EM Reagent 0.25 mm silica gel 60 F254 plates and visualized by UV light. Compound identities were confirmed by ¹H and F (NMR) spectroscopy which were recorded on a Bruker 400 MHz spectrometer using the corresponding residual solvent peak (CDCl₃,1H δ=7.27; CD₃OD, 1H δ=3.31; DMSO-d₆, 1H δ=2.50) as an internal standard. The chemical shifts for ¹H-NMR is reported to the second decimal place. Proton coupling constants are expressed in hertz (Hz). Standard abbreviations were used to denote spin multiplicity for ¹H NMR data.

### LCMS Methods:

### Method: 5_95AB_6min-220-MS1500

LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-1500.

### Experimental for Largest Scale Run:

### General Procedure for Preparation of E1—Notebook Page: ET43318-89, ET43587-151.

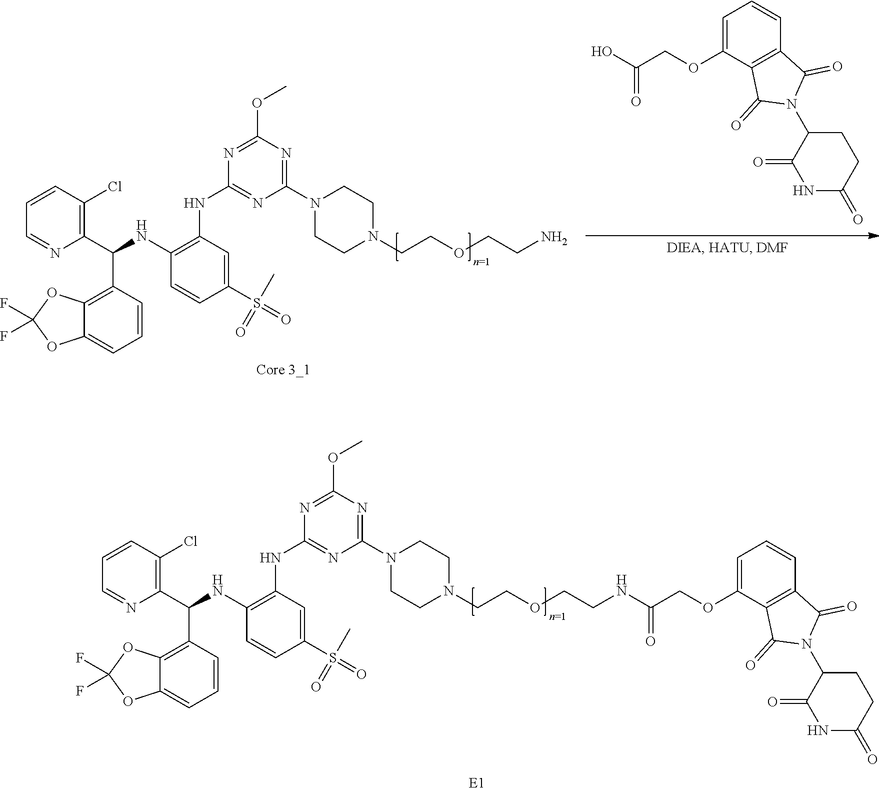

To a solution of Core 3_1 (100 mg, 133.65 μmol, 1 eq) in DMF (0.5 mL) were added DIEA (51.82 mg, 400.96 μmol, 3 eq), HATU (76.23 mg, 200.48 μmol, 1.5 eq) at 20° C. After stirring for 30 mins, 2-[2-(2,6-dioxo-3-piperidyl)-1,3-dioxo-isoindolin-4-yl]oxyacetic acid (59.65 mg, 133.65 μmol, 1 eq, TFA) was added. The reaction mixture was stirred for 12 hrs. LCMS showed the starting material was consumed and 69.4% peak (Rt=0.701 min) with desired Ms was detected. The mixture was filtered to give crude product, which was purified by Prep-HPLC (column: Waters Xbridge BEH C18 100*30 mm*10 um; mobile phase: [water (10 mM NH₄HCO₃)-ACN]; B %: 30%-60%, 10 mins) then Prep-HPLC (column: Phenomenex Luna 80*30 mm*3 um; mobile phase: [water (0.1% TFA)-ACN]; B %: 25%-45%, 8 mins) to give E1 (13.2 mg, yield 37.25%) as a white solid.

¹H NMR (ET43587-151-P1A, 400 MHz, DMSO-d6) δ 1.96-2.08 (m, 1H), 2.56-2.61 (m, 1H), 2.82-2.92 (m, 1H), 3.07 (s, 5H), 3.22-3.42 (m, 6H), 3.54 (br t, J=5.38 Hz, 5H), 3.78 (br d, J=15.26 Hz, 5H), 4.38-4.68 (m, 2H), 4.79 (s, 2H), 5.10 (dd, J=12.69, 5.44 Hz, 1H), 6.36 (br d, J=7.25 Hz, 1H), 6.80 (br d, J=8.76 Hz, 1H), 6.95-7.01 (m, 1H), 7.09-7.26 (m, 3H), 7.32 (d, J=8.25 Hz, 1H), 7.41 (d, J=8.50 Hz, 1H), 7.44-7.53 (m, 3H), 7.81 (t, 1=7.88 Hz, 1H), 7.99-8.05 (m, 2H), 8.55 (br d, J=4.00 Hz, 1H), 9.39-9.54 (m, J H), 9.72-9.88 (m, 1H), 11.12 (s, 1H)

LCMS (ESI+): RT=2.355 min, m/z 531.8, 1062.2 (M/2+H. M+H)⁺.

5_95AB_6min-220-MS1500: LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-1500.

### General Procedure for Preparation of E3—Notebook Page: ET43587-104.

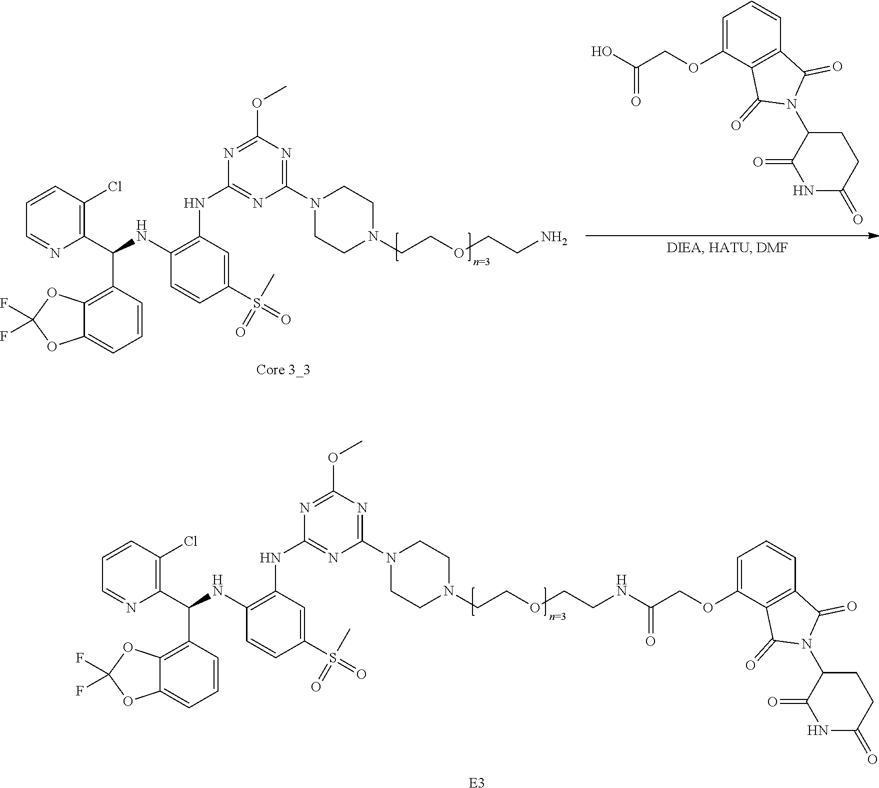

To a solution of 2-[2-(2,6-dioxo-3-piperidyl)-1,3-dioxo-isoindolin-4-yl]oxyacetic acid (47.68 mg, 143.49 μmol, 1 eq) and Compound Core 3_3 (120 mg, 143.49 μmol, 1 eq) in DMF (2 mL) were added DIEA (37.09 mg, 286.98 μmol, 2 eq) and HATU (65.47 mg, 172.19 μmol, 1.2 eq) at 0° C. The mixture was stirred at 20° C. for 12 hrs. LCMS showed all the starting material was consumed and a new peak with desired product MS was detected. The reaction mixture was purified by Prep-HPLC (column: Phenomenex luna C18 100*40 mm*5 um; mobile phase: [water (0.1% TFA)-ACN]; B %: 35%-65%, 8 mins) to give E3 (32.4 mg, yield 17.77%) as a white solid.

¹H NMR (ET43587-104-P1H, 400 MHz, DMSO-d6) δ 2.00-2.11 (m, 1H), 2.62 (br d, J=2.63 Hz, 1H), 2.90 (ddd, J=17.23, 13.91, 5.25 Hz, 1H), 2.98-3.15 (m, 5H), 3.23-3.38 (m, 6H), 3.40-3.69 (m, 13H), 3.81 (br d, J=3.50 Hz, 5H), 4.49-4.72 (m, 2H), 4.79 (s, 2H), 5.12 (dd, J=12.88, 5.38 Hz, 1H), 6.37 (br s, 1H), 6.81 (d, J=8.76 Hz, 1H), 6.96 (br s, 1H), 7.12-7.27 (m, 2H), 7.33 (dd, J=7.88, 1.13 Hz, 1H), 7.40 (d, J=8.50 Hz, 1H), 7.45-7.55 (m, 3H), 7.81 (dd, J=8.38, 7.38 Hz, 1H), 7.99-8.08 (m, 2H), 8.53-8.59 (m, 1H), 9.47 (br s, 1H), 10.57-10.81 (m, 1H), 11.12 (s, 1H)

LCMS (ESI+): RT=2.354 min, m/z 575.8, 1150.3 (M/2+H, M+H)⁺.

5_95AB_6min-220-MS1500: LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-1500.

### General Procedure for Preparation of E5—Notebook Page: ET43587-140.

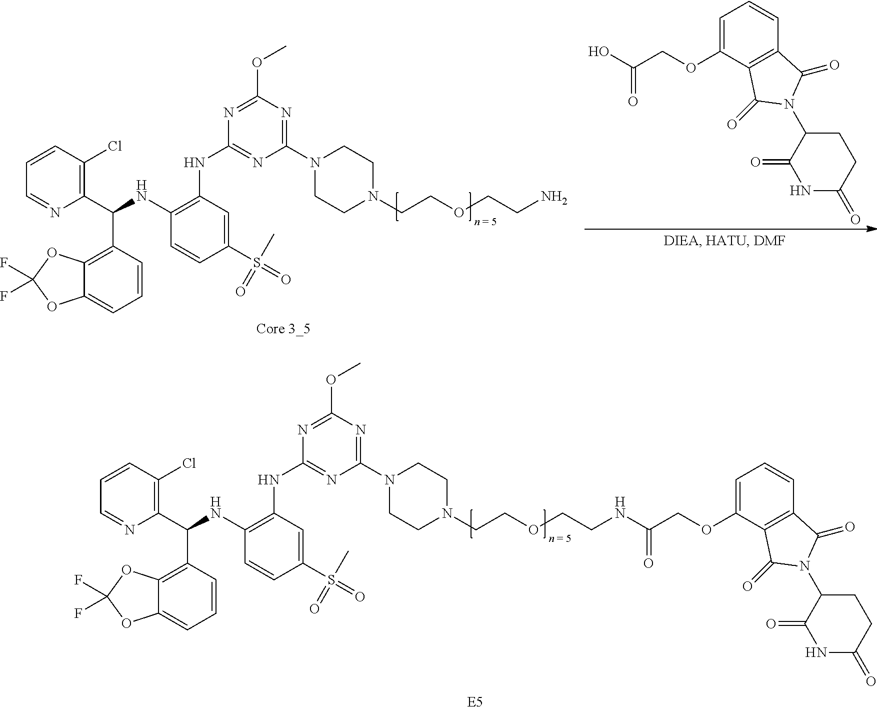

To a solution of 2-[2-(2,6-dioxo-3-piperidyl)-1,3-dioxo-isoindolin-4-yl]oxyacetic acid (46.73 mg, 140.63 μmol, 1 eq) and Compound Core 35 (130 mg, 140.63 μmol, 1 eq) in DMF (2 mL) were added DIEA (36.35 mg, 281.26 μmol, 48.99 μL, 2 eq) and HATU (53.47 mg, 140.63 μmol, 1 eq) at 0° C. The mixture was stirred at 20° C. for 12 hrs. LCMS showed all the starting material was consumed and a new peak with desired product MS was detected. The mixture was purified by Prep-HPLC (column. Phenomenex Luna 80*30 mm*3 um; mobile phase. [water (0.1% TFA)-ACN]; B %: 20%-55%, 8 mins) to give E5 (33.2 mg, yield 19.02%) as a white solid.

¹H NMR (ET43587-140-P1H, 400 MHz, DMSO-d6) δ 1.99-2.08 (m, 1H), 2.57-2.62 (m, 1H), 2.89 (ddd, J=17.13, 14.07, 5.44 Hz, 1H), 3.07 (s, 5H), 3.20-3.38 (m, 6H), 3.40-3.62 (m, 21H), 3.75 (br s, 5H), 4.40-4.66 (m, 2H), 4.78 (s, 2H), 5.11 (dd, J=12.88, 5.38 Hz, 1H), 6.36 (br d, J=7.13 Hz, 1H), 6.81 (d, J=8.76 Hz, 1H), 6.91-7.00 (m, 1H), 7.10-7.26 (m, 2H), 7.32 (dd, J=7.88, 1.13 Hz, 1H), 7.39 (d, J=8.50 Hz, 1H), 7.45-7.52 (m, 3H), 7.81 (dd, J=8.32, 7.44 Hz, 1H), 7.92-8.08 (m, 3H), 8.55 (br d, J=4.13 Hz, 1H), 9.47 (br dd, J=4.82, 2.56 Hz, 1H), 9.73-9.90 (m, 1H), 11.11 (s, 1H)

LCMS (ESI+): RT=2.367 min, m/z 619.8, 1238.6 (M/2+H, M+H)⁺.

5_95AB_6min-220-MS1500: LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-1500.

### Synthesis of G2, G3 and G5

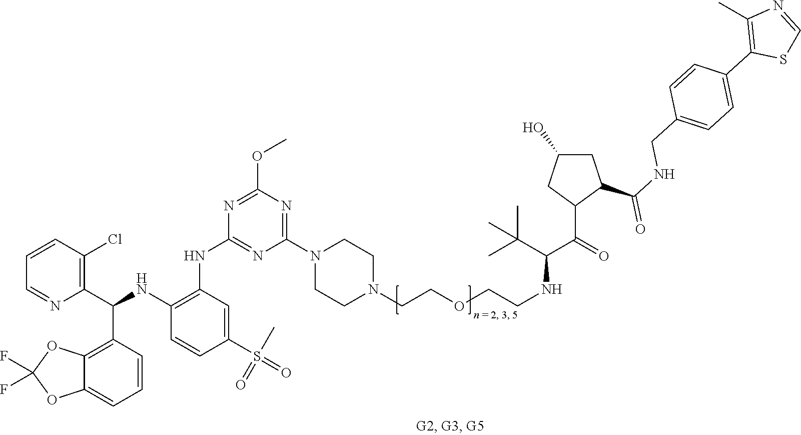

| Compound ID | Synonym | Synonym |
|---|---|---|
| NUCC-0226655 | G2 | ET43587-298 |
| NUCC-0226653 | G3 | ET43587-205 |
| NUCC-0226599 | G5 | ET43587-185 |

### Original Scheme

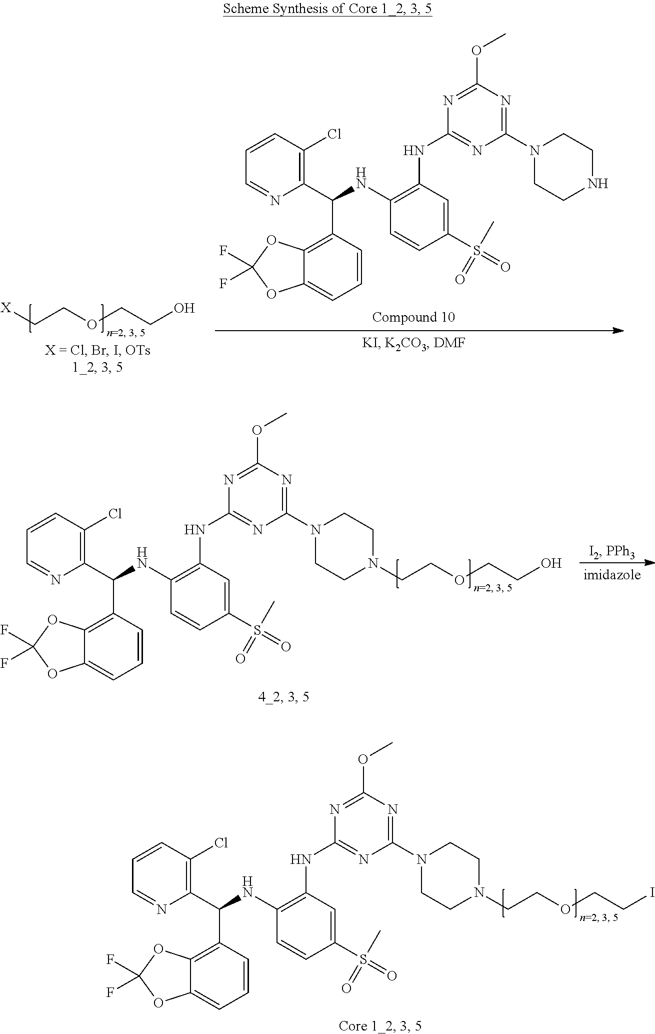

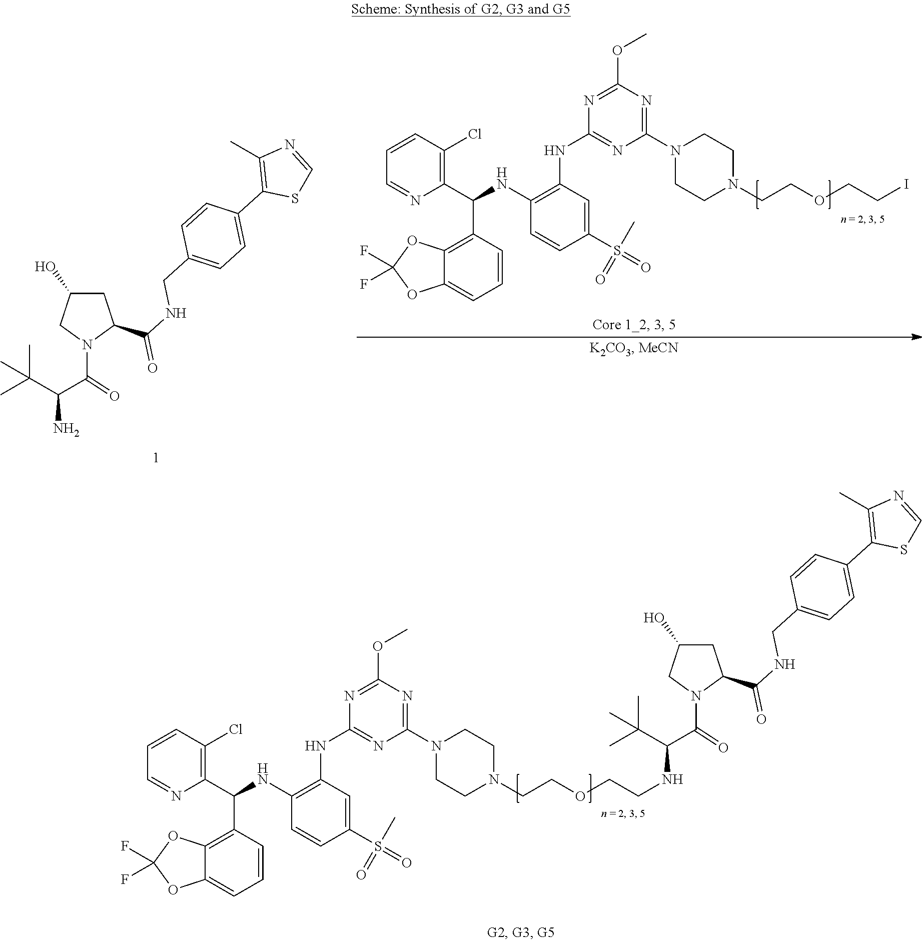

### Chemical Synthesis

All chemical reagents were obtained from commercial suppliers and used without further purification, unless otherwise stated. Reactions were run without taking precautions to exclude air or moisture, unless otherwise noted. Normal phase column chromatography was performed using silica gel columns and ACS grade solvents. Analytical TLC was performed on EM Reagent 0.25 mm silica gel 60 F254 plates and visualized by UV light. Compound identities were confirmed by ¹H and F (NMR) spectroscopy which were recorded on a Bruker 400 MHz spectrometer using the corresponding residual solvent peak (CDCl₃,1H δ=7.27; CD₃OD, 1H δ=3.31; DMSO-d₆, 1H δ=2.50) as an internal standard. The chemical shifts for ¹H-NMR is reported to the second decimal place. Proton coupling constants are expressed in hertz (Hz). Standard abbreviations were used to denote spin multiplicity for ¹H NMR data.

### LCMS Methods:

### Method 1: 5_95AB_2 min:

LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### Method 2: 5_95AB_6min-ELSD-MS1500

LC/MS: The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1500.

### Method 3: 5_95AB_6min-220-MS1500

LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-1500.

### Experimentals for Largest Scale Run:

### General Procedure for Preparation of Compound 4_2—Notebook Page: ET43587-270.

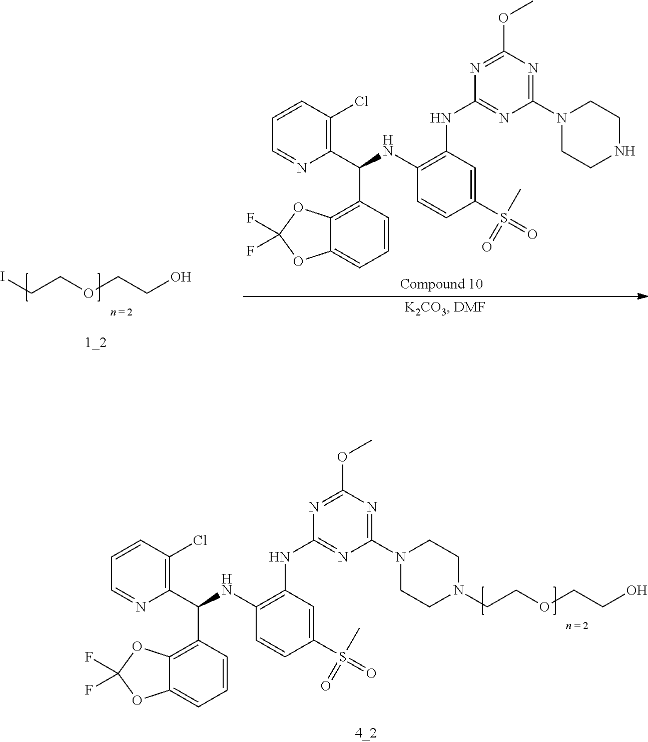

To a solution of Compound 10 (762.58 mg, 1.15 mmol, 1 eq) and Compound 1_2 (300 mg, 1.15 mmol, 1 eq) in DMF (14 mL) was added K₂CO₃ (797.15 mg, 5.77 mmol, 5 eq). The mixture was stirred at 60° C. for 6 hrs. LCMS showed all the starting material was consumed and a new peak with desired product MS was detected. The reaction mixture was diluted with water (20 mL) and extracted with ethyl acetate (3×20 mL). The organic layer was separated and the combined organic layer was washed with brine (20 mL), dried over Na₂SO₄, filtered and concentrated under reduced pressure to give a residue, which was purified by chromatography on silica gel (eluted with ethyl acetate/methanol=20/1 to 1/1) to give the Compound 42 (400 mg, yield 39.34%) as a yellow solid.

LCMS (ESI+): RT=0.690 min, m/z 793.2 (M+H)⁺.

5_95AB_2 min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### General Procedure for Preparation of Core 1_2—Notebook Page: ET43587-289.

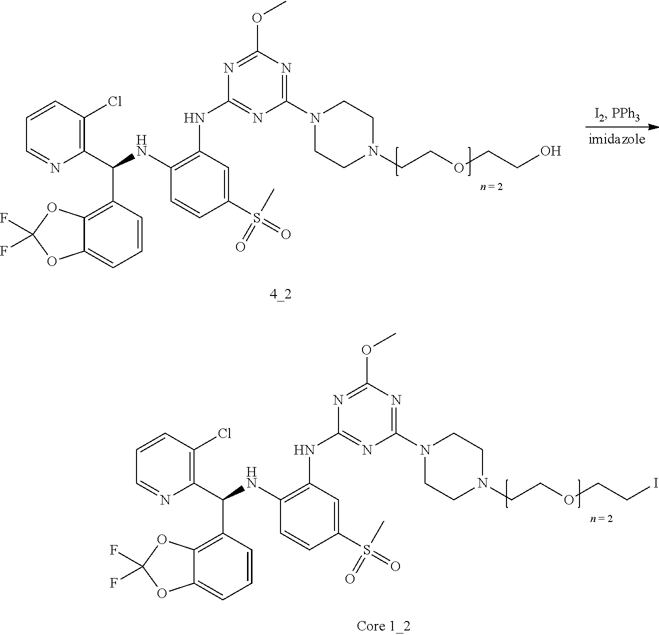

To a solution of Compound 4_2 (400 mg, 504.26 μmol, 1 eq) and I₂ (319.97 mg, 1.26 mmol, 2.5 eq) in DCM (8 mL) were added imidazole (85.83 mg, 1.26 mmol, 2.5 eq) and PPh₃ (330.66 mg, 1.26 mmol, 2.5 eq) at 0° C. The mixture was stirred at 20° C. for 3 hrs. LCMS showed all the starting material was consumed and a new peak with desired product MS was detected. The reaction mixture was diluted with water (60 mL) and extracted with ethyl acetate (3-30 mL). The organic layer was separated and the combined organic layer was washed with brine (2×30 mL), dried over Na₂SO₄, filtered and concentrated under reduced pressure to give a residue, which was purified by chromatography on silica gel (eluted with petroleum ether/ethyl acetate=50/1 to 5/1) to give Compound Core 1_2 (280 mg, yield 55.33%) as a yellow solid.

LCMS (ESI+): RT=0.723 min, m/z 903.2 (M+H)⁺.

5_95AB_2 min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### General Procedure for Preparation of G2—Notebook Page: ET43587-298.

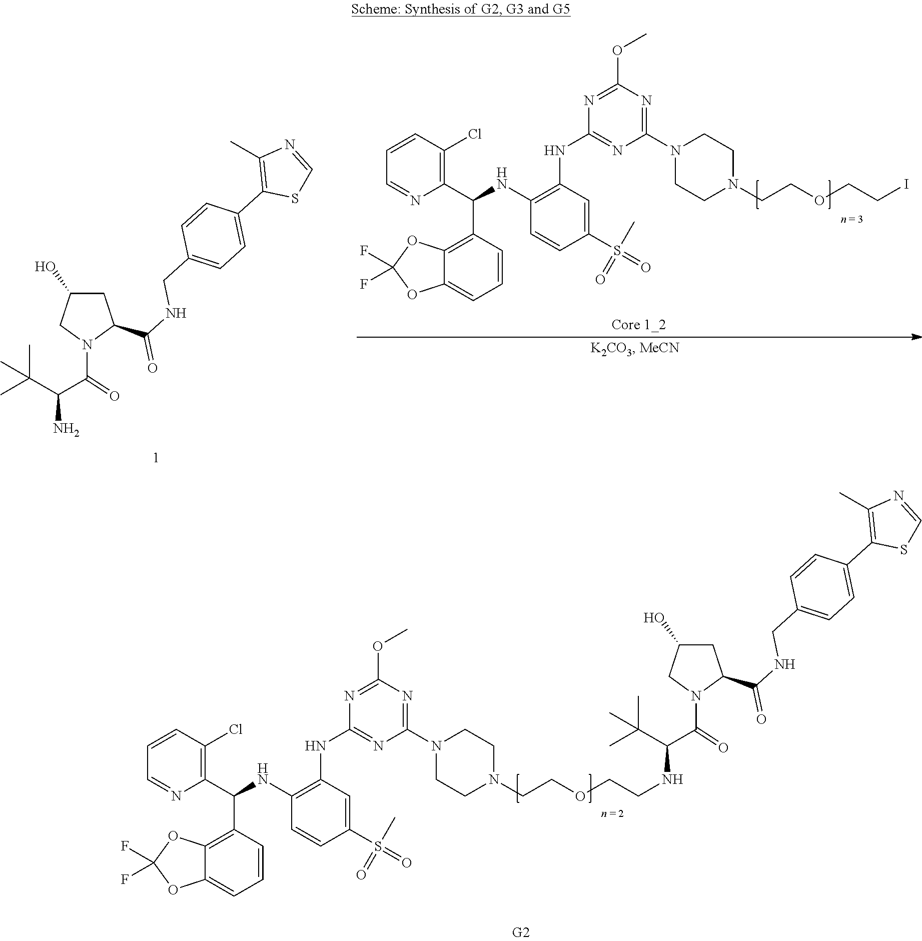

To a solution of Compound 1 (42.91 mg, 99.65 μmol, 1 eq) and Compound Core 1_2 (90 mg, 99.65 μmol, 1 eq) in ACN (2 mL) was added K₂CO₃ (41.32 mg, 298.96 μmol, 3 eq). The mixture was stirred at 90′C for 12 hrs. LCMS showed all the starting material was consumed and a new peak with desired product MIS was detected. The mixture was filtered and purified by Prep-HPLC (column: Phenomenex Luna 80*30 mm*3 um; mobile phase: [water (TFA)-ACN]; B %: 30%-50%, 8 mins) to give G2 (37.8 mg, yield 27.82%) as a yellow solid.

¹H NMR (ET43587-298-p1a, 400 MHz, DMSO-d6) δ 1.07 (s, 9H), 1.93 (ddd, J=12.91, 8.91, 4.32 Hz, 1H), 2.16 (br dd, J=12.38, 7.75 Hz, 1H), 2.44 (s, 3H), 2.97 (br s, 1H), 3.01-3.11 (m, 6H), 3.23-3.35 (m, 4H), 3.68 (br d, J=4.13 Hz, 16H), 4.17-4.28 (m, 2H), 4.34-4.51 (m, 3H), 4.59 (br t, J=8.44 Hz, 2H), 6.36 (d, J=7.38 Hz, 1H), 6.81 (br d, J=8.63 Hz, 1H), 6.96 (br s, 1H), 7.12-7.18 (m, 1H), 7.20-7.24 (m, 1H), 7.32 (d, J=7.75 Hz, 1H), 7.39 (s, 4H), 7.48 (dd, J=8.07, 4.69 Hz, 2H), 8.04 (d, J=8.00 Hz, 2H), 8.32 (br s, 1H), 8.56 (br d, J=4.00 Hz, 1H), 8.74 (br t, 0.1=5.69 Hz, 2H), 8.99 (s, 1H), 9.50 (br d, J=2.00 Hz, 1H), 10.07 (br s, 1H)

LCMS (ESI+): RT=2.240 min, m/z 603.2, 1205.3 (M/2+H, M+H)⁺.

5_95AB_6min_MS1500-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1500.

### General Procedure for Preparation of Compound 4_3—Notebook Page: ET43587

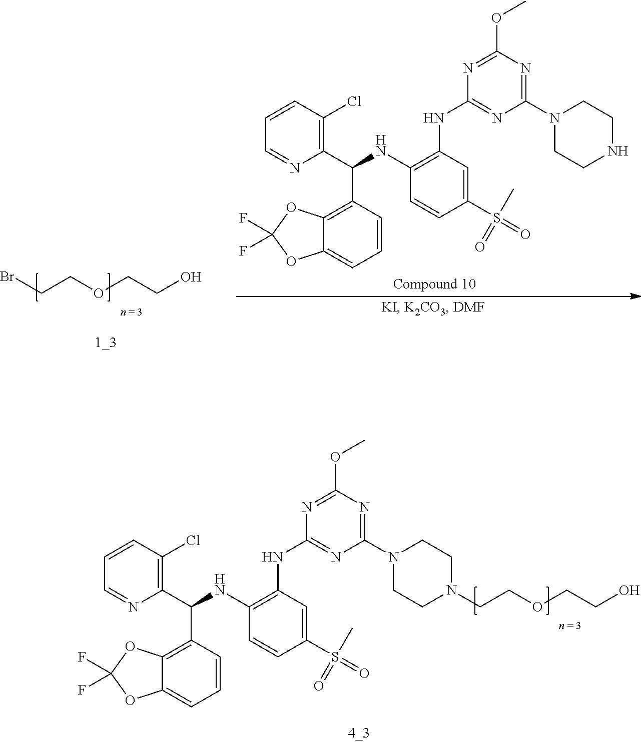

To a solution of Compound 10 (1 g, 1.29 mmol, 1 eq) and Compound 1_3 (331.73 mg, 1.29 mmol, 1 eq) in DMF (6 mL) was added KI (214.17 mg, 1.29 mmol, 1 eq) and K₂CO₃ (534.94 mg, 3.87 mmol, 3 eq). The mixture was stirred at 60° C. for 12 hrs. LCMS showed all the starting material was consumed and a new peak with desired product MS was detected. The reaction mixture was diluted with water (20 mL) and extracted with ethyl acetate (3×20 mL). The organic layer was separated and the combined organic layer was washed with brine (2×20 mL), dried over Na₂SO₄, filtered and concentrated under reduced pressure to give a residue, which was purified by chromatography on silica gel (eluted with ethyl acetate/methanol=I/O to 3/1) to give Compound 43 (600 mg, yield 49.99%) as a yellow solid.

¹H NMR (ET43587-46-p1a, 400 MHz, CHLOROFORM-d) δ 2.46-2.67 (m, 5H), 2.99 (s, 3H), 3.57-3.75 (m, 17H), 3.87 (br s, 5H), 6.25 (br d, J=7.13 Hz, 1H), 6.77 (br d, J=7.13 Hz, 1H), 6.86-7.03 (m, 4H), 7.21-7.25 (m, 1H), 7.61 (dd, J=8.63, 1.88 Hz, 1H), 7.70 (d, J=8.13 Hz, 1H), 8.10 (br s, 1H), 8.44-8.50 (m, 1H)

### General Procedure for Preparation of Core 1_3—Notebook Page: ET43587-68.

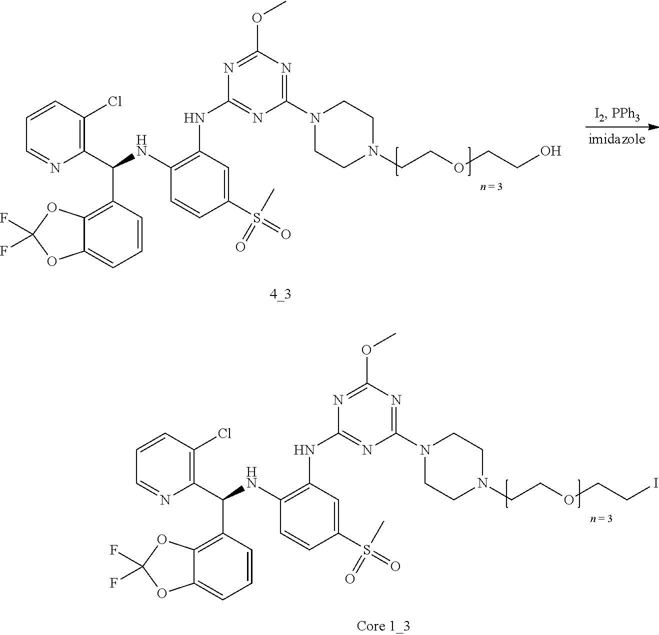

To a solution of Compound 43 (600 mg, 716.60 μmol, 1 eq), I₂ (454.70 mg, 1.79 mmol, 2.5 eq) and imidazole (195.14 mg, 2.87 mmol, 4 eq) in DCM (12 mL) was added PPh₃ (469.89 mg, 1.79 mmol, 2.5 eq) at 0° C. The mixture was stirred at 20° C. for 12 hrs. TLC (ethyl acetate/methanol=3/1) showed the starting material was consumed and a new spot was generated. The reaction mixture was diluted with water (20 mL) and extracted with DCM (3×20 mL). The organic layer was separated and the combined organic layer was washed with brine (20 mL), dried over Na₂SO₄, filtered and concentrated under reduced pressure to give a residue, which was purified by chromatography on silica gel (eluted with ethyl acetate/methanol=50/1 to 3/1) to give Core 13 (450 mg, yield 59.67%) as a yellow solid.

¹H NMR (ET43587-68-p1a, 400 MHz, CHLOROFORM-d) δ 2.50-3.11 (m, 9H), 3.28-3.30 (m, 2H), 3.56-4.08 (m, 19H), 6.23 (dd, J=7.09, 2.57 Hz, 1H), 6.77-6.85 (m, 1H), 6.88-7.02 (m, 4H), 7.17-7.24 (m, 1H), 7.44-7.52 (m, 1H), 7.61 (dd, J=8.60, 1.69 Hz, 1H), 7.64-7.72 (m, 2H), 8.43 (br t, J=5.52 Hz, 1H)

General Procedure for Preparation of G3-Notebook Page. ET43587-205.

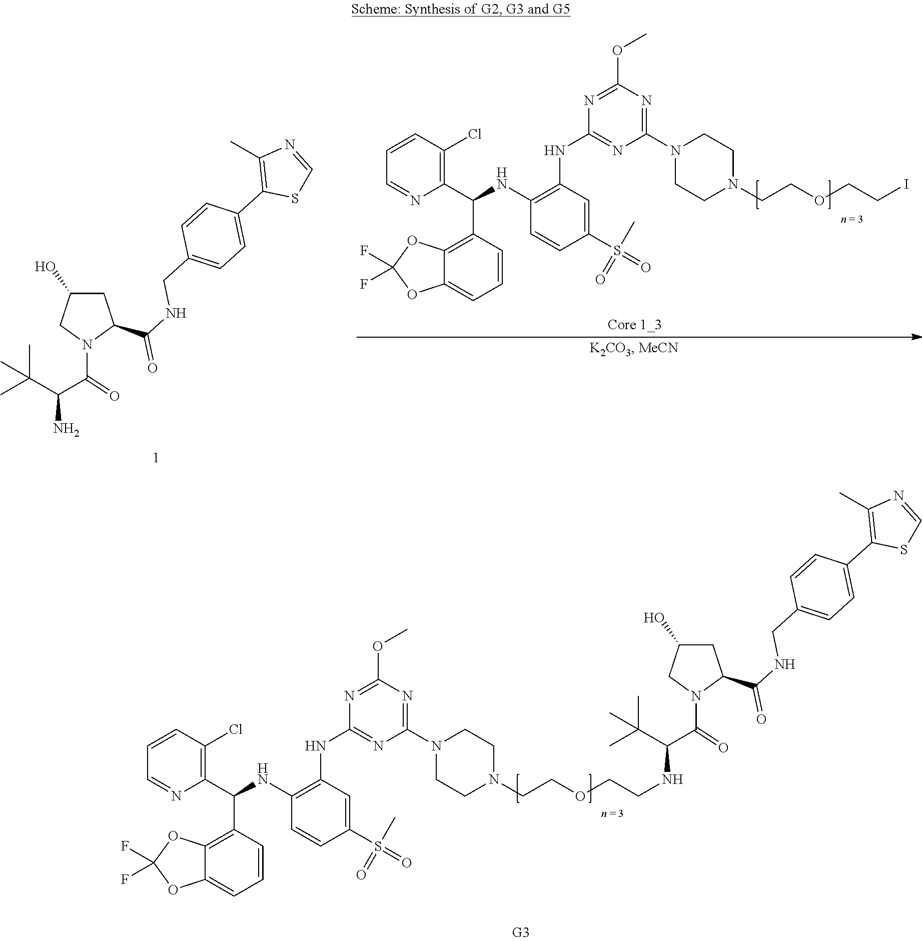

To a solution of Compound 1 (45.46 mg, 105.58 μmol, 1 eq) and Core 13 (100 mg, 105.58 μmol, 1 eq) in MeCN (2 mL) was added K₂CO₃ (43.77 mg, 316.73 μmol, 3 eq). The mixture was stirred at 90° C. for 12 hrs. LCMS showed all the starting material was consumed and a new peak with desired product MS was detected. The reaction mixture was purified by Prep-HPLC (column: Phenomenex Luna 80*30 mm*3 um; mobile phase: [water (0.1% TFA)-ACN]; B %: 25%-50%, 8 mins) to give G3 (56.2 mg, yield 37.12%) as a yellow solid.

¹H NMR (ET43587-205-p1a, 400 MHz, DMSO-d₆) δ 1.07 (s, 9H), 1.93 (ddd, J=13.01, 9.01, 4.50 Hz, 1H), 2.16 (br dd, J=12.63, 8.13 Hz, 1H), 2.43-2.45 (m, 3H), 2.89-2.97 (m, 1H), 3.00-3.15 (m, 6H), 3.30 (br s, 4H), 3.47-3.60 (m, 1H), 3.67-3.84 (m, 9H), 4.22-4.26 (m, 2H), 4.36-4.48 (m, 3H), 4.60 (br t, J=8.44 Hz, 2H), 6.36 (br d, J=7.13 Hz, 1H), 6.80 (br d, J=8.63 Hz, 1H), 6.97 (br s, 1H), 7.12-7.25 (m, 2H), 7.29-7.42 (m, 5H), 7.48 (dd, J=8.13, 4.50 Hz, 2H), 8.04 (dd, J=8.13, 1.25 Hz, 2H), 8.29 (br d, J=3.63 Hz, 1H), 8.56 (br d, J=3.50 Hz, 1H), 8.76 (br t, J=6.00 Hz, 2H), 9.00 (s, 1H), 9.51 (br s, 1H), 9.98 (br d, J=3.25 Hz, 1H)

LCMS (ESI+): RT=2.260 min, m/z 625.2, 1249.3 (M/2+H, M+H)⁺.

5_95AB_6min_MS1500-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1500.

### General Procedure for Preparation of Compound 4_5—Notebook Page: ET43587

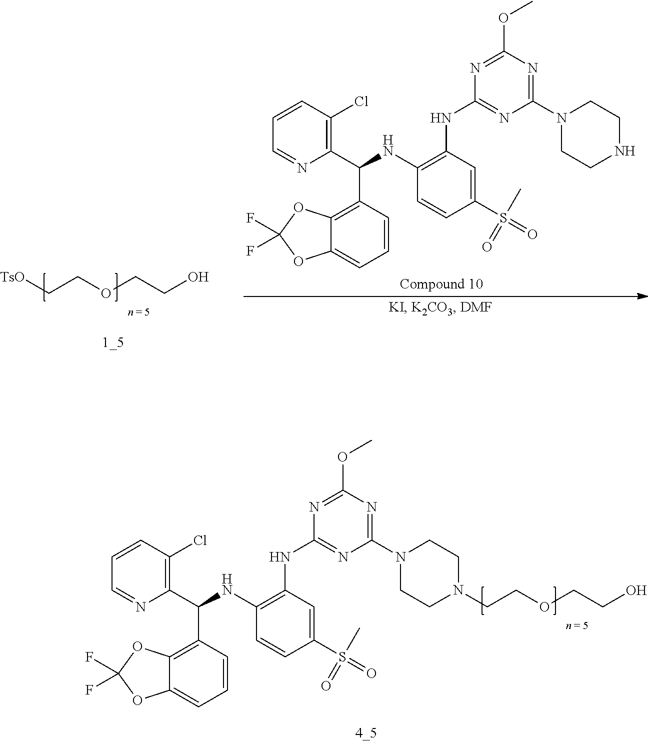

To a solution of Compound 10 (1.60 g, 2.06 mmol, 1 eq, TFA) and Compound 15 (900 mg, 2.06 mmol, 1 eq) in DMF (20 mL) were added KI (34.23 mg, 206.18 μmol, 0.1 eq) and K₂CO₃ (1.42 g, 10.31 mmol, 5 eq). The mixture was stirred at 60° C. for 12 hrs. TLC (ethyl acetate/methanol=1/3) showed the starting material was consumed and a new spot was generated. The reaction mixture was diluted with water (50 mL) and extracted with ethyl acetate (3×40 mL). The organic layer was separated and the combined organic layer was washed with brine (40 mL), dried over Na₂SO₄, filtered and concentrated under reduced pressure to give a residue, which was purified by chromatography on silica gel (eluted with petroleum ether/ethyl acetate=20/1 to 0/1) to give Compound 45 (600 mg, yield 28.3%) as a yellow solid.

LCMS (ESI+): RT=0.688 min, m/z 925.4 (M+H)⁺.

5_95AB_2 min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### General Procedure for Preparation of Core 1_5—Notebook Page: ET43587-165.

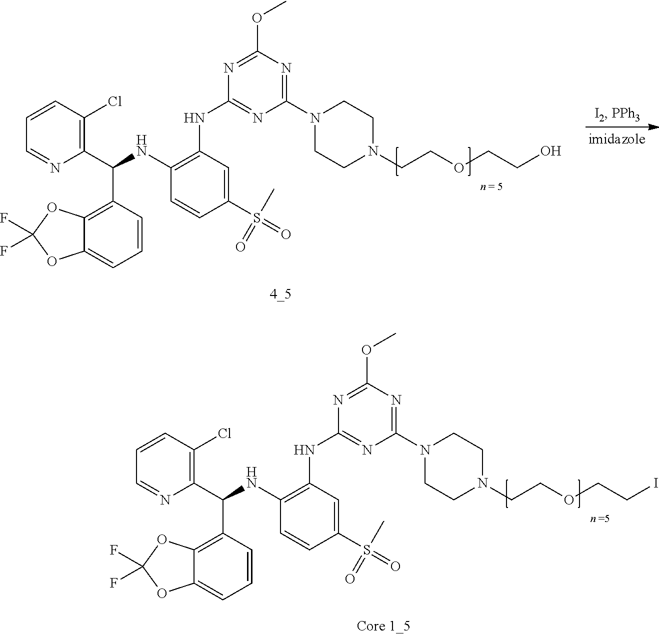

To a solution of Compound 4_5 (600 mg, 648.37 μmol, 1 eq) and I₂ (411.41 mg, 1.62 mmol, 2.5 eq) in DCM (12 mL) were added imidazole (110.35 mg, 1.62 mmol, 2.5 eq) and PPh₃ (425.15 mg, 1.62 mmol, 2.5 eq) at 0° C. The mixture was stirred at 20° C. for 12 hrs. LCMS showed all the starting material was consumed and a new peak with desired product MS was detected. The reaction mixture was diluted with water (30 mL) and extracted with dichloromethane (2×30 mL). The organic layer was separated and the combined organic layer was washed with brine (30 mL), dried over Na₂SO₄, filtered and concentrated under reduced pressure to give a residue, which was purified by chromatography on silica gel (eluted with ethyl acetate/methanol=20/1 to 1/1) to give Compound Core 15 (430 mg, yield 57.65%) as a yellow solid.

¹H NMR (ET43587-165-p1a, 400 MHz, CHLOROFORM-d) δ 2.99 (s, 3H), 3.28 (t, J=6.82 Hz, 2H), 3.58-3.84 (m, 27H), 3.89 (br s, 6H), 6.25 (d, J=7.25 Hz, 1H), 6.74 (br d, J=7.25 Hz, 1H), 6.88-6.99 (m, 5H), 7.25 (dd, J=8.19, 4.82 Hz, 1H), 7.61 (dd, J=8.57, 2.06 Hz, 1H), 7.71 (dd, J=8.00, 1.38 Hz, 1H), 8.12 (br s, 1H), 8.49 (dd, J=4.63, 1.25 Hz, 1H)

### General Procedure for Preparation of G5—Notebook Page: ET43587-185.

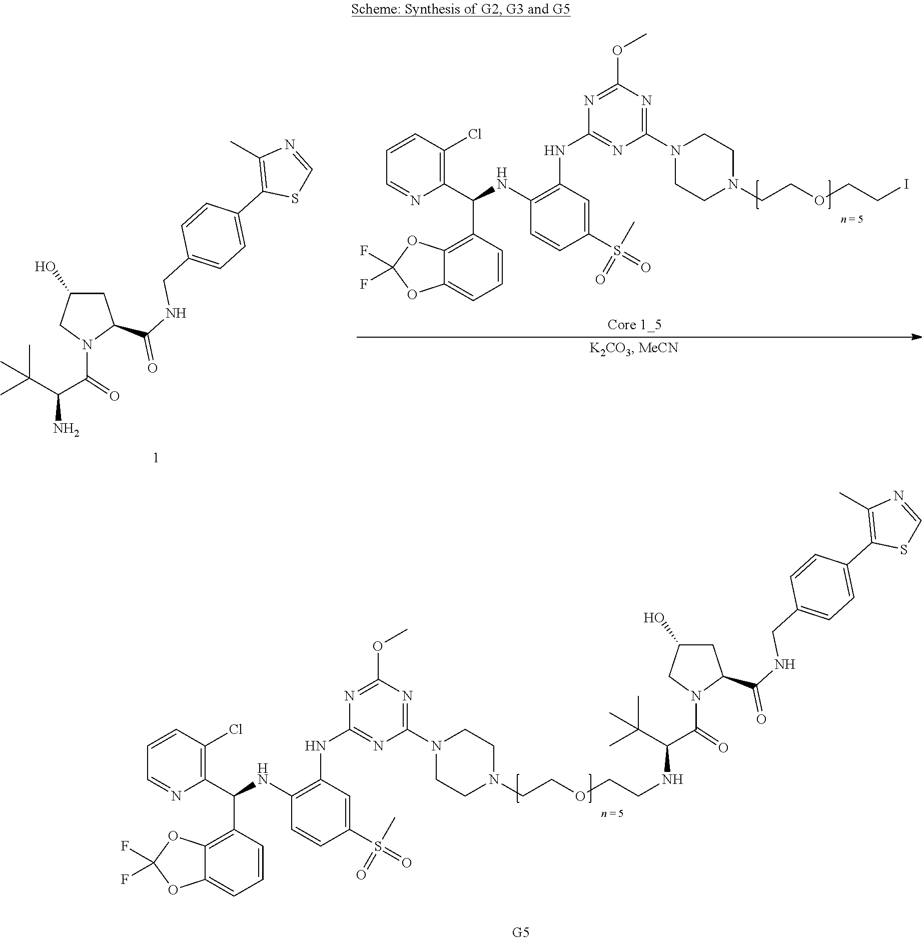

To a solution of Compound 1 (41.59 mg, 96.59 μmol, 1 eq) and Compound Core 1_5 (100 mg, 96.59 μmol, 1 eq) in ACN (2 mL) was added K₂CO₃ (40.05 mg, 289.77 μmol, 3 eq).

The mixture was stirred at 90° C. for 12 hrs. LCMS showed all the starting material was consumed and a new peak with desired product MS was detected. The mixture was filtered and purified by Prep-HPLC (column: Phenomenex Luna 80*30 mm*3 um; mobile phase: [water (0.1% TFA)-ACN]; B %: 25%-55%, 8 mins) to give G5 (32.4 mg, yield 23.1%) as a yellow solid.

¹H NMR (ET43587-185-PIA, 400 MHz, DMSO-d6) δ 1.07 (s, 9H), 1.88-1.97 (m, 1H), 2.11-2.19 (m, 1H), 2.42-2.45 (m, 3H), 2.89-2.96 (m, 1H), 3.07 (s, 6H), 3.20-3.37 (m, 4H), 3.45-3.59 (m, 19H), 3.66-3.89 (m, 9H), 4.23 (br dd, J=15.95, 5.32 Hz, 2H), 4.36-4.49 (m, 3H), 4.60 (br t, J=8.38 Hz, 2H), 6.36 (br d, J=7.13 Hz, 1H), 6.81 (br d, J=8.63 Hz, 1H), 6.89-7.02 (m, 1H), 7.09-7.24 (m, 2H), 7.32 (d, J=8.00 Hz, 1H), 7.39 (s, 4H), 7.48 (dd, J=8.00, 4.63 Hz, 2H), 7.92-8.09 (m, 2H), 8.19-8.36 (m, 1H), 8.56 (br d, J=3.50 Hz, 1H), 8.74 (br t, J=5.94 Hz, 2H), 8.99 (s, 1H), 9.48 (br d, J=2.13 Hz, 1H), 9.91 (br d, J=2.00 Hz, 1H)

LCMS (ESI+): RT=2.257 min, m/z 669.5, 1337.7 (M/2+H. M+H)⁺.

5_95AB_6min-220-MSI500: LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-1500.

### Synthesis of I2, I3 and I5

| 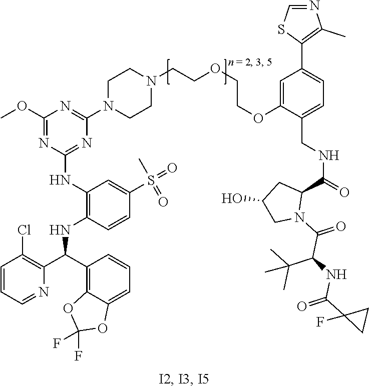 | | |
|---|---|---|
| Compound ID | Synonym | Synonym |
| NUCC-0226654 | I2 | ET43587-297 |
| NUCC-0226593 | I3 | ET43587-161 |
| NUCC-0226598 | I5 | ET43587-170 |

### Original Scheme

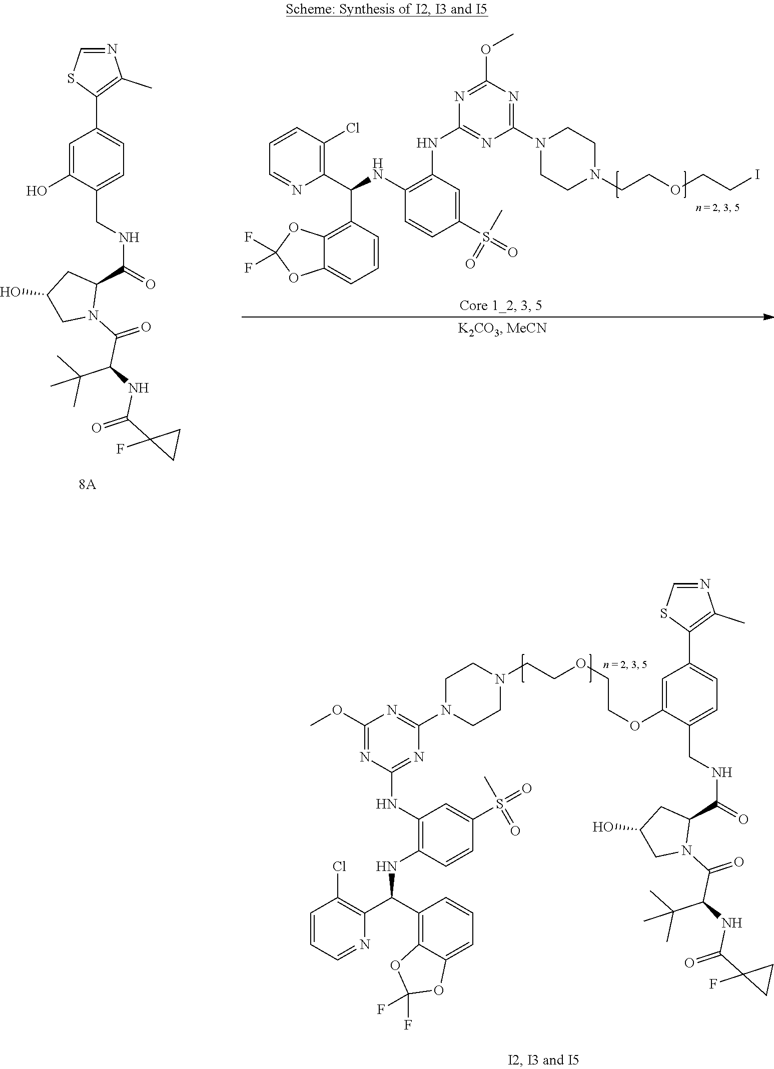

### Chemical Synthesis

All chemical reagents were obtained from commercial suppliers and used without further purification, unless otherwise stated. Reactions were run without taking precautions to exclude air or moisture, unless otherwise noted. Normal phase column chromatography was performed using silica gel columns and ACS grade solvents. Analytical TLC was performed on EM Reagent 0.25 mm silica gel 60 F254 plates and visualized by UV light. Compound identities were confirmed by ¹H and F (NMR) spectroscopy which were recorded on a Bruker 400 MHz spectrometer using the corresponding residual solvent peak (CDCl₃, 1H δ=7.27: CD₃OD, 1H δ=3.31; DMSO-d₆, 1H δ=2.50) as an internal standard. The chemical shifts for ¹H-NMR is reported to the second decimal place. Proton coupling constants are expressed in hertz (Hz). Standard abbreviations were used to denote spin multiplicity for ¹H NMR data.

### LCMS Methods:

### Method 1:5_95AB 6 Min_MS1500-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1500.

### Method 2:5_95AB_6min-220-MS1500

LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-1500.

### Experimentals for Largest Scale Run:

### General Procedure for Preparation of 12—Notebook Page: ET43587-297.

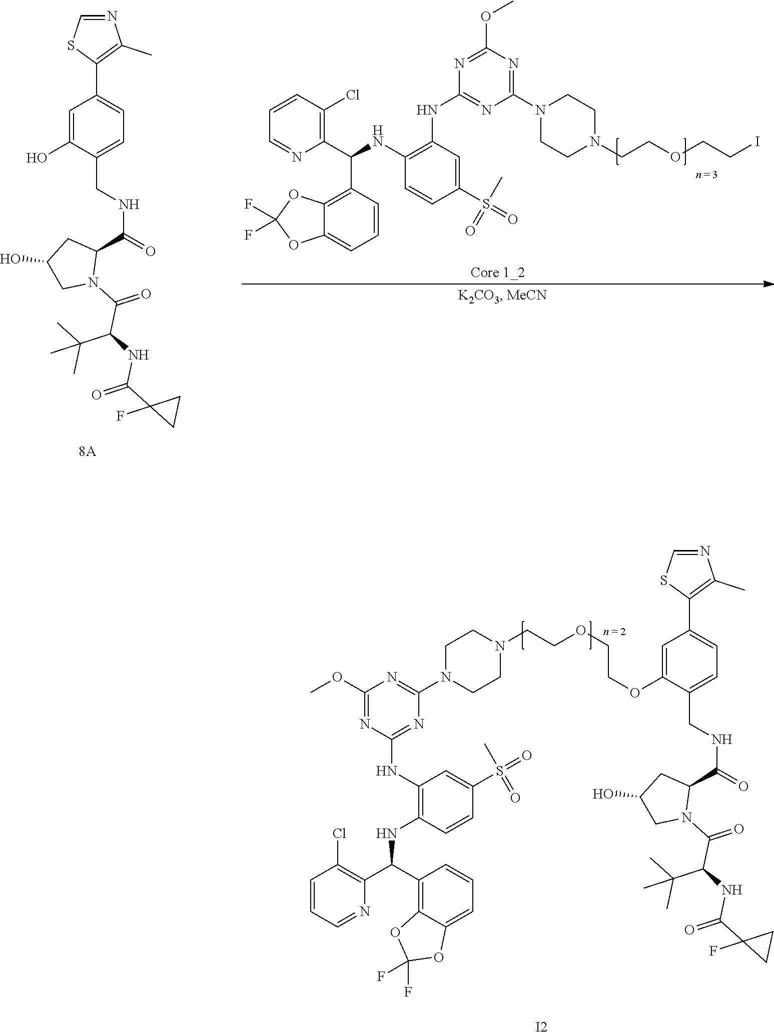

To a solution of Compound 8A (53.08 mg, 99.65 μmol, 1 eq) and Compound Core 1_2 (90 mg, 99.65 μmol, 1 eq) in DMF (2 mL) was added K₂CO₃ (41.32 mg, 298.96 μmol, 3 eq). The mixture was stirred at 60° C. for 12 hrs. LCMS showed all the starting material was consumed and a new peak with desired product MS was detected. The mixture was purified by Prep-HPLC (column: Phenomenex Luna 80*30 mm*3 um; mobile phase: [water (TFA)-ACN]; B %: 35%-65%, 8 mins) to give 12 (22.6 mg, yield 15.68%) as a white solid.

¹H NMR (ET43587-297-p1a, 400 MHz, DMSO-d6) δ 0.94 (s, 9H), 1.18-1.25 (m, 2H), 1.31-1.41 (m, 2H), 1.85-1.93 (m, 1H), 2.06-2.13 (m, 1H), 2.45 (s, 3H), 3.06 (s, 6H), 3.30 (br s, 4H), 3.78 (br d, J=17.13 Hz, 15H), 4.15-4.25 (m, 3H), 4.26-4.36 (m, 2H), 4.39-4.70 (m, 4H), 6.35 (br d, J=7.25 Hz, 1H), 6.76-6.83 (m, 1H), 6.98 (br d, J=7.63 Hz, 2H), 7.03 (s, 1H), 7.11-7.17 (m, 1H), 7.20-7.24 (m, 1H), 7.25-7.35 (m, 2H), 7.41 (d, J=7.63 Hz, 1H), 7.47 (dd, J=8.00, 4.50 Hz, 2H), 8.03 (dd, J=8.13, 1.00 Hz, 2H), 8.49-8.58 (m, 2H), 8.97 (s, 1H), 9.48 (br s, 1H), 9.81 (br d, J=1.38 Hz, 1H)

LCMS (ESI+): RT=2.501 min, m/z 654.2, 1307.0 (M/2+H, M+H)⁺.

5_95AB_6min_MS1500-220-254-ELSD: LC/MS: The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1500.

### General Procedure for Preparation of 13—Notebook Page: ET43587-161.

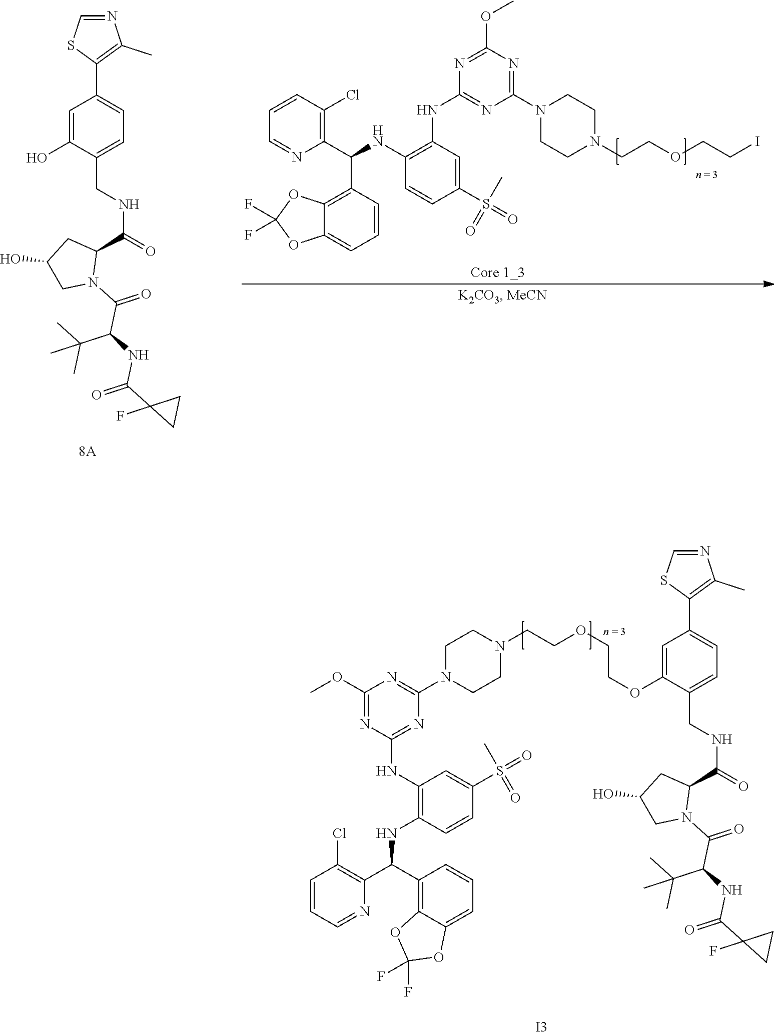

To a solution of Compound 8A (56.23 mg, 105.58 μmol, 1 eq) and Core 13 (100 mg, 105.58 μmol, 1 eq) in DMF (2 mL) was added K₂CO₃ (43.77 mg, 316.73 μmol, 3 eq). The mixture was stirred at 60° C. for 12 hrs. LCMS showed all the starting material was consumed and a new peak with desired product MS was detected. The mixture was purified by Prep-HPLC (column: Phenomenex Luna 80*30 mm*3 um; mobile phase: [water (0.1% TFA)-ACN]; B %: 30%-60%, 8 mins) to give 13 (11.3 mg, yield 6.95%) as a white solid.

¹H NMR (ET43587-161-P1C, 400 MHz, DMSO-d6) δ 0.96 (s, 9H), 1.22 (br dd, J=8.32, 2.69 Hz, 2H), 1.32-1.42 (m, 2H), 1.87-1.96 (m, 1H), 2.06-2.15 (m, 1H), 2.45 (s, 3H), 2.95-3.10 (m, 5H), 3.17-3.37 (m, 4H), 3.46-3.67 (m, 13H), 3.73-3.85 (m, 7H), 4.21-4.34 (m, 5H), 4.49-4.64 (m, 4H), 6.36 (br d, J=7.25 Hz, 1H), 6.81 (br d, J=8.75 Hz, 1H), 6.90-7.05 (m, 3H), 7.12-7.18 (m, 1H), 7.20-7.25 (m, 1H), 7.25-7.35 (m, 2H), 7.41 (d, J-=7.75 Hz, 1H), 7.44-7.54 (m, 2H), 8.03 (d, J=8.00 Hz, 2H), 8.47-8.60 (m, 2H), 8.97 (s, 1H), 9.49 (br d, J=2.75 Hz, 1H), 9.77 (br d, J-=3.25 Hz, 1H)

LCMS (ESI+): RT=2.488 min, m/z 676.3, 1251.7 (M/2+H, M+1)*.

5_95AB_6min-220-MS1500: LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-1500.

### General Procedure for Preparation of 15—Notebook Page: ET43587-170.

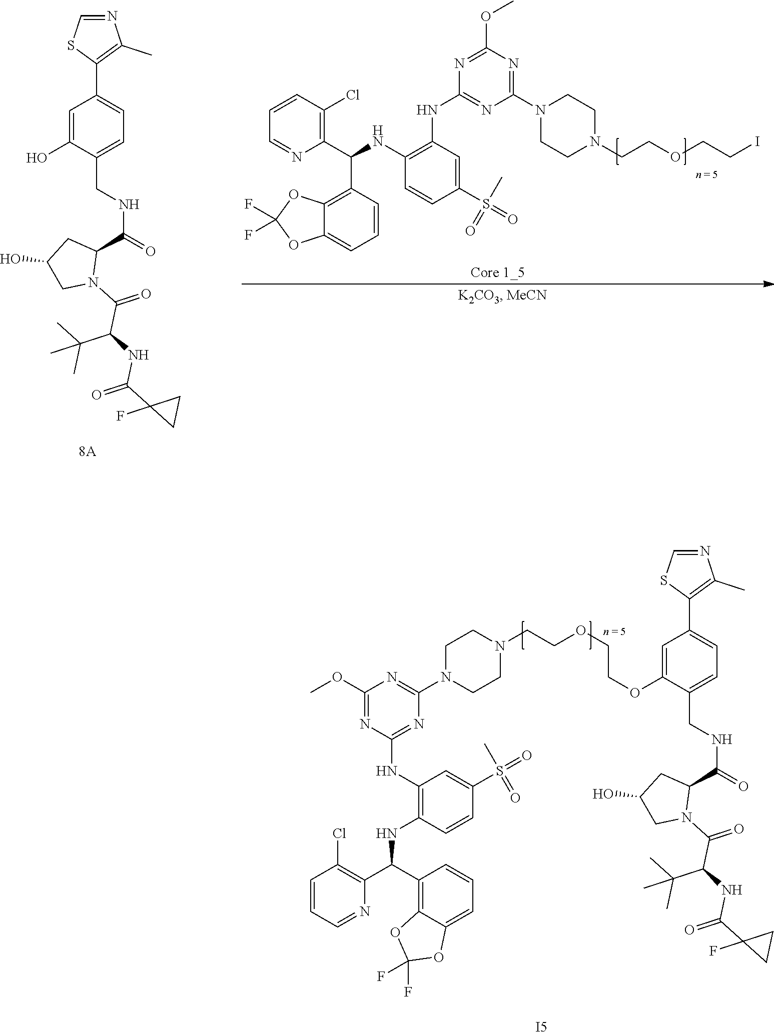

To a solution of Compound 8A (56.59 mg, 106.25 μmol, 1 eq) and Compound 15 (110 mg, 106.25 μmol, 1 eq) in DMF (2 mL) was added K₂CO₃ (44.05 mg, 318.75 μmol, 3 eq). The mixture was stirred at 60° C. for 12 hrs. LCMS showed all the starting material was consumed and a new peak with desired product MS was detected. The mixture was purified by Prep-HPLC (column: Phenomenex Luna 80*30 mm*3 um; mobile phase: [water (0.1% TFA)-ACN]; B %: 30%-55%, 8 mins) to give 15 (33.6 mg, yield 19.09%) as a white solid.

¹H NMR (ET43587-170-P1A, 400 MHz, DMSO-d6) δ 0.95 (s, 9H), 1.18-1.25 (m, 2H), 1.31-1.41 (m, 2H), 1.91 (ddd, J=12.91, 8.79, 4.32 Hz, 1H), 2.05-2.13 (m, 1H), 2.45 (s, 3H), 3.07 (s, 5H), 3.20-3.35 (m, 4H), 3.45-3.65 (m, 21H), 3.70-3.88 (m, 7H), 4.10-4.42 (m, 6H), 4.48-4.62 (m, 3H), 6.36 (br d, J=7.00 Hz, 1H), 6.81 (br d, J=8.76 Hz, 1H), 6.91-7.06 (m, 3H), 7.12-7.17 (m, 1H), 7.20-7.24 (m, 1H), 7.25-7.34 (m, 2H), 7.41 (d, J=7.88 Hz, 1H), 7.44-7.53 (m, 2H), 7.92-8.08 (m, 2H), 8.45-8.60 (m, 2H), 8.98 (s, 1H), 9.47 (br s, 1H), 9.81 (br dd, J=7.50, 2.88 Hz, 1H)

LCMS (ESI+): RT=2.511 min, m/z 720.2, 1439.7 (M/2+H, M+H)⁺.

5_95AB_6min_MS1500-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1500.

### Synthesis of J1, J2, J3 and J4

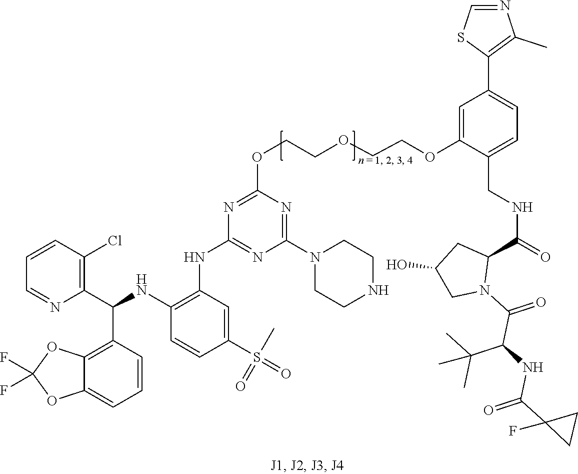

| Compound ID | Synonym | Synonym |
|---|---|---|
| NUCC-0226706 | J1 | ET43587-387 |
| NUCC-0226704 | J2 | ET43587-370 |
| NUCC-0226705 | J3 | ET43587-393 |
| NUCC-0226707 | J4 | ET43587-392 |

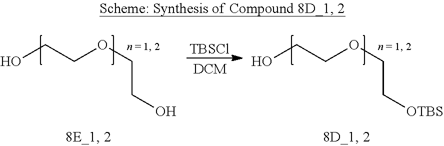

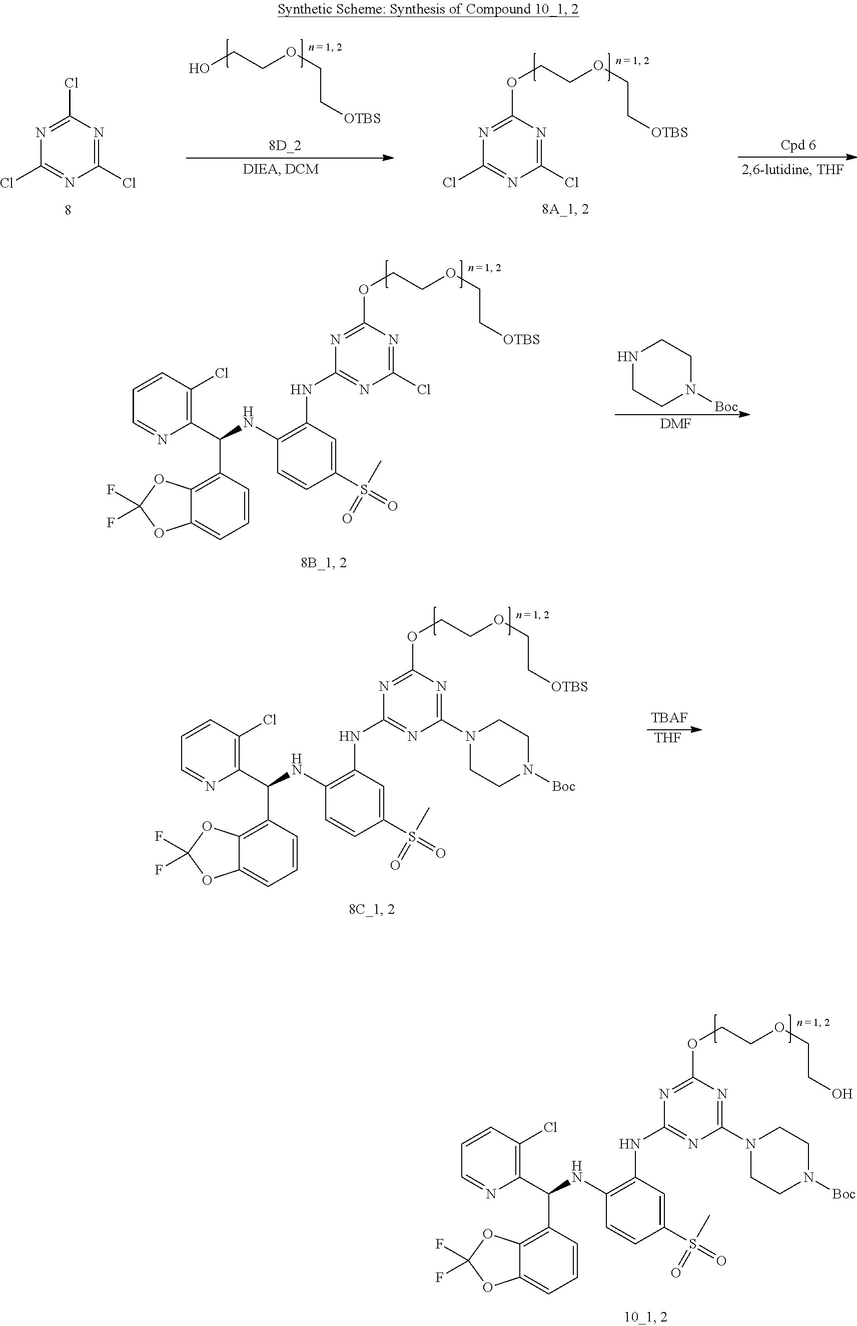

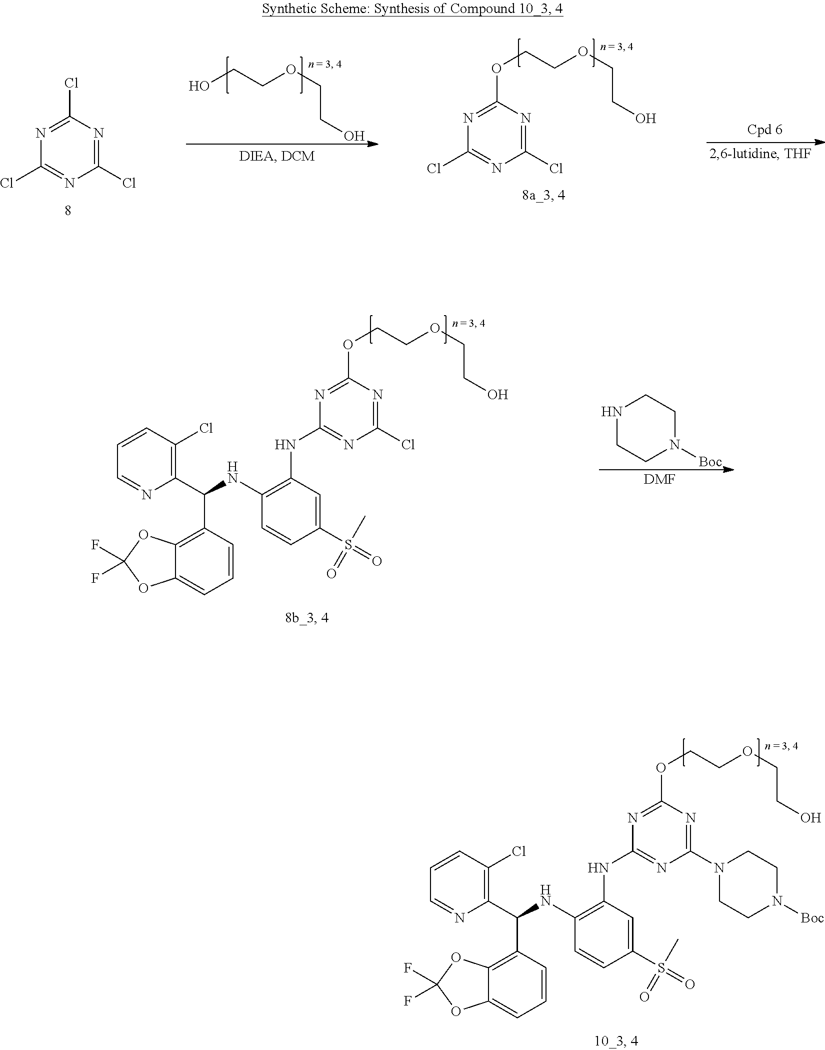

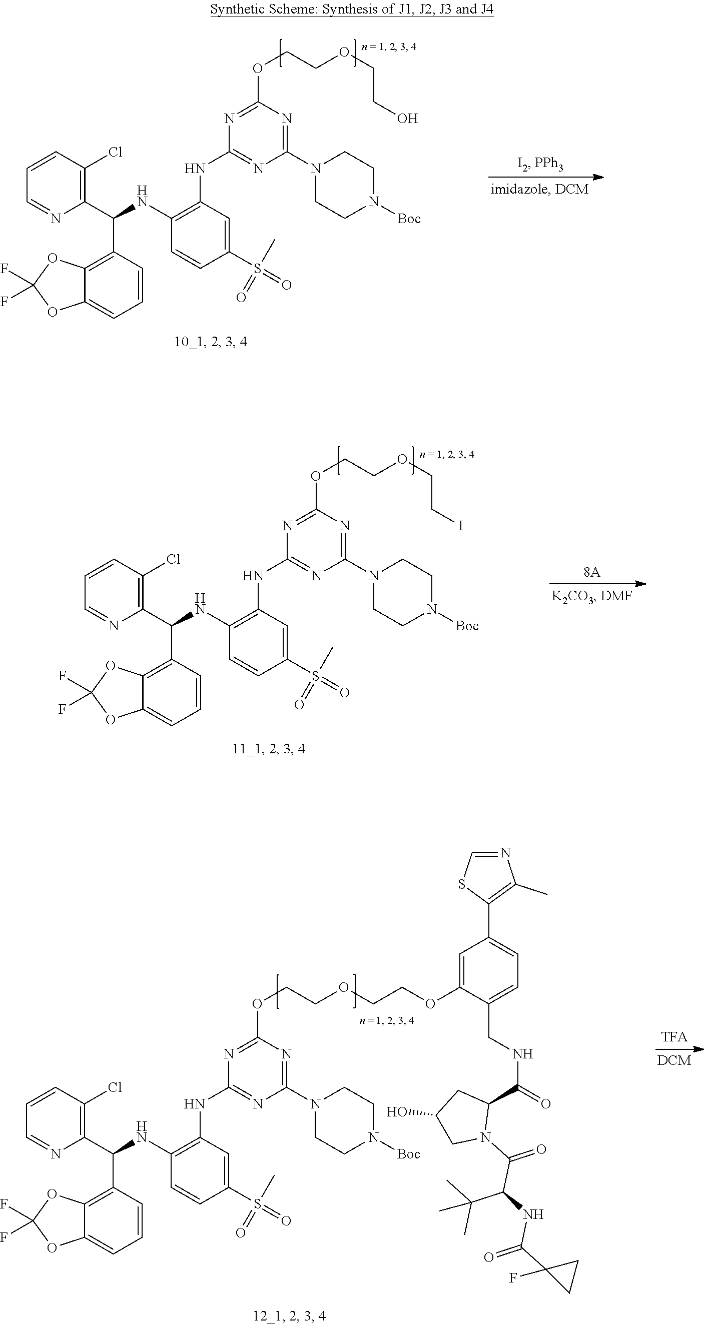

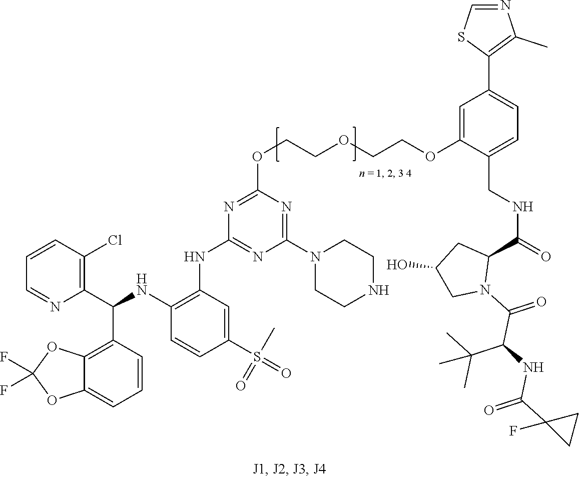

### Chemical Synthesis

All chemical reagents were obtained from commercial suppliers and used without further purification, unless otherwise stated. Reactions were run without taking precautions to exclude air or moisture, unless otherwise noted. Normal phase column chromatography was performed using silica gel columns and ACS grade solvents. Analytical TLC was performed on EM Reagent 0.25 mm silica gel 60 F254 plates and visualized by UV light. Compound identities were confirmed by ¹H and F (NMR) spectroscopy which were recorded on a Bruker 400 MHz spectrometer using the corresponding residual solvent peak (CDC3,1H δ=7.27; CD₃OD, 1H δ=3.31; DMSO-d₆, 1H δ=2.50) as an internal standard. The chemical shifts for ¹H-NMR is reported to the second decimal place. Proton coupling constants are expressed in hertz (Hz). Standard abbreviations were used to denote spin multiplicity for ¹H NMR data.

### LCMS Methods:

### Method 1: 5_95AB_2min

LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### Method 2: 5_95AB6min_MS1500-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1500.

### Method 2: 5_95AB_6min-220-MS2000

LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-2000.

### Experimental for Largest Scale Run:

### General Procedure for Preparation of Compound 8D_1—Notebook Page: ET42365-298.

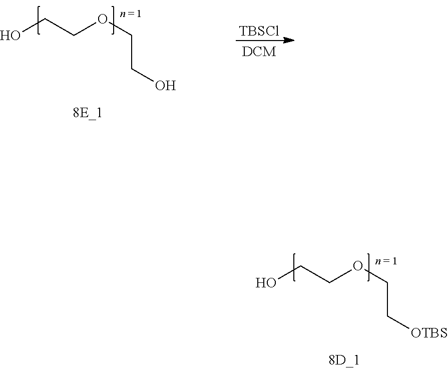

To a solution of Compound 8E_1 (29.92 g, 281.94 mmol, 26.71 mL, 5 eq), Et₃N (5.71 g, 56.40 mmol, 7.85 mL, 1 eq) and DMAP (1.38 g, 11.28 mmol, 0.2 eq) in DCM (150 mL) was added a solution of TBSCl (8.5 g, 56.40 mmol, 6.91 mL, 1 eq) in DCM (25 mL) dropwise at 0° C., the reaction was stirred at 25° C. for 3 hrs. TLC (Petroleum ether/Ethyl acetate=1/1) showed about 60% of the starting material was remaining and a new spot was generated. The reaction was diluted with water (200 mL) and was extracted with DCM (3×100 mL). The combined organic layer were washed with brine (150 mL) and dried over Na₂SO₄ and concentrated to give crude product.

The crude product was purified by silica gel chromatography (eluting with 0 to 50% ethyl acetate in petroleum ether) to give Compound 8D_1 (9 g, yield 72.41%) as a colorless oil.

¹H NMR (ET42365-298-P1A, 400 MHz, CHLOROFORM-d) δ 0.09 (s, 6H), 0.91 (s, 9H), 2.14 (br s, 1H), 3.57-3.66 (m, 4H), 3.70-3.76 (m, 2H), 3.76-3.82 (m, 2H)

### General Procedure for Preparation of Compound 8A_1—Notebook Page: ET43587-326.

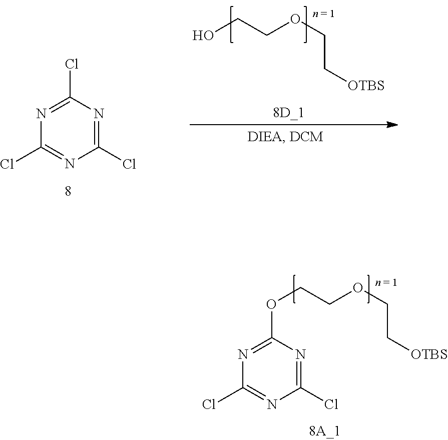

To a solution of Compound 8 (500 mg, 2.71 mmol, 1 eq) in DCM (10 mL) were added Compound 8D_1 (597.52 mg, 2.71 mmol, 1 eq) and DIEA (350.42 mg, 2.71 mmol, 472.27 μL, 1 eq) in DCM (5 mL) dropwise at 0° C. The mixture was stirred at 25° C. for 12 hrs. LCMS showed all the starting material was consumed and a new peak with desired product MS was detected. The reaction mixture was diluted with water (20 mL) and extracted with ethyl acetate (3×20 mL). The organic layer was separated and the combined organic layer was washed with brine (20 mL), dried over Na₂SO₄, filtered and concentrated under reduced pressure to give a residue, which was purified by chromatography on silica gel (eluted with petroleum ether/ethyl acetate=50/1 to 4/1) to give Compound 8A_1 (570 mg, yield 54.22%) as a colorless oil.

¹H NMR (ET43587-326-p1a, 400 MHz, CHLOROFORM-d) δ 0.06 (s, 6H), 0.89 (s, 9H), 3.58-3.64 (m, 2H), 3.74-3.80 (m, 2H), 3.88 (dd, J=5.32, 3.94 Hz, 2H), 4.65 (dd, J-=5.50, 3.88 Hz, 2H)

### General Procedure for Preparation of Compound 8B_1—Notebook Page: ET43587-345.

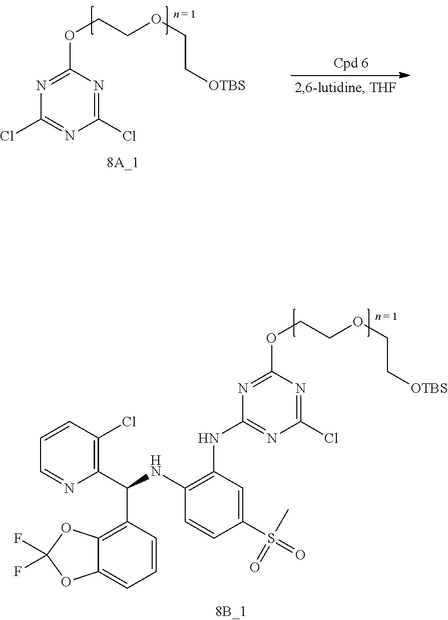

To a solution of Compound 8A_1 (570 mg, 1.55 mmol, 1 eq) and 2,6-lutidine (165.82 mg, 1.55 mmol, 180.24 μL, 1 eq) in THF (12 mL) was added Compound 6 (724.04 mg, 1.55 mmol, 1 eq) in THF (6 mL) dropwise. The mixture was stirred at 25° C. for 12 hrs. LCMS showed all the starting material was consumed and a new peak with desired product MS was detected. The reaction mixture was diluted with water (30 mL) and extracted with ethyl acetate (3×30 mL). The organic layer was separated and the combined organic layer was washed with brine (2-30 mL), dried over Na₂SO₄, filtered and concentrated under reduced pressure to give a residue, which was purified by chromatography on silica gel (eluted with petroleum ether/ethyl acetate=50/1 to 1/1) to give Compound 8B1 (660 mg, yield 48%) as a yellow oil.

¹H NMR (ET43587-345-P1S, 400 MHz, CHLOROFORM-d) δ 0.03 (br s, 6H), 0.87 (br s, 9H), 3.02 (s, 3H), 3.45-3.93 (m, 6H), 4.30-4.61 (m, 2H), 6.26 (br s, 1H), 6.83 (br d, J=2.38 Hz, 1H), 6.89-7.05 (m, 4H), 7.25 (s, 1H), 7.60 (br s, 1H), 7.67-7.76 (m, 2H), 7.94 (br s, 1H), 8.47 (dd, J=4.63, 1.25 Hz, 1H)

### General Procedure for Preparation of Compound 8C_1—Notebook Page: ET43587-351.

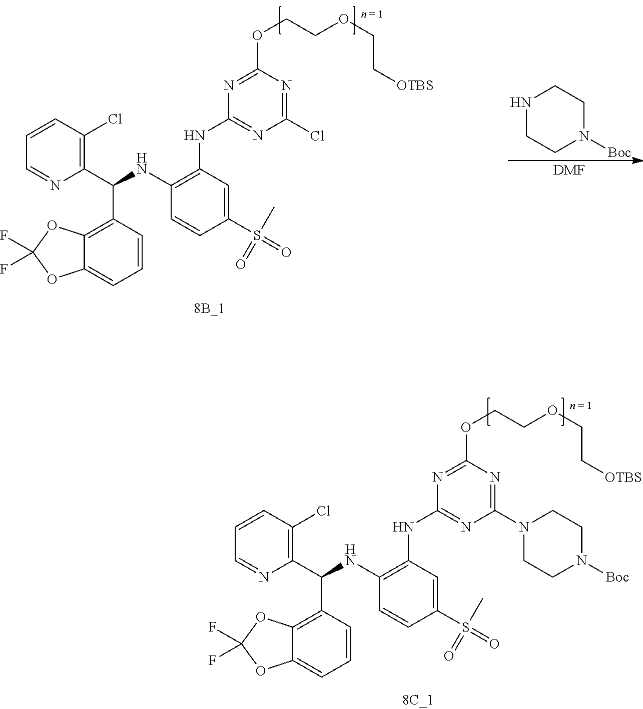

To a solution of Compound 8B_1 (660 mg, 825.27 μmol, 1 eq) and tert-butyl piperazine-1-carboxylate (153.71 mg, 825.27 μmol, 1 eq) in DMF (12 mL) was added TEA (83.51 mg, 825.27 μmol, 114.87 μL, 1 eq). The mixture was stirred at 90° C. for 2 hrs. LCMS showed all the starting material was consumed and a new peak with desired product MS was detected. The reaction mixture was diluted with water (20 mL) and extracted with ethyl acetate (3×20 mL). The organic layer was separated and the combined organic layer was washed with brine (2×20 mL), dried over Na₂SO₄, filtered and concentrated under reduced pressure to give a residue, which was purified by chromatography on silica gel (eluted with petroleum ether/ethyl acetate=20/1 to 0/1) to give Compound SC_1 (570 mg, yield 65.47%) as a yellow oil.

LCMS (ESI+): RT=0.971 min, m/z 949.4 (M+H)⁺.

5_95AB_2min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### General Procedure for Preparation of Compound 10_1—Notebook Page: ET43587-352.

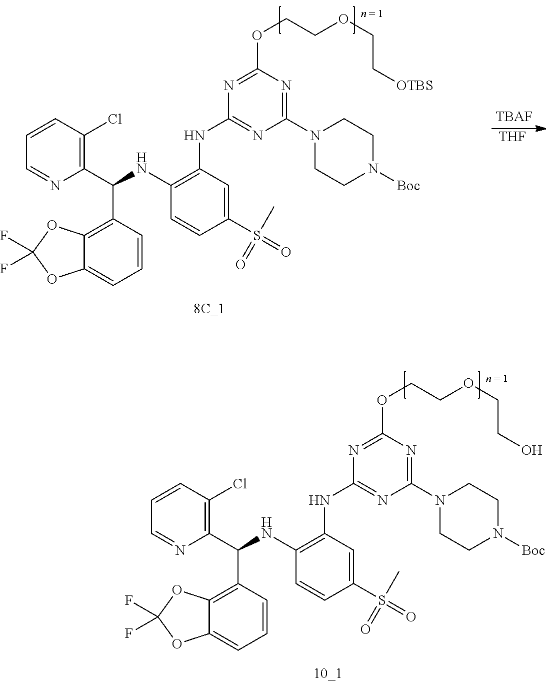

To a solution of Compound 8C1 (570 mg, 540.27 μmol, 1 eq) in THF (8 mL) was added TBAF (1 M, 1080.55 μL, 2 eq) at 0° C. The mixture was stirred at 25° C. for 12 hrs. TLC (ethyl acetate/methanol=2/1) showed the starting material was consumed and a new spot was generated. The reaction mixture was diluted with water (20 mL) and extracted with ethyl acetate (3×20 mL). The organic layer was separated and the combined organic layer was washed with brine (20 mL), dried over Na₂SO₄, filtered and concentrated under reduced pressure to give a residue, which was purified by chromatography on silica gel (eluted with ethyl acetate/methanol=I/O to 2/1) to give Compound 10_1 (450 mg, yield 85.33%) as a colorless oil.

¹H NMR (ET43587-352-P1A, 400 MHz, CHLOROFORM-d) δ 1.48 (s, 9H), 3.43 (br s, 4H), 3.54-3.90 (m, 10H), 4.47 (br s, 2H), 6.26 (d, J=7.25 Hz, 1H), 6.72 (br d, J=7.38 Hz, 1H), 6.87-7.03 (m, 4H), 7.22-7.26 (m, 1H), 7.62 (dd, J=8.57, 1.94 Hz, 1H), 7.71 (dd, J=8.00, 1.25 Hz, 1H), 8.10 (br s, 1H), 8.47-8.53 (m, 1H)

### General Procedure for Preparation of Compound 11_1—Notebook Page: ET43587-364.

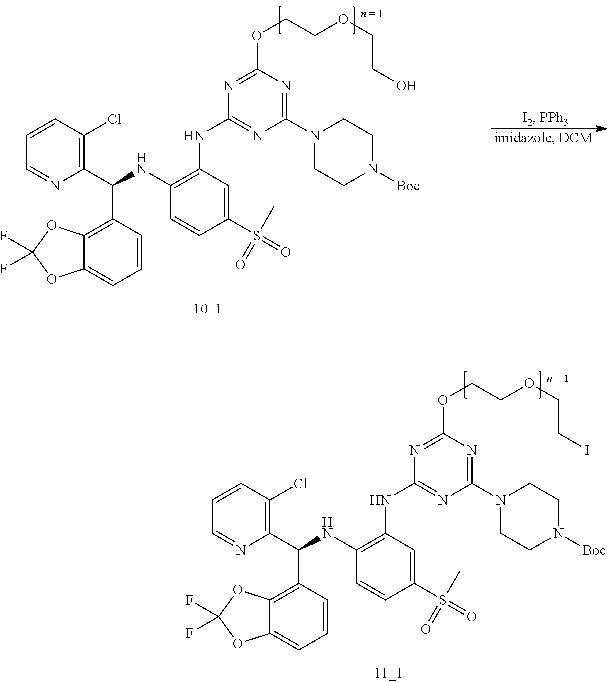

To a solution of Compound 101 (450 mg, 538.75 μmol, 1 eq) and 12 (410.21 mg, 1.62 mmol, 3 eq) in DCM (9 mL) were added imidazole (110.03 mg, 1.62 mmol, 3 eq) and PPh₃ (423.92 mg, 1.62 mmol, 3 eq) at 0° C. The mixture was stirred at 25° C. for 12 hrs. LCMS showed all the starting material was consumed and a new peak with desired product MS was detected. The reaction mixture was diluted with water (30 mL) and extracted with ethyl acetate (3×30 mL). The organic layer was separated and the combined organic layer was washed with brine (2-30 mL), dried over Na₂SO₄, filtered and concentrated under reduced pressure to give a residue, which was purified by chromatography on silica gel (eluted with petroleum ether/ethyl acetate=20/1 to 0/1) to give Compound 11_1 (200 mg, yield 31.42%) as a yellow solid.

¹H NMR (ET43587-364-p1a, 400 MHz, CHLOROFORM-d) δ 1.48 (s, 9H), 2.99 (s, 3H), 3.25 (br t, J=6.84 Hz, 2H), 3.43 (br s, 4H), 3.57-3.84 (m, 8H), 4.45 (br s, 2H), 6.25 (d, J=7.28 Hz, 1H), 6.75 (br d, J=7.28 Hz, 1H), 6.87-7.02 (m, 5H), 7.24 (dd, J=8.03, 4.64 Hz, 1H), 7.63 (dd, J=8.53, 2.13 Hz, 1H), 8.10 (br s, 1H), 8.49 (dd, J=4.58, 1.19 Hz, 1H)

### General Procedure for Preparation of Compound 12_1—Notebook Page: ET43587-374.

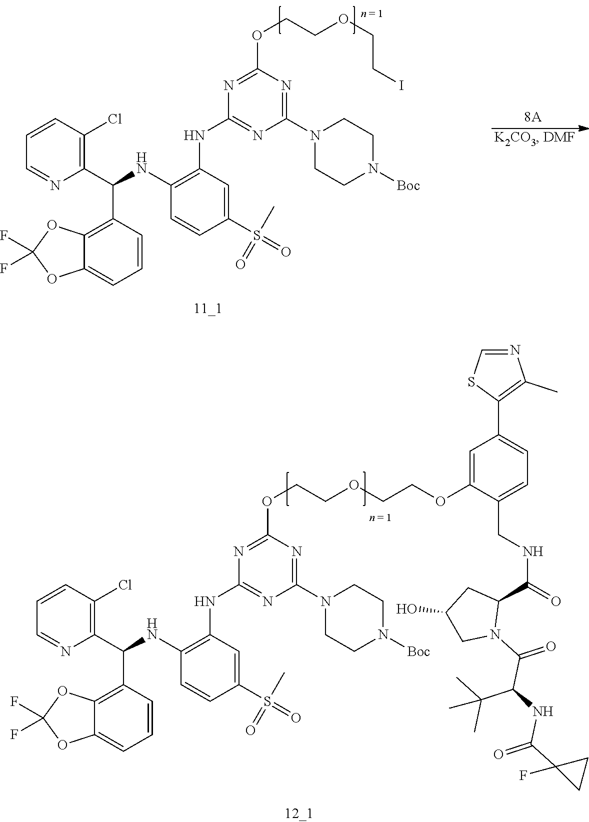

To a solution of Compound 11_1 (130 mg, 82.52 μmol, 1 eq) and Compound 8A (43.95 mg, 82.52 μmol, 1 eq) in DMF (2 mL) was added K₂CO₃ (17.11 mg, 123.79 μmol, 1.5 eq). The mixture was stirred at 60° C. for 12 hrs. LCMS showed all the starting material was consumed and a new peak with desired product MS was detected. The reaction mixture was purified by Prep-HPLC (column: Phenomenex Luna 80*30 mm*3 um; mobile phase: [water (TFA)-ACN]; B %: 55%-80%, 8 mins) to give Compound 12_1 (53 mg, yield 41.68%) as a yellow solid.

LCMS (ESI+): RT=0.870 min, m/z 1349.5 (M+H)⁺.

5_95AB_2min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### General Procedure for Preparation of J1—Notebook Page: ET43587-387.

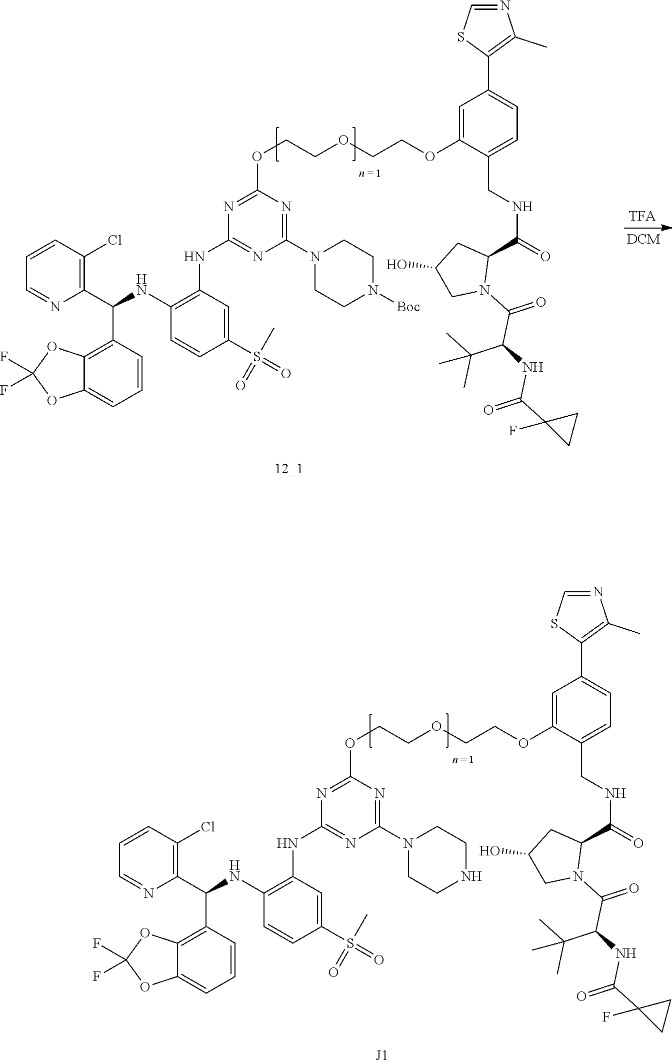

To a solution of Compound 12_1 (53 mg, 39.26 μmol, 1 eq) in DCM (1 mL) was added TFA (0.4 mL) dropwise at 0° C. The mixture was stirred at 25° C. for 12 hrs. LCMS showed all the starting material was consumed and a new peak with desired product MS was detected. The reaction mixture was concentrated under reduce pressure to give a residue, which was purified by Prep-HPLC (column: Phenomenex Luna 80*30 mm*3 um; mobile phase: [water (TFA)-ACN]; B %: 30%-65%, 8 mins) to give Compound J1 (34.1 mg, yield 63.68%) as a white solid.

¹H NMR (ET43587-387-p1a, 400 MHz, DMSO-d6) δ 0.94 (s, 9H), 1.21 (br dd, J=8.44, 2.19 Hz, 2H), 1.29-1.42 (m, 2H), 1.91 (ddd, J=12.85, 8.85, 4.32 Hz, 1H), 2.08 (br dd, J=12.51, 7.75 Hz, 1H), 2.44 (s, 3H), 3.07 (s, 7H), 3.82 (br s, 11H), 4.14-4.24 (m, 3H), 4.26-4.46 (m, 4H), 4.48-4.62 (m, 2H), 6.35 (d, J=7.25 Hz, 1H), 6.81 (d, J=9.01 Hz, 1H), 6.90-7.07 (m, 3H), 7.10-7.34 (m, 4H), 7.38-7.53 (m, 3H), 8.01 (dd, J=8.13, 1.13 Hz, 2H), 8.47-8.58 (m, 2H), 8.79 (br s, 2H), 8.97 (s, 1H), 9.42 (br s, 1H)

LCMS (ESI+): RT=2.470 min, m/z 625.3, 1249.5 (M/2+H, M+H)⁺.

5_95AB_6min_MS1500-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1500.

### General Procedure for Preparation of Compound 8D_2—Notebook Page: ET42365-296.

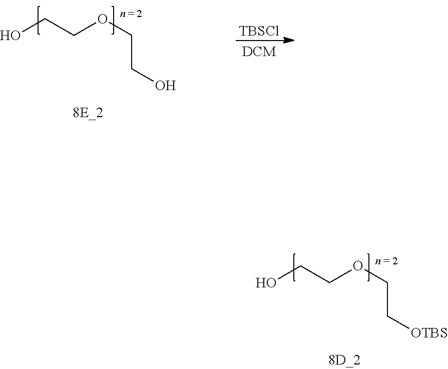

To a solution of Compound 8E_2 (29.89 g, 199.04 mmol, 26.69 mL, 5 eq), Et₃N (4.03 g, 39.81 mmol, 5.54 mL, 1 eq) and DMAP (486.33 mg, 3.98 mmol, 0.1 eq) in DCM (150 mL) was added a solution of TBSCl (6 g, 39.81 mmol, 4.88 mL, 1 eq) in DCM (30 mL) dropwise at 0° C., the reaction was stirred at 25° C. for 3 hrs. TLC (petroleum ether/ethyl acetate=1/1) showed about 60% of the starting material was remaining and a new spot was generated. The reaction was diluted with water (200 mL) and was extracted with DCM (3-100 mL). The combined organic layer were washed with brine (150 mL) and dried over Na₂SO₄ and concentrated to give crude product. The crude product was purified by silica gel chromatography (eluting with 0 to 50% ethyl acetate in petroleum ether) to give Compound SD_2 (8 g, yield 76%) as a colorless oil.

¹H NMR (ET42365-296-P1A, 400 MHz, CHLOROFORM-d) δ 0.08 (s, 6H), 0.90 (s, 9H), 2.23 (s, 1H), 3.58 (t, J=5.32 Hz, 2H), 3.60-3.65 (m, 2H), 3.68 (s, 4H), 3.72-3.76 (m, 2H), 3.76-3.81 (m, 2H)

### General Procedure for Preparation of Compound 8A2—Notebook Page: ET43587-321.

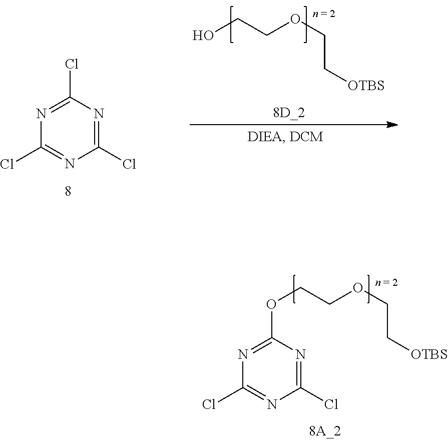

To a solution of Compound 8 (500 mg, 2.71 mmol, 1 eq) and DIEA (350.41 mg, 2.71 mmol, 472.25 μL, 1 eq) in DCM (10 mL) was added Compound 8D_2 (716.96 mg, 2.71 mmol, 1 eq) in DCM (5 mL) dropwise at 0° C. The mixture was stirred at 25° C. for 12 hrs. LCMS showed all the starting material was consumed and a new peak was detected. The reaction mixture was diluted with water (30 mL) and extracted with ethyl acetate (3-30 mL). The organic layer was separated and the combined organic layer was washed with brine (30 mL), dried over Na₂SO₄, filtered and concentrated under reduced pressure to give a residue, which was purified by chromatography on silica gel (eluted with petroleum ether/ethyl acetate=20/1 to 3/1) to give Compound 8A_2 (800 mg, yield 64.39%) as a yellow oil.

¹H NMR (ET43587-321-p1a, 400 MHz, CHLOROFORM-d) δ 0.07 (s, 6H), 0.90 (s, 9H), 3.53-3.58 (m, 2H), 3.65-3.70 (m, 4H), 3.74-3.78 (m, 2H), 3.86-3.89 (m, 2H), 4.63-4.67 (m, 2H)

### General Procedure for Preparation of Compound 8B_2—Notebook Page: ET43587-323.

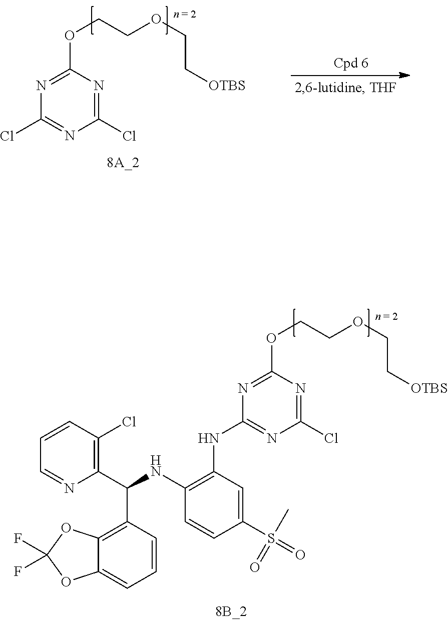

To a solution of Compound 8A_2 (300 mg, 727.48 μmol, 1 eq) and 2,6-lutidine (77.95 mg, 727.48 μmol, 84.73 μL, 1 eq) in THF (6 mL) was added Compound 6 (340.37 mg, 727.48 μmol, 1 eq) in THF (2 mL) dropwise. The mixture was stirred at 25° C. for 12 hrs. LCMS showed all the starting material was consumed and a new peak with desired product MS was detected. The reaction mixture was diluted with water (10 mL) and extracted with ethyl acetate (3×10 mL). The organic layer was separated and the combined organic layer was washed with brine (10 mL), dried over Na₂SO₄, filtered and concentrated under reduced pressure to give a residue, which was purified by chromatography on silica gel (eluted with petroleum ether/ethyl acetate=20/1 to 1/1) to give Compound 8B_2 (430 mg, yield 66.55%) as a yellow oil.

¹H NMR (ET43587-323-p1a, 400 MHz, CHLOROFORM-d) δ 0.05 (br s, 6H), 0.88 (br s, 9H), 3.02 (s, 3H), 3.49-3.91 (m, 10H), 4.30-4.65 (m, 2H), 6.26 (br s, 1H), 6.84 (br d, J=5.50 Hz, 1H), 6.89-7.02 (m, 4H), 7.25 (s, 1H), 7.54 (br d, J=7.75 Hz, 1H), 7.72 (td, J=8.19, 1.63 Hz, 2H), 7.94 (br s, 1H), 8.47 (dd, J=4.75, 1.38 Hz, 1H)

### General Procedure for Preparation of Compound 8C_2—Notebook Page: ET43587

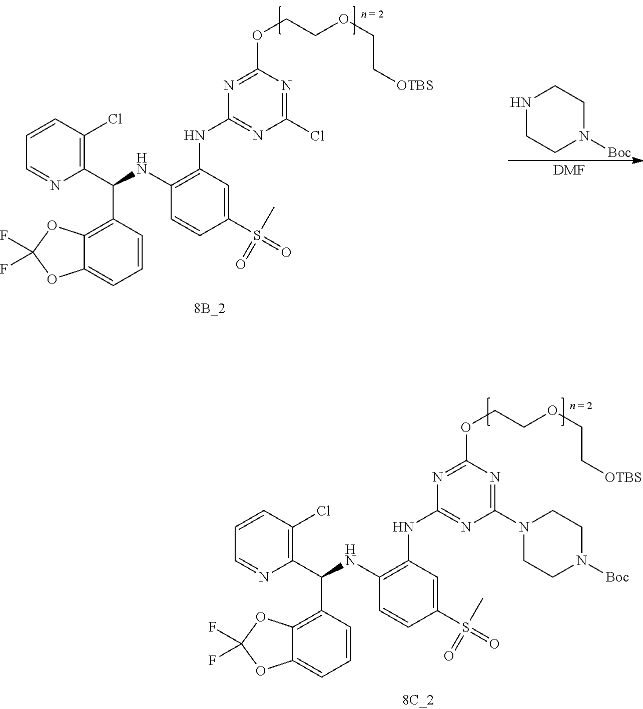

To a solution of Compound 8B_2 (430 mg, 509.60 μmol, 1 eq) and tert-butyl piperazine-1-carboxylate (94.91 mg, 509.60 μmol, 1 eq) in DMF (9 mL) was added TEA (51.57 mg, 509.60 μmol, 1 eq). The mixture was stirred at 90° C. for 2 hrs. LCMS showed all the starting material was consumed and a new peak with desired product MS was detected. The reaction mixture was diluted with water (20 mL) and extracted with ethyl acetate (3×20 mL). The organic layer was separated and the combined organic layer was washed with brine (2×20 mL), dried over Na₂SO₄, filtered and concentrated under reduced pressure to give a residue, which was purified by chromatography on silica gel (eluted with petroleum ether/ethyl acetate=20/1 to 0/1) to give Compound 8C_2 (400 mg, yield 71.1%) as a yellow oil.

¹H NMR (ET43587-325-p1a, 400 MHz, CHLOROFORM-d) δ 0.07 (s, 6H), 0.90 (s, 9H), 1.47 (s, 9H), 2.99 (s, 3H), 3.42 (br s, 4H), 3.52-3.86 (m, 14H), 4.44 (br s, 2H), 6.24 (d, J=7.15 Hz, 1H), 6.79 (br d, J=6.65 Hz, 1H), 6.86-7.03 (m, 4H), 7.23 (dd, J=8.09, 4.71 Hz, 1H), 7.62 (dd, J=8.60, 1.94 Hz, 1H), 7.69 (d, J=8.16 Hz, 1H), 8.08 (br s, 1H), 8.49 (br d, J=4.14 Hz, 1H)

### General Procedure for Preparation of Compound 10_2—Notebook Page: ET43587-335.

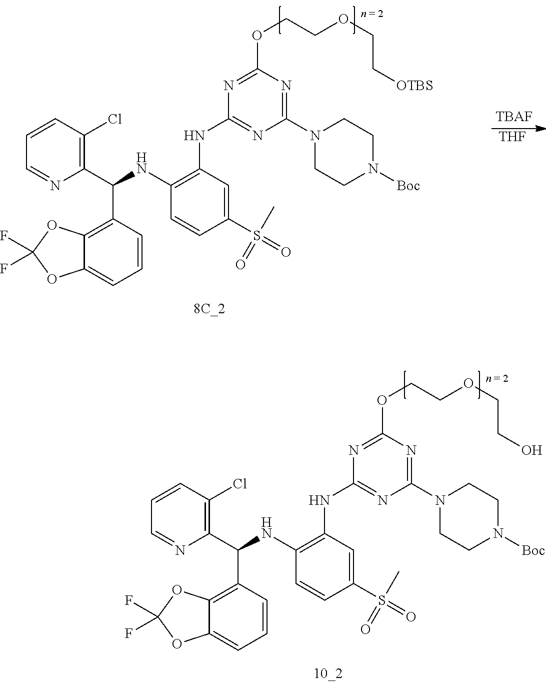

To a solution of Compound 8C_2 (400 mg, 402.58 μmol, 1 eq) in THF (8 mL) was added TBAF (1 M, 805.17 μL, 2 eq) at 0° C. The mixture was stirred at 25° C. for 12 hrs. LCMS showed all the starting material was consumed and anew peak with desired product MS was detected. The reaction mixture was diluted with water (10 mL) and extracted with ethyl acetate (3×10 mL). The organic layer was separated and the combined organic layer was washed with brine (10 mL), dried over Na₂SO₄, filtered and concentrated under reduced pressure to give a residue, which was purified by chromatography on silica gel (eluted with ethyl acetate/methanol=I/O to 2/1) to give Compound 10_2 (320 mg, yield 85.88%) as a colorless oil.

¹H NMR (ET43587-335-p1a, 400 MHz, CHLOROFORM-d) δ 1.48 (s, 9H), 2.99 (s, 3H), 3.35-3.46 (m, 4H), 3.58-3.85 (m, 14H), 4.46 (br s, 2H), 6.26 (d, J=7.38 Hz, 1H), 6.72 (br d, J=7.13 Hz, 1H), 6.84-7.01 (m, 4H), 7.21-7.26 (m, 1H), 7.62 (dd, J=8.63, 2.00 Hz, 1H), 7.71 (d, J=8.13 Hz, 1H), 8.10 (br s, 1H), 8.48-8.54 (m, 1H)

### General Procedure for Preparation of Compound 11_2—Notebook Page: ET43587-344.

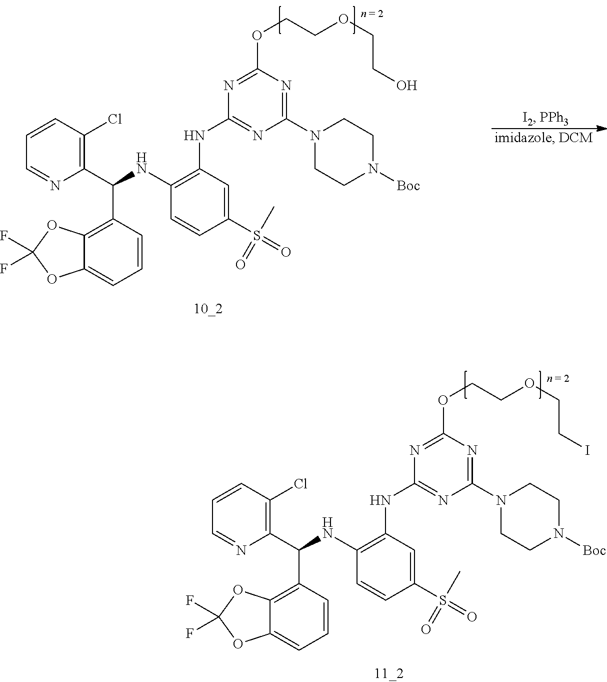

To a solution of Compound 102 (300 mg, 341.17 μmol, 1 eq) and 12 (216.48 mg, 852.93 μmol, 2.5 eq) in DCM (8 mL) were added imidazole (58.07 mg, 852.93 μmol, 2.5 eq) and PPh₃ (223.71 mg, 852.93 μmol, 2.5 eq) at 0° C. The mixture was stirred at 25° C. for 12 hrs. LCMS showed all the starting material was consumed and a new peak with desired product MS was detected. The reaction mixture was diluted with water (20 mL) and extracted with dichloromethane (2×20 mL). The organic layer was separated and the combined organic layer was washed with brine (20 mL), dried over Na₂SO₄, filtered and concentrated under reduced pressure to give a residue, which was purified by prep-TLC (Petroleum ether/ethyl acetate=1/1) to give Compound 11_2 (140 mg, yield 33.19%) as a yellow solid.

¹H NMR (ET43587-344-p1b, 400 MHz, CHLOROFORM-d) δ 1.48 (s, 9H), 2.99 (s, 3H), 3.26 (t, J=6.90 Hz, 2H), 3.43 (br s, 4H), 3.60-3.84 (m, 12H), 4.46 (br s, 2H), 6.26 (d, J=7.53 Hz, 1H), 6.73 (br d, J=7.53 Hz, 2H), 6.88-7.02 (m, 4H), 7.22-7.26 (m, 1H), 7.62 (dd, J=8.60, 2.07 Hz, 1H), 8.09 (br s, 1H), 8.49-8.57 (m, 1H)

### General Procedure for Preparation of Compound 12_2—Notebook Page: ET43587-361.

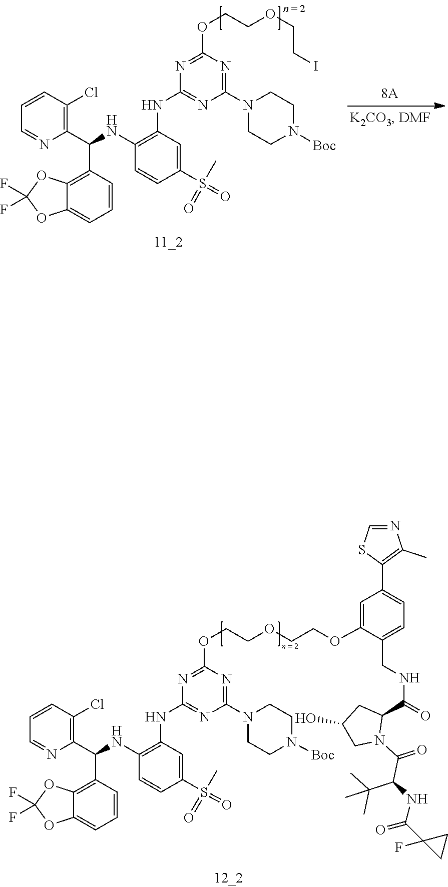

To a solution of Compound 11_2 (100 mg, 101.09 μmol, 1 eq) and Compound 8A (53.84 mg, 101.09 μmol, 1 eq) in DMF (2 mL) was added K₂CO₃ (41.91 mg, 303.27 μmol, 3 eq). The mixture was stirred at 60° C. for 12 hrs. LCMS showed all the starting material was consumed and a new peak with desired product MS was detected. The mixture was purified by Prep-HPLC (column: Phenomenex Luna 80*30 mm*3 um; mobile phase: [water (TFA)-ACN]; B %: 60%-80%, 8 min) to give Compound 12_2 (30 mg, yield 17.71%) as a white solid.

LCMS (ESI+): RT=0.865 min, m/z 1393.6 (M+H)⁺.

5_95AB_2 min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### General Procedure for Preparation of J2—Notebook Page: ET43587-370.

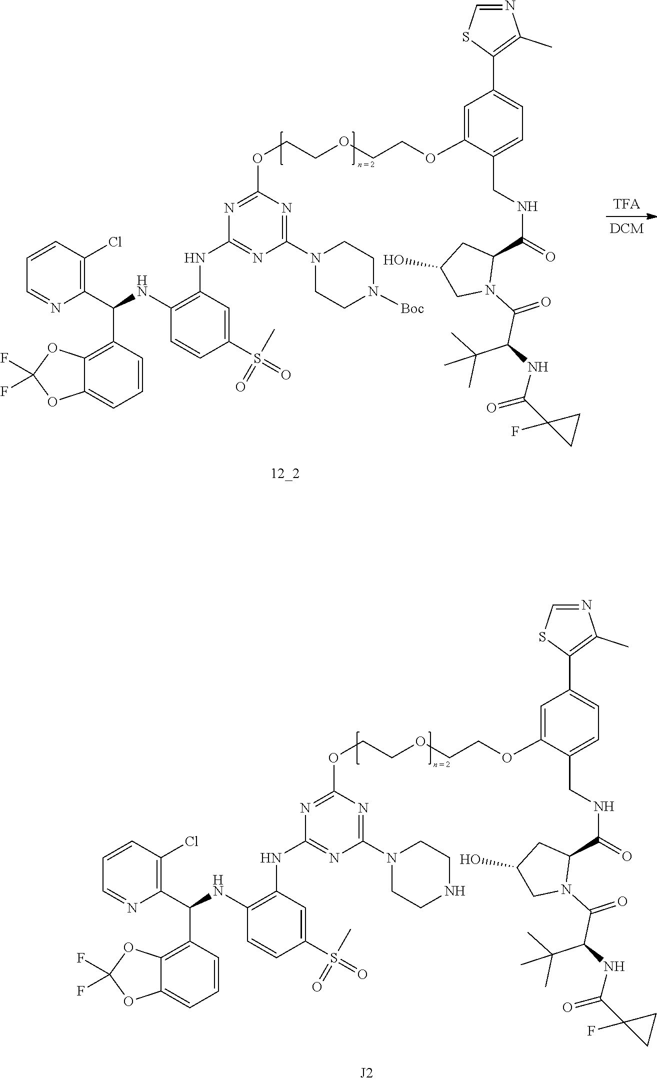

To a solution of Compound 122 (40 mg, 28.70 μmol, 1 eq) in DCM (1 mL) was added TFA (0.3 mL). The mixture was stirred at 25° C. for 5 hrs. LCMS showed all the starting material was consumed and a new peak with desired product MS was detected. The reaction mixture was purified by Prep-HPLC (column: Phenomenex Luna 80*30 mm*3 um; mobile phase: [water (TFA)-ACN]; B %: 25%-60%, 8 min) to give J2 (28.2 mg, yield 69.80%) as a yellow solid.

¹H NMR (ET43587-370-P1A, 400 MHz, DMSO-d6) δ 0.95 (s, 9H), 1.17-1.26 (m, 2H), 1.29-1.42 (m, 2H), 1.85-1.96 (m, 1H), 2.03-2.12 (m, 1H), 2.45 (s, 3H), 2.99-3.20 (m, 7H), 3.52-3.71 (m, 9H), 3.74-3.87 (m, 6H), 4.16-4.34 (m, 7H), 4.49-4.61 (m, 2H), 6.35 (d, J=7.38 Hz, 1H), 6.80 (br d, J=8.63 Hz, 1H), 6.91-7.06 (m, 3H), 7.10-7.16 (m, 1H), 7.21 (br d, J=10.13 Hz, 1H), 7.25-7.33 (m, 2H), 7.37-7.51 (m, 3H), 8.01 (d, J=8.13 Hz, 2H), 8.45-8.57 (m, 2H), 8.79 (br s, 2H), 8.97 (s, 1H), 9.41 (br s, 1H)

LCMS (ESI+): RT=2.447 min, m/z 647.2, 1293.3 (M/2+H, M+H)⁺.

5_95AB_6min_MS1500-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1500.

### General Procedure for Preparation of Compound 8a 3—Notebook Page: ET43587-366.

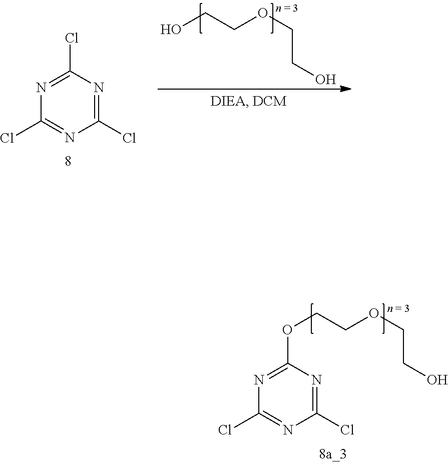

To a solution of Compound 8 (2 g, 10.85 mmol, 1 eq) and DIEA (1.40 g, 10.85 mmol, 1.89 mL, 1 eq) in DCM (40 mL) was added 2-[2-[2-(2-hydroxyethoxy)ethoxy]ethoxy]ethanol (2.11 g, 10.85 mmol, 1.86 mL, 1 eq) in DCM (10 mL) dropwise at 0° C. The mixture was stirred at 25° C. for 12 hrs. TLC (petroleum ether/ethyl acetate=0/1) showed the starting material was consumed and a new spot was generated. The reaction mixture was filtered and concentrated under reduced pressure to give a residue, which was purified by chromatography on silica gel (eluted with petroleum ether/ethyl acetate=10/1 to 0/1) to give Compound 8a_3 (1.6 g, yield 40.96%) as a colorless oil.

¹H NMR (ET43587-366-P1A, 400 MHz, CHLOROFORM-d) δ 3.58-3.74 (m, 12H), 3.84-3.90 (m, 2H), 4.63-4.69 (m, 2H)

### General Procedure for Preparation of Compound 8b 3—Notebook Page: ET43587-369.

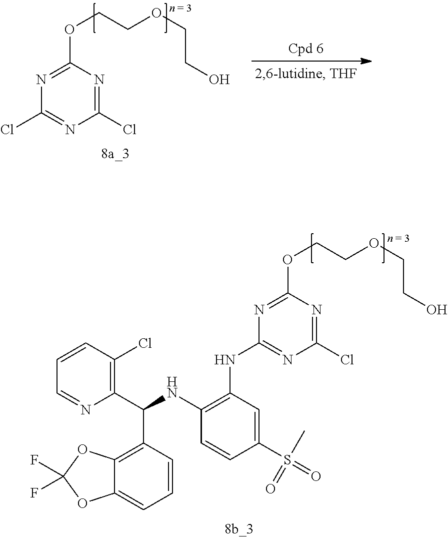

To a solution of Compound 8a_3 (500 mg, 1.46 mmol, 1 eq) and 2,6-lutidine (156.57 mg, 1.46 mmol, 170.19 μL, 1 eq) in THF (10 mL) was added Compound 6 (683.67 mg, 1.46 mmol, 1 eq) in THF (5 mL) dropwise at 0° C. The mixture was stirred at 25° C. for 12 hrs. TLC (petroleum ether/ethyl acetate=0/1) showed the starting material was consumed and a new spot was generated. The reaction mixture was diluted with water (20 mL) and extracted with ethyl acetate (3×20 mL). The organic layer was separated and the combined organic layer was washed with brine (2×20 mL), dried over Na₂SO₄, filtered and concentrated under reduced pressure to give a residue, which was purified by chromatography on silica gel (eluted with petroleum ether/ethyl acetate=50/1 to 5/1) to give Compound 8b_3 (730 mg, yield 58.12%) as a yellow solid.

¹H NMR (ET43587-369-p1a, 400 MHz, CHLOROFORM-d) δ 3.02 (s, 3H), 3.45-3.79 (m, 14H), 3.80-3.91 (m, 1H), 4.29-4.63 (m, 2H), 6.27 (d, J=7.25 Hz, 1H), 6.83 (br d, J=7.25 Hz, 1H), 6.88-7.05 (m, 4H), 7.25 (d, J=4.88 Hz, 1H), 7.65-7.74 (m, 2H), 7.76-8.01 (m, 2H), 8.47 (dd, J=4.69, 1.31 Hz, 1H)

### General Procedure for Preparation of Compound 10_3—Notebook Page: ET43587-373.

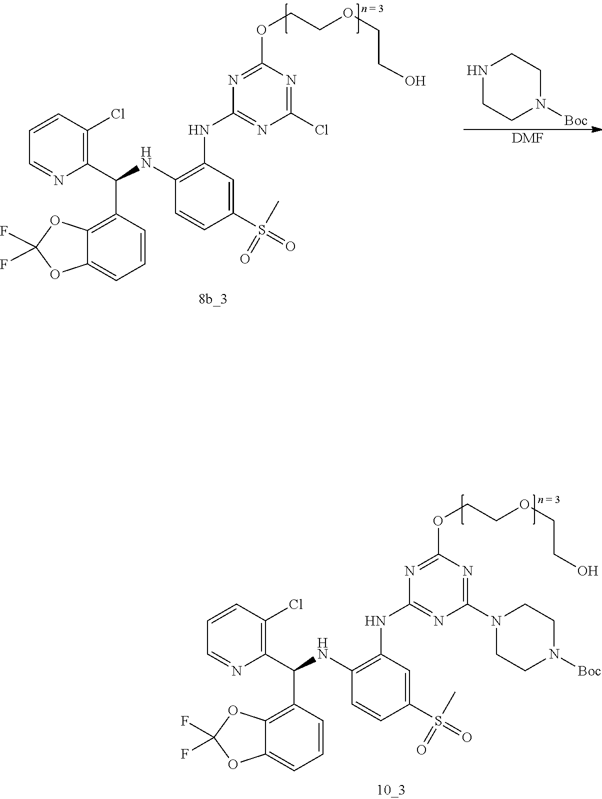

To a solution of Compound 8b_3 (730 mg, 943.66 μmol, 1 eq) and tert-butyl piperazine-1-carboxylate (175.76 mg, 943.66 μmol, 1 eq) in DMF (15 mL) was added TEA (95.49 mg, 943.66 μmol, 1 eq). The mixture was stirred at 90° C. for 2 hrs. LCMS showed all the starting material was consumed and a new peak with desired product MS was detected. The reaction mixture was diluted with water (20 mL) and extracted with ethyl acetate (3×20 mL). The organic layer was separated and the combined organic layer was washed with brine (2×20 mL), dried over Na₂SO₄, filtered and concentrated under reduced pressure to give (720 mg, yield 74.37%) as a yellow solid, which was used to the next step directly without further purification.

LCMS (ESI+): RT=0.822 min, m/z 923.3 (M+H)⁺.

5_95AB_2 min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### General Procedure for Preparation of Compound 11_3—Notebook Page: ET43587-377.

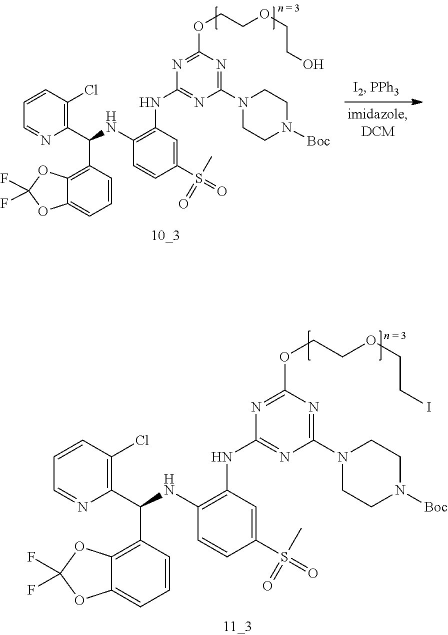

To a solution of Compound 10_3 (650 mg, 703.94 μmol, 1 eq) and 12 (536.00 mg, 2.11 mmol, 3 eq) in DCM (13 mL) were added imidazole (143.77 mg, 2.11 mmol, 3 eq) and PPh₃ (553.91 mg, 2.11 mmol, 3 eq) at 0° C. The mixture was stirred at 25° C. for 12 hrs. LCMS showed all the starting material was consumed and a new peak with desired product MS was detected. The reaction mixture was diluted with water (20 mL) and extracted with DCM (3×20 mL). The organic layer was separated and the combined organic layer was washed with brine (20 mL), dried over Na₂SO₄, filtered and concentrated under reduced pressure to give a residue, which was purified by chromatography on silica gel (eluted with petroleum ether/ethyl acetate=20/1 to 0/1) to give Compound 11_3 (210 mg, yield 24.54%) as a yellow solid.

¹H NMR (ET43587-377-p1a, 400 MHz, CHLOROFORM-d) δ 1.48 (s, 9H), 2.99 (s, 3H), 3.26 (t, J=6.88 Hz, 2H), 3.42 (br s, 4H), 3.59-3.83 (m, 16H), 4.45 (br s, 2H), 6.25 (d, J=7.25 Hz, 1H), 6.75 (br d, J=7.25 Hz, J H), 6.88-7.02 (m, 4H), 7.24 (dd, J=8.13, 4.75 Hz, 1H), 7.62 (dd, J=8.57, 2.06 Hz, J H), 8.09 (br s, 1H), 8.49 (dd, J=4.69, 1.19 Hz, 1H)

### General Procedure for Preparation of Compound 12_3—Notebook Page: ET43587-390.

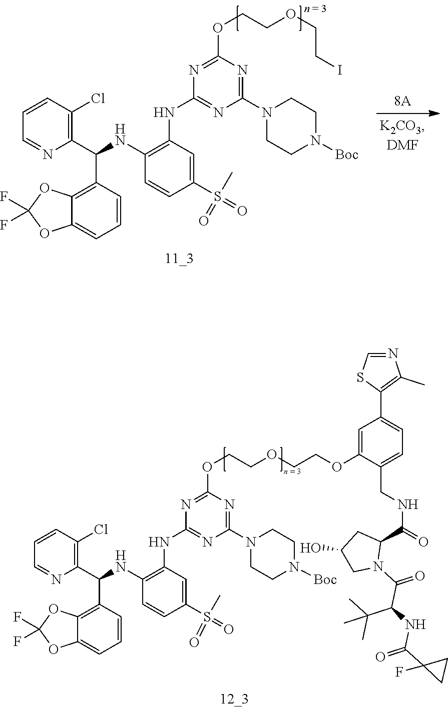

To a solution of Compound 11_3 (190 mg, 110.33 μmol, 1 eq) and Compound 8A (47.01 mg, 88.26 μmol, 0.8 eq) in DMF (1 mL) was added K₂CO₃ (22.87 mg, 165.49 μmol, 1.5 eq). The mixture was stirred at 60° C. for 12 hrs. LCMS showed all the starting material was consumed and a new peak with desired product MS was detected. The mixture was purified by Prep-HPLC (column: Phenomenex Luna 80*30 mm*3 um; mobile phase: [water (TFA)-ACN]; B %: 50%-80%, 8 mins) to give Compound 12_3 (65 mg, yield 38.92%) as a white solid.

LCMS (ESI+): RT=0.868 min, m/z 1437.6 (M+H)⁺.

5_95AB_2 min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### General Procedure for Preparation of J3—Notebook Page: ET43587-393.

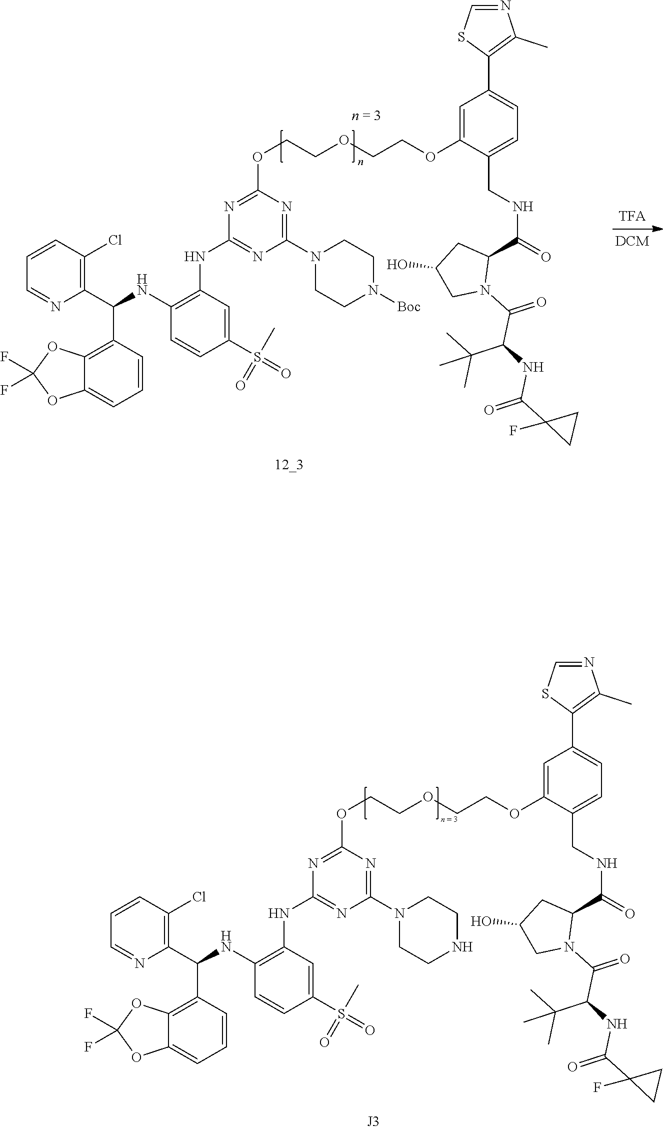

To a solution of Compound 123 (65 mg, 45.20 μmol, 1 eq) in DCM (1.5 mL) was added TFA (0.4 mL) dropwise at 0° C. The mixture was stirred at 25° C. for 12 hrs. LCMS showed all the starting material was consumed and a new peak with desired product MS was detected. The reaction mixture was concentrated under reduced pressure to give a residue, which was purified by Prep-HPLC (column: Phenomenex Luna 80*30 mm*3 um; mobile phase: [water (TFA)-ACN]; B %: 25%-55%, 8 mins) to give J3 (36.4 mg, yield 55.46%) as a yellow solid.

¹H NMR (ET43587-393-P1A, 400 MHz, DMSO-d₆) δ 0.95 (s, 9H), 1.21 (br dd, J=8.13, 2.13 Hz, 2H), 1.28-1.44 (m, 2H), 1.92 (td, J=8.41, 4.57 Hz, 1H), 2.06-2.12 (m, 1H), 2.45 (s, 3H), 2.95-3.25 (m, 7H), 3.45-3.71 (m, 13H), 3.76-3.93 (m, 6H), 4.20-4.33 (m, 7H), 4.51 (br t, J=8.25 Hz, 1H), 4.59 (br d, J=9.13 Hz, 1H), 6.35 (br d, J=7.13 Hz, 1H), 6.81 (br d, J=8.88 Hz, 1H), 6.90-7.05 (m, 3H), 7.12-7.24 (m, 2H), 7.26-7.33 (m, 2H), 7.40 (d, J=7.75 Hz, 1H), 7.43-7.52 (m, 2H), 8.02 (d, J=8.00 Hz, 2H), 8.46-8.58 (m, 2H), 8.79 (br d, J=1.00 Hz, 2H), 8.97 (s, 1H), 9.42 (br s, 1H)

LCMS (ESI+): RT=2.457 min, m/z 669.2, 1337.5 (M/2+H, M+H)⁺.

5_95AB6min_MS1500-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1500.

### General Procedure for Preparation of Compound 8a 4—Notebook Page: ET43587-349.

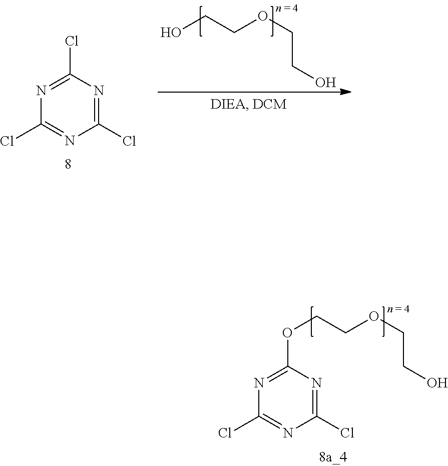

To a solution of Compound 8 (2 g, 10.85 mmol, 1 eq) in DCM (40 mL) were added 2-[2-[2-[2-(2-hydroxyethoxy)ethoxy]ethoxy]ethoxy]ethanol (2.58 g, 10.85 mmol, 1 eq) and DIEA (1.40 g, 10.85 mmol, 1.89 mL, 1 eq) in DCM (15 mL) dropwise at 0° C. The mixture was stirred at 25° C. for 12 hrs. LCMS showed all the starting material was consumed and a new peak was detected. The reaction mixture was filtered and concentrated under reduced pressure to give a residue, which was purified by chromatography on silica gel (eluted with petroleum ether/ethyl acetate=50/1 to 0/1) to give Compound 8a_4 (1.2 g, yield 27.22%) as a colorless oil.

¹H NMR (ET43587-349-p1a, 400 MHz, CHLOROFORM-d) δ 2.46 (br s, 1H), 3.59-3.76 (m, 16H), 3.83-3.90 (m, 2H), 4.61-4.69 (m, 2H)

### General Procedure for Preparation of Compound 8b 4—Notebook Page: ET43587-353.

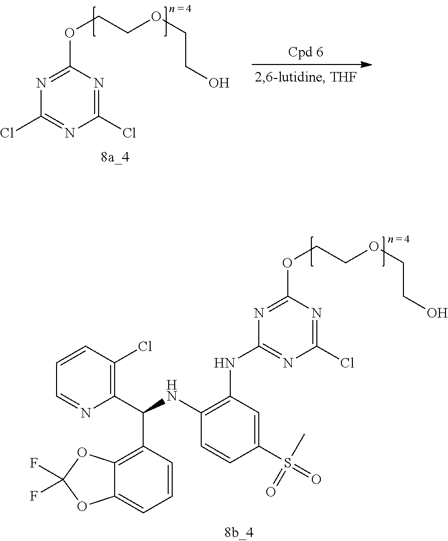

To a solution of Compound 8a_4 (500 mg, 1.29 mmol, 1 eq) in THF (10 mL) were added Compound 6 (605.70 mg, 1.29 mmol, 1 eq) and 2,6-lutidine (138.72 mg, 1.29 mmol, 1 eq) in THF (5 mL) dropwise at 0° C. The mixture was stirred at 25° C. for 12 hrs. TLC (petroleum ether/ethyl acetate=0/1) showed the starting material was consumed and a new spot was generated. The reaction mixture was diluted with water (30 mL) and extracted with ethyl acetate (3×30 mL).

The organic layer was separated and the combined organic layer was washed with brine (2×30 mL), dried over Na₂SO₄, filtered and concentrated under reduced pressure to give a residue, which was purified by chromatography on silica gel (eluted with petroleum ether/ethyl acetate=50/1 to 0/1) to give Compound 8b_4 (600 mg, yield 51.02%) as a yellow solid.

¹H NMR (ET43587-353-P1A, 400 MHz, CHLOROFORM-d) δ 3.02 (s, 3H), 3.53-3.79 (m, 18H), 3.86 (br d, J=6.88 Hz, 1H), 4.30-4.76 (m, 2H), 6.27 (br s, 1H), 6.74-7.11 (m, 5H), 7.64-7.85 (m, 3H), 8.48 (br d, J=2.50 Hz, 1H)

### General Procedure for Preparation of Compound 10_4—Notebook Page: ET43587-354.

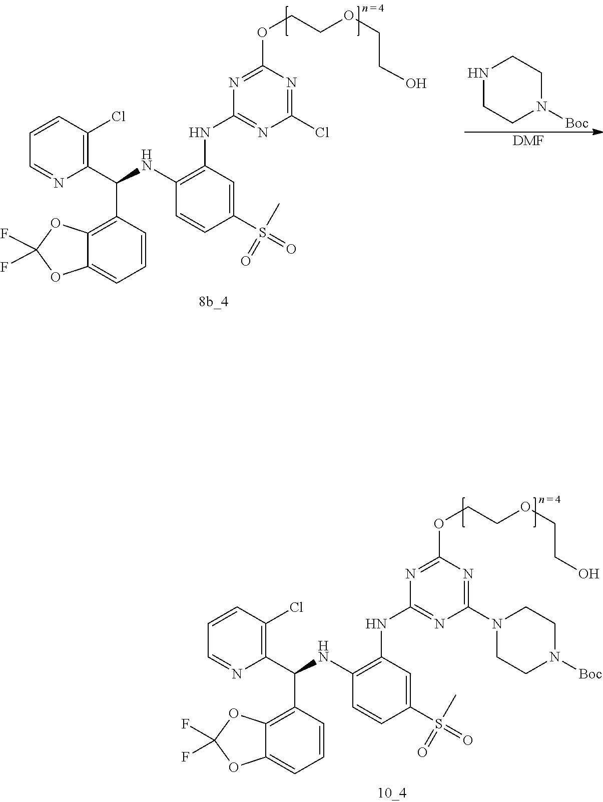

To a solution of Compound 8b_4 (600 mg, 733.82 μmol, 1 eq) and tert-butyl piperazine-1-carboxylate (136.67 mg, 733.82 μmol, 1 eq) in DMF (12 mL) was added TEA (74.26 mg, 733.82 μmol, 1 eq). The mixture was stirred at 90° C. for 2 hrs. LCMS showed all the starting material was consumed and a new peak with desired product MS was detected. The reaction mixture was diluted with water (20 mL) and extracted with ethyl acetate (3×20 mL). The organic layer was separated and the combined organic layer was washed with brine (2×20 mL), dried over Na₂SO₄, filtered and concentrated under reduced pressure to give Compound 10_4 (580 mg, yield 73.53%) as a yellow solid, which was used to the next step directly without further purification.

¹H NMR (ET43587-354-p1a, 400 MHz, CHLOROFORM-d) δ 1.45-1.51 (m, 9H), 2.99 (s, 3H), 3.41 (br s, 4H), 3.60-3.79 (m, 22H), 4.44 (br s, 2H), 6.25 (d, J=7.25 Hz, 1H), 6.74 (br d, J=7.13 Hz, 1H), 6.89 (d, J=8.75 Hz, 1H), 6.92-7.01 (m, 3H), 7.24 (dd, J=8.00, 4.75 Hz, 1H), 7.62 (dd, J=8.63, 2.13 Hz, 1H), 7.70 (dd, J=8.07, 1.44 Hz, 1H), 8.08 (br s, 1H), 8.49 (dd, J=4.63, 1.13 Hz, 1H)

### General Procedure for Preparation of Compound 11_4—Notebook Page: ET43587-371.

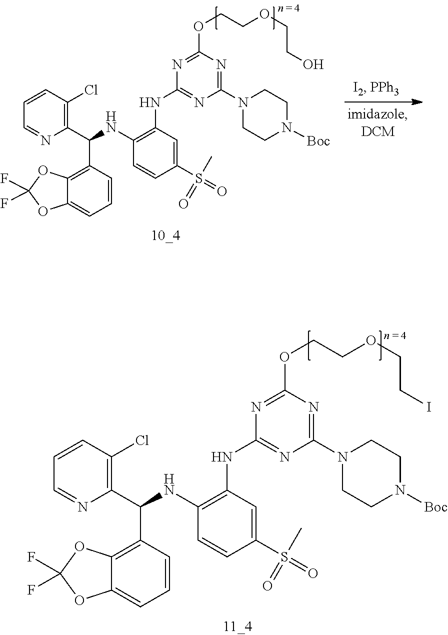

To a solution of Compound 104 (550 mg, 568.52 μmol, 1 eq) and 12 (432.88 mg, 1.71 mmol, 3 eq) in DCM (11 mL) were added imidazole (116.11 mg, 1.71 mmol, 3 eq) and PPh₃ (447.35 mg, 1.71 mmol, 3 eq) at 0° C. The mixture was stirred at 25° C. for 12 hrs. LCMS showed all the starting material was consumed and a new peak with desired product MS was detected. The reaction mixture was diluted with water (30 mL) and extracted with ethyl acetate (3×30 mL). The organic layer was separated and the combined organic layer was washed with brine (2-30 mL), dried over Na₂SO₄, filtered and concentrated under reduced pressure to give a residue, which was purified by chromatography on silica gel (eluted with petroleum ether/ethyl acetate=20/1 to 0/1) to give Compound 114 (180 mg, yield 23.51%) as a yellow solid.

¹H NMR (ET43587-371-p1a, 400 MHz, CHLOROFORM-d) δ 1.48 (s, 9H), 2.99 (s, 3H), 3.26 (t, J=6.94 Hz, 2H), 3.42 (br d, J=1.00 Hz, 4H), 3.62-3.81 (m, 20H), 4.43 (br s, 2H), 6.25 (d, J=7.25 Hz, 1H), 6.75 (br d, J=7.13 Hz, 1H), 6.86-7.03 (m, 5H), 7.24 (dd, J=8.00, 4.75 Hz, 1H), 7.62 (dd, J=8.63, 2.00 Hz, 1H), 8.09 (br s, 1H), 8.49 (dd, J=4.63, 1.25 Hz, 1H)

### General Procedure for Preparation of Compound 12_4—Notebook Page: ET43587-378.

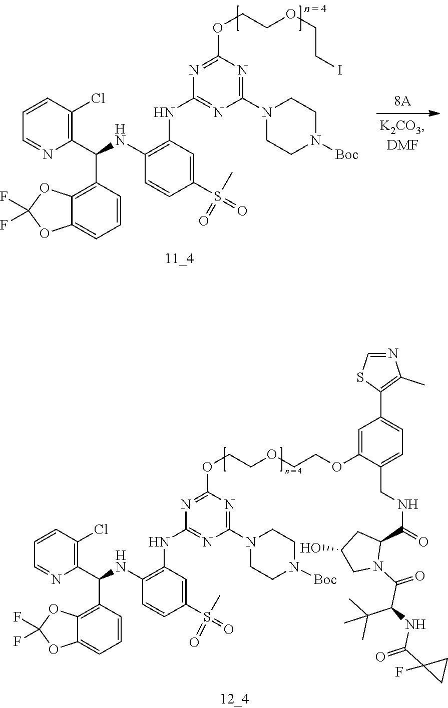

To a solution of Compound 114 (180 mg, 133.66 μmol, 1 eq) and Compound 8A (42.72 mg, 80.20 μmol, 0.6 eq) in DMF (2 mL) was added K₂CO₃ (27.71 mg, 200.50 μmol, 1.5 eq). The mixture was stirred at 60° C. for 12 hrs. LCMS showed about 20% starting material was remained and a new peak with desired product MS was detected. The mixture was purified by Prep-HPLC (column: Phenomenex Luna 80*30 mm*3 um; mobile phase: [water (TFA)-ACN]; B %: 50%-80%, 8 min) to give Compound 12_4 (20 mg, yield 9.59%) as a white solid.

LCMS (ESI+): RT=3.072 min, m/z 741.3 (M/2+H)⁺.

5_95AB_6min-220-MS2000: LC/MS (The gradient was 5% B in 0.40 min and 5-95% B at 0.4-3.0 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) as well as positive electrospray ionization. MS range was 100-2000.

### General Procedure for Preparation of J4—Notebook Page: ET43587-392.

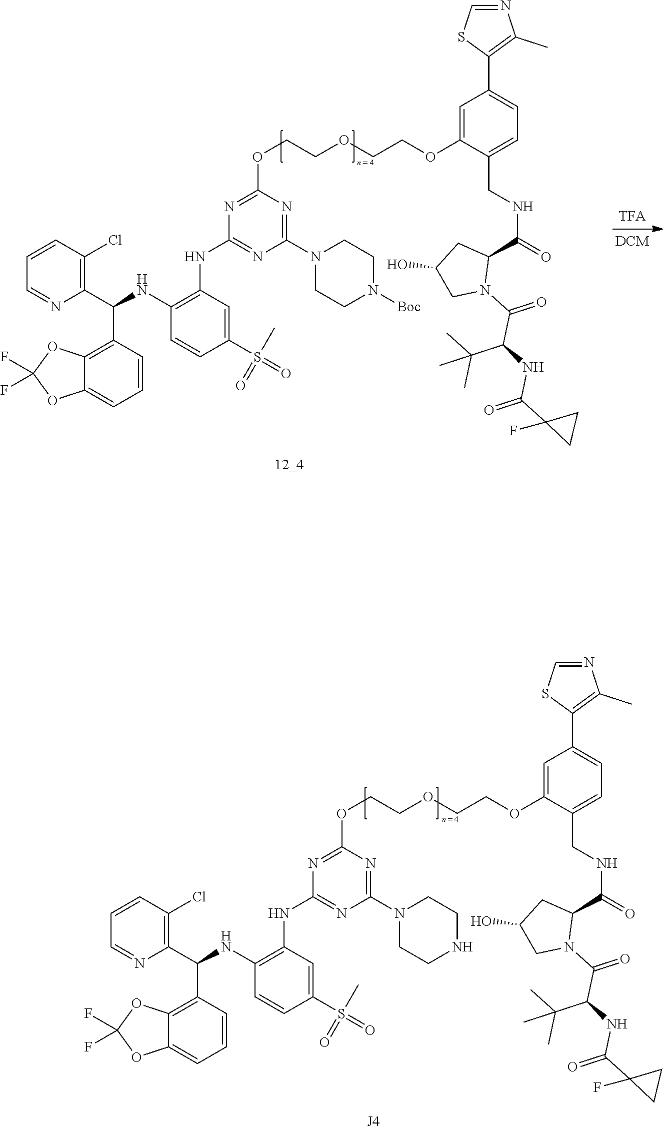

To a solution of Compound 124 (20 mg, 13.49 μmol, 1 eq) in DCM (0.6 mL) was added TFA (154 mg, 1.35 mmol, 0.1 mL, 100.09 eq). The mixture was stirred at 25° C. for 12 hrs. LCMS showed all the starting material was consumed and a new peak with desired product MS was detected. The reaction mixture was purified by Prep-HPLC (column: Phenomenex Luna 80*30 mm*3 um; mobile phase: [water (TFA)-ACN]; B %: 25%-55%, 8 mins) to give J4 (10.5 mg, yield 52.01%) as a white solid.

¹H NMR (ET43587-392-P1A, 400 MHz, DMSO-d6) δ 0.95 (s, 9H), 1.21 (br dd, J=8.07, 2.56 Hz, 2H), 1.29-1.42 (m, 2H), 1.91 (ddd, J=12.63, 8.63, 4.25 Hz, 1H), 2.09 (br dd, J=12.51, 8.13 Hz, 1H), 2.45 (s, 3H), 3.00-3.19 (m, 7H), 3.43-3.56 (m, 11H), 3.57-3.72 (m, 8H), 3.75-3.81 (m, 4H), 4.16-4.23 (m, 3H), 4.26-4.38 (m, 4H), 4.51 (br t, J=8.19 Hz, 1H), 4.59 (br d, J=9.01 Hz, 1H), 6.35 (br d, J=7.38 Hz, 1H), 6.81 (br d, J=8.75 Hz, 1H), 6.91-7.05 (m, 3H), 7.11-7.33 (m, 4H), 7.36-7.53 (m, 3H), 8.02 (d, J=8.00 Hz, 2H), 8.46-8.59 (m, 2H), 8.79 (br s, 2H), 8.97 (s, 1H), 9.42 (br s, 1H)

LCMS (ESI+): RT=2.459 min, m/z 691.3, 1381.5 (M/2+H, M+H)⁺.

5_95AB_6min_MS1500-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1500.

### Synthesis of K1, K3 and K5

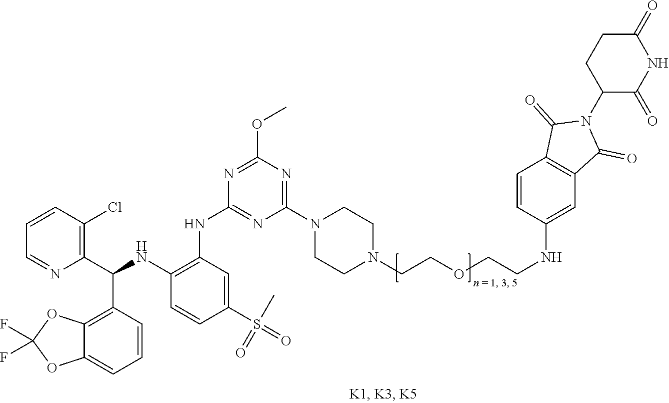

| Compound ID | Synonym | Synonym |
|---|---|---|
| NUCC-0226601 | K1 | ET43587-193 |
| NUCC-0226600 | K3 | ET43587-190 |
| NUCC-0226602 | K5 | ET43587-197 |

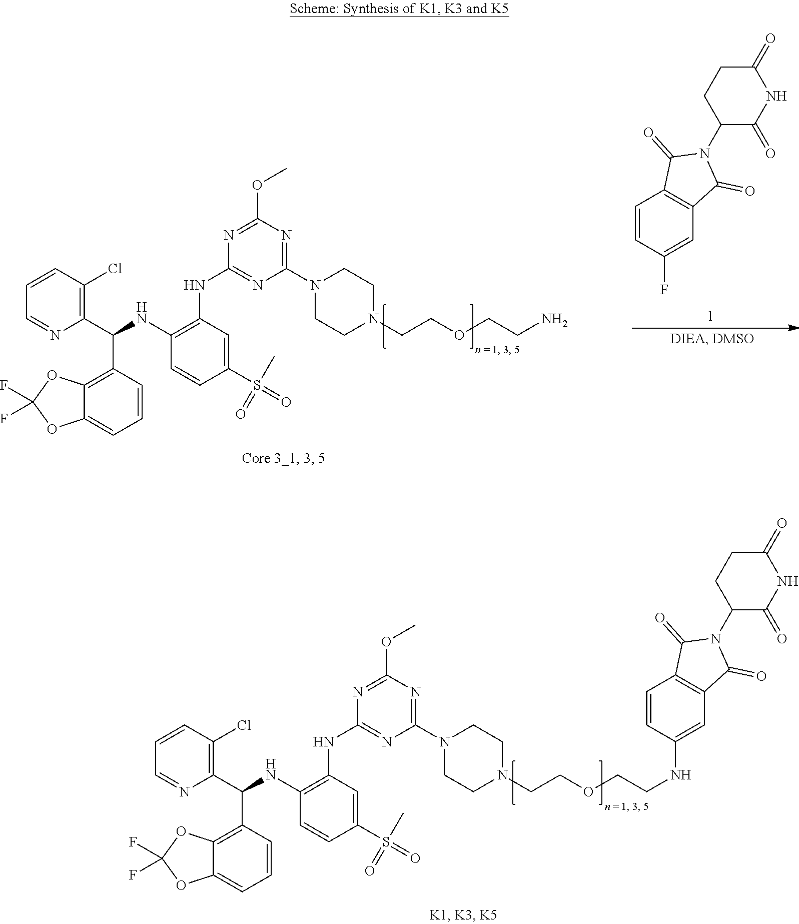

### Chemical Synthesis

All chemical reagents were obtained from commercial suppliers and used without further purification, unless otherwise stated. Reactions were run without taking precautions to exclude air or moisture, unless otherwise noted. Normal phase column chromatography was performed using silica gel columns and ACS grade solvents. Analytical TLC was performed on EM Reagent 0.25 mm silica gel 60 F254 plates and visualized by UV light. Compound identities were confirmed by ¹H and F (NMR) spectroscopy which were recorded on a Bruker 400 MHz spectrometer using the corresponding residual solvent peak (CDCl₃,1H δ=7.27; CD₃OD, 1H δ=3.31; DMSO-d₆, 1H 5=2.50) as an internal standard. The chemical shifts for ¹H-NMR is reported to the second decimal place. Proton coupling constants are expressed in hertz (Hz). Standard abbreviations were used to denote spin multiplicity for ¹H NMR data.

### LCMS Methods:

### Method: 5_95AB_6min_MS1500-220-254-ELSD

LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1500.

### Experimentals for Largest Scale Run:

### General Procedure for Preparation of KI-Notebook Page: ET43587-193.

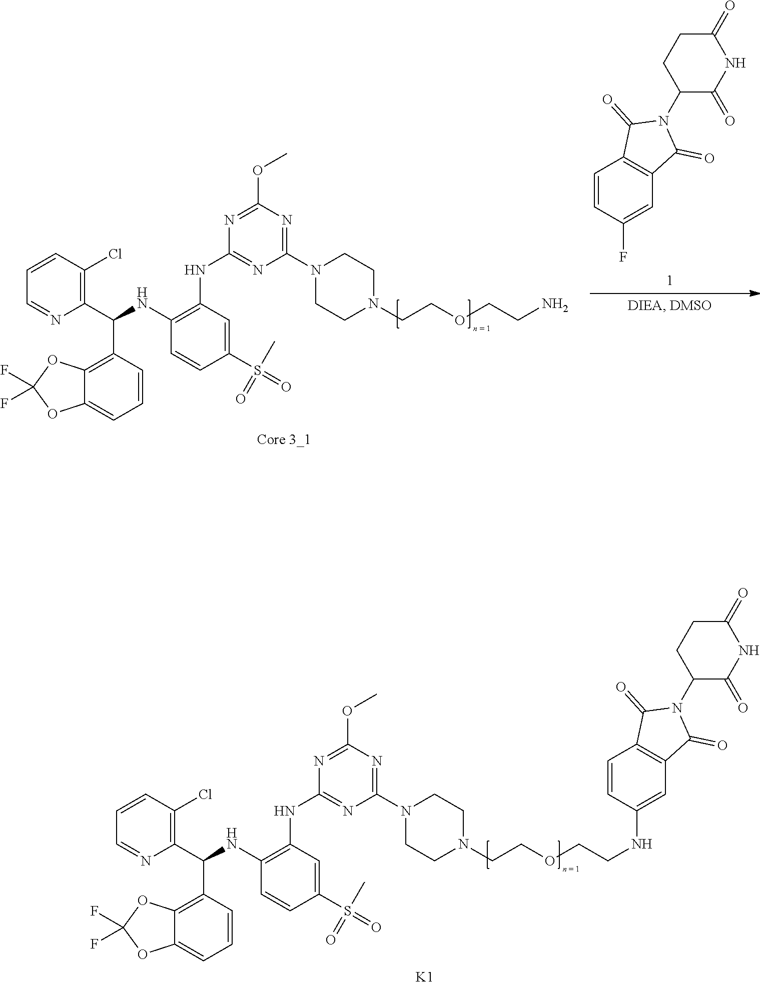

To a solution of Compound 1 (55.38 mg, 200.48 μmol, 1 eq) and Compound Core 3_1 (150 mg, 200.48 μmol, 1 eq) in DMSO (4 mL) was added DIEA (77.73 mg, 601.44 μmol, 3 eq).

The mixture was stirred at 130° C. for 3 hrs. LCMS showed all the starting material was consumed and a new peak with desired product MS was detected. The mixture was purified by Prep-HPLC (column: Phenomenex Luna 80*30 mm*3 um; mobile phase: [water (0.1% TFA)-ACN]; B %: 30%-55%, 8 mins) to give KI (62.1 mg, yield 27.42%) as a yellow solid.

¹H NMR (ET43587-193-P1B, 400 MHz, DMSO-d6) δ 1.93-2.04 (m, 1H), 2.54-2.62 (m, 2H), 2.83-2.92 (m, 1H), 3.07 (s, 5H), 3.21-3.36 (m, 4H), 3.42 (br s, 2H), 3.54 (br d, J=3.88 Hz, 1H), 3.65 (br t, J=5.13 Hz, 2H), 3.74-3.85 (m, 6H), 4.44-4.64 (m, 2H), 5.03 (dd, J=12.94, 5.32 Hz, 1H), 6.36 (br d, J=6.88 Hz, 1H), 6.81 (br d, J=8.75 Hz, 1H), 6.87-7.04 (m, 3H), 7.08-7.19 (m, 2H), 7.19-7.25 (m, 1H), 7.33 (dd, J=7.88, 1.13 Hz, 1H), 7.41-7.63 (m, 3H), 8.04 (dd, J=8.13, 1.38 Hz, 2H), 8.55 (br d, J=3.13 Hz, 1H), 9.35-9.60 (m, 1H), 9.72-9.91 (m, 1H), 11.07 (s, 1H)

LCMS (ESI+): RT=2.382 min, m/z 502.6, 1004.0 (M/2+H, M+H)⁺.

5_95AB_6min_MS1500-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1500.

### General Procedure for Preparation of K3—Notebook Page: ET43587-190.

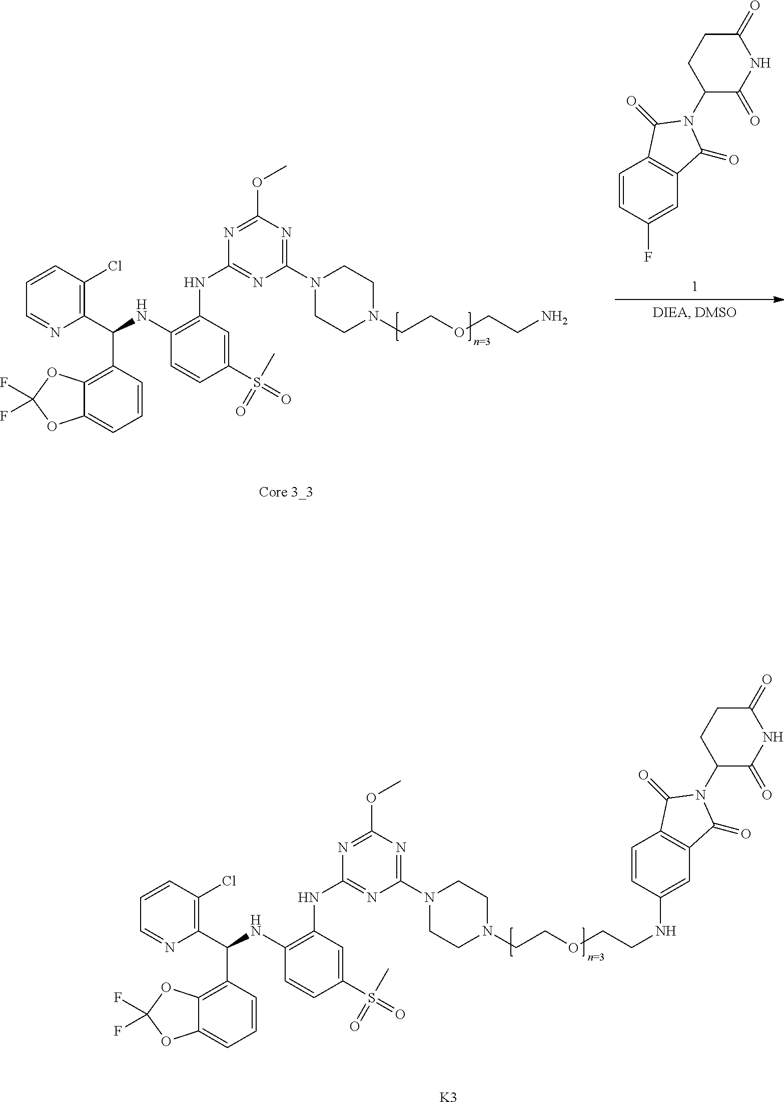

To a solution of Compound 1 (33.03 mg, 119.57 μmol, 1 eq) and Compound Core 3_3 (100 mg, 119.57 μmol, 1 eq) in DMSO (3 mL) was added DIEA (46.36 mg, 358.72 μmol, 3 eq). The reaction mixture was stirred at 130° C. for 2 hrs. LCMS showed all the starting material was consumed and a new peak with desired product MS was detected. The mixture was purified by Prep-HPLC (column: Phenomenex Luna 80*30 mm*3 um; mobile phase: [water (0.1% TFA)-ACN]; B %: 30%-55%, 8 mins) to give K3 (28.9 mg, yield 19.81%) as a yellow solid.

¹H NMR (ET43587-190-P1A, 400 MHz, DMSO-d6) δ 1.94-2.04 (m, 1H), 2.54-2.60 (m, 2H), 2.82-2.91 (m, 1H), 2.97-3.11 (m, 5H), 3.22-3.36 (m, 6H), 3.56 (br s, 11H), 3.70-3.88 (m, 6H), 4.35-4.66 (m, 2H), 5.02 (dd, J=12.82, 5.32 Hz, 1H), 6.35 (br d, J=7.25 Hz, 1H), 6.76-6.89 (m, 2H), 6.98 (s, 2H), 7.07-7.26 (m, 3H), 7.32 (dd, J=7.88, 1.13 Hz, 1H), 7.42-7.58 (m, 3H), 8.03 (dd, J=8.13, 1.38 Hz, 2H), 8.55 (br d, J=3.88 Hz, 1H), 9.36-9.60 (m, 1H), 9.75-9.90 (m, 1H), 11.07 (s, 1H)

LCMS (ESI+): RT=2.392 min, m/z 546.7, 1092.1 (M/2+H, M+H)⁺.

5_95A B_6 min_MS1500-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1500.

### General Procedure for Preparation of K5—Notebook Page: ET43587-197.

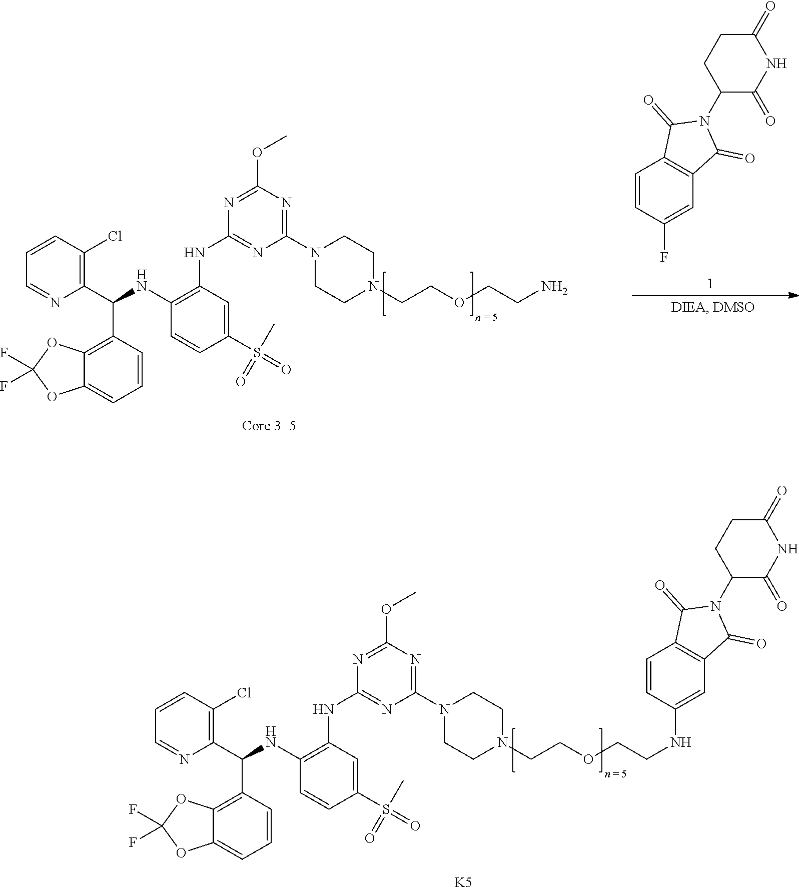

To a solution of Compound 1 (44.82 mg, 162.27 μmol, 1 eq) and Compound Core 3_5 (150 mg, 162.27 μmol, 1 eq) in DMSO (4 mL) was added DIEA (62.91 mg, 486.80 μmol, 3 eq). The mixture was stirred at 130° C. for 3 hrs. LCMS showed all the starting material was consumed and a new peak with desired product MS was detected. The mixture was purified by Prep-HPLC (column: Phenomenex Luna 80*30 mm*3 um; mobile phase: [water (0.1% TFA)-ACN]; B %: 30%-55%, 8 mins) to give K5 (39.1 mg, yield 18.20%) as a yellow solid.

¹H NMR (ET43587-197-P1A, 400 MHz, DMSO-d6) δ 1.94-2.06 (m, 1H), 2.54-2.62 (m, 2H), 2.83-2.92 (m, 1H), 3.08 (s, 5H), 3.24-3.38 (m, 6H), 3.48-3.59 (m, 19H), 3.72-3.87 (m, 6H), 4.41-4.68 (m, 2H), 5.03 (dd, J=12.82, 5.32 Hz, 1H), 6.36 (br d, J=7.38 Hz, 1H), 6.81 (d, J=8.76 Hz, 1H), 6.89 (dd, J=8.44, 2.06 Hz, 1H), 6.93-7.02 (m, 2H), 7.11-7.19 (m, 2H), 7.23 (br d, J=9.63 Hz, 1H), 7.33 (dd, J=7.82, 1.19 Hz, 1H), 7.48 (dd, J=8.13, 4.75 Hz, 2H), 7.56 (d, J=8.38 Hz, 1H), 8.04 (dd, J=8.13, 1.38 Hz, 2H), 8.56 (br d, J=4.13 Hz, 1H), 9.49 (br d, J=2.50 Hz, 1H), 9.75-9.91 (m, 1H), 11.07 (s, 1H)

LCMS (ESI+): RT=2.416 min, m/z 590.7, 1180.2 (M/2+H, M+H)⁺.

5_95AB_6min_MS1500-220-254-ELSD: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1500.

### Synthesis of M3 and M5

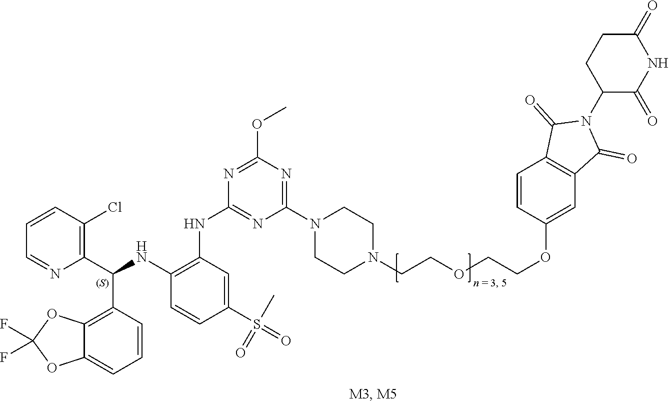

| Compound ID | Synonym | Synonym |
|---|---|---|
| NUCC-0226553 | M3 | ET43587-59 |
| NUCC-0226575 | M5 | ET43587-94 |

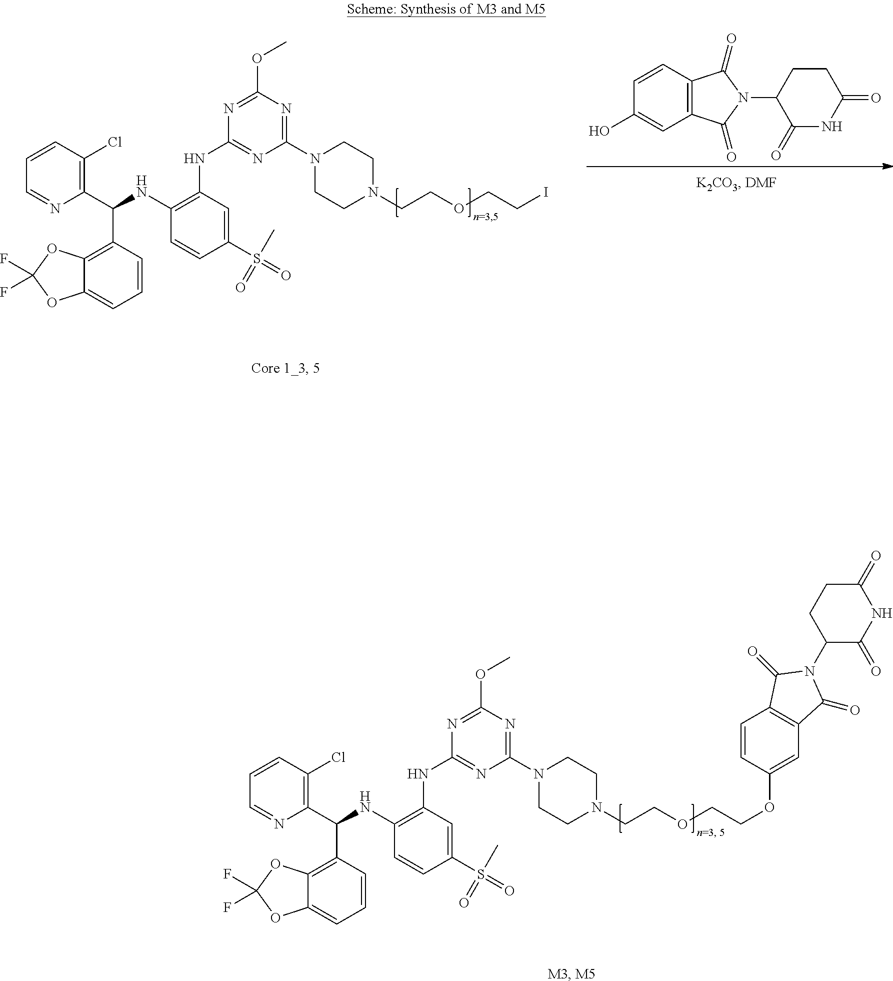

### Chemical Synthesis

All chemical reagents were obtained from commercial suppliers and used without further purification, unless otherwise stated. Reactions were run without taking precautions to exclude air or moisture, unless otherwise noted. Normal phase column chromatography was performed using silica gel columns and ACS grade solvents. Analytical TLC was performed on EM Reagent 0.25 mm silica gel 60 F254 plates and visualized by UV light. Compound identities were confirmed by ¹H and F (NMR) spectroscopy which were recorded on a Bruker 400 MHz spectrometer using the corresponding residual solvent peak (CDCl₃,H δ=7.27; CD₃OD, 1H δ=3.31; DMSO-d₆, 1H δ=2.50) as an internal standard. The chemical shifts for ¹H-NMR is reported to the second decimal place. Proton coupling constants are expressed in hertz (Hz). Standard abbreviations were used to denote spin multiplicity for ¹H NMR data.

### LCMS Methods:

### Method: 5_95AB_6min-ELSD-MS1500

LC/MS: The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (Sum particles). Detection methods are diode array (DAD) and evaporative light scattering (ELSD) detection as well as positive electrospray ionization. MS range was 100-1500.

### Experimentals for Largest Scale Run:

### General Procedure for Preparation of M3—Notebook Page: ET43587-59.

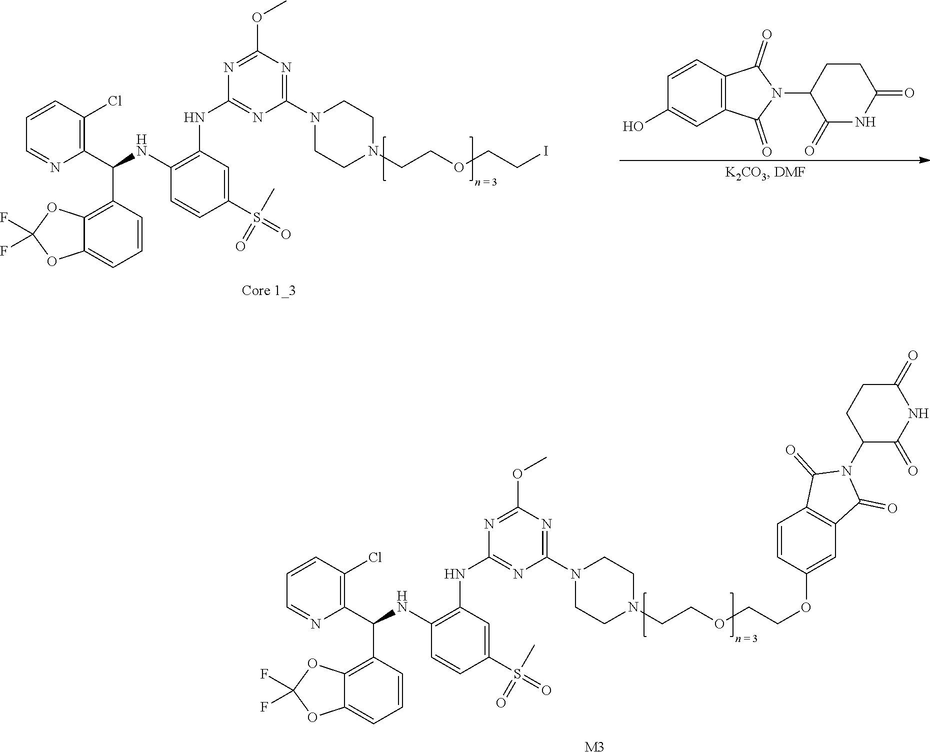

To a solution of 2-(2,6-dioxo-3-piperidyl)-5-hydroxy-isoindoline-1,3-dione (43.43 mg, 158.36 μmol, 1 eq) and Compound Core 1_3 (150 mg, 158.36 μmol, 1 eq) in DMF (3 mL) was added K₂CO₃ (65.66 mg, 475.09 μmol, 3 eq). The mixture was stirred at 60° C. for 4 hrs. LCMS showed all the starting material was consumed and a new peak with desired product MS was detected. The reaction mixture was purified by Prep-HPLC (column: Waters Xbridge Prep OBD C18 150*40 mm*10 um; mobile phase: [water (NH₄HCO₃)-ACN]; B %: 30%-65%, 8 mins) to give M3 (16.2 mg, yield 8.33%) as a white solid.

¹H NMR (ET43587-59-P1A, 400 MHz, DMSO-d₆) δ 2.00-2.08 (m, 1H), 2.31-2.47 (m, 7H), 2.82-2.94 (m, 1H), 3.06 (s, 3H), 3.43-3.69 (m, 16H), 3.78 (br d, J=2.13 Hz, 4H), 4.06-4.12 (m, 1H), 4.26-4.31 (m, 1H), 5.11 (dd, J=12.94, 5.44 Hz, 1H), 6.33 (d, J=7.38 Hz, 1H), 6.80 (d, J=8.50 Hz, 1H), 6.96 (br d, J=7.13 Hz, 1H), 7.10-7.17 (m, 1H), 7.19-7.26 (m, 1H), 7.29-7.50 (m, 4H), 7.81 (d, J=8.38 Hz, 1H), 7.92 (br s, 1H), 7.99-8.06 (m, 1H), 8.49-8.55 (m, 1H), 9.25 (br s, 1H), 11.11 (s, 1H)

LCMS (ESI+): RT=2.376 min, m/z 547.2, 1093.3 (M/2+H. M+H)⁺.

5_95AB_6min-ELSD-MS1500: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1500.

### General Procedure for Preparation of M5—Notebook Page: ET43587-94.

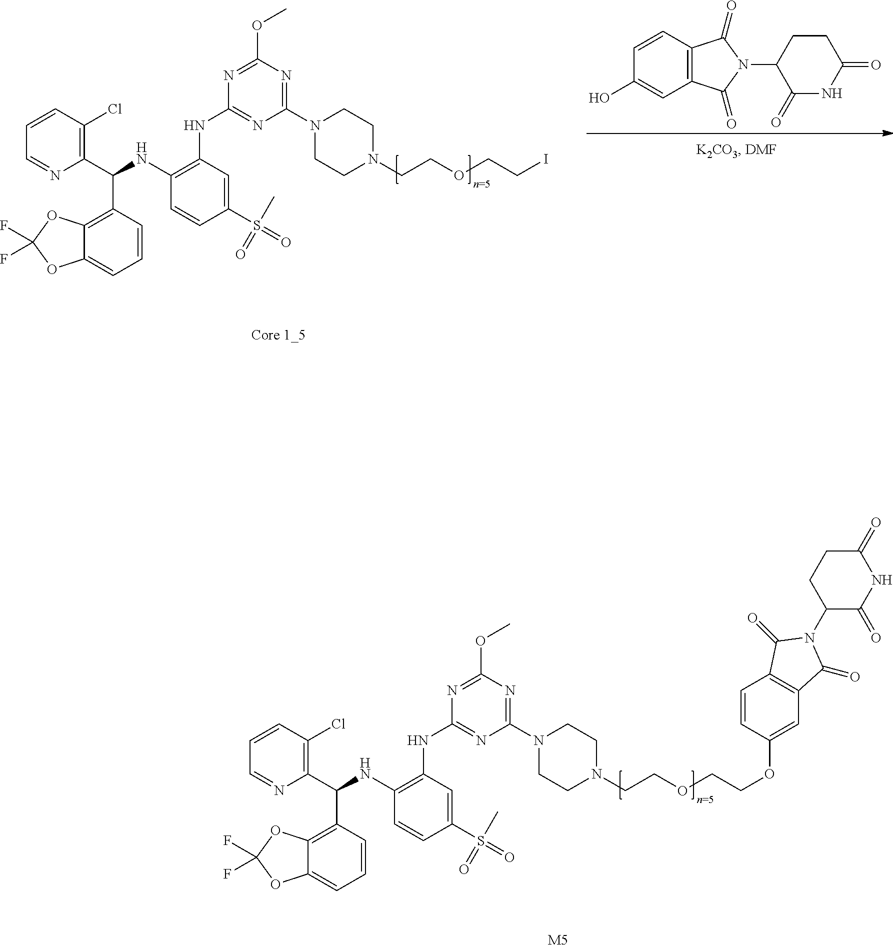

To a solution of 2-(2,6-dioxo-3-piperidyl)-5-hydroxy-isoindoline-1,3-dione (31.79 mg, 115.91 μmol, 1 eq) and Compound Core 1_5 (120 mg, 115.91 μmol, 1 eq) in DMF (2.5 mL) was added K₂CO₃ (80.1 mg, 579.55 μmol, 5 eq). The mixture was stirred at 60° C. for 12 hrs. LCMS showed all the starting material was consumed and a new peak with desired product MS was detected. The mixture was purified by Prep-HPLC (column: Phenomenex luna C18 100*40 mm*5 um; mobile phase: [water (0.1% TFA)-ACN]; B %: 40%-70%, 8 mins) to give M5 (50.4 mg, yield 32.32%) as a yellow solid.

¹H NMR (ET43587-94-P1A, 400 MHz, DMSO-d6) δ 1.99-2.08 (m, 1H), 2.83-2.95 (m, 1H), 3.07 (s, 5H), 3.31 (br s, 4H), 3.41-3.64 (m, 19H), 3.69-3.89 (m, 7H), 4.26-4.33 (m, 2H), 4.39-4.68 (m, 3H), 5.11 (dd, J=12.84, 5.26 Hz, 1H), 6.36 (br d, J-=7.34 Hz, 1H), 6.81 (br d, J=8.80 Hz, 1H), 6.91-6.99 (m, 1H), 7.11-7.18 (m, 1H), 7.22 (br d, J=7.09 Hz, 1H), 7.29-7.39 (m, 2H), 7.42-7.52 (m, 3H), 7.82 (d, J=8.31 Hz, 1H), 8.03 (d, J=8.19 Hz, 2H), 8.55 (br d, J=3.91 Hz, 1H), 9.39-9.53 (m, 1H), 9.72-9.87 (m, 1H), 11.10 (s, 1H)

LCMS (ESI+): RT=2.498 min, m/z 591.3, 1181.5 (M/2+H, M+H)⁺.

5_95AB_6min-ELSD-MS1500: LC/MS (The gradient was 5% B in 0.40 min and 5-95% B at 0.40-3.00 min, hold on 95% B for 1.00 min, and then 95-5% B in 0.01 min, the flow rate was 1.0 ml/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection as well as positive electrospray ionization. MS range was 100-1500.

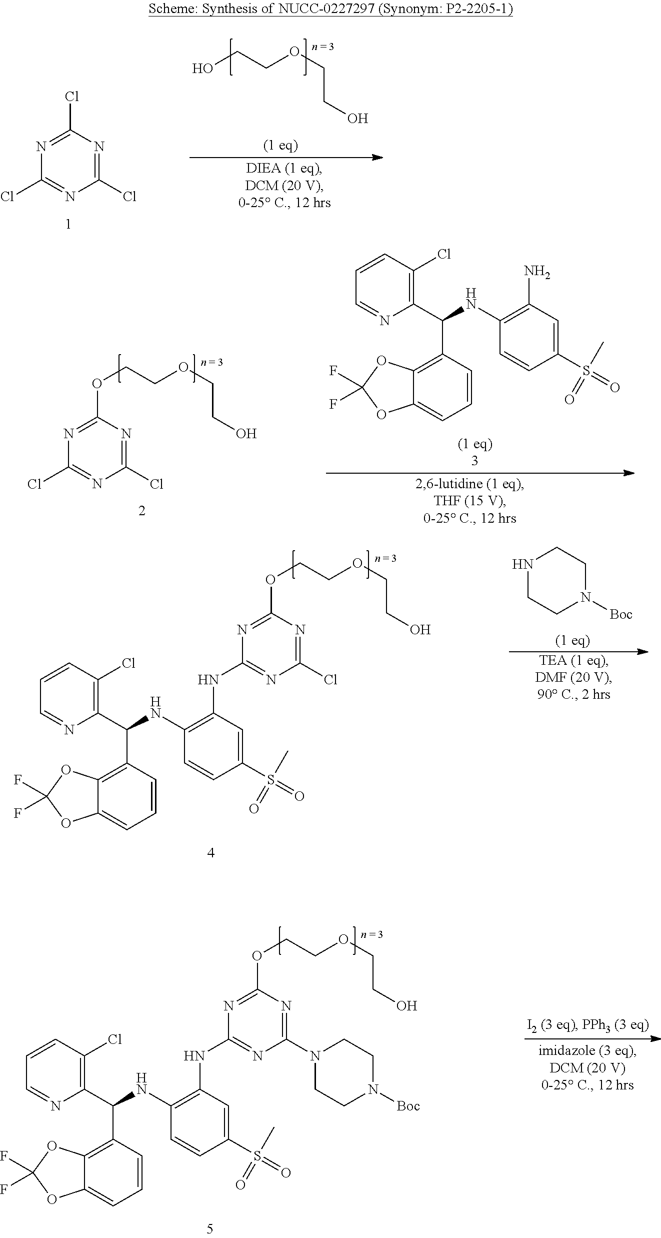

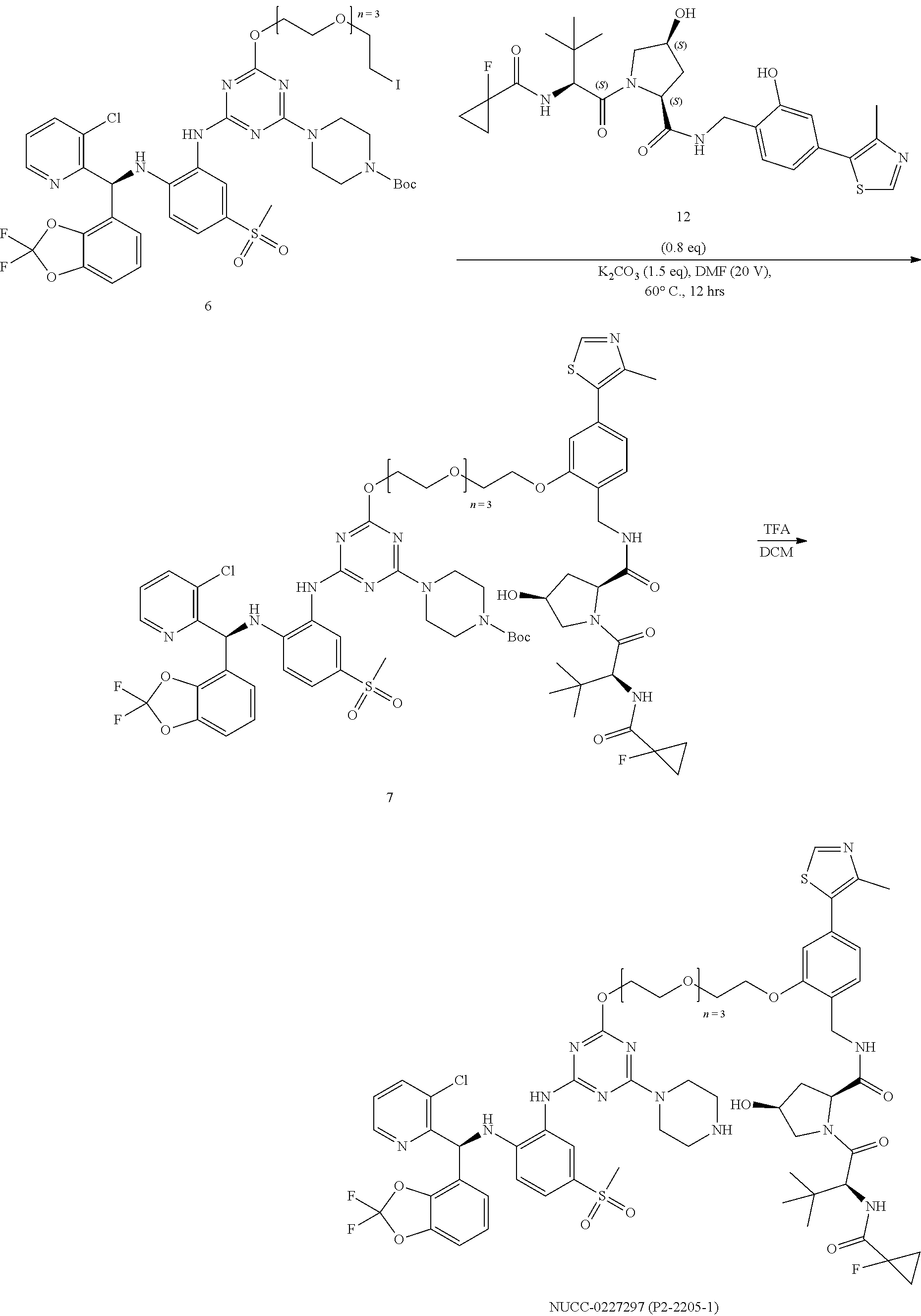

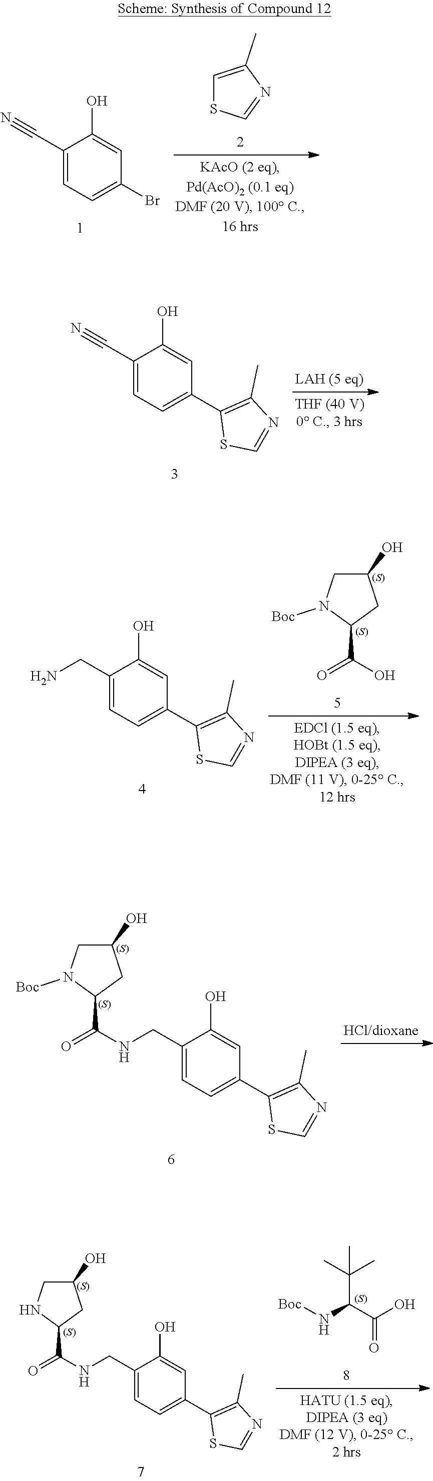

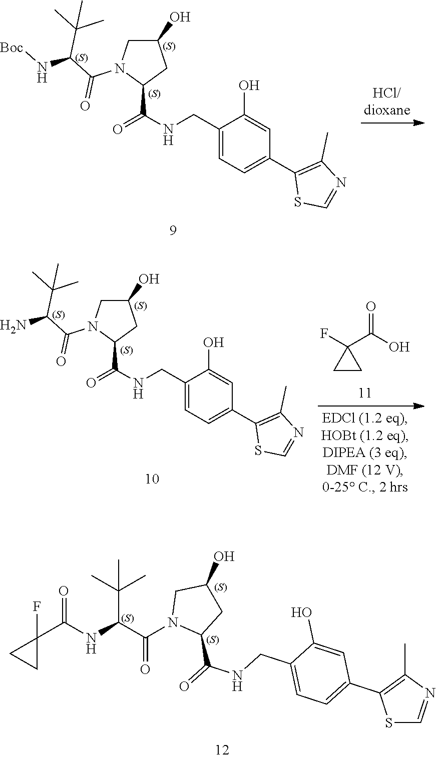

### Chemical Synthesis

All chemical reagents were obtained from commercial suppliers and used without further purification, unless otherwise stated. Reactions were run without taking precautions to exclude air or moisture, unless otherwise noted. Normal phase column chromatography was performed using silica gel columns and ACS grade solvents. Analytical TLC was performed on EM Reagent 0.25 mm silica gel 60 F254 plates and visualized by UV light. Compound identities were confirmed by ¹H and F (NMR) spectroscopy which were recorded on a Bruker 400 MHz spectrometer using the corresponding residual solvent peak (CDC3, 1H δ=7.27; CD₃OD, 1H δ=3.31; DMSO-d₆, 1H δ=2.50) as an internal standard. The chemical shifts for ¹H-NMR is reported to the second decimal place. Proton coupling constants are expressed in hertz (Hz). Standard abbreviations were used to denote spin multiplicity for ¹H NMR data.

### LCMS Methods:

### Method: 5_95AB_6min-220-254-ELSD-MS2000

LC/MS (The gradient was 5% B in 0.40 min and 5-95% B in 2.60 min, hold on 95% B in 1.00 min, and then 95-5% B in 0.01 min, the flow rate was 1.0 mL/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection. MS mode was positive electrospray ionization. MS range was 100-2000.

### SFC Methods:

### Method 1: OD_MeOH_IPAm_5_50_34_35_3min

Column: Chiralcel OD-3, 50×4.6 mm I.D., 3 um
Mobile phase: A: CO₂ B: MeOH (0.1% IPAm, v/v)
Gradient:
Time A % B %
0.0 95 5
0.2 95 5
1.2 50 50
2.2 50 50
2.6 95 5
3.0 95 5
Flow rate: 3.4 mL/min
Column temp.: 35° C.
ABPR: 1800 psi

### Method 2: OD_MeOH_IPAm_5_50_34_35_3 min

Column: Chiralcel OD-3, 50×4.6 mm I.D., 3 um
Mobile phase: A: CO₂ B: MeOH (0.1% IPAm, v/v)
Gradient:
Time A % B %
0.0 95 5
0.2 95 5
1.2 50 50
2.2 50 50
2.6 95 5
3.0 95 5
Flow rate: 3.4 mL/min
Column temp.: 35° C.
ABPR: 1800 psi

### Experimentals for Largest Scale Run:

### General Procedure for Preparation of Compound 2—Notebook Page: ET51978-425.

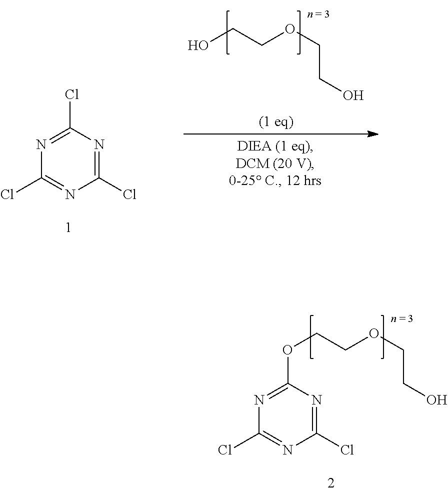

To a solution of Compound 1 (5 g, 27.11 mmol, 1 eq) and DIEA (3.50 g, 27.11 mmol, 4.72 mL, 1 eq) in DCM (100 mL) was added 2-[2-[2-(2-hydroxyethoxy)ethoxy]ethoxy]ethanol (5.27 g, 27.11 mmol, 4.66 mL, 1 eq) at 0° C. The reaction mixture was stirred at 25° C. for 12 hrs. TLC (petroleum ether/ethyl acetate=0/1) showed the starting material was consumed and a new spot was generated. The reaction mixture was filtered and concentrated under reduced pressure to give a residue, which was purified by chromatography on silica gel (eluted with petroleum ether/ethyl acetate=10/1 to 0/1) to give Compound 2 (1.5 g, yield 16.17%) as a colorless oil. 1H NMR (ET51978-425-P1A, 400 MHz, CHLOROFORM-d) δ 3.57-3.76 (m, 12H), 3.85-3.90 (m, 2H), 4.61-4.70 (m, 2H)

### General Procedure for Preparation of Compound 4—Notebook Page: ET51978-456.

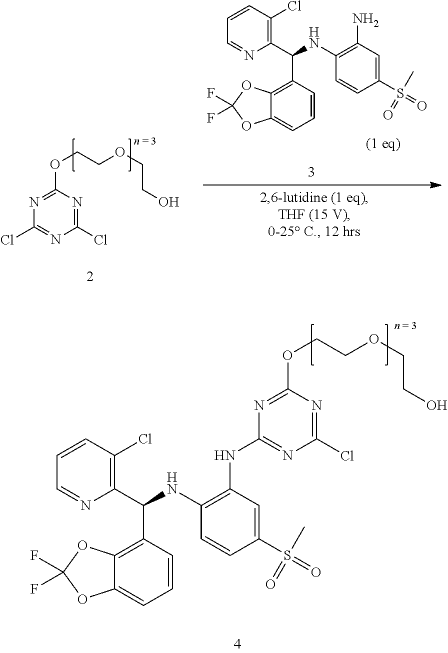

To a solution of Compound 2 (500 mg, 1.46 mmol, 1 eq) and 2,6-lutidine (156.58 mg, 1.46 mmol, 1 eq) in THF (5 mL) was added Compound 3 (683.67 mg, 1.46 mmol, 1 eq) in THF (2.5 mL) dropwise at 0° C. The reaction mixture was stirred at 25° C. for 12 hrs. LCMS showed all the starting material was consumed and a new peak with desired product MS was detected. The reaction mixture was diluted with water (30 mL) and extracted with ethyl acetate (3×20 mL). The organic layer was separated and the combined organic layer was washed with brine (2×20 mL), dried over Na₂SO₄, filtered and concentrated under reduced pressure to give a residue, which was purified by chromatography on silica gel (eluted with petroleum ether/ethyl acetate=50/1 to 5/1) to give Compound 4 (390 mg, yield 34.50%) as a yellow solid.

1H NMR (ET51978-456-P1A, 400 MHz, CHLOROFORM-d) δ 3.02 (s, 3H), 3.39-3.93 (m, 15H), 4.29-4.68 (m, 2H), 6.26 (br d, J=6.68 Hz, 1H), 6.72-7.09 (m, 5H), 7.24 (br dd, J=7.81, 4.71 Hz, 1H), 7.70 (br t, J=6.68 Hz, 2H), 7.78-8.26 (m, 2H), 8.42 (br s, 1H)

### General Procedure for Preparation of Compound 5—Notebook Page: ET51978-457.

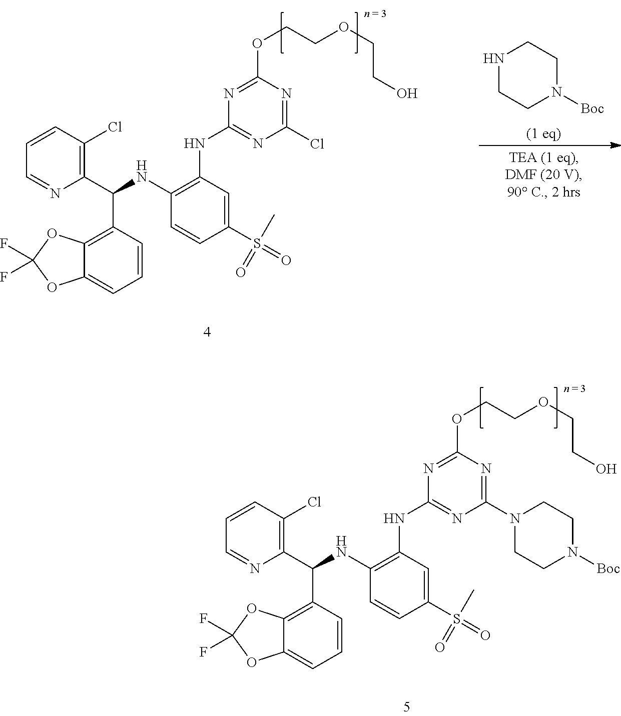

To a solution of Compound 4 (390 mg, 504.14 umol, 1 eq) and tert-butyl piperazine-1-carboxylate (93.90 mg, 504.14 umol, 1 eq) in DMF (7.8 mL) was added TEA (51.01 mg, 504.14 umol, 1 eq). The reaction mixture was stirred at 90° C. for 2 hrs. LCMS showed all the starting material was consumed and a new peak with desired product MS was detected. The reaction mixture was diluted with water (30 mL) and extracted with ethyl acetate (3×20 mL). The organic layer was separated and the combined organic layer was washed with brine (2×20 mL), dried over Na₂SO₄, filtered and concentrated under reduced pressure to give Compound 5 (340 mg, yield 73.04%) as a yellow oil, which was used to the next step directly without further purification. 1H NMR (ET51978-457-P1A, 400 MHz, CHLOROFORM-d) δ 1.44-1.50 (m, 9H), 2.99 (s, 3H), 3.41 (br s, 4H), 3.57-3.85 (m, 18H), 4.43 (br s, 2H), 6.24 (d, J=7.34 Hz, 1H), 6.75 (br s, 1H), 6.86-7.00 (m, 3H), 7.22 (dd, J=8.01, 4.71 Hz, 1H), 7.61 (dd, J=8.62, 1.77 Hz, 1H), 7.69 (d, J=7.95 Hz, 1H), 8.08 (br s, 1H), 8.48 (br s, 1H)

### General Procedure for Preparation of Compound 6—Notebook Page: ET51978-461.

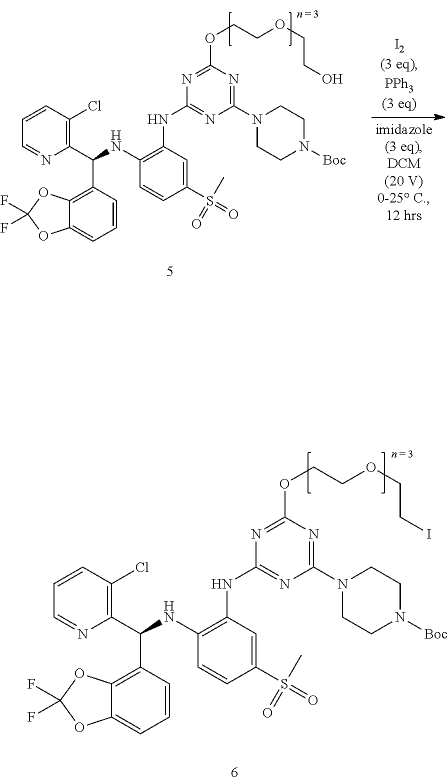

To a solution of Compound 5 (340 mg, 368.21 umol, 1 eq) and 12 (280.37 mg, 1.10 mmol, 3 eq) in DCM (6.8 mL) was added PPh₃ (289.73 mg, 1.10 mmol, 3 eq) and imidazole (75.20 mg, 1.10 mmol, 3 eq) at 0° C. The reaction mixture was stirred at 25° C. for 12 hrs. LCMS showed all starting material was consumed and 46% of product with desired MS was detected. The reaction mixture was diluted with water (20 mL) and extracted with DCM (3-15 mL). The organic layer was separated and the combined organic layer was washed with brine (2×15 mL), dried over Na₂SO₄, filtered and concentrated under reduced pressure to give a residue, which was purified by chromatography on silica gel (eluted with petroleum ether/ethyl acetate=50/1 to 0/1) to give Compound 6 (200 mg, yield 52.57%) as a yellow solid.

1H NMR (ET51978-461-P1A, 400 MHz, CHLOROFORM-d) δ 1.47 (s, 9H), 2.99 (s, 3H), 3.26 (t, J=6.91 Hz, 2H), 3.42 (br s, 4H), 3.60-3.83 (m, 16H), 4.43 (br s, 2H), 6.24 (br d, J=7.27 Hz, 1H), 6.75 (br d, J=7.03 Hz, 1H), 6.87-7.00 (m, 4H), 7.23 (br dd, J=7.99, 4.65 Hz, 1H), 7.62 (br d, J=7.39 Hz, 1H), 7.69 (br d, J=7.87 Hz, 1H), 8.09 (br s, 1H), 8.47 (br d, J=4.17 Hz, 1H)

### General Procedure for Preparation of Compound 7—Notebook Page: ET51978-476, 477.

The reactions were conducted in parallel but combined for purification. ET51978-477 was taken as an example.

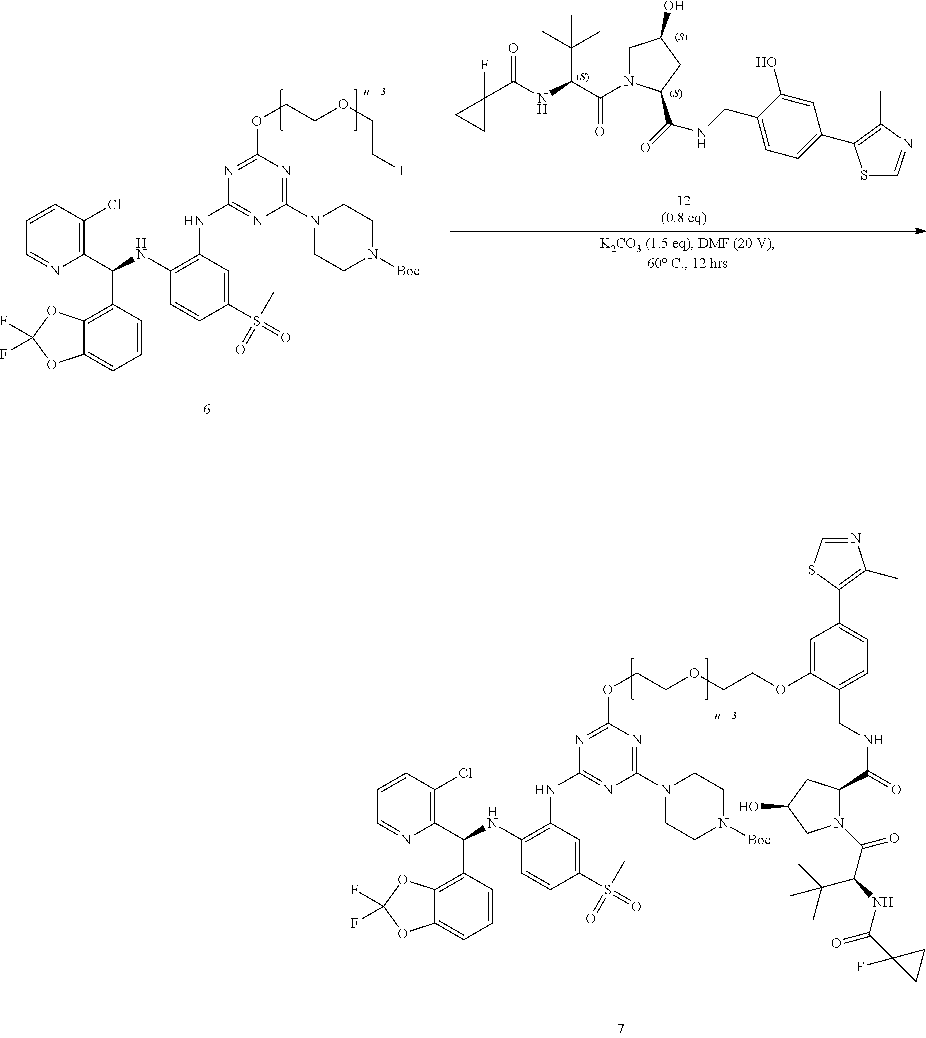

To a solution of Compound 6 (100 mg, 96.78 umol, 1 eq) and Compound 12 (41.24 mg, 77.42 umol, 0.8 eq) in DMF (2 mL) was added K₂CO₃ (20.06 mg, 145.17 umol, 1.5 eq). The reaction mixture was stirred at 60° C. for 12 hrs. LCMS showed all starting material was consumed and 40% of product with desired MS was detected. One additional reaction was set up as detailed above. After cooling to 25° C., both two reaction mixtures were combined. The combined reaction mixture was filtered and concentrated under reduced pressure to give a residue, which was purified by Prep-HPLC (column: Phenomenex Luna 80*30 mm*3 um; mobile phase: [water (TFA)-ACN]; B %: 40%-85%, 8 min) to give Compound 7 (63 mg, yield 22.63%) as a white solid.

LCMS (ESI+): RT=0.906 min, m/z 1437.4 (M+H)⁺.

5_100AB_2min: LC/MS (The column used for chromatography was a Halo C18 3.0*30 mm, 5 um. Detection methods are diode array (DAD). MS mode was positive electrospray ionization. MS range was 50-2000. Mobile phase A was 0.04% TFA in water, and mobile phase B was 0.02% TFA in HPLC grade acetonitrile. The gradient was 5-100% B in 1.30 min. 5% B in 0.01 min, 5-100% B (0.01-0.80 min) with a hold at 100% B for 0.50 min. The flow rate was 1.5 mL/min (0.00-1.30 min).

### General Procedure for Preparation of NUCC-0227297—Notebook Page: ET51978-484.

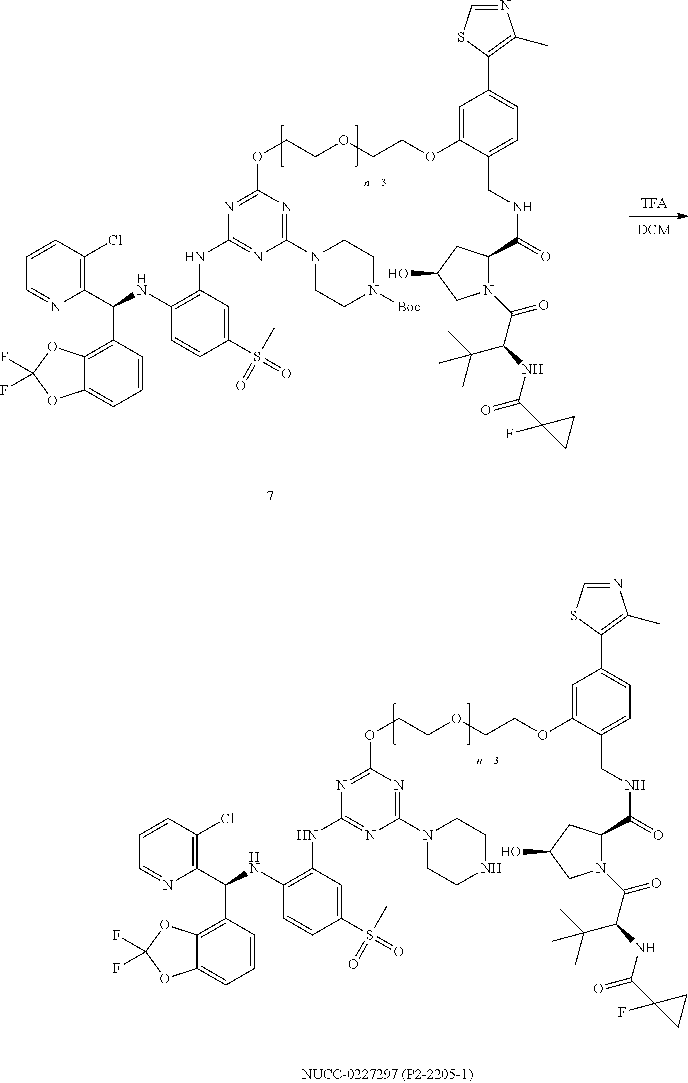

To a solution of Compound 7 (63 mg, 43.81 umol, 1 eq) in DCM (2 mL) was added TFA (149.86 mg, 1.31 mmol, 30 eq). The reaction mixture was stirred at 25° C. for 2 hrs. LCM S showed about 3% of Compound 7 was remaining and 75% of product with desired MS was detected. The reaction mixture was concentrated under reduced pressure to give a residue, which was purified by Prep-HPLC (column: Phenomenex Luna 80*30 mm*3 um; mobile phase: [water (TFA)-ACN]; B %: 30%-60%, 8 min) to give NUCC-0227297 (25.4 mg, yield 39.93%, TFA salt) as a white solid.

1H NMR (400 MHz, DMSO-d6) δ 0.97 (s, 9H), 1.21 (br d, J=8.80 Hz, 2H), 1.31-1.40 (m, 2H), 1.71-1.79 (m, 1H), 2.32-2.37 (m, 1H), 2.45 (s, 3H), 3.08 (s, 7H), 3.48 (br d, J=5.26 Hz, 13H), 3.78 (br d, J=3.79 Hz, 2H), 3.82-3.91 (m, 4H), 4.16-4.27 (m, 5H), 4.31-4.36 (m, 2H), 4.41-4.46 (m, 1H), 4.55 (d, J=8.80 Hz, 1H), 6.36 (d, J=7.34 Hz, 1H), 6.82 (br d, J=8.93 Hz, 1H), 6.96 (br dd, J=7.76, 1.28 Hz, 2H), 7.04 (s, 1H), 7.12-7.17 (m, 1H), 7.22 (br d, J=7.95 Hz, 1H), 7.29-7.33 (m, 2H), 7.40 (d, J=7.82 Hz, 1H), 7.45-7.52 (m, 2H), 8.02 (d, J=8.07 Hz, 1H), 8.52-8.59 (m, 2H), 8.79 (br s, 2H), 8.98 (s, 1H), 9.42 (br d, J=1.59 Hz, 1H)

LCMS (ESI+): RT=2.467 min, m/z 1337.4 (M+H)⁺.

5_95AB_6min-220-254-ELSD-MS2000

LC/MS (The gradient was 5% B in 0.40 min and 5-95% B in 2.60 min, hold on 95% B in 1.00 min, and then 95-5% B in 0.01 min, the flow rate was 1.0 mL/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection. MS mode was positive electrospray ionization. MS range was 100-2000.

### General Procedure for Preparation of Compound 3—Notebook Page: ET51978-428.

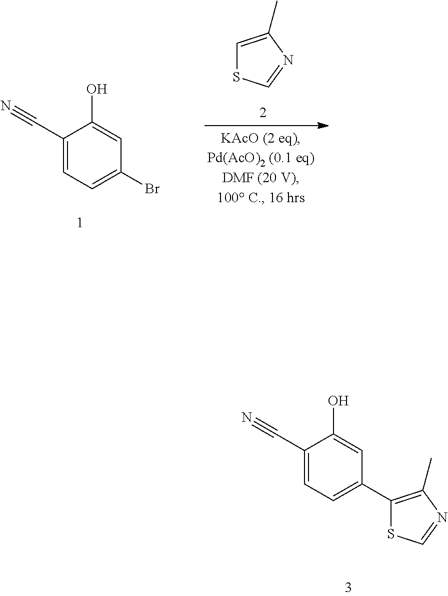

To a solution of Compound 1 (9 g, 45.45 mmol, 1 eq), Compound 2 (9.01 g, 90.90 mmol, 8.27 mL, 2 eq) and KOAc (8.92 g, 90.90 mmol, 2 eq) in DMF (180 mL) was added Pd(OAc)₂ (1.02 g, 4.55 mmol, 0.1 eq) under argon protection. The reaction mixture was stirred at 100° C. for 16 hrs. LCMS showed all the starting material was consumed and a new peak with desired product MS was detected. The reaction mixture was filtered, diluted with water (600 mL) and extracted with ethyl acetate (3×300 mL). The organic layer was separated and the combined organic layer was washed with brine (2×300 mL), dried over Na₂SO₄, filtered and concentrated under reduced pressure to give a residue, which was purified by chromatography on silica gel (eluted with petroleum ether/THF=50/1 to 1/1) to give Compound 3 (8.3 g, yield 84.44%) as a yellow solid.

¹H NMR (ET51978-428-P1A2, 400 MHz, METHANOL-d₄) δ 2.52 (s, 3H), 7.00-7.12 (m, 2H), 7.54-7.65 (m, 1H), 8.94 (s, 1H)

### General Procedure for Preparation of Compound 4—Notebook Page: ET51978-434

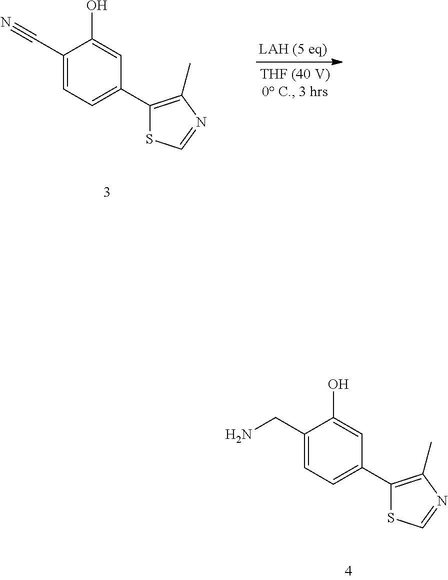

To a solution of Compound 3 (4 g, 18.50 mmol, 1 eq) in THF (160 mL) was added LiAlH. (3.51 g, 92.48 mmol, 5 eq) at 0° C. The reaction mixture was stirred at 0° C. for 3 hrs. LCMS showed all the starting material was consumed and 50% of product with desired MS was detected. After the reaction mixture was cooled to 0° C., the reaction mixture was quenched by addition of water (4 mL), followed by aqueous NaOH (4 mL, 4 M) and water (12 mL). The reaction mixture was extracted with ethyl acetate (3×20 mL). The organic layer was separated and the combined organic layer was washed with brine (2×20 mL), dried over Na₂SO₄, filtered and concentrated under reduced pressure to give a residue, which was purified by column chromatography on silica gel eluted with ethyl acetate/NH₃·H₂O=30/1 to afford Compound 4 (940 mg, yield 23.07%) as a yellow oil.

¹H NMR (ET51978-434-P1A, 400 MHz, METHANOL-d₄) δ 2.48 (s, 3H), 3.90 (s, 2H), 6.80-6.88 (m, 2H), 7.19 (d, J=7.63 Hz, 1H), 8.83 (s, 1H)

### General Procedure for Preparation of Compound 6—Notebook Page: ET51978-446.

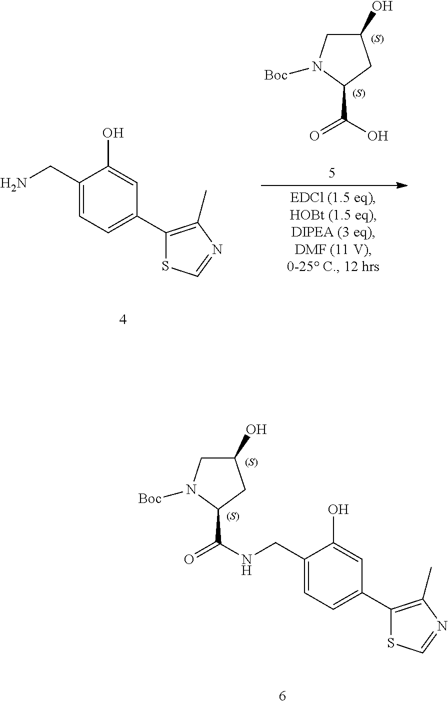

To a solution of Compound 4 (940 mg, 4.27 mmol, 1 eq), Compound 5 (986.74 mg, 4.27 mmol, 1 eq) and DIEA (1.65 g, 12.80 mmol, 2.23 mL, 3 eq) in DMF (10.3 mL) was added EDCI (1.23 g, 6.40 mmol, 1.5 eq) and HOBt (864.87 mg, 6.40 mmol, 1.5 eq) at 0° C. The reaction mixture was stirred at 25° C. for 12 hrs. LCMS showed all the starting material was consumed and a new peak with desired product MS was detected. The reaction mixture was diluted with water (60 mL) and extracted with ethyl acetate (3-30 mL). The organic layer was separated and the combined organic layer was washed with brine (2×30 mL), dried over Na₂SO₄, filtered and concentrated under reduced pressure to give a residue, which was purified by chromatography on silica gel (eluted with petroleum ether/ethyl acetate=50/1 to 10/1) to give Compound 6 (930 mg, yield 50.27%) as a white solid.

¹H NMR (ET51978-446-P1A, 400 MHz, METHANOL-d₄) δ 1.29 (s, 6H), 1.49 (br s, 3H), 2.00-2.07 (m, 2H), 2.37-2.47 (m, 1H), 2.50 (s, 3H), 3.47 (dd, J=11.32, 0.95 Hz, 1H), 3.59 (br dd, J=10.97, 4.65 Hz, 1H), 4.25 (br d, J=6.44 Hz, 1H), 4.29-4.45 (m, 3H), 6.91-6.96 (m, 2H), 7.34 (br d, J=7.39 Hz, 1H), 8.87 (s, 1H)

### General Procedure for Preparation of Compound 7—Notebook Page: ET51978-451.

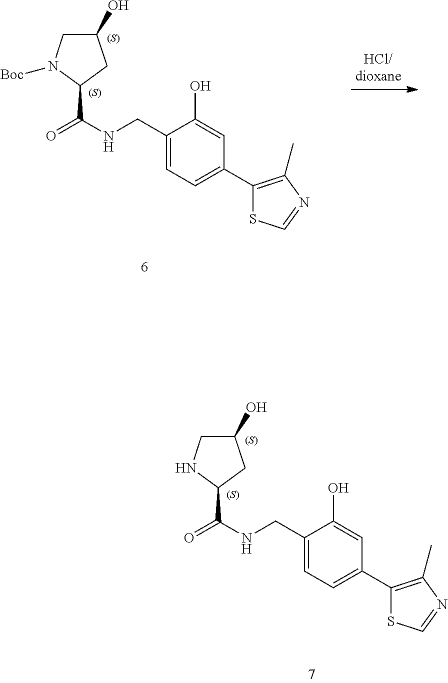

To a solution of Compound 6 (920 mg, 2.12 mmol, 1 eq) in dioxane (9 mL) was added HCl/dioxane (4 M, 9 mL, 16.96 eq) dropwise at 0° C. The reaction mixture was stirred at 25° C. for 2 hrs. LCMS showed all the starting material was consumed and a new peak with desired product MS was detected. The reaction mixture was concentrated under reduced pressure to give Compound 7 (720 mg, yield 91.73%, HCl) as a yellow solid, which was used to the next step directly without further purification.

¹H NMR (ET51978-451-P1A, 400 MHz, METHANOL-d₄) δ 1.60 (s, 1H), 2.13-2.22 (m, 1H), 2.56-2.68 (m, 4H), 3.33-3.43 (m, 2H), 4.40 (dd, J=10.07, 4.71 Hz, 1H), 4.47-4.57 (m, 3H), 6.97-7.03 (m, 2H), 7.40 (d, J=7.99 Hz, 1H), 9.80 (s, 1H)

### General Procedure for Preparation of Compound 9—Notebook Page: ET51978-470.

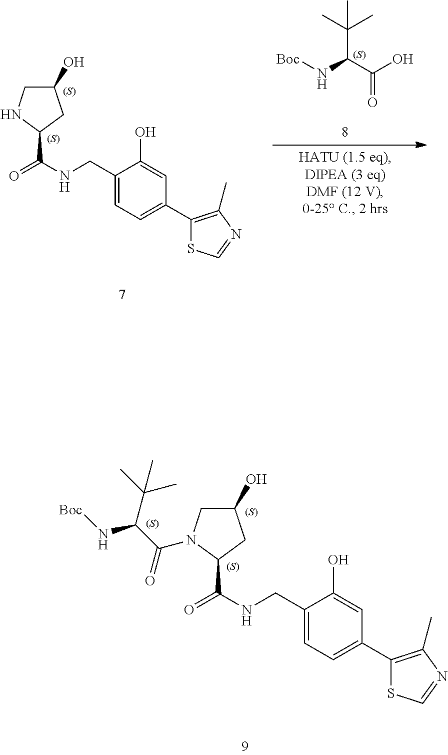

To a solution of Compound 8 (427.73 mg, 1.85 mmol, 0.95 eq) in DMF (5.76 mL) was added HATU (1.11 g, 2.92 mmol, 1.5 eq) and DIEA (503.18 mg, 3.89 mmol, 678.14 uL, 2 eq) successively at 0° C. After stirred at 0° C. for 30 min, a mixture of Compound 7 (720 mg, 1.95 mmol, 1 eq, HCl) and DIEA (251.59 mg, 1.95 mmol, 339.07 uL, 1 eq) in DMF (2.88 mL) was added to the reaction mixture. The reaction mixture was stirred at 25° C. for 2 hrs. LCMS showed all the starting material was consumed and a new peak with desired product MS was detected. The reaction mixture was diluted with water (30 mL) and extracted with ethyl acetate (3×20 mL). The organic layer was separated and the combined organic layer was washed with brine (2×20 mL), dried over Na₂SO₄, filtered and concentrated under reduced pressure to give a residue, which was purified by chromatography on silica gel (eluted with petroleum ether/ethyl acetate=50/1 to 10/1) to give Compound 9 (660 mg, yield 62.02%) as a white solid.

¹H NMR (ET51978-470-P1A, 400 MHz, METHANOL-d₄) δ 1.00 (s, 9H), 1.40-1.49 (m, 9H), 1.97-2.01 (m, 1H), 2.37-2.52 (m, 4H), 3.71 (dd, J=10.55, 3.28 Hz, 1H), 3.99 (br dd, J=10.49, 4.89 Hz, 1H), 4.23 (s, 1H), 4.30-4.48 (m, 3H), 4.55 (dd, J=9.18, 4.17 Hz, 1H), 6.83-6.95 (m, 2H), 7.35 (d, J=7.63 Hz, 1H), 8.85 (s, 1H)

### SFC Method:

OD_MeOH_IPAm_5_50_34_35_3min
Column: Chiralcel OD-3, 50×4.6 mm I.D., 3 um
Mobile phase: A: CO₂ B: MeOH (0.1% IPAm, v/v)
Gradient:
Time A % B %
0.0 95 5
0.2 95 5
1.2 50 50
2.2 50 50
2.6 95 5
3.0 95 5
Flow rate: 3.4 mL/min
Column temp.: 35° C.
ABPR: 1800 psi

### General Procedure for Preparation of Compound 10—Notebook Page: ET51978-472.

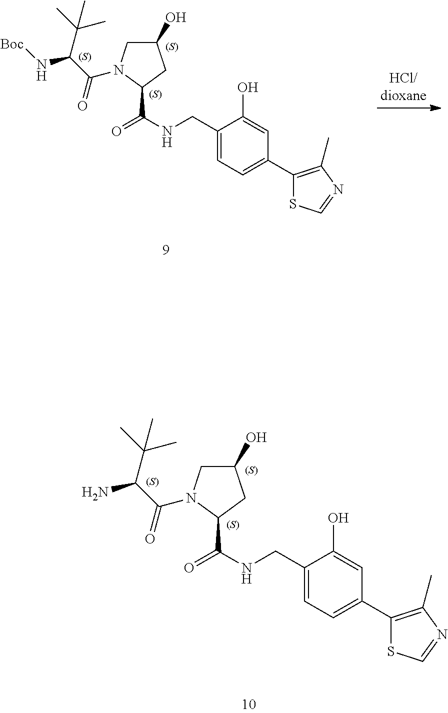

To a solution of Compound 9 (660 mg, 1.21 mmol, 1 eq) in dioxane (3.3 mL) was added HCl/dioxane (4 M, 3.30 mL, 10.93 eq) dropwise at 0° C. The reaction mixture was stirred at 25° C. for 2 hrs. LCMS showed all the starting material was consumed and a new peak with desired product MS was detected. The reaction mixture was concentrated under reduced pressure to give Compound 10 (530 mg, yield 90.89%, HCl) as a white solid, which was used to the next step directly without further purification.

LCMS (ESI+): RT=0.691 min, m/z 445.2 (M−H)*.

NEG5-95CD_2 min: LC/MS (The column used for chromatography was Xbridge-C18 2.1*50 mm, (5 um particles). Detection methods are diode array (DAD). MS mode was negative electrospray ionization. MS range was 50-2000. Mobile phase A was 10 mM ammonium bicarbonate in water, and mobile phase B was HPLC grade acetonitrile. The gradient was 5-95% B in 1.50 min 0.5% B in 0.01 min, 5-95% B (0.01-0.70 min), 95% B for 0.46 min. 95-5% B (1.61-1.50 min) with a hold at 5% B for 0.11 min. The flow rate was 1.5 mL/min.

### General Procedure for Preparation of Compound 12—Notebook Page: ET51978-475.

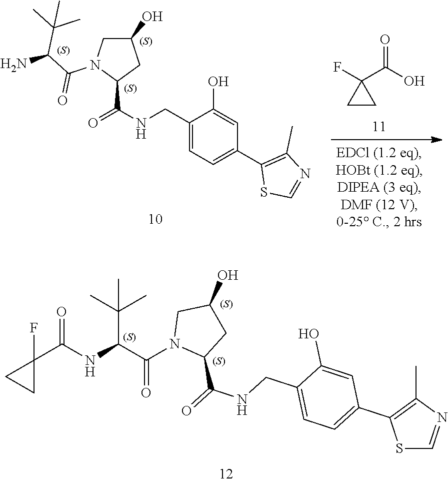

To a solution of Compound 10 (530 mg, 1.10 mmol, 1 eq, HCl), Compound 11 (91.36 mg, 877.81 umol, 0.8 eq), DIEA (425.44 mg, 3.29 mmol, 573.37 uL, 3 eq) and HOBt (177.92 mg, 1.32 mmol, 1.2 eq) in DMF (6.36 mL) was added EDCI (252.42 mg, 1.32 mmol, 1.2 eq) at 0° C.

The reaction mixture was stirred at 25° C. for 2 hrs. LCMS showed all the starting material was consumed and a new peak with desired product MS was detected. The reaction mixture was diluted with water (30 mL) and extracted with ethyl acetate (3×20 mL). The organic layer was separated and the combined organic layer was washed with brine (2×20 mL), dried over Na₂SO₄, filtered and concentrated under reduced pressure to give a residue, which was purified by chromatography on silica gel (eluted with petroleum ether/ethyl acetate=50/1 to 8/1) to give Compound 12 (350 mg, yield 59.89%) as a white solid.

¹H NMR (ET51978-475-P1A, 400 MHz, METHANOL-d₄) δ 1.03-1.05 (m, 9H), 1.27-1.38 (m, 4H), 1.99 (br t, J=4.47 Hz, 1H), 2.48 (s, 4H), 3.72 (dd, J=10.49, 3.46 Hz, 1H), 3.97 (dd, J=10.61, 5.01 Hz, 1H), 4.34-4.44 (m, 3H), 4.55 (dd, J=9.12, 4.47 Hz, 1H), 4.64-4.70 (m, 1H), 6.87-6.91 (m, 2H), 7.36 (d, J=7.87 Hz, 1H), 8.85 (s, 1H)

### SFC Method:

OD_MeOH_IPAm_5_50_34_35_3min
Column: Chiralcel OD-3, 50×4.6 mm I.D., 3 um
Mobile phase: A: CO₂ B: MeOH (0.1% IPAm, v/v)
Gradient:
Time A % B %
0.0 95 5
0.2 95 5
1.2 50 50
2.2 50 50
2.6 95 5
3.0 95 5
Flow rate: 3.4 mL/min
Column temp.: 35° C.
ABPR: 1800 psi

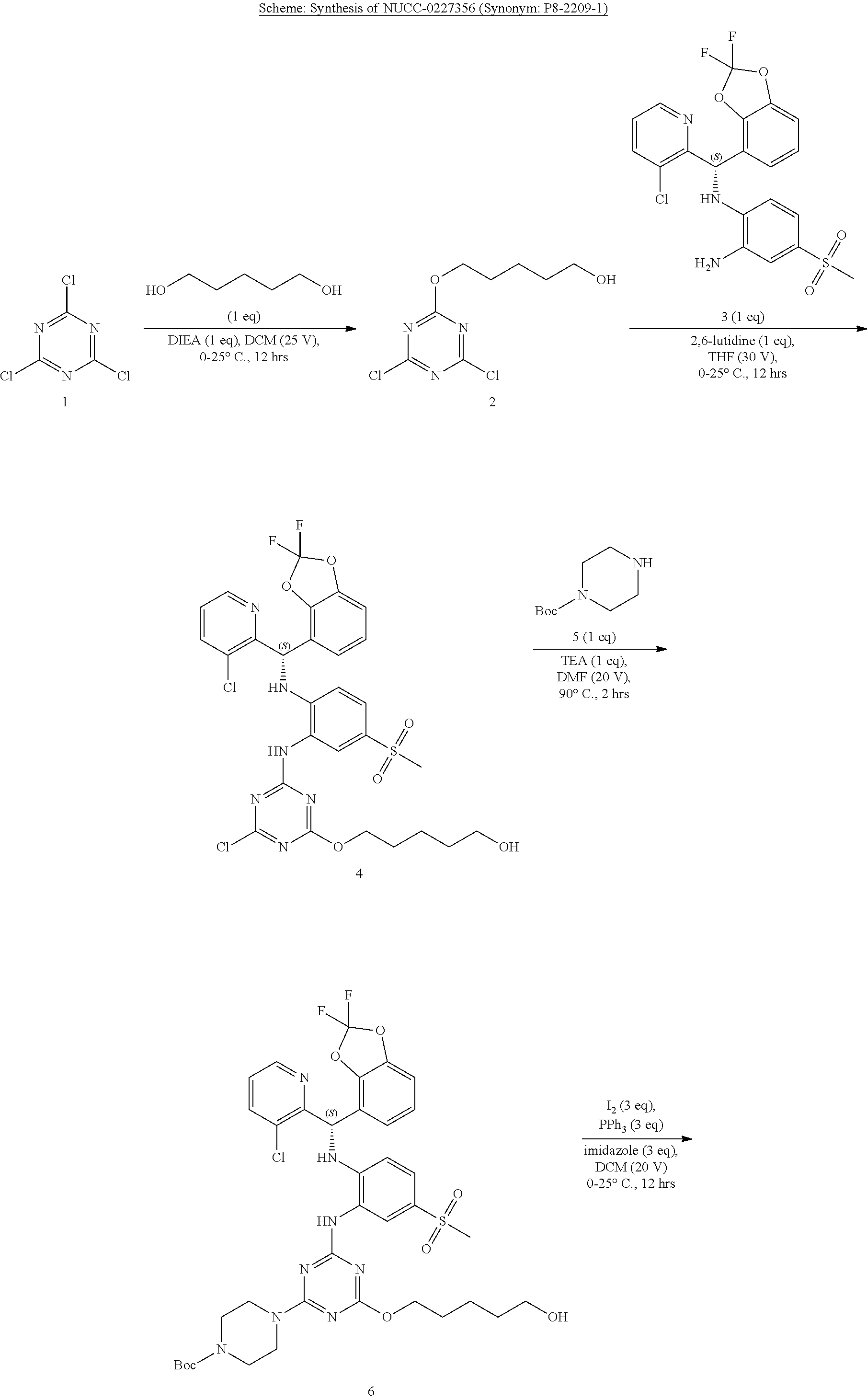

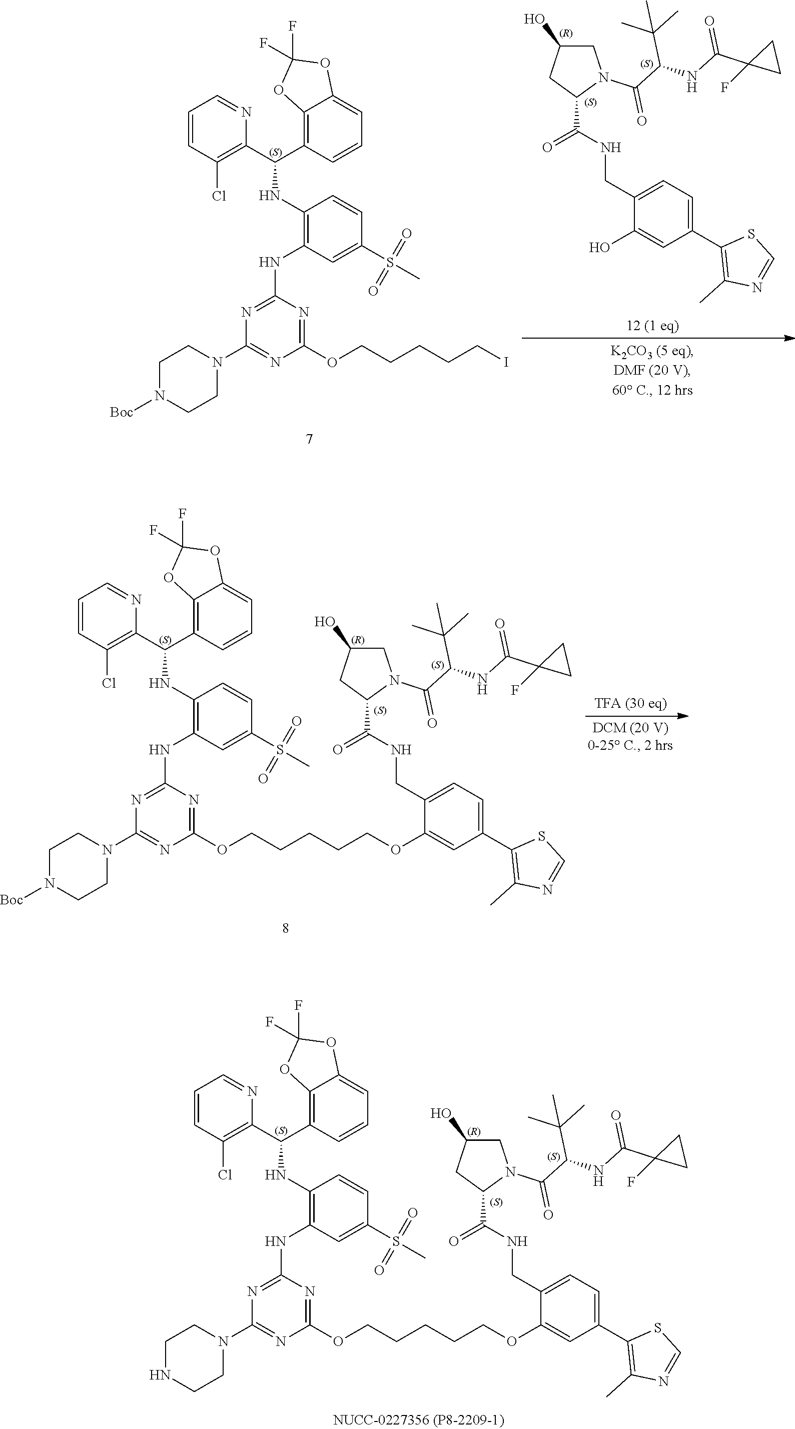

### Experimentals for Largest Scale Run:

### General Procedure for Preparation of Compound 2—Notebook Page: ET51978-507.

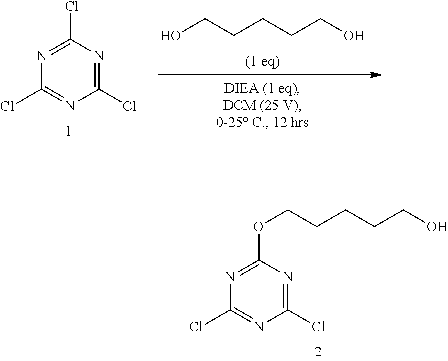 [SMILES: Clc1nc(Cl)nc(Cl)n1 ; OCCCCCO ; OCCCCCOc1nc(Cl)nc(Cl)n1]

To a solution of Compound 1 (500 mg, 2.71 mmol, 1 eq) and DIEA (350.42 mg, 2.71 mmol, 1 eq) in DCM (10 mL) was added pentane-1,5-diol (282.38 mg, 2.71 mmol, 1 eq) in DCM (2.5 mL) dropwise at 0° C. The reaction mixture was stirred at 25° C. for 12 hrs. TLC (petroleum ether/ethyl acetate=3/1, Rf=0.5) showed the starting material was consumed and a new spot was generated. The reaction mixture was filtered and concentrated under reduced pressure to give a residue, which was purified by chromatography on silica gel (eluted with petroleum ether/ethyl acetate=50/1 to 5/1) to give Compound 2 (200 mg, yield 29.26%) as a colorless oil. 1H NMR (ET51978-507-P1B, 400 MHz, CHLOROFORM-d) δ 1.51-1.60 (m, 3H), 1.61-1.70 (m, 2H), 1.80-1.85 (m, 2H), 3.69 (t, 0.1=6.32 Hz, 2H), 4.51 (t, J=6.57 Hz, 2H)

### General Procedure for Preparation of Compound 4—Notebook Page: ET51978-508.

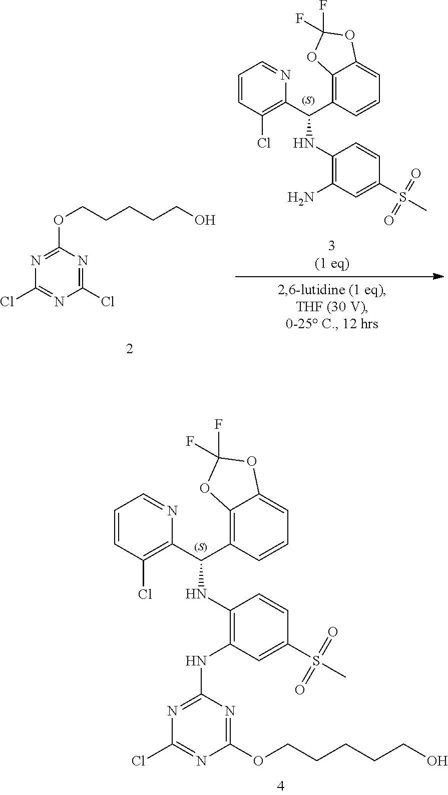

To a solution of Compound 2 (200 mg, 793.34 umol, 1 eq) in THF (4 mL) was added Compound 3 (371.18 mg, 793.34 umol, 1 eq) and 2,6-lutidine (85.01 mg, 793.34 umol, 1 eq) in THF (2 mL) dropwise at 0° C. The reaction mixture was stirred at 25° C. for 12 hrs. LCMS showed all the starting material was consumed and a new peak with desired product MS was detected. The reaction mixture was diluted with water (30 mL) and extracted with ethyl acetate (3×10 mL).

The organic layer was separated and the combined organic layer was washed with brine (2×10 mL), dried over Na₂SO₄, filtered and concentrated under reduced pressure to give a residue, which was purified by chromatography on silica gel (eluted with petroleum ether/ethyl acetate=50/1 to 3/1) to give Compound 4 (400 mg, yield 73.77%) as a yellow solid. 1H NMR (ET51978-508-P1A, 400 MHz, CHLOROFORM-d) δ 1.35-1.71 (m, 7H), 3.03 (s, 3H), 3.51-3.70 (m, 2H), 4.19-4.42 (m, 2H), 6.23 (br d, J=5.87 Hz, 1H), 6.82-7.01 (m, 5H), 7.19-7.26 (m, 1H), 7.62-7.77 (m, 2H), 7.98 (br d, J=15.04 Hz, 1H), 8.42 (br s, 1H)

### General Procedure for Preparation of Compound 6—Notebook Page: ET51978-512.

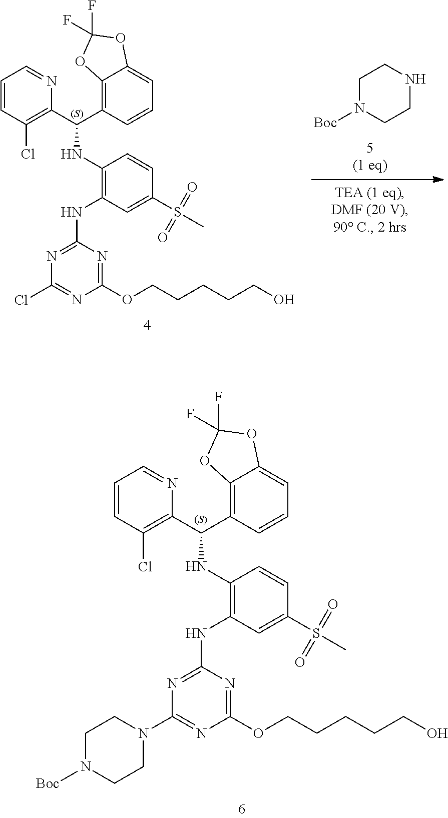

To a solution of Compound 4 (350 mg, 512.06 umol, 1 eq) and Compound 5 (95.37 mg, 512.06 umol, 1 eq) in DMF (7 mL) was added TEA (51.82 mg, 512.06 umol, 1 eq). The reaction mixture was stirred at 90° C. for 2 hrs. LCMS showed all the starting material was consumed and a new peak with desired product MS was detected. The reaction mixture was diluted with water (60 mL) and extracted with ethyl acetate (3-30 mL). The organic layer was separated and the combined organic layer was washed with brine (2-30 mL), dried over Na₂SO₄, filtered and concentrated under reduced pressure to give a residue, which was purified by chromatography on silica gel (eluted with petroleum ether/ethyl acetate=50/1 to 5/1) to give Compound 6 (360 mg, yield 84.37%) as a yellow solid.

1H NMR (ET51978-512-PIA, 400 MHz, CHLOROFORM-d) δ 1.33-1.44 (m, 2H), 1.47 (s, 9H), 1.52-1.59 (m, 2H), 1.62-1.69 (m, 2H), 2.96-3.03 (m, 3H), 3.41 (br s, 4H), 3.53-3.86 (m, 6H), 4.19-4.35 (m, 2H), 6.19 (br d, J=7.00 Hz, 1H), 6.81 (br d, J=7.13 Hz, 1H), 6.84-7.03 (m, 4H), 7.17 (dd, J=8.00, 4.75 Hz, 1H), 7.57-7.69 (m, 2H), 8.12 (br s, 1H), 8.38 (br d, J=4.13 Hz, 1H)

### General Procedure for Preparation of Compound 7—Notebook Page: ET51978-534.

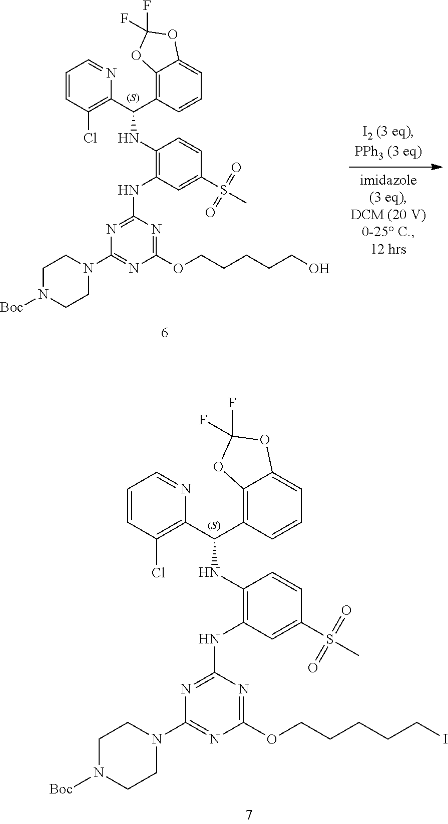

To a solution of Compound 6 (360 mg, 432.02 μmol, 1 eq) and I₂ (328.95 mg, 1.30 mmol, 3 eq) in DCM (7.2 mL) were added imidazole (88.24 mg, 1.30 mmol, 3 eq) and PPh₃ (339.94 mg, 1.30 mmol, 3 eq) at 0° C. The reaction mixture was stirred at 25° C. for 12 hrs. LCMS showed all starting material was consumed and 43% of product with desired MS was detected. The reaction mixture was diluted with water (10 mL) and extracted with DCM (3×10 mL). The organic layer was separated and the combined organic layer was washed with brine (2×10 mL), dried over Na₂SO₄, filtered and concentrated under reduced pressure to give a residue, which was purified by chromatography on silica gel (eluted with petroleum ether/ethyl acetate=50/1 to 0/1) to give Compound 7 (350 mg, yield 85.89%) as a yellow solid.

1H NMR (ET51978-534-P1A, 400 MHz, CHLOROFORM-d) δ 1.38-1.53 (m, 11H), 1.61-1.72 (m, 2H), 1.80 (dt, J=14.63, 7.17 Hz, 2H), 2.92-3.04 (m, 3H), 3.41 (br d, J=4.41 Hz, 4H), 3.54-3.87 (m, 4H), 4.13 (q, J=7.15 Hz, 1H), 4.19-4.31 (m, 2H), 6.18 (br d, J=6.79 Hz, 1H), 6.79 (br d, J=7.03 Hz, 1H), 6.85-7.00 (m, 4H), 7.15-7.22 (m, 1H), 7.63-7.69 (m, 2H), 8.13 (br s, 1H), 8.38 (br d, J=4.05 Hz, 1H)

### General Procedure for Preparation of Compound 8—Notebook Page: ET51978-547.

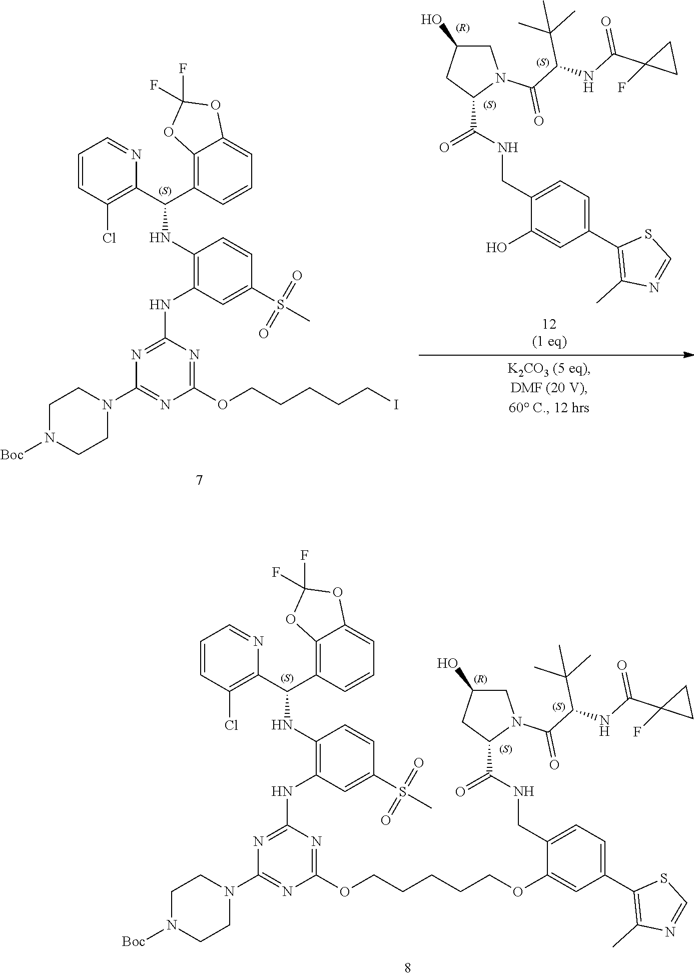

To a solution of Compound 7 (200 mg, 212.05 μmol, 1 eq) and Compound 12 (112.94 mg, 212.05 μmol, 1 eq) in DMF (4 mL) was added K₂CO₃ (146.53 mg, 1.06 mmol, 5 eq). The reaction mixture was stirred at 60° C. for 12 hrs. LCMS showed all starting material was consumed and 44% of product with desired MIS was detected. The reaction mixture was filtered and concentrated under reduced pressure to give a residue, which was purified by Prep-HPLC (column: Phenomenex Luna 80*30 mm*3 um; mobile phase: [water (TFA)-ACN]; B %: 55%-90%/o, 8 min) to give Compound 8 (65 mg, yield 22.74%) as a yellow solid.

LCMS (ESI+): RT=0.889 min, m/z 674.9 (M/2+H)⁺.

5_95AB_2 min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### General Procedure for Preparation of NUCC-0227356—Notebook Page: ET51978-551.

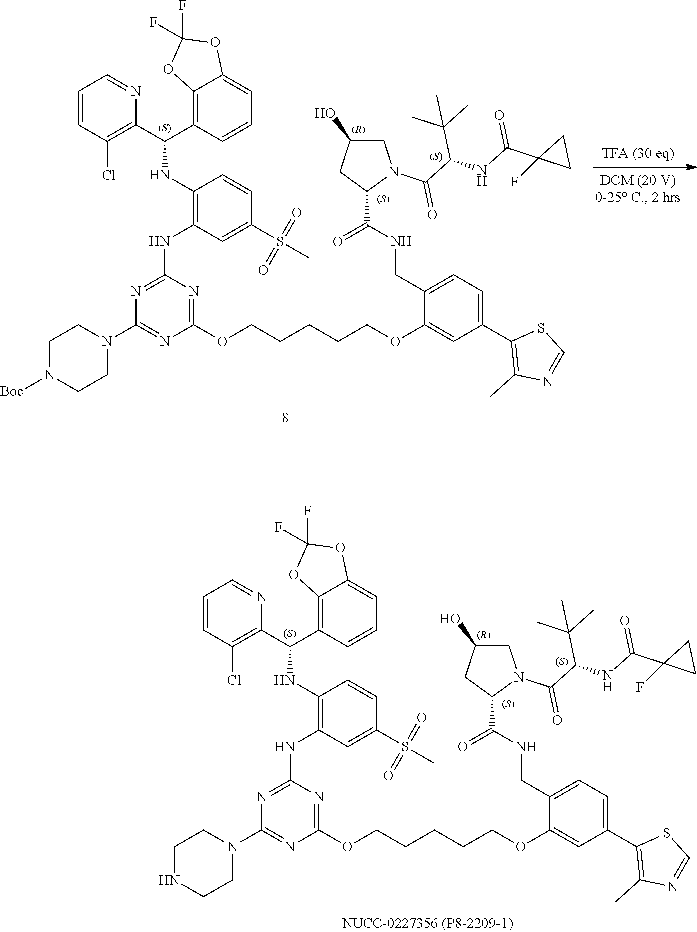

To a solution of Compound 8 (65 mg, 48.22 μmol, 1 eq) in DCM (1.3 mL) was added TFA (164.96 mg, 1.45 mmol, 30 eq) dropwise at 0° C. The reaction mixture was stirred at 25° C. for 2 hrs. LCMS showed all the starting material was consumed and a new peak with desired product MS was detected. The reaction mixture was concentrated under reduced pressure to give a residue, which was purified by Prep-HPLC (column: Phenomenex Luna 80*30 mm*3 um; mobile phase: [water(TFA)-ACN]; B %: 30%-65%, 8 min) to give NUCC-0227356 (24.7 mg, yield 35.66%, 94.8% purity, TFA salt) as a yellow solid.

1H NMR (ET51978-551-P1A, 400 MHz, DMSO-d₆) δ 0.94 (s, 9H), 1.17-1.26 (m, 2H), 1.31-1.41 (m, 2H), 1.47-1.62 (m, 2H), 1.78 (br d, J=7.13 Hz, 4H), 1.87-1.99 (m, 1H), 2.04-2.17 (m, 1H), 2.45 (s, 3H), 3.07 (s, 7H), 3.57-3.68 (m, 2H), 3.82-4.09 (m, 6H), 4.15-4.38 (m, 7H), 4.52 (br t, J=8.25 Hz, 2H), 4.59 (br d, J=9.26 Hz, 2H), 6.35 (br d, J=7.25 Hz, 1H), 6.80 (br d, J=8.88 Hz, 1H), 6.91-7.03 (m, 3H), 7.08-7.36 (m, 4H), 7.37-7.51 (m, 3H), 7.51-7.74 (m, 1H), 7.99-8.06 (m, 1H), 8.53 (br s, 2H), 8.78-8.92 (m, 2H), 8.95-9.00 (m, 1H), 9.32-9.53 (m, 1H)

LCMS (ESI+): RT=2.546 min, m/z 624.3, 1247.2 (M/2+H, M+H)⁺.

5_95AB_6min-220-254-ELSD-MS2000

LC/MS (The gradient was 5% B in 0.40 min and 5-95% B in 2.60 min, hold on 95% B in 1.00 min, and then 95-5% B in 0.01 min, the flow rate was 1.0 mL/min. Mobile phase A was 0.04% trifluoroacetic acid in water, mobile phase B was 0.02% trifluoroacetic acid in acetonitrile. The column used for chromatography was a Luna C18 50*2.0 mm column (5 um particles). Detection methods are diode array (DAD) and evaporative light scattering (ELSD) detection. MS mode was positive electrospray ionization. MS range was 100-2000.

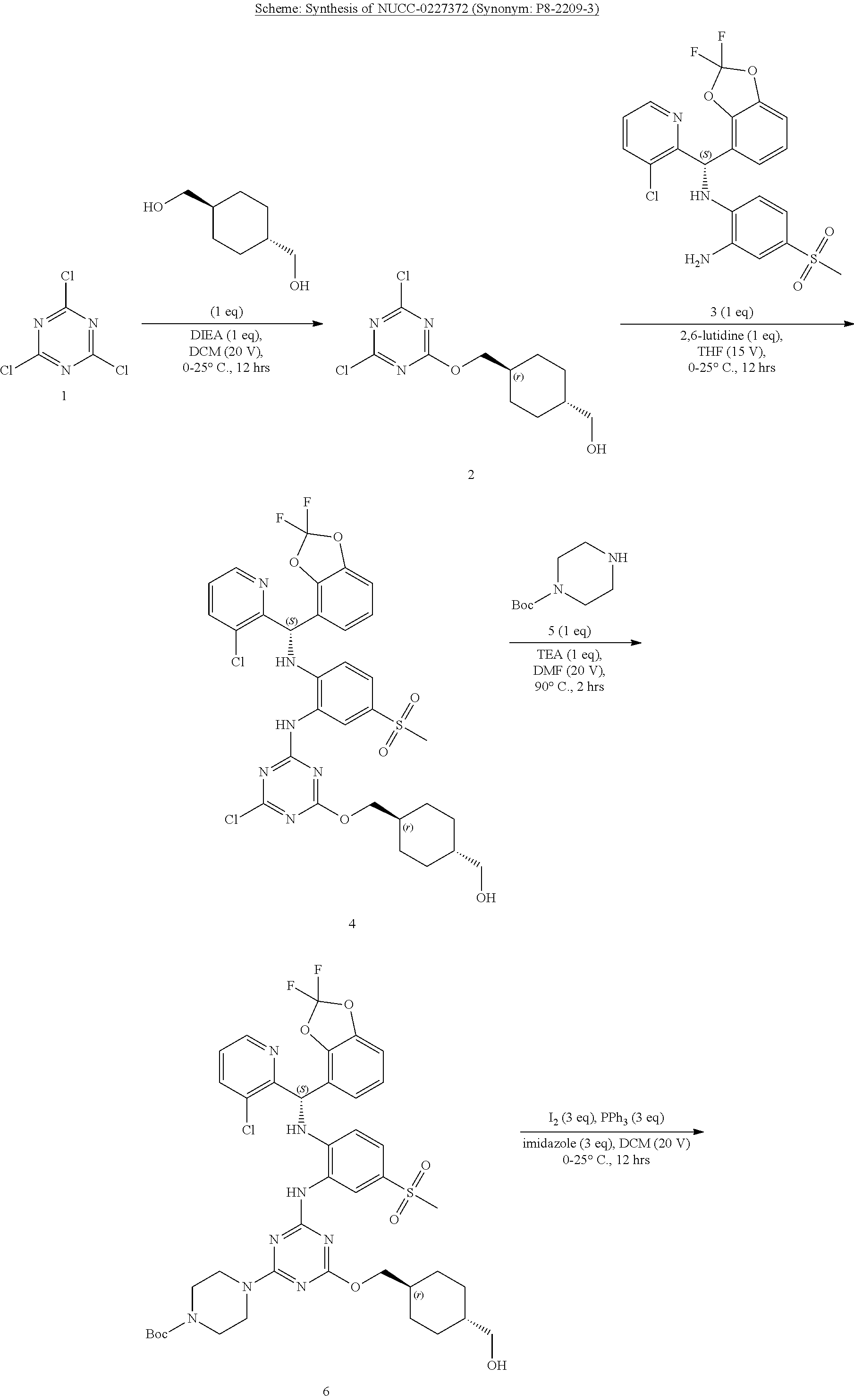

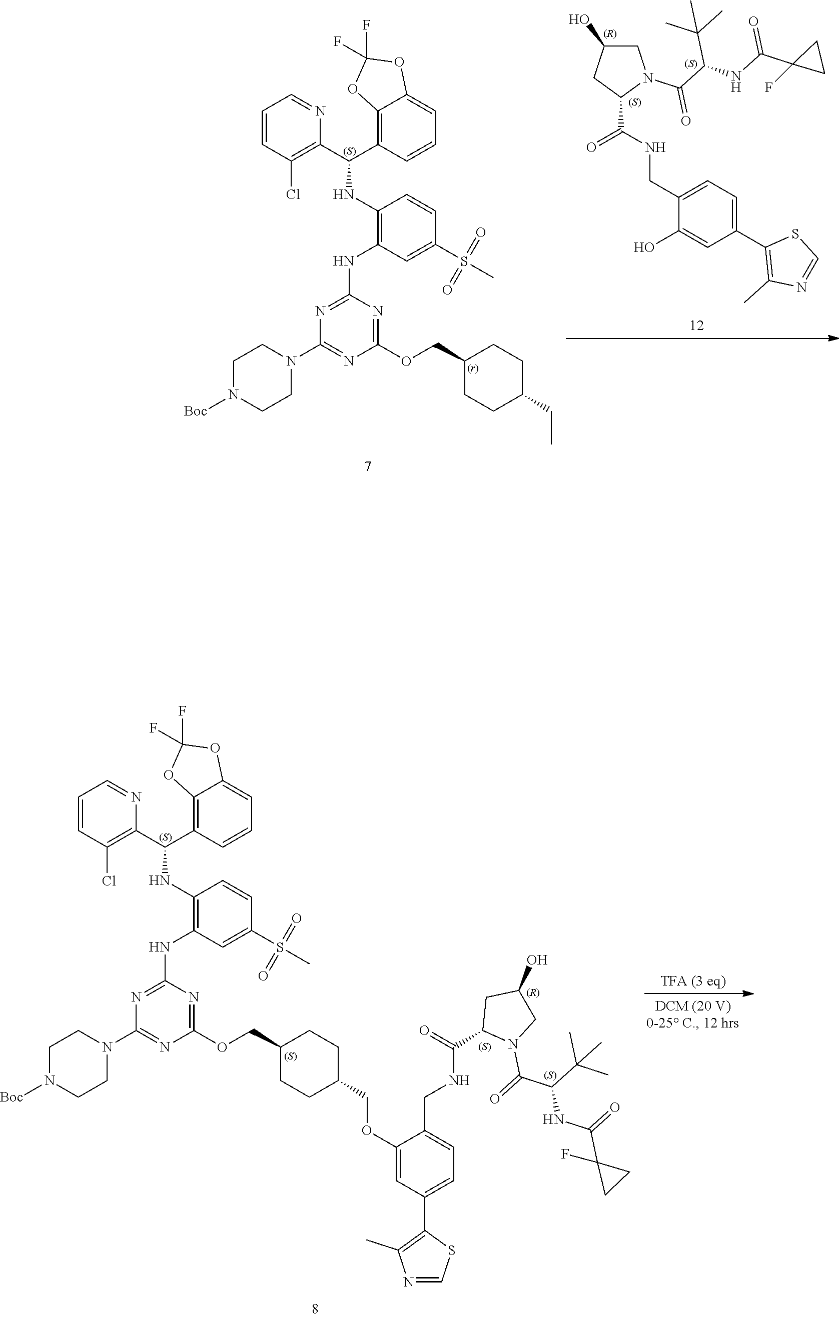

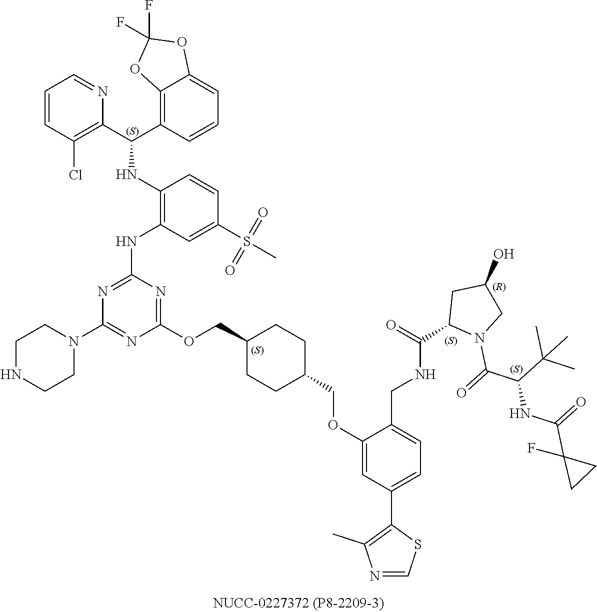

### Experimentals for Largest Scale Run:

### General Procedure for Preparation of Compound 2—Notebook Page: ET51978-550.

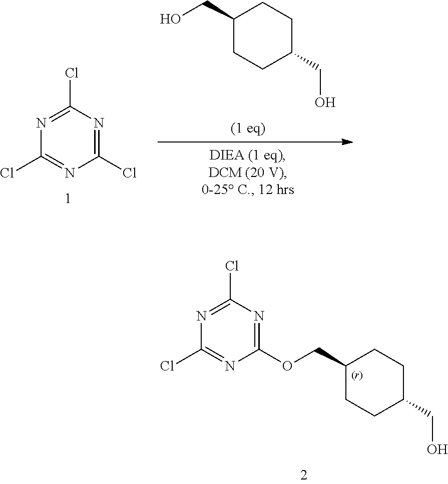 [SMILES: Clc1nc(Cl)nc(Cl)n1 ; OC[C@H]1CC[C@H](CO)CC1 ; OC[C@H]1CC[C@H](COc2nc(Cl)nc(Cl)n2)CC1]

To a solution of Compound 1 (1 g, 5.42 mmol, 1 eq) and DIEA (700.84 mg, 5.42 mmol, 944.53 μL, 1 eq) in DCM (20 mL) was added [4-(hydroxymethyl)cyclohexyl] methanol (782.01 mg, 5.42 mmol, 1 eq) at 0° C. The reaction mixture was stirred at 25° C. for 12 hrs. TLC (petroleum ether/ethyl acetate=0/1) showed the starting material was consumed and a new spot was generated. The reaction mixture was filtered and concentrated under reduced pressure to give a residue, which was purified by chromatography on silica gel (eluted with petroleum ether/ethyl acetate=50/1 to 5/1) to give Compound 2 (650 mg, yield 41.03%) as a white solid.

1H NMR (ET51978-550-P1A, 400 MHz, CHLOROFORM-d) δ 0.95-1.22 (m, 4H), 1.33-1.60 (m, 2H), 1.73-2.02 (m, 5H), 3.49 (d, J=6.32 Hz, 2H), 4.28-4.36 (m, 2H)

### General Procedure for Preparation of Compound 4—Notebook Page: ET51978-555.

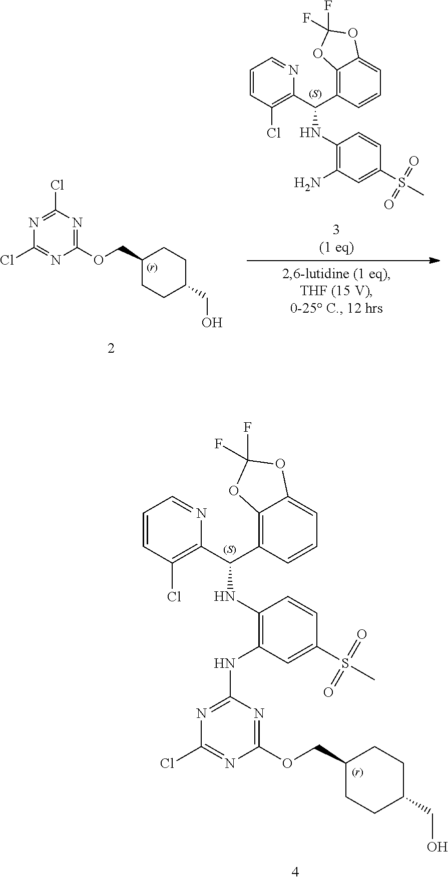

To a solution of Compound 2 (200 mg, 684.55 μmol, 1 eq) in THF (2 mL) was added Compound 3 (320.28 mg, 684.55 μmol, 1 eq) and 2,6-lutidine (73.35 mg, 684.55 μmol, 1 eq) in THF (1 mL) at 0° C. The reaction mixture was stirred at 25° C. for 12 hrs. LCMS showed about 17% of Compound 2 was remaining and 55% of product with desired MS was detected. The reaction mixture was diluted with water (10 mL) and extracted with ethyl acetate (3-10 mL). The organic layer was separated and the combined organic layer was washed with brine (2×10 mL), dried over Na₂SO₄, filtered and the filtrate was concentrated under reduced pressure to give a residue, which was purified by chromatography on silica gel (eluted with petroleum ether/ethyl acetate=50/1 to 0/1) to give Compound 4 (290 mg, yield 58.55%) as a yellow solid.

1H NMR (ET51978-555-P1A, 400 MHz, CHLOROFORM-d) δ 0.83-1.12 (m, 4H), 1.36-1.48 (m, 1H), 1.62 (br d, J=3.79 Hz, 1H), 1.70-1.88 (m, 4H), 3.02 (br s, 3H), 3.45 (br d, J=5.38 Hz, 2H), 3.97-4.12 (m, 2H), 6.22 (br d, J=6.24 Hz, 1H), 6.75-7.08 (m, 5H), 7.18-7.26 (m, 1H), 7.70 (br d, J=7.46 Hz, 2H), 7.93-8.03 (m, 1H), 8.30-8.46 (m, 1H)

### General Procedure for Preparation of Compound 6—Notebook Page: ET51978-557.

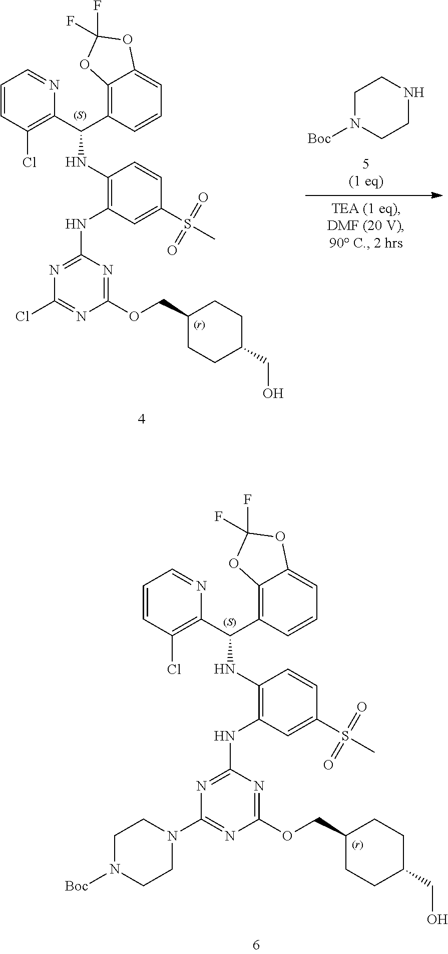

To a solution of Compound 4 (290 mg, 400.79 μmol, 1 eq) and Compound 5 (74.65 mg, 400.79 μmol, 1 eq) in DMF (5.8 mL) was added TEA (40.56 mg, 400.79 μmol, 1 eq). The reaction mixture was stirred at 90° C. for 2 hrs. LCMS showed all the starting material was consumed and a new peak with desired product MS was detected. The reaction mixture was diluted with water (60 mL) and extracted with ethyl acetate (3×30 mL). The organic layer was separated and the combined organic layer was washed with brine (2×30 mL), dried over Na₂SO₄, filtered and the filtrate was concentrated under reduced pressure to give a residue, which was purified by chromatography on silica gel (eluted with petroleum ether/ethyl acetate=50/1 to 5/1) to give Compound 6 (290 mg, yield 82.85%) as a yellow solid.

¹H NMR (ET51978-557-P1A, 400 MHz, CHLOROFORM-d) δ 0.71-1.00 (m, 4H), 1.29-1.38 (m, 1H), 1.41-1.51 (m, 10H), 1.52-1.78 (m, 5H), 3.02 (s, 3H), 3.17-3.52 (m, 6H), 3.56-3.85 (m, 4H), 3.91-4.07 (m, 2H), 5.98-6.18 (m, 1H), 6.74-6.98 (m, 5H), 7.05-7.16 (m, 1H), 7.54-7.68 (m, 2H), 8.06-8.39 (m, 2H)

### General Procedure for Preparation of Compound 7—Notebook Page: ET51978-566.

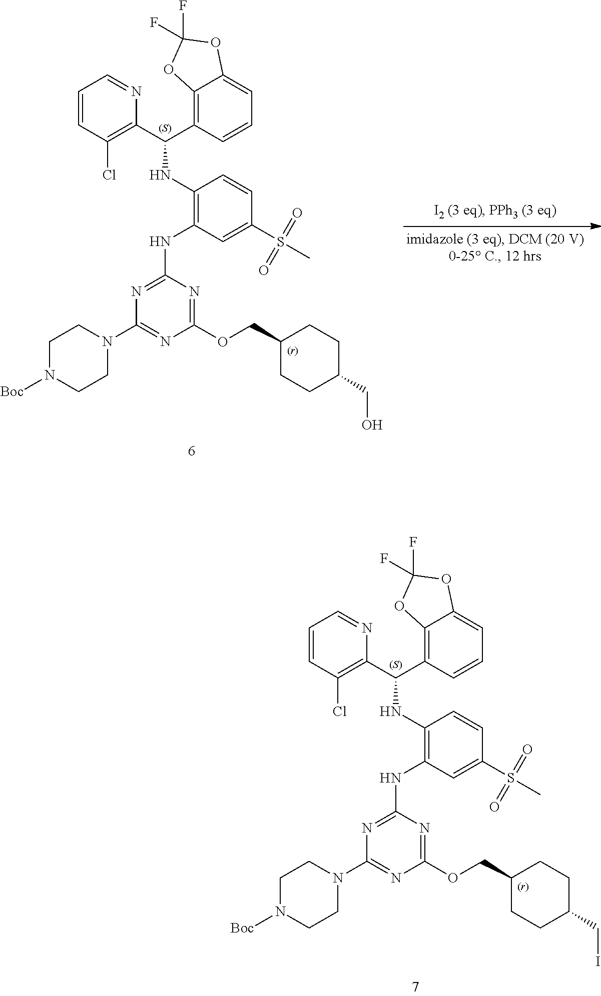

To a solution of Compound 6 (290 mg, 332.05 μmol, 1 eq) and 12 (252.83 mg, 996.15 μmol, 3 eq) in DCM (5.8 mL) were added imidazole (67.82 mg, 996.15 μmol, 3 eq) and PPh₃ (261.28 mg, 996.15 μmol, 3 eq) at 0° C. The reaction mixture was stirred at 25° C. for 12 hrs. LCMS showed all starting material was consumed and 65% of product with desired MS was detected. The reaction mixture was diluted with water (10 mL) and extracted with DCM (3×10 mL). The organic layer was separated and the combined organic layer was washed with brine (2×10 mL), dried over Na₂SO₄, filtered and concentrated under reduced pressure to give a residue, which was purified by chromatography on silica gel (eluted with petroleum ether/ethyl acetate=50/1 to 0/1) to give Compound 7 (220 mg, yield 67.38%) as a pink solid.

1H NMR (ET51978-566-P1B, 400 MHz, CHLOROFORM-d) δ 0.91-1.08 (m, 4H), 1.35-1.43 (m, 1H), 1.47 (s, 9H), 1.61-1.71 (m, 1H), 1.80-1.99 (m, 4H), 3.00 (s, 3H), 3.11 (d, J=6.24 Hz, 2H), 3.30-3.49 (m, 4H), 3.59-3.84 (m, 4H), 4.04-4.13 (m, 2H), 6.21 (br d, J=5.99 Hz, 1H), 6.75 (br d, J=4.77 Hz, 1H), 6.87-7.00 (m, 4H), 7.21 (dd, J=7.89, 4.58 Hz, 1H), 7.60-7.70 (m, 2H), 8.13 (br s, 1H), 8.43 (br d, J=1.47 Hz, 1H)

### General Procedure for Preparation of Compound 8—Notebook Page: ET51978-617.

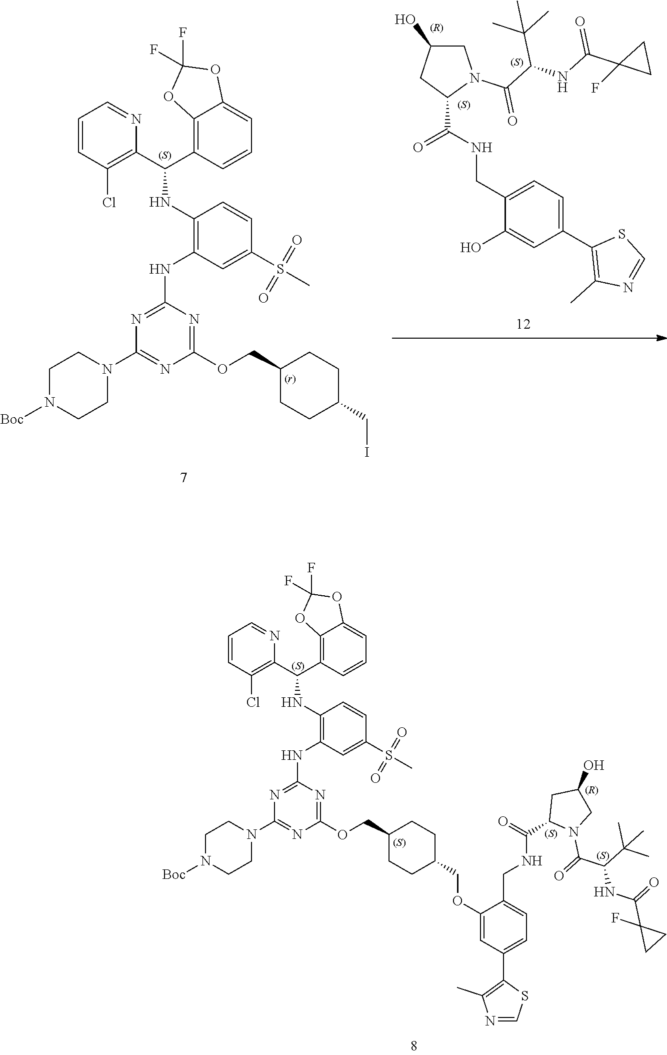

To a solution of Compound 7 (220 mg, 223.75 μmol, 1 eq) and Compound 12 (95.34 mg, 179.00 μmol, 0.8 eq) in DMSO (4.4 mL) was added K₂CO₃ (92.77 mg, 671.24 μmol, 3 eq). The reaction mixture was stirred at 60° C. for 1 hr. LCMS showed all starting material was consumed and 31% of product with desired MS was detected. The reaction mixture was filtered and concentrated under reduced pressure to give a residue, which was purified by Prep-HPLC (column: Phenomenex Luna C18 75*30 mm*3 um; mobile phase: [H₂O (0.1% TFA)-ACN]; gradient: 60%-90%/o B over 8.0 min) to give Compound 8 (40 mg, yield 11.33%) as a white solid.

LCMS (ESI+): RT=0.932 min, m/z 695.1 (M/2+H)⁺.

5_95AB_2 min: LC/MS (The column used for chromatography was a Kinetex EVO C18 2.1*30 mm, 5 um. Detection methods are diode array (DAD). MS mode was positive electrospray ionization. MS range was 100-1000. Mobile phase A was 0.04% trifluoroacetic acid in water, and mobile phase B was 0.02% trifluoroacetic acid in HPLC grade acetonitrile. The gradient was 5-95% B in 1.50 min. 5% B in 0.01 min, 5-95% B (0.01-0.70 min), 95% B (0.70-1.16 min), 95-5% B (1.16-1.50 min). The flow rate was 1.5 mL/min.

### General Procedure for Preparation of NUCC-0227372—Notebook Page: ET51978-630.

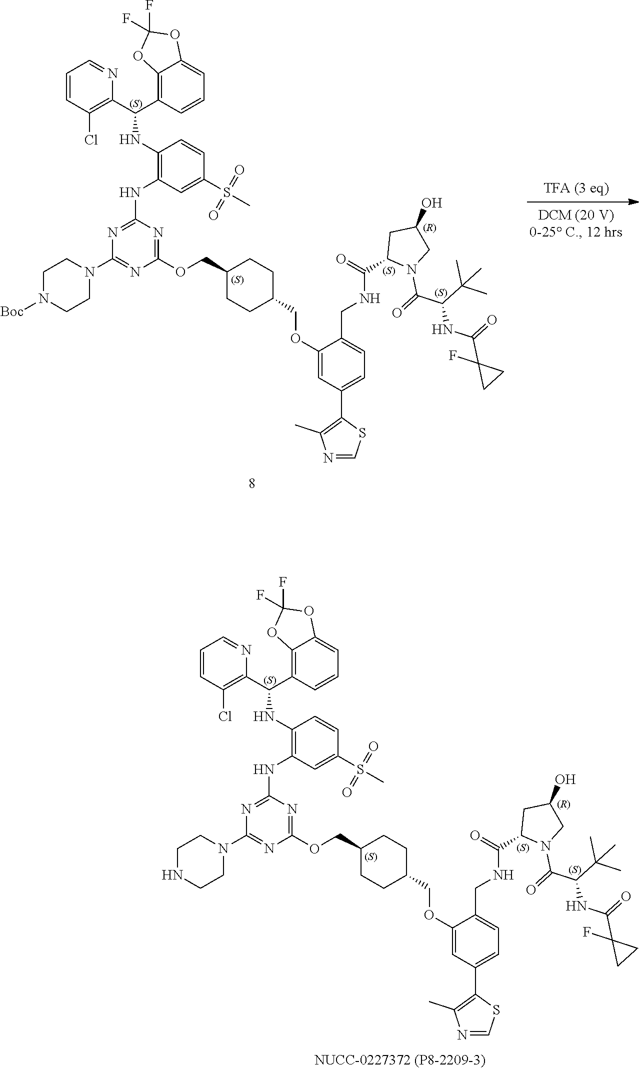

To a solution of Compound 8 (40 mg, 25.36 μmol, 1 eq) in DCM (0.8 mL) was added TFA (8.68 mg, 76.08 μmol, 3 eq) dropwise at 0° C. The reaction mixture was stirred at 25° C. for 12 hrs. LCMS showed all the starting material was consumed and a new peak with desired product MS was detected. The reaction mixture was concentrated under reduced pressure to give a residue, which was purified by Prep-HPLC (column: Phenomenex Luna C18 75*30 mm*3 um; mobile phase: [H₂O (0.1% TFA)-ACN]; gradient: 40%-70% B over 8.0 min) to give NUCC-0227372 (19.1 mg, yield 53.72%, TFA salt) as a white solid.

1H NMR (ET51978-630-P1A, 400 MHz, DMSO-d₆) δ 0.96 (s, 9H), 1.02-1.26 (m, 6H), 1.27-1.46 (m, 2H), 1.48-1.87 (m, 5H), 1.87-2.03 (m, 3H), 2.05-2.15 (m, 1H), 2.46 (s, 3H), 3.03-3.19 (m, 7H), 3.54-3.71 (m, 3H), 4.15-4.43 (m, 8H), 4.47-4.71 (m, 3H), 6.36 (d, J=7.15 Hz, 1H), 6.82 (br d, J=8.82 Hz, 1H), 6.92-7.05 (m, 3H), 7.16 (br d, J=7.99 Hz, 1H), 7.21-7.26 (m, 1H), 7.27-7.35 (m, 2H), 7.42 (d, J=7.75 Hz, 1H), 7.48 (dd, J=8.05, 4.71 Hz, 2H), 7.82-8.09 (m, 2H), 8.48-8.59 (m, 2H), 8.81 (br d, J=10.19 Hz, 2H), 8.99 (s, 1H), 9.42 (br s, 1H)

LCMS (ESI+): RT=3.375 min, m/z 644.3 (M/2+H)⁺.

5_95CD_6 min-220-254-MS2000

LC/MS (The gradient was 5% B in 0.40 min and 5-95% B at 0.40-3.40 min, hold on 95% B for 0.45 min, and then 95-5% B in 0.01 min, the flow rate was 0.8 mL/min. Mobile phase A was H₂O+10 mM NH₄HCO₃, mobile phase B was acetonitrile. The column used for chromatography was a Xbridge C18 2.1*50 mm column (5 um particles). Detection methods are diode array (DAD) detection. MS mode was positive electrospray ionization. MS range was 100-2000.

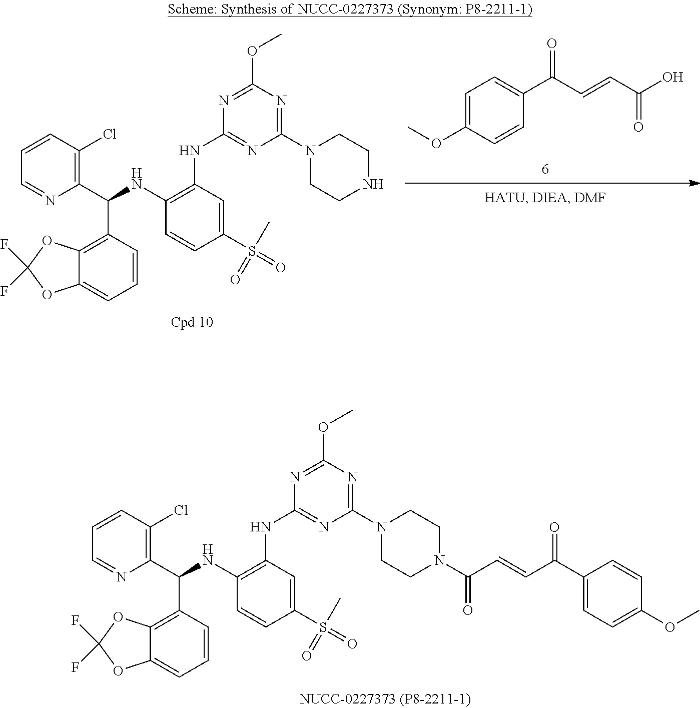

### General Procedure for Preparation of NUCC-0227373—Notebook Page: ET43587-1341.

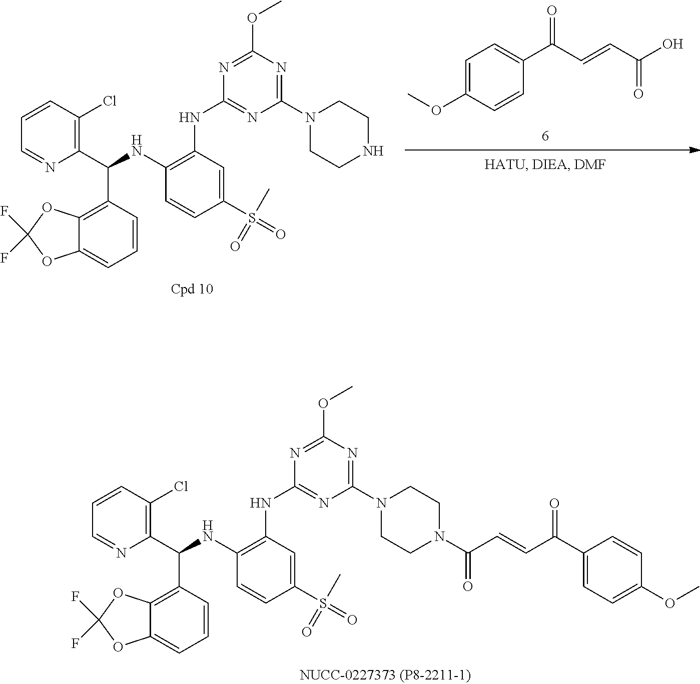

To a solution of Cpd 10 (150 mg, 226.90 μmol, 1 eq), Compound 6 (51.46 mg, 249.59 μmol, 1.1 eq) and DIEA (87.98 mg, 680.71 μmol, 3 eq) in DMF (3 mL) was added HATU (103.53 mg, 272.28 μmol, 1.2 eq) at 0° C., the mixture was stirred at 20° C. for one hour. LCMS showed the starting material was consumed and a new peak with desired product MS was detected. The reaction mixture was diluted with water (20 mL) and extracted with ethyl acetate (3×20 mL). The organic layer was separated and the combined organic layer was washed with brine (2×20 mL), dried over Na₂SO₄, filtered and the filtrate was concentrated under reduced pressure to give a residue, which was purified by Prep-HPLC (column: Phenomenex Luna C18 100*30 mm*3 um; mobile phase: [H₂O (0.04% HCl)-ACN]; gradient: 45%-70% B over 8.0 min) to give NUCC-0227373 (62.3 mg, yield 31.07%) as a yellow solid.

¹H NMR (400 MHz, DMSO-d₆) δ 3.08 (s, 3H), 3.56-3.82 (m, 9H), 3.84-3.88 (m, 3H), 4.48 (br s, 3H), 6.36 (br s, 1H), 6.81 (d, J=8.88 Hz, 1H), 6.97 (br s, 1H), 7.07-7.18 (m, 3H), 7.20-7.26 (m, 1H), 7.32 (br d, J=7.75 Hz, 1H), 7.38-7.53 (m, 3H), 7.79-7.94 (m, 1H), 8.00-8.08 (m, 3H), 8.54 (br d, J=3.88 Hz, 1H), 9.38 (br s, 1H)

LCMS (ESI+): RT=3.323 min, m/z 849.2 (M+H)⁺.

5_95CD 6 min-220-254-ELSD:

LC/MS (The gradient was 5% B in 0.40 min and 5-95% B at 0.40-3.40 min, hold on 95% B for 0.45 min, and then 95-5% B in 0.01 min, the flow rate was 0.8 ml/min. Mobile phase A was H₂O+10 mM NH₄HCO₃, mobile phase B was acetonitrile. The column used for chromatography was an Xbridge C18 2.1*50 mm column (5 um particles). Detection methods are diode array (DAD) and evaporative light scattering (ELSD) detection. MS mode was positive electrospray ionization. MS range was 100-1000.

### Example 2

### Methods

### Knockdown of DOT1L by RNAi

LNCaP cells were seeded to 10 cm dish in RPMI1640 containing 10% FBS, 10 U/ml penicillin, and 10 μg/ml streptomycin (complete RPMI media). 70-80% confluent of cells were transfected with final 0.1 μM siDOT1L (BioNEER Cat #1043628) or 0.1 μM siCtrl (Dharmacon Cat #D-001210-01-05) using RNAiMAX reagent (Life technologies #13778-075) after 24 h. 72 h after transfection, nuclear protein was extracted using NE-PER Nuclear and Cytoplasmic Extraction Reagents (Thermo Scientific 78833) followed by western blotting.

### Screening of PROTAC Degrader of DOT1L

LNCaP cells were cultured in complete RPMI1640 media. 70-80% confluent of cells were treated with 1 μM PROTAC degraders of DOT1L (Group A-D), 1 μM parent compound 534, 1 μM EPZ5676, and DMSO (Vehicle) for 48 h. Then, nuclear protein was extracted using NE-PER Nuclear and Cytoplasmic Extraction Reagents (Thermo Scientific 78833) followed by western blotting. Protein band intensity was measured by Image J.

### Time-Course Analysis of DOT1L Degradation by PROTAC Degrader of DOT1L

LNCaP cells were culturedincompleteRPMIl640 media. 70-80% confluent of cells were treated with 1 μM 705 (Group D), 1 μM 588 (Group A), 1 μM 593 (Group C), 1uM 534 (parent), 1 μM EPZ5676, and DMSO for 1 h, 2 h, 6 h, 24 h, 48 h, and 72 h. Then, nuclear protein was extracted using NE-PER Nuclear and Cytoplasmic Extraction Reagents (Thermo Scientific 78833) followed by western blotting. Protein band intensity was measured by Image J.

### Dose-Response Analysis of DOT1 L Degradation by 705 Treatment

LNCaP cells were cultured in complete RPMI1640 media. 70-80% confluent of cells were treated with 1 nM 705, 10 nM 705, 50 nM 705, 100 nM 705, 250 nM 705, 500 nM 705, 1 μM 705, and 10 μM 705, with or without 1 μM MG132 for 1 h. Then, nuclear protein was extracted using NE-PER Nuclear and Cytoplasmic Extraction Reagents (Thermo Scientific 78833) followed by western blotting. Protein band intensity was measured by Image J.

### Western Blotting

Nuclear protein extracts were separated by Bolt™ 4-12% Bis-Tris Plus Gels (Invitrogen) and transferred to PVDF membranes by wet transfer. The membranes were blocked with 5% Blotting-Grade Blocker (Bio-Rad) in Tris-buffered saline with 0.5% Tween 20 (TBST) for 1 h at room temperature, and then incubated overnight at 4° C. in blocking buffer with primary antibody including rabbit DOT1L (D1W4Z) mAb1:3000 (CellSignalingTechnology, Cat #77087) and goat lamin B(C-20) pAb 1:1000 (Santa Cruz Biotechnology, Cat #sc-6216). After incubation with HRP-conjugated goat anti-rabbit secondary antibody 1:10,000 (Bio-Rad, Cat #170-6515) or HRP-conjugated donkey anti-goat secondary antibody 1:3000 (Santa Cruz Biotechnology, Cat #sc-2020) for 1 h, the membranes were detected using Super Signal West Femto (Thermo Fisher Scientific, Cat #34096).

### Viability Assay for MV4-11 and Jurkatcells

MV4-11 and Jurkat cell lines (1200 cells/well) were seeded to 96 well culture plate in 50 ul of IMDM 10% FBS 1% P/S for MV4-11 cells and RPMI 10% FBS 1% P/S for Jurkat cells. After 24 h, 50 ul of media containing 2× drugs were added. Cells were treated with 1 μM and 5 μM of small compounds for 4 days. Viability was measured by CellTiter-Glo Luminescent Cell Viability Assay(PromegaG7571).

We claim:

1. A compound, or a pharmaceutically acceptable salt thereof, the compound having a formula I(a) or I(b): 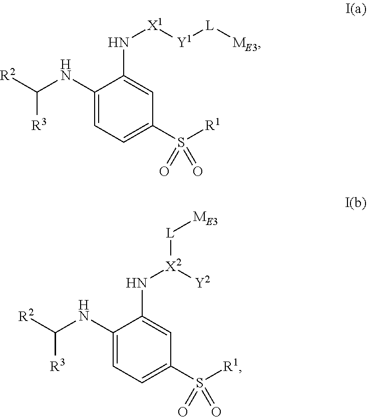
wherein
R¹ is selected from the group consisting of hydrogen, alkyl, cycloalkyl, heterocyclyl, aryl, and heteroaryl;
R² and R³ are each independently an optionally substituted aryl or an optionally substituted heteroaryl;
X¹ is an optionally substituted heteroarylene;
Y¹ is an optionally substituted heterocyclylene;
X² is an optionally substituted trivalent heteroaryloxyl;
Y² is an optionally substituted heterocyclyl;
L is a bond or a linker; and
M_{E3} is a moiety that binds to an E3 ubiquitin ligase.

2. The compound of claim 1, wherein R¹ is alkyl.

3. The compound of claim 1, wherein R² is pyridinyl substituted with one or more halo.

4. The compound of claim 1, wherein R³ is 1,3-benzodioxlyl substituted with one or more halo.

5. The compound of claim 1, wherein X¹ is 1,3,5-triazinylene substituted with alkoxy; or
X² is a trivalent 1,3,5-triazin-2-oxyl.

6. The compound of claim 1, wherein Y¹ is piperazinylene; or
Y² is piperazinyl.

7. The compound of claim 1, wherein the compound has a formula I(aa) or I(bb):

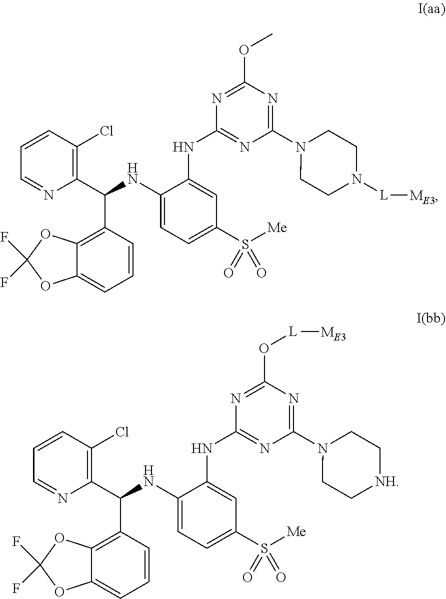

8. The compound of claim 1, wherein
L is a bond or a linker selected from —(CH₂—C(O)NH)ₘ—(CH₂)₂—(OCH₂)ₙ—, 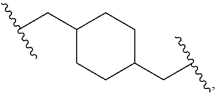 [SMILES: *CC1CCC(C*)CC1]
and —(CH₂)ₚ—;
m is 0 or 1;
n is an integer selected from 1 to 20; and
p is an integer selected from 1 to 10.

9. The compound of claim 1, wherein M_{E3} is a moiety that binds to an E3 ubiquitin ligase selected from Von Hippel-Lindau (VHL) E3 ubiquitin ligase and cereblon (CRBN) E3 ubiquitin ligase.

10. The compound of claim 1, wherein M_{E3} is selected from the group consisting of

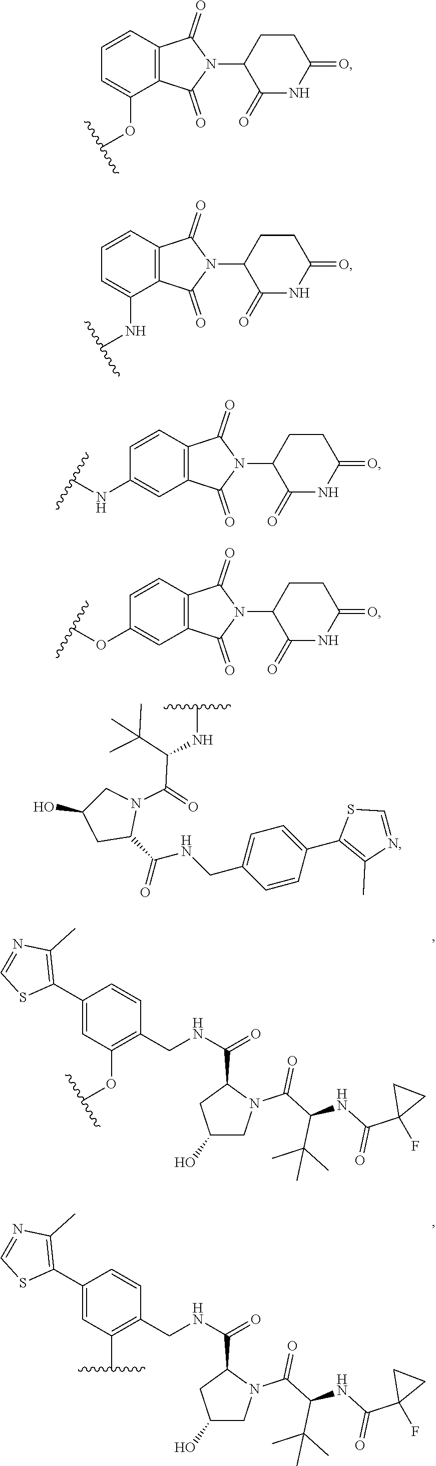

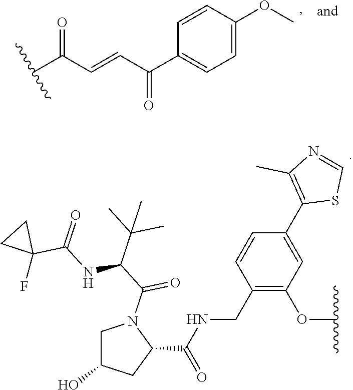 [SMILES: *C(=O)/C=C/C(=O)c1ccc(OC)cc1 ; *Oc1cc(-c2scnc2C)ccc1CNC(=O)[C@@H]1C[C@H](O)CN1C(=O)[C@@H](NC(=O)C1(F)CC1)C(C)(C)C]

11. The compound of claim 1, wherein the compound is selected from the group consisting of

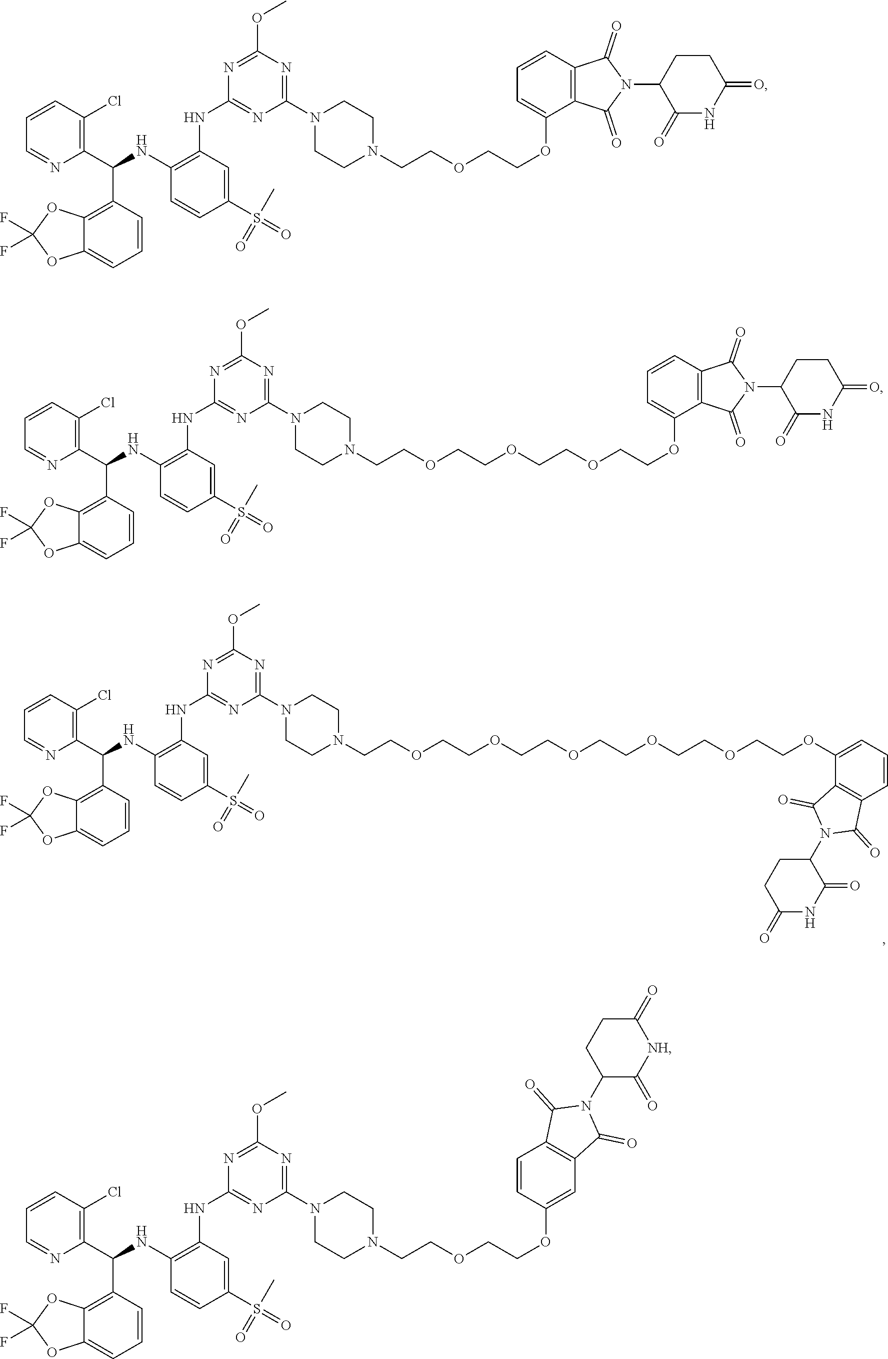

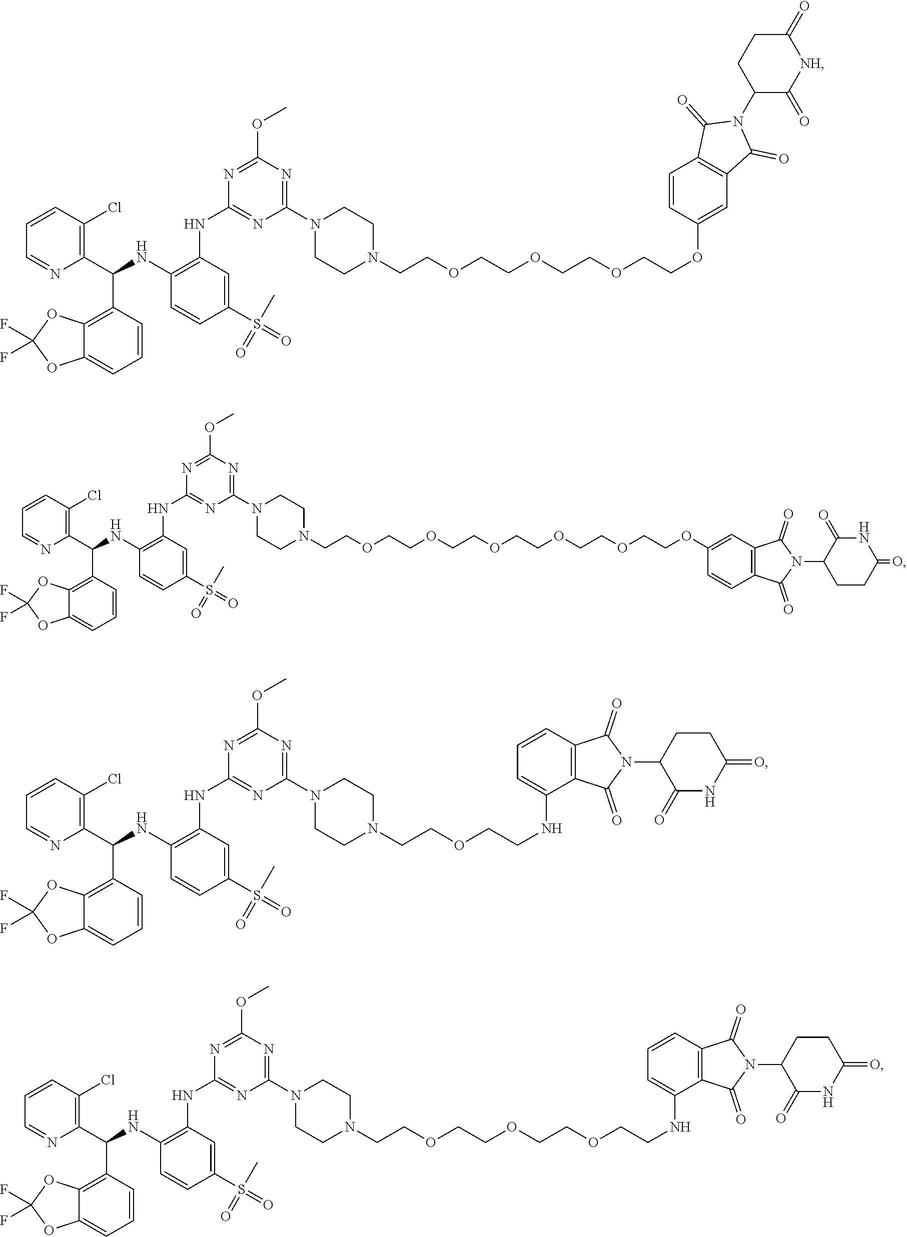

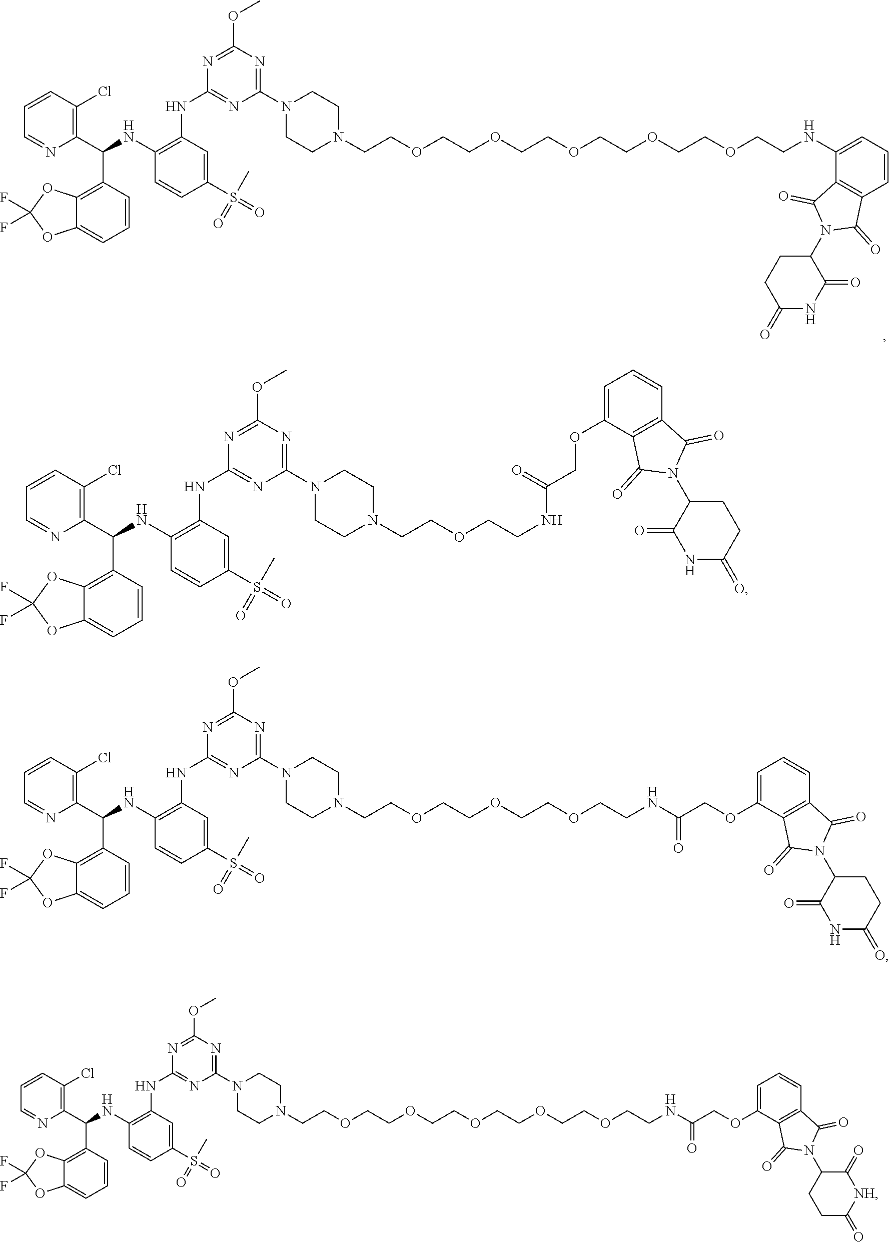

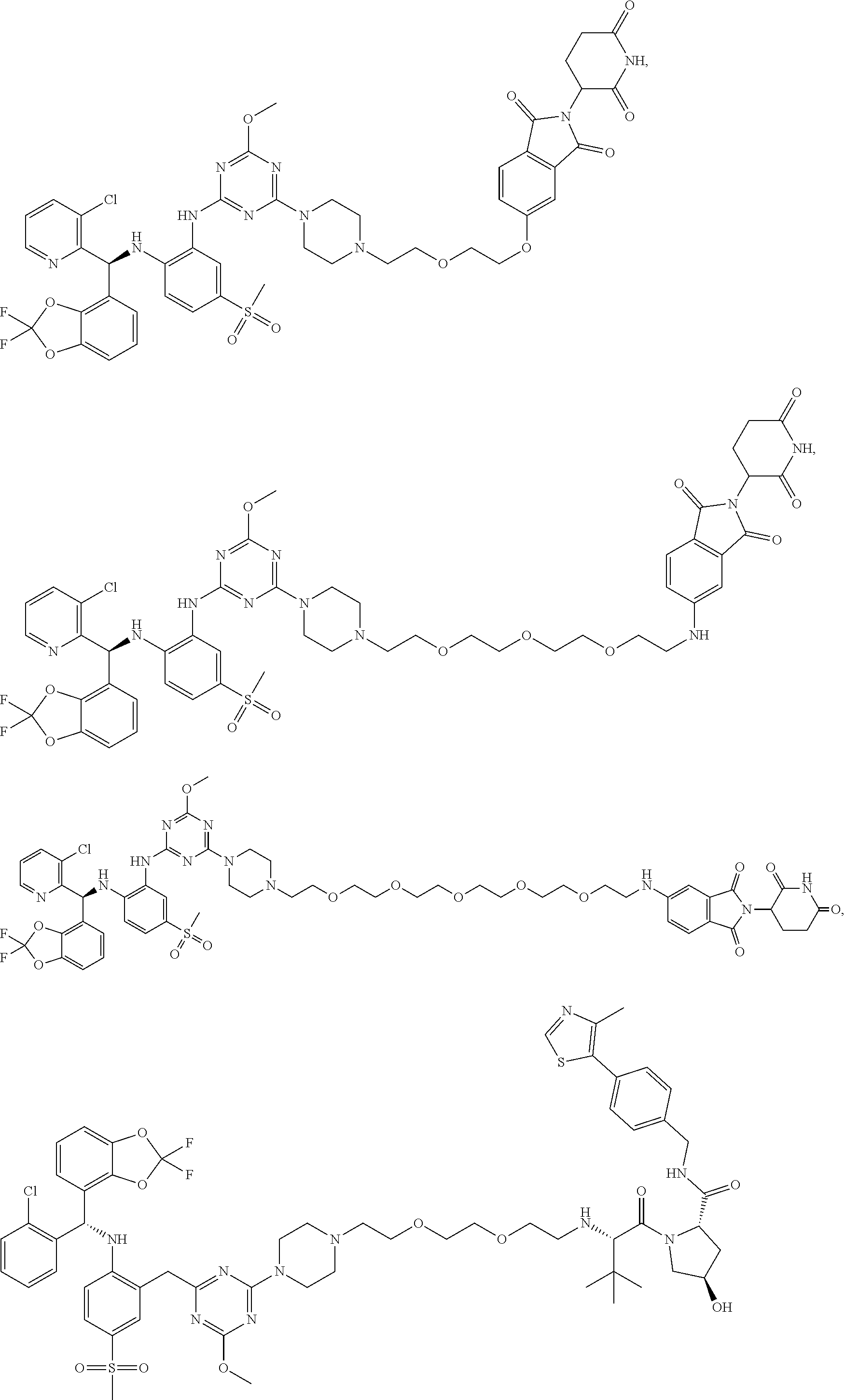

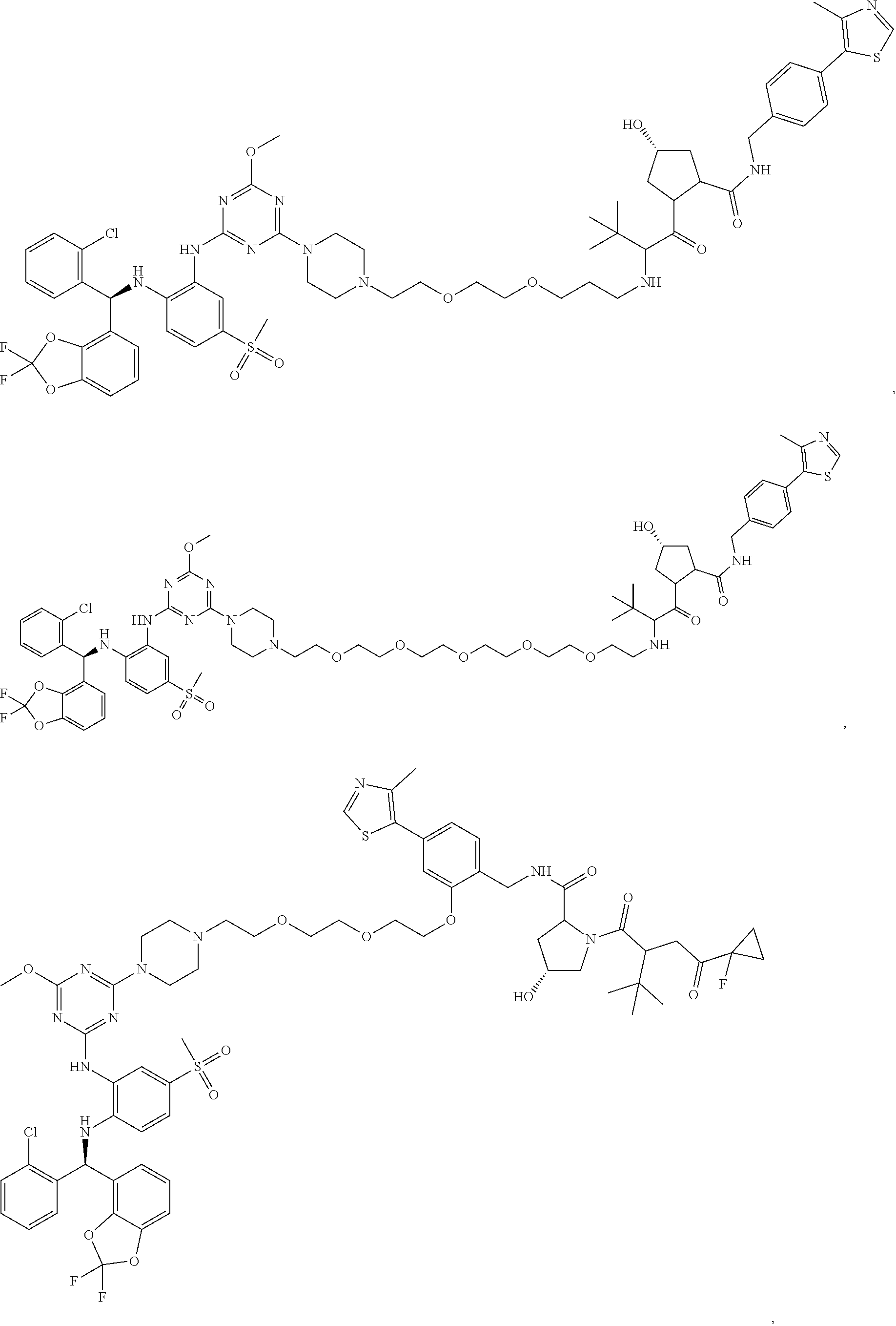 [SMILES: COc1nc(Nc2cc(S(C)(=O)=O)ccc2N[C@H](c2ccccc2Cl)c2cccc3c2OC(F)(F)O3)nc(N2CCN(CCOCCOCCCNC(C(=O)C3C[C@H](O)CC3C(=O)NCc3ccc(-c4scnc4C)cc3)C(C)(C)C)CC2)n1 ; COc1nc(Nc2cc(S(C)(=O)=O)ccc2N[C@H](c2ccccc2Cl)c2cccc3c2OC(F)(F)O3)nc(N2CCN(CCOCCOCCOCCOCCOCCNC(C(=O)C3C[C@H](O)CC3C(=O)NCc3ccc(-c4scnc4C)cc3)C(C)(C)C)CC2)n1 ; COc1nc(Nc2cc(S(C)(=O)=O)ccc2N[C@H](c2ccccc2Cl)c2cccc3c2OC(F)(F)O3)nc(N2CCN(CCOCCOCCOc3cc(-c4scnc4C)ccc3CNC(=O)C3C[C@@H](O)CN3C(=O)C(CC(=O)C3(F)CC3)C(C)(C)C)CC2)n1]

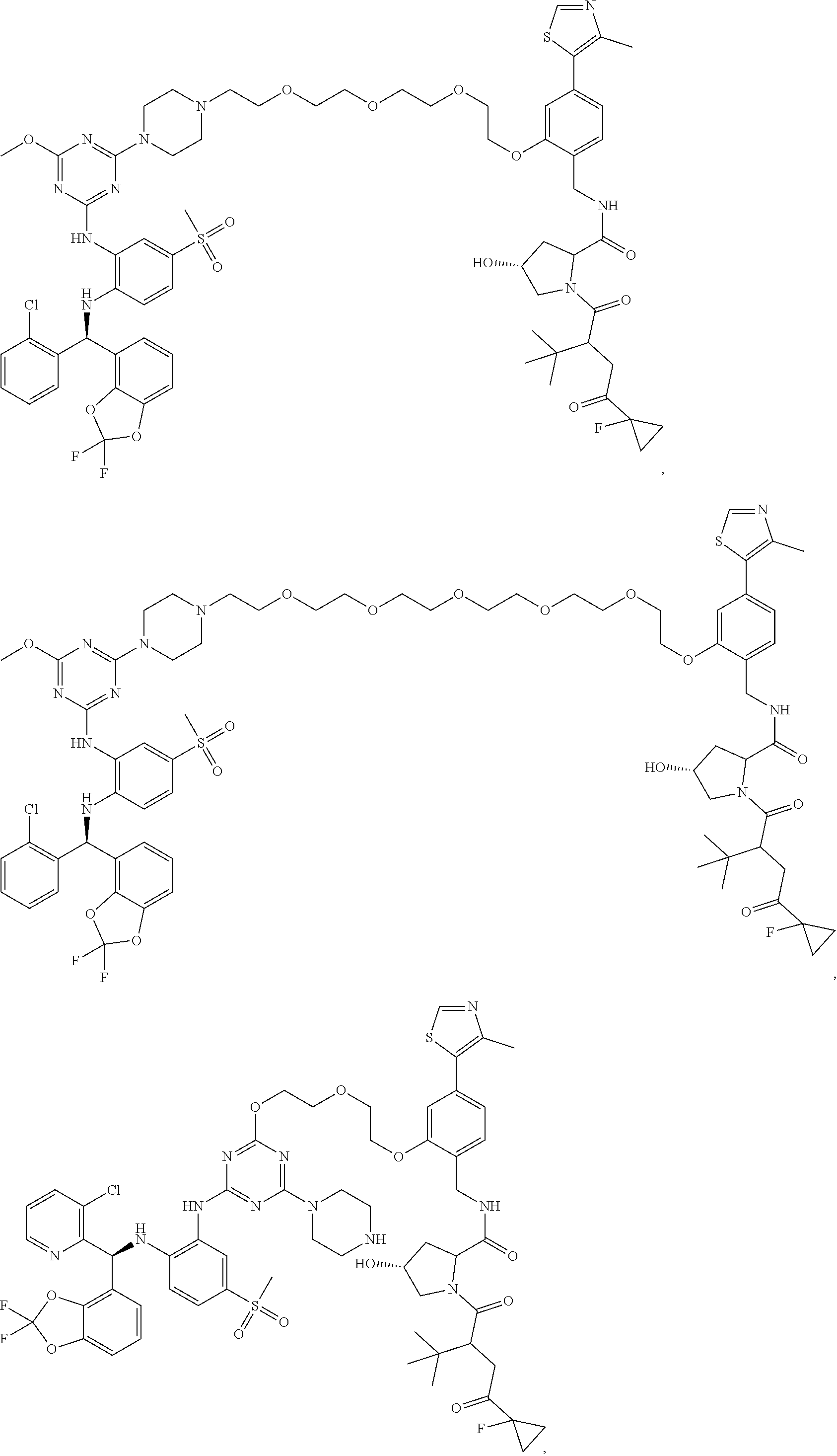

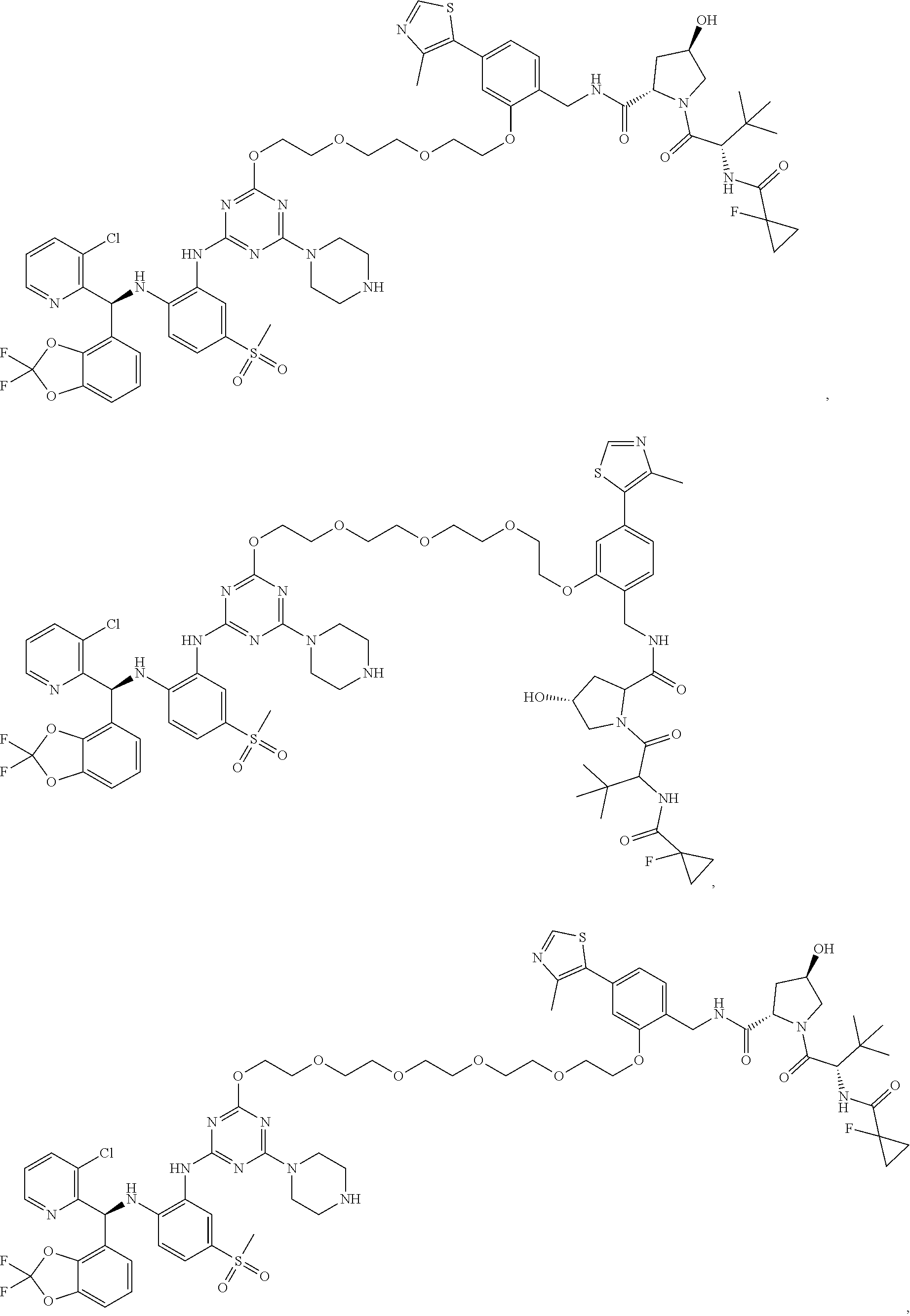

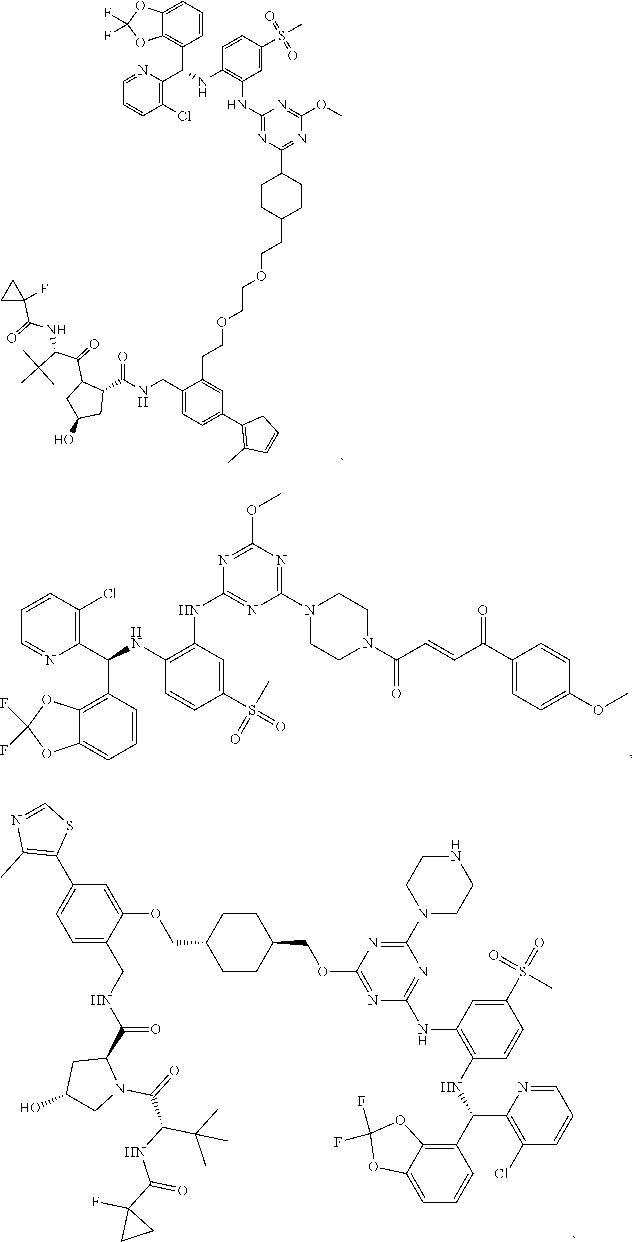

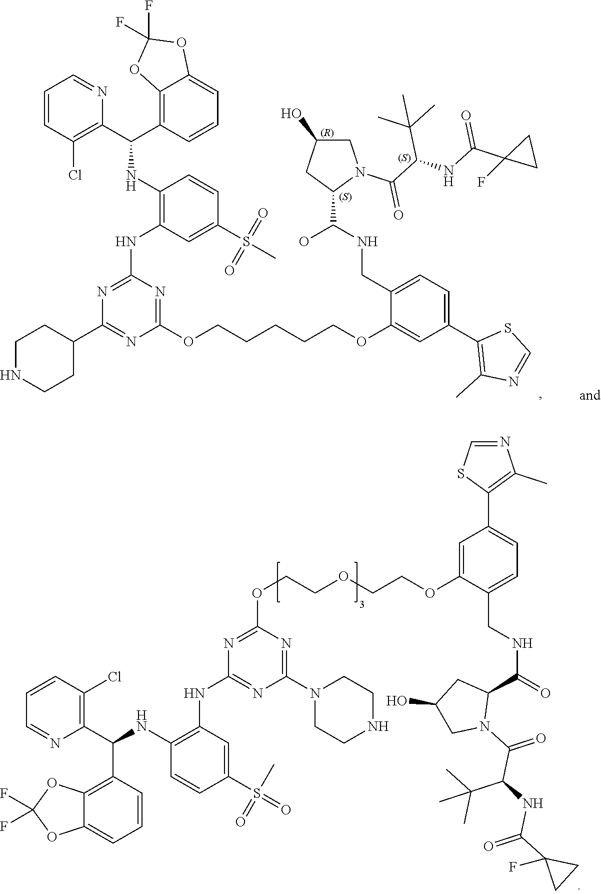

12. The compound of claim 1, wherein the compound is selected from the group consisting of 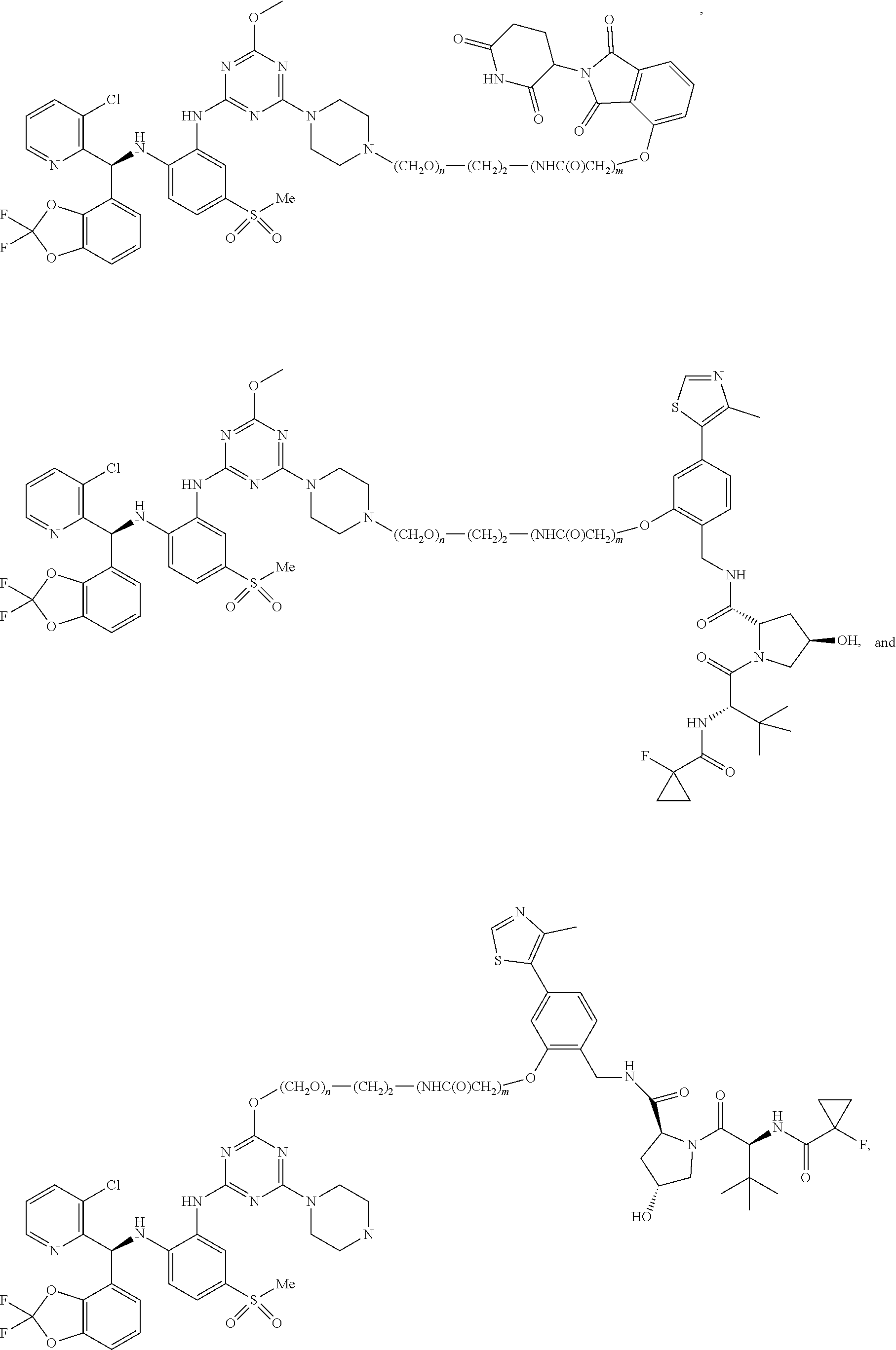
and wherein m is 0 or 1 and n is an integer selected from 1 to 20.

13. The compound of claim 12, wherein the compound is selected from the group consisting of

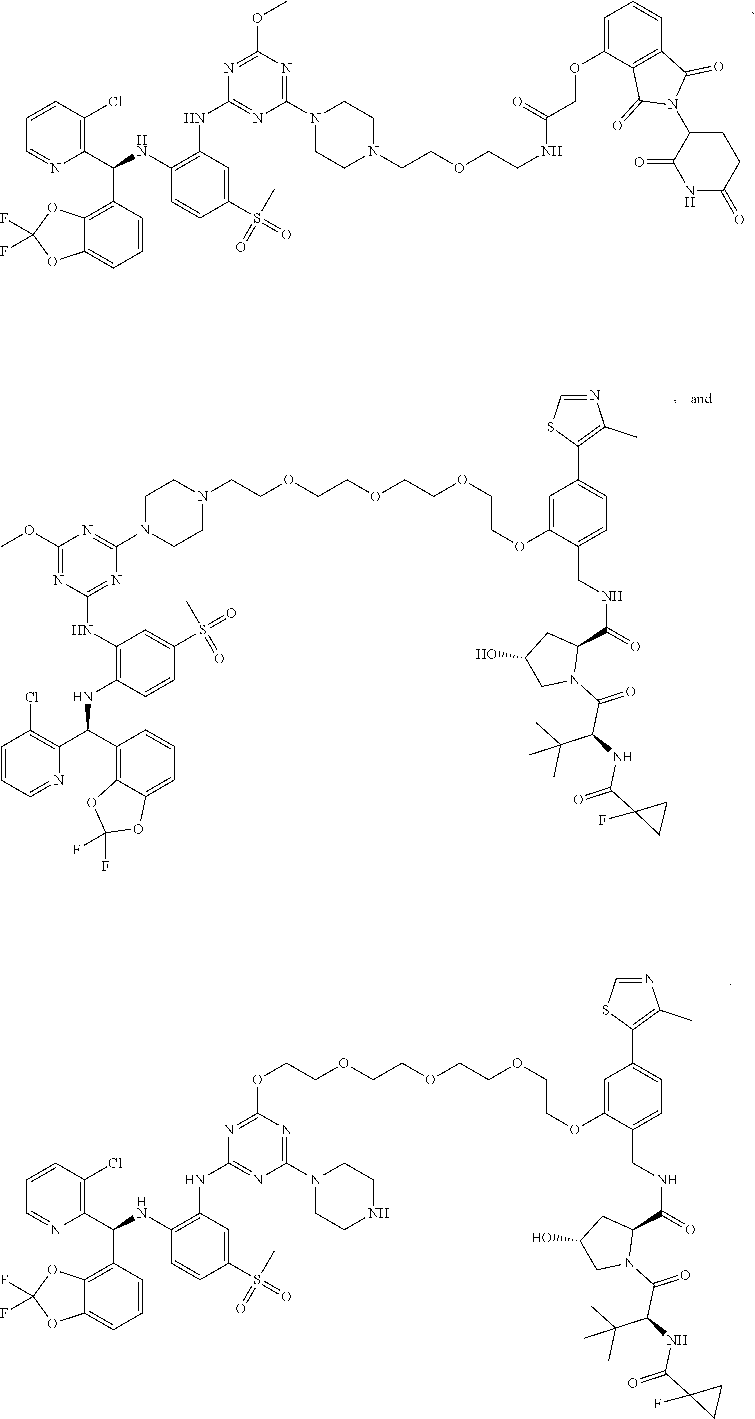 [SMILES: COc1nc(Nc2cc(S(C)(=O)=O)ccc2N[C@@H](c2cccc3c2OC(F)(F)O3)c2ncccc2Cl)nc(N2CCN(CCOCCNC(=O)COc3cccc4c3C(=O)N(C3CCC(=O)NC3=O)C4=O)CC2)n1 ; COc1nc(Nc2cc(S(C)(=O)=O)ccc2N[C@@H](c2cccc3c2OC(F)(F)O3)c2ncccc2Cl)nc(N2CCN(CCOCCOCCOCCOc3cc(-c4scnc4C)ccc3CNC(=O)[C@@H]3C[C@@H](O)CN3C(=O)[C@@H](NC(=O)C3(F)CC3)C(C)(C)C)CC2)n1 ; Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCOc2nc(Nc3cc(S(C)(=O)=O)ccc3N[C@@H](c3cccc4c3OC(F)(F)O4)c3ncccc3Cl)nc(N3CCNCC3)n2)c1]

14. A pharmaceutical composition comprising a therapeutically effective amount of the compound of claim 1 and a pharmaceutically acceptable carrier, excipient, or diluent.

15. A method of treating a disease or disorder associated with DOT1L activity in a subject in need thereof, the method comprising administering to the subject the compound of claim 1.

16. The method of claim 15, wherein the disease or disorder is a cell proliferative disease or disorder.

17. The method of claim 16, wherein the cell proliferative disease or disorder is leukemia.

18. The method of claim 17, wherein the leukemia is MLL-rearranged leukemia.

19. The method of claim 17, wherein the leukemia is acute myeloid leukemia or acute lymphoblastic leukemia.

20. A method of inhibiting cell proliferation, the method comprising contacting cells with the compound of claim 1, or a pharmaceutically acceptable salt thereof.

21. The method of claim 20, wherein the cells have mixed lineage leukemia (MLL) rearrangements.

22. The method of claim 20, wherein the cells are leukemia cells.

23. The method of claim 20, wherein the cells are acute myeloid leukemia cells or acute lymphoblastic leukemia cells.

24. The method of claim 20, wherein the contact is in vivo.
